(19) United States
(12) Patent Application Publication
(10) Pub. No.: US 2009/0326229 A1
(43) Pub. Date: Dec. 31, 2009

(54) ALPHA-CARBOLINE DERIVATIVES AND METHODS FOR PREPARATION THEREOF

(75) Inventors: Masahiro Mizuno, Osaka (JP); Hideya Mizufune, Osaka (JP); Misayo Sera, Osaka (JP); Masahiro Mineno, Osaka (JP); Tsuyoshi Ueda, Osaka (JP)

Correspondence Address: SUGHRUE MION, PLLC, 2100 PENNSYLVANIA AVENUE, N.W., SUITE 800, WASHINGTON, DC, 20037, US

(73) Assignee: Takeda Phamaceutical Company Limited, Osaka-shi, Osaka (JP)

(21) Appl. No.: 12/376,068

(22) PCT Filed: Aug. 2, 2007
(86) PCT No.: PCT/JP2007/065571

§ 371 Date: Feb. 2, 2009

(30) Foreign Application Priority Data
Aug. 2, 2006 (JP) 2006-211472
Apr. 13, 2007 (JP) 2007-106067

(51) Int. Cl. C07D 471/02; C07D 213/72

(52) U.S. Cl. 546/84; 546/304

(57) ABSTRACT

To provide methods for preparing alpha-carboline derivatives in few steps, as well as conveniently and industrially advantageously. A method for preparation of a compound represented by Formula (II) or a salt thereof, comprising subjecting a compound represented by Formula (I) or a salt thereof to a ring closure reaction in the presence of a palladium catalyst, a ligand, and a base; a method for preparation of a compound represented by Formula (IX) or a salt thereof, comprising subjecting a compound represented by Formula (VII) or a salt thereof to a ring closure reaction in the presence of a palladium catalyst, a ligand, and a base, and subsequently to an aromatization reaction; and methods for preparation of compounds represented by Formulae (XV), (XVII), and (XIX) or a salt thereof, comprising subjecting respective compounds represented by Formulae (II) and (IX) or a salt thereof to a reaction for introducing a leaving group when necessary, and subsequently to a coupling reaction: wherein the symbols respectively represent the same meaning as defined in the present specification.

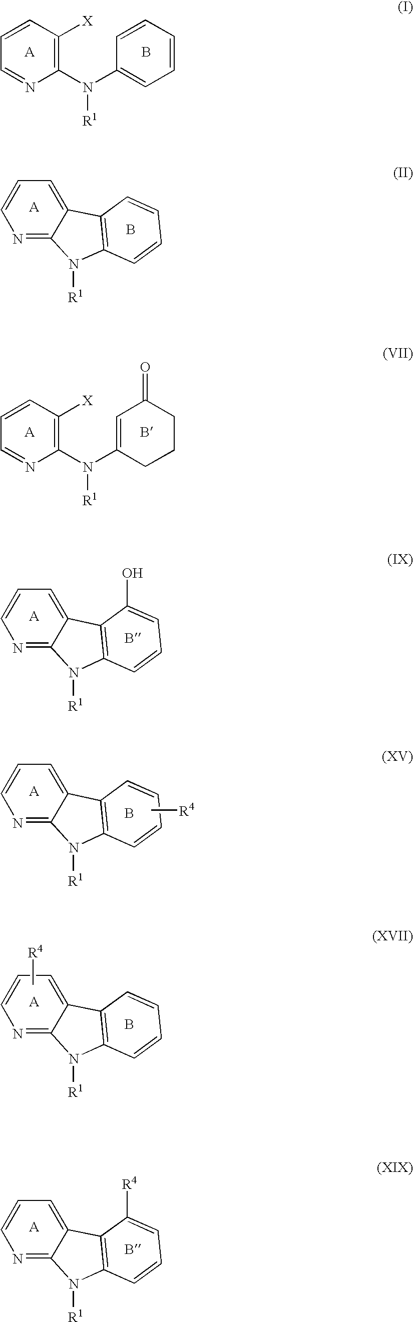

### TECHNICAL FIELD

The present invention relates to α-carboline derivatives which are useful for pharmaceutical products, agrochemicals, food products, cosmetic products, and chemical products, or as intermediates thereof, and methods for preparation thereof.

### BACKGROUND OF THE INVENTION

α-Carboline derivatives are useful for pharmaceutical products, agrochemicals, food products, cosmetic products, and chemical products, or as intermediates thereof.

For example, Patent Document 1 (French Patent No. 2876377) describes α-carboline derivatives (A) and (B) having a CDK1/CDK5 (Cyclin-Dependent Kinase) inhibitory action and a GSK-3 (Glycogen Synthase Kinase) inhibitory action:

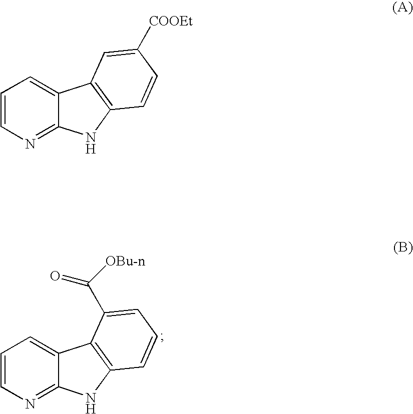

Non-Patent Document 1 (Tetrahedron, Vol. 56, p. 3189 (2000)) describes a carboline derivative (C) having an antitumor activity:

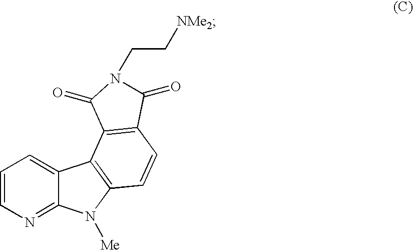

Patent Document 2 (JP-W No. 2003-507480) describes a carboline derivative (D) having an inhibitory action against platelet-derived growth factor receptor (PDGFR) kinases and vascular endothelial growth factor receptor (VEGFR) kinases:

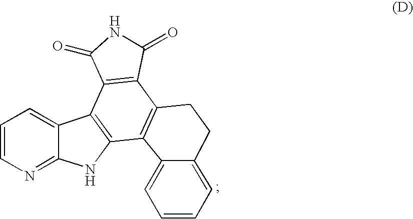

Patent Documents 3 (International Patent Application Publication No. WO 95/07910) and 4 (International Patent Application Publication No. WO 96/04906), and Non-Patent Document 2 (Bioorg. Med. Chem. Lett., Vol. 13, p. 3835 (2003)) describe a carboline derivative (E) having an antivirus action and a CDK-4 (Cyclin-Dependent Kinase) inhibitory action:

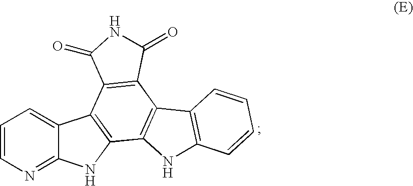

Non-Patent Document 3 (J. Med. Chem., Vol. 48, p. 6194 (2005)) describes a carboline derivative (F) having an antitumor activity and a tyrosine kinase inhibitory action:

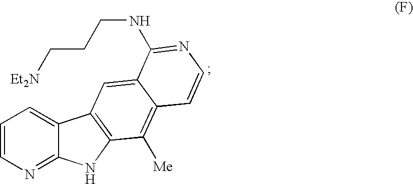

Patent Document 5 (U.S. Pat. No. 5,532,261) describes a carboline derivative (H) as an intermediate of a carboline derivative (G) having an antibacterial activity:

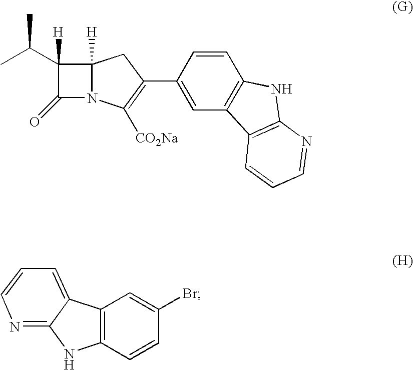

Non-Patent Document 4 (Bioorg. Med. Chem. Lett., Vol. 12, p. 209 (2002)) describes a carboline derivative (J) as an intermediate of a carboline derivative (I) having a β-3 agonist activity:

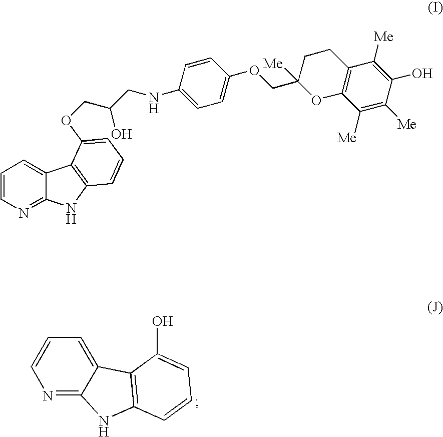

and Patent Document 6 (International Patent Application Publication No. WO 2006/131552) describes a carboline derivative (L) having a CDK1 (Cyclin-Dependent Kinase) inhibitory action:

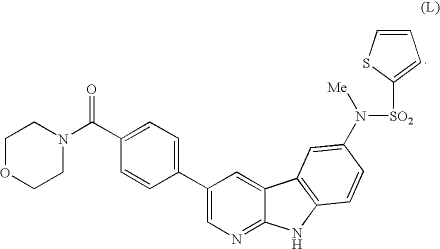

For methods for synthesizing these carboline derivatives, the respective α-carboline derivatives are prepared according to Patent Document 1 as shown by the following reaction scheme:

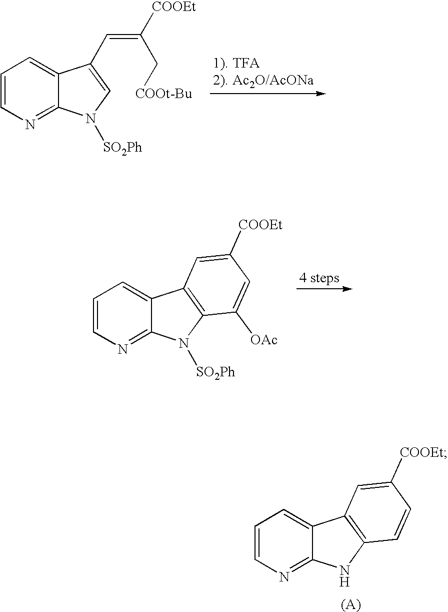

according to Non-Patent Document 1 as shown by the following reaction scheme:

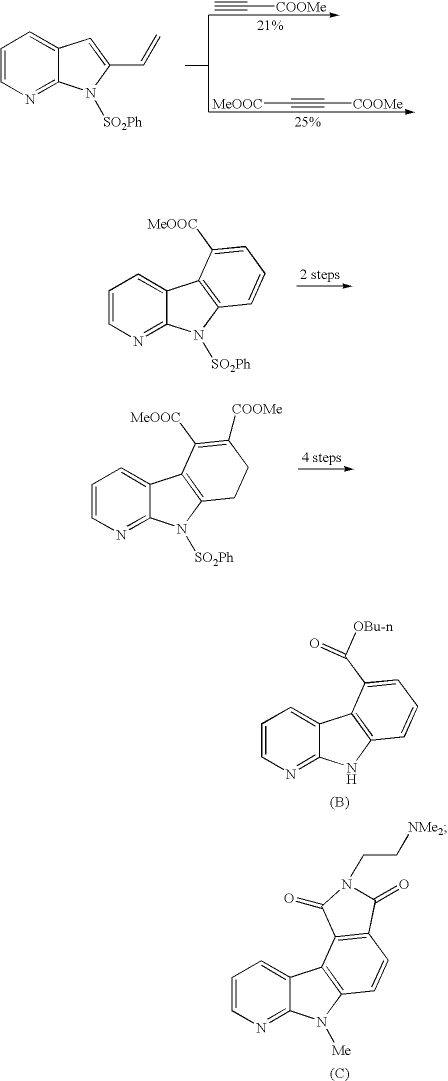

according to Patent Document 2 as shown by the following reaction scheme:

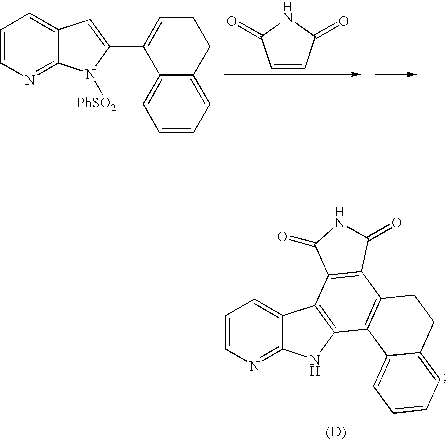

according to Patent Document 3 and Non-Patent Document 2 as shown by the following reaction scheme:

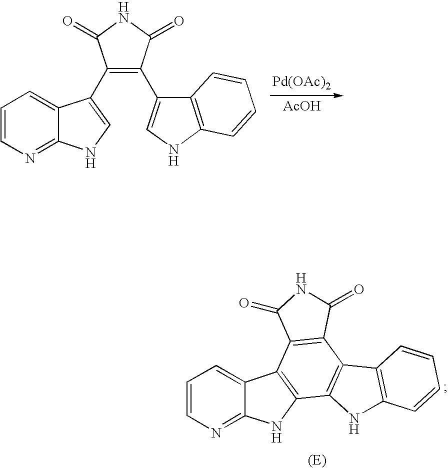

and according to Patent Document 6 as shown by the following reaction scheme:

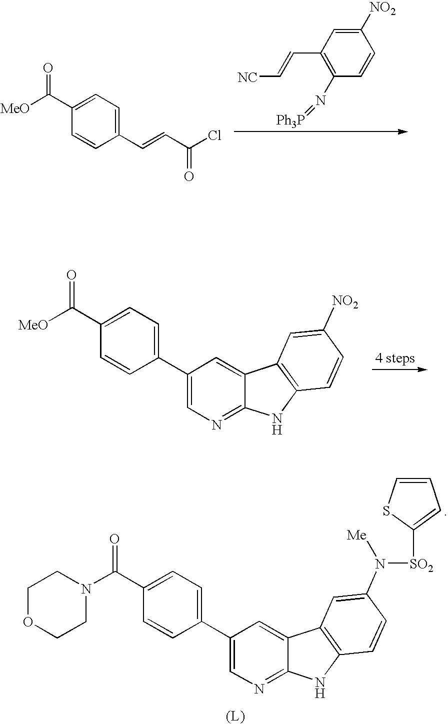

However, all of these require multiple steps in the preparation of the starting compounds upon establishing the carboline skeleton, and thus, they are not very efficient.

An α-carboline derivative is prepared according to Non-Patent Document 5 (Tetrahedron, Vol. 37, p. 2097 (1981)) as shown by the following reaction scheme:

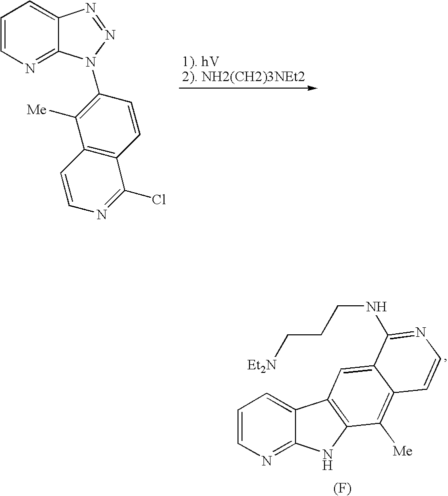

but the method requires a photoirradiation device upon establishing the carboline skeleton.

An α-carboline derivative is prepared according to Patent Document 5 as shown by the following reaction scheme:

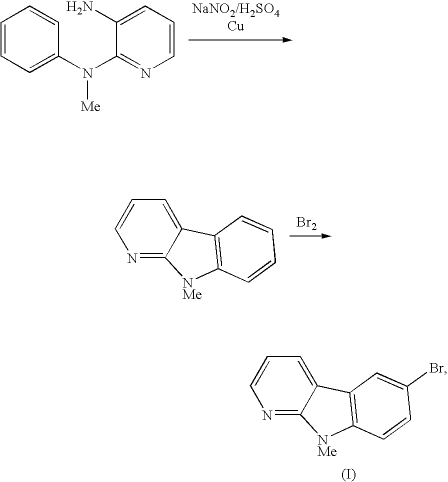

but the method requires a diazotization reaction of high risk upon establishing the carboline skeleton. In the case of the current method, a substituent on the nitrogen of a biarylamine product is required.

An α-carboline derivative is prepared according to Non-Patent Document 4 as shown by the following reaction scheme:

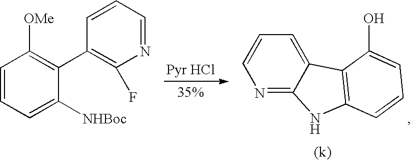

but multiple steps are required in the preparation of the starting compound upon establishing the carboline skeleton, thus the method being not very efficient. Furthermore, there is no description on other 5-hydroxycarboline derivatives.

An α-carboline derivative is prepared according to Non-Patent Document 6 (J. Chem. Soc., Perkin Transactions 1, p. 1262 (1993)) as shown by the following reaction scheme:

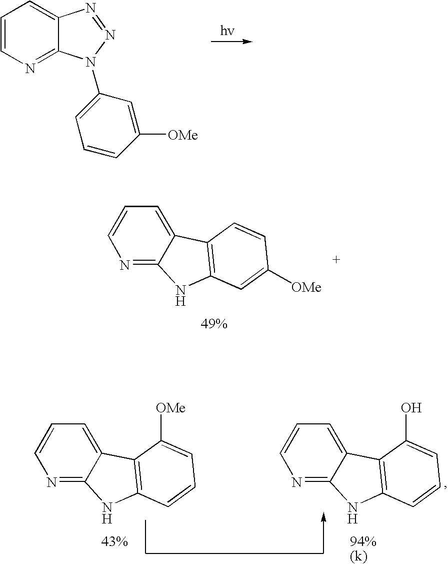

but a photoirradiation device is required upon establishing the carboline skeleton. Furthermore, the method yields a mixture of regioisomers, thus requiring an operation of separating 5-hydroxycarboline derivatives. Moreover, there is no description on other 5-hydroxycarboline derivatives.

Meanwhile, methods for preparing α-carboline derivatives are described in Non-Patent Document 7 (J. Chem. Soc., Perkin Transactions 1, p. 1505 (1999)) as shown by the following reaction scheme:

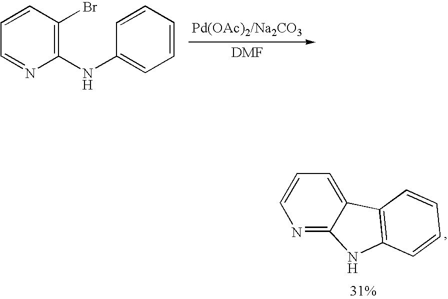

and in Non-Patent Document 8 (Synlett, p. 615 (2003)) as shown by the following reaction scheme:

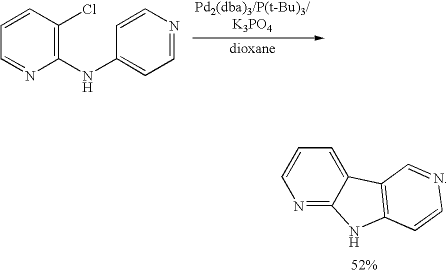

However, no description can be found concerning substituents on the carboline, and the yield is also low.

A method for preparing an azaindole derivative is described in Non-Patent Documents 9 (Tetrahedron, Vol. 55, p. 1959 (1999)) and 10 (Synlett, p. 2571 (2005)) as shown by the following reaction scheme:

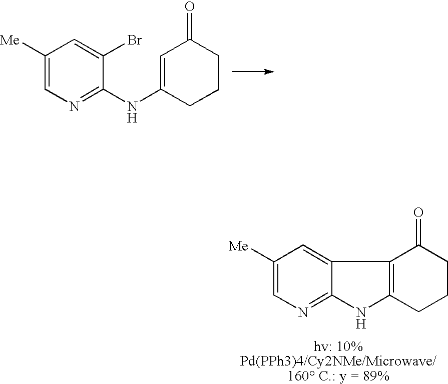

but no description is found on the derivatization to α-carboline. Further, a photoirradiation device or a microwave irradiation device is required as a reaction apparatus. It is also described that a desired product cannot be obtained under the conditions involving palladium acetate/triphenylphosphine/sodium hydrogen carbonate/N,N-dimethylformamide (reflux temperature).

### DISCLOSURE OF THE INVENTION

### Problems to be Solved by the Invention

Simple and convenient methods for preparing α-carboline derivatives which are useful for pharmaceutical products, agrochemicals, food products, cosmetic products, and chemical products, or as intermediates thereof, are being desired. It is also desirable to provide, based on the development of the novel compounds using this method, novel intermediates for establishing efficient methods for preparing the useful compounds described above.

### Means to Solve the Problems

Under such circumstances, the inventors of the present invention devotedly conducted an investigation on the syntheses of α-carboline derivatives. As a result, they found that (1) an α-carboline derivative (II) can be unexpectedly conveniently prepared by subjecting an N-arylaminopyridine or N-heteroarylaminopyridine derivative (I) having various substituents to a ring closure reaction in the presence of a palladium catalyst; that (2) an α-carboline derivative (IX) can be unexpectedly conveniently prepared by subjecting an N-pyridylenamine derivative (VII) to a ring closure reaction in the presence of a palladium catalyst, and subsequently aromatizing the resulting product; and that (3) α-carboline derivatives (XV), (XVII), and (XIX) can be unexpectedly conveniently prepared by subjecting α-carboline derivatives (II) and (IX) to a reaction for introducing a leaving group when necessary, and subsequently to a coupling reaction. Since the methods for preparation of the present invention do not necessitate any expensive starting compounds or special reaction apparatuses such as those presented by the aforementioned publications, α-carboline derivatives can be prepared in few steps, conveniently as well as industrially advantageously.

Furthermore, the inventors also found that carboline derivatives (XI), (XIII), and a tetrahydrocarboline derivative (XII), N-arylaminopyridine or N-heteroarylaminopyridine derivative (XX), which are obtained by the present methods for preparation, serve as novel intermediates for establishing efficient methods for preparation of known pharmaceutical products, thus completing the invention.

Thus, the invention provides the following.

[1] A method for preparation of a compound represented by the following formula:

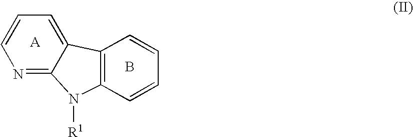

wherein ring A represents a pyridine ring which may be substituted; R¹ represents a hydrogen atom, a C₁₋₁₀ alkyl group which may be substituted, or an acyl group; ring B represents a benzene ring which may be substituted, or a pyridine ring which may be substituted; and at least one of ring A and ring B is substituted;

or a salt thereof, by subjecting a compound represented by the following formula:

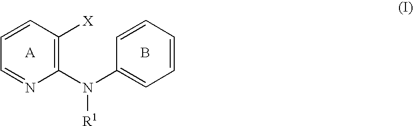

wherein ring A, R¹, and ring B, respectively represent the same meaning as defined above; X represents a leaving group; and at least one of ring A and ring B is substituted;

or a salt thereof to a ring closure reaction in the presence of a palladium catalyst, a ligand, and a base.

[2] The method according to [1] above, wherein X is a halogen atom, a C₁₋₄ alkanesulfonyloxy group which may be halogenated, or a benzenesulfonyloxy group which may be substituted.

[3] A method for preparation of a compound represented by the following formula:

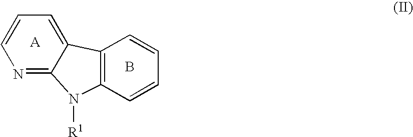

wherein ring A represents a pyridine ring which may be substituted; R¹ represents a hydrogen atom, a C₁₋₁₀ alkyl group which may be substituted, or an acyl group; ring B represents a benzene ring which may be substituted, or a pyridine ring which may be substituted; and at least one of ring A and ring B is substituted;

or a salt thereof, by reacting a compound represented by the following formula:

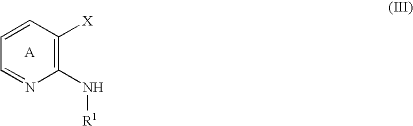

wherein ring A and R¹ respectively represent the same meaning as defined above; and X represents a leaving group;

with a compound represented by the following formula:

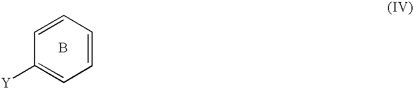

wherein ring B represents the same meaning as defined above; and Y represents a halogen atom, a C₁₋₄ alkanesulfonyloxy group which may be halogenated, or a benzenesulfonyloxy group which may be substituted;

in the presence of a metal catalyst to obtain a compound represented by the following formula:

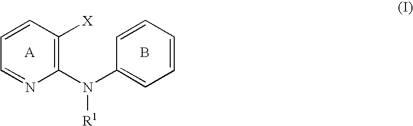

wherein the symbols respectively represent the same meaning as defined above; and at least one of ring A and ring B is substituted;

and subsequently subjecting the compound represented by Formula (I) or a salt thereof to a ring closure reaction in the presence of a palladium catalyst, a ligand, and a base.

[4] A method for preparation of a compound represented by the following formula:

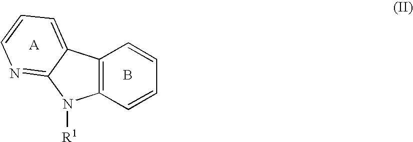

wherein ring A represents a pyridine ring which may be substituted; R¹ represents a hydrogen atom, a C₁₋₁₀ alkyl group which may be substituted, or an acyl group; ring B represents a benzene ring which may be substituted, or a pyridine ring which may be substituted; and at least one of ring A and ring B is substituted;

or a salt thereof, by reacting a compound represented by the following formula:

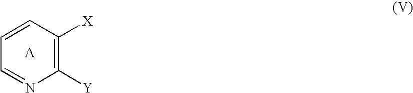

wherein ring A represents the same meaning as defined above; X represents a leaving group; and Y represents a halogen atom, a C₁₋₄ alkanesulfonyloxy group which may be halogenated, or a benzenesulfonyloxy group which may be substituted;

with a compound represented by the following formula:

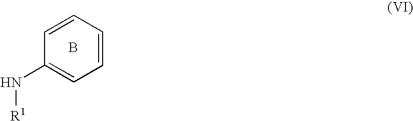

wherein the symbols respectively represent the same meaning as defined above;

to obtain a compound represented by the following formula:

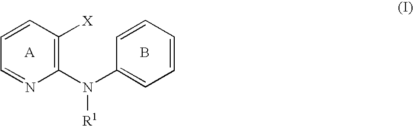

wherein the symbols respectively represent the same meaning as defined above; and at least one of ring A and ring B is substituted;

and subsequently subjecting the compound represented by Formula (I) or a salt thereof to a ring closure reaction in the presence of a palladium catalyst, a ligand, and a base.

[5] The method according to [1] above, wherein the substituent of ring A or ring B is a halogen atom, a hydroxyl group, an amino group which may be substituted, a C₁₋₁₀ alkoxycarbonyl group which may be substituted, an aminocarbonyl group which may have one or two substituents on the nitrogen atom, a C₆₋₁₀ aryl group which may be substituted, or a C₅₋₁₀ heteroaryl group which may be substituted.

[6] The method according to [1] above, wherein the ligand is 2-(dicyclohexylphosphino)biphenyl or 1,1′-bis(diphenylphosphino)ferrocene.

[7] The method according to [1] above, wherein the base is 1,5-diazabicyclo[4.3.0]non-5-ene (DBN) or 1,8-diazabicyclo[5.4.0]undec-7-ene (DBU).

[8] A method for preparation of a compound represented by the following formula:

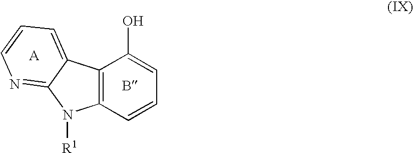

wherein ring A represents a pyridine ring which may be substituted; R¹ represents a hydrogen atom, a C₁₋₁₀ alkyl group which may be substituted, or an acyl group; ring B″ represents a benzene ring which may be substituted; and at least one of ring A and ring B″ is substituted;

or a salt thereof, by subjecting a compound represented by the following formula:

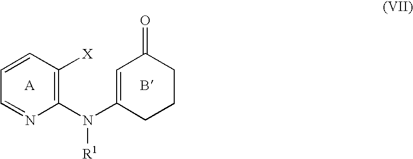

wherein ring A and R¹ respectively represent the same meaning as defined above; X represents a leaving group; ring B′ represents a cyclohexenone ring which may be substituted; and at least one of ring A and ring B′ is substituted;

or a salt thereof to a ring closure reaction in the presence of a palladium catalyst, a ligand, and a base, to obtain a compound represented by the following formula:

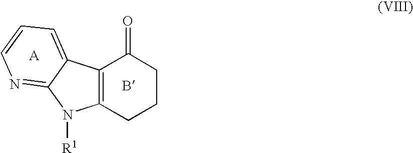

wherein the symbols respectively represent the same meaning as defined above; and at least one of ring A and ring B′ is substituted;

or a salt thereof, and subsequently aromatizing ring B′ of the compound represented by Formula (VIII) or a salt thereof.

[9] A method for preparation of a compound represented by the following formula:

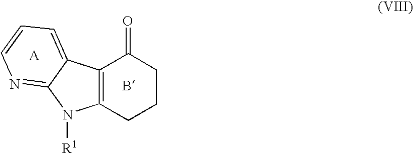

wherein ring A represents a pyridine ring which may be substituted; R¹ represents a hydrogen atom, a C₁₋₁₀ alkyl group which may be substituted, or an acyl group; ring B′ represents a cyclohexenone ring which may be substituted; and at least one of ring A and ring B′ is substituted;

or a salt thereof, by reacting a compound represented by the following formula:

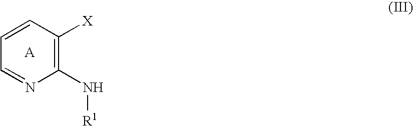

wherein ring A and R¹ respectively represent the same meaning as defined above; and X represents a leaving group;

with a compound represented by the following formula:

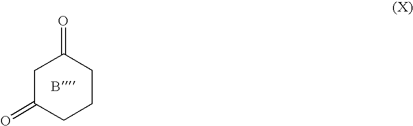

wherein ring B″″ represents a 1,3-cyclohexanedione ring which may be substituted;

to obtain a compound represented by the following formula:

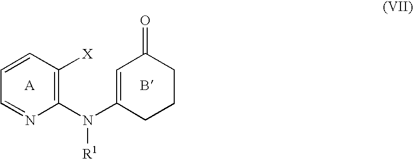

wherein the symbols respectively represent the same meaning as defined above; and at least one of ring A and ring B′ is substituted;

and subsequently subjecting the compound represented by Formula (VII) to a ring closure reaction in the presence of a palladium catalyst, a ligand, and a base.

[10] A method for preparation of a compound represented by the following formula:

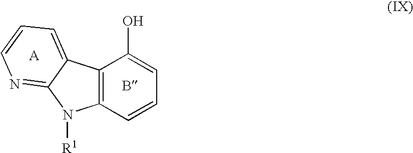

wherein ring A represents a pyridine ring which may be substituted; R¹ represents a hydrogen atom, a C₁₋₁₀ alkyl group which may be substituted, or an acyl group; ring B″ represents a benzene ring which may be substituted; and at least one of ring A and ring B″ is substituted;

or a salt thereof, by reacting a compound represented by the following formula:

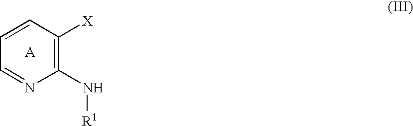

wherein ring A and R¹ respectively represent the same meaning as defined above; and X represents a leaving group;

with a compound represented by the following formula:

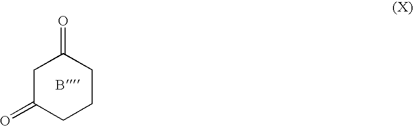

wherein ring B″″ represents a 1,3-cyclohexanedione ring which may be substituted;

to obtain a compound represented by the following formula:

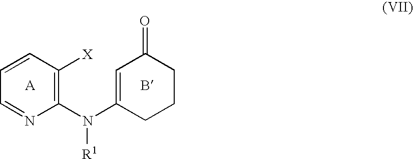

wherein ring A, R¹, and X respectively represent the same meaning as defined above; ring B′ represents a cyclohexenone ring which may be substituted; and at least one of ring A and ring B′ is substituted;

subsequently subjecting the compound represented by Formula (VII) to a ring closure reaction in the presence of a palladium catalyst, a ligand, and a base to obtain a compound represented by the following formula:

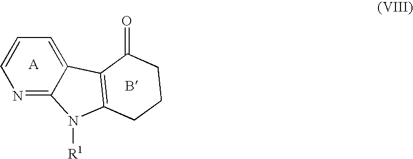

wherein the symbols respectively represent the same meaning as defined above; and at least one of ring A and ring B′ is substituted;

or a salt thereof, and subsequently aromatizing ring B′ of the compound represented by Formula (VIII) or a salt thereof.

[11] The method according to [8] above, wherein the base is cesium carbonate, tripotassium phosphate, or 1,5-diazabicyclo[2.2.2]octane (DABCO).

[12] A compound represented by the following formula:

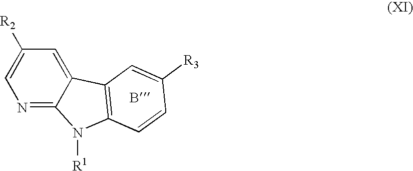

wherein ring B′″ represents a benzene ring which may be further substituted in addition to R³; R² represents a halogen atom, a nitro group, a C₁₋₁₀ alkyl group which may be substituted, an amino group which may be substituted, or a C₁₋₁₀ alkylthio group which may be substituted; R³ represents a halogen atom, a C₁₋₁₀ alkoxy group which may be substituted, an amino group which may be substituted, or a C₁₋₁₀ alkylthio group which may be substituted;

or a salt thereof.

[13] A compound represented by the following formula:

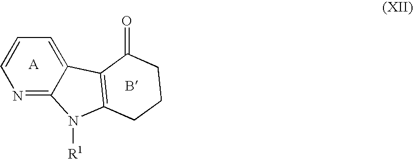

wherein ring A represents a pyridine ring which may be substituted; ring B′ represents a cyclohexenone ring which may be substituted; and at least one of ring A and ring B′ is substituted;

or a salt thereof, provided that the following compounds are excluded:

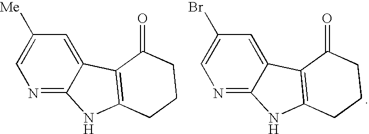

[14] A compound represented by the following formula:

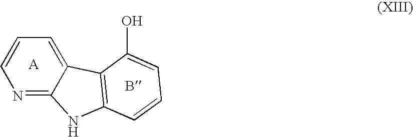

wherein ring A represents a pyridine ring which may be substituted; ring B″ represents a benzene ring which may be substituted; and at least one of ring A and ring B″ is substituted;

or a salt thereof.

[15] A method for preparation of a compound represented by the following formula:

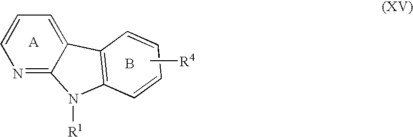

wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B represents a benzene ring which may be substituted or a pyridine ring which may be substituted; and at least one of ring A and ring B is substituted;

or a salt thereof, by reacting a compound represented by the following formula:

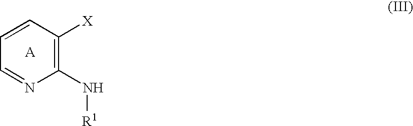

wherein ring A and R¹ respectively represent the same meaning as defined above; and X represents a leaving group;

with a compound represented by the following formula:

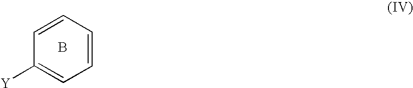

wherein ring B represents the same meaning as defined above; and Y represents a halogen atom, a C₁₋₄ alkanesulfonyloxy group which may be halogenated, or a benzenesulfonyloxy group which may be substituted;

in the presence of a metal catalyst to obtain a compound represented by the following formula:

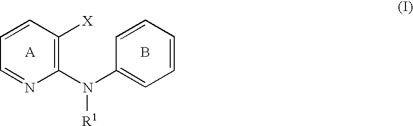

wherein the symbols respectively represent the same meaning as defined above; and at least one of ring A and ring B is substituted;

subsequently subjecting the compound represented by Formula (I) or a salt thereof to a ring closure reaction in the presence of a palladium catalyst, a ligand, and a base, to obtain a compound represented by the following formula:

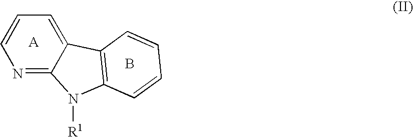

wherein the symbols respectively represent the same meaning as defined above; and at least one of ring A and ring B is substituted;

or a salt thereof, then subsequently subjecting the compound represented by Formula (II) or a salt thereof to a reaction for introducing a leaving group when necessary, to obtain a compound represented by the following formula:

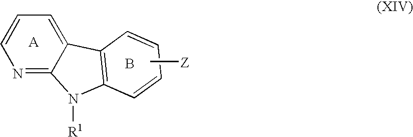

wherein Z represents a leaving group; other symbols respectively represent the same meaning as defined above; and at least one of ring A and ring B is substituted;

or a salt thereof, and subsequently subjecting the compound represented by Formula (II) or a salt thereof or the compound represented by Formula (XIV) or a salt thereof, to a coupling reaction.

[16] A method for preparation of a compound represented by the following formula:

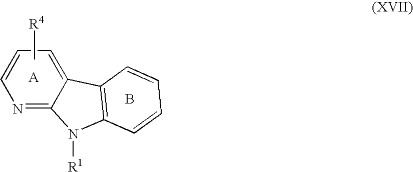

wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B represents a benzene ring which may be substituted or a pyridine ring which may be substituted; and at least one of ring A and ring B is substituted;

or a salt thereof, by reacting a compound represented by the following formula:

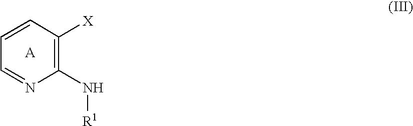

wherein ring A and R¹ respectively represent the same meaning as defined above; and X represents a leaving group;

with a compound represented by the following formula:

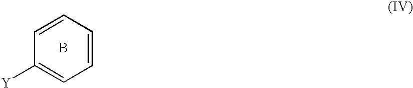

wherein ring B represents the same meaning as defined above; and Y represents a halogen atom, a C₁₋₄ alkanesulfonyloxy group which may be halogenated, or a benzenesulfonyloxy group which may be substituted;

in the presence of a metal catalyst to obtain a compound represented by the following formula:

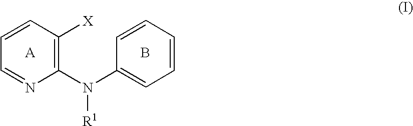

wherein the symbols respectively represent the same meaning as defined above; and at least one of ring A and ring B is substituted;

subsequently subjecting the compound represented by Formula (I) or a salt thereof to a ring closure reaction in the presence of a palladium catalyst, a ligand, and a base, to obtain a compound represented by the following formula:

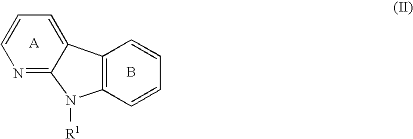

wherein the symbols respectively represent the same meaning as defined above; and at least one of ring A and ring B is substituted;

or a salt thereof, then subsequently subjecting the compound represented by Formula (II) or a salt thereof to a reaction for introducing a leaving group when necessary, to obtain a compound represented by the following formula:

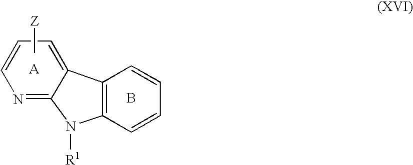

wherein Z represents a leaving group; other symbols respectively represent the same meaning as defined above; and at least one of ring A and ring B is substituted;

or a salt thereof, and subsequently subjecting the compound represented by Formula (II) or a salt thereof or the compound represented by Formula (XVI) or a salt thereof, to a coupling reaction.

[17] A method for preparation of a compound represented by the following formula:

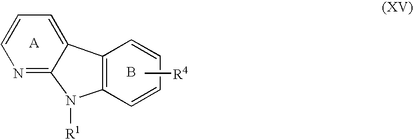

wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B represents a benzene ring which may be substituted or a pyridine ring which may be substituted; and at least one of ring A and ring B is substituted;

or a salt thereof, by reacting a compound represented by the following formula:

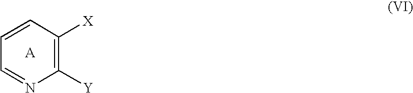

wherein ring A represents the same meaning as defined above; X represents a leaving group; and Y represents a halogen atom, a C₁₋₄ alkanesulfonyloxy group which may be halogenated, or a benzenesulfonyloxy group which may be substituted;

with a compound represented by the following formula:

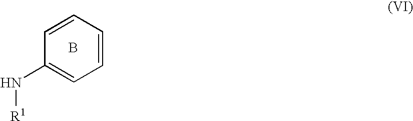

wherein the symbols respectively represent the same meaning as defined above;

to obtain a compound represented by the following formula:

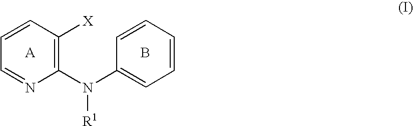

wherein the symbols respectively represent the same meaning as defined above; and at least one of ring A and ring B is substituted;

subsequently subjecting the compound represented by Formula (I) or a salt thereof to a ring closure reaction in the presence of a palladium catalyst, a ligand, and a base, to obtain a compound represented by the following formula:

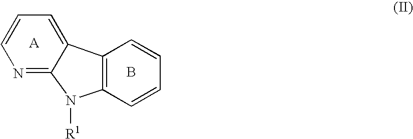

wherein the symbols respectively represent the same meaning as defined above; and at least one of ring A and ring B is substituted;

or a salt thereof, then subsequently subjecting the compound represented by Formula (II) or a salt thereof to a reaction for introducing a leaving group when necessary, to obtain a compound represented by the following formula:

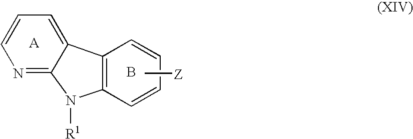

wherein Z represents a leaving group; other symbols respectively represent the same meaning as defined above; and at least one of ring A and ring B is substituted;

or a salt thereof, and subsequently subjecting the compound represented by Formula (II) or a salt thereof or the compound represented by Formula (XIV) or a salt thereof, to a coupling reaction.

[18] A method for preparation of a compound represented by the following formula:

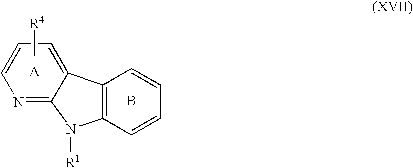

wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B represents a benzene ring which may be substituted or a pyridine ring which may be substituted; and at least one of ring A and ring B is substituted;

or a salt thereof, by reacting a compound represented by the following formula:

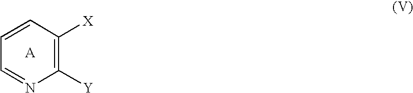

wherein ring A represents the same meaning as defined above; X represents a leaving group; and Y represents a halogen atom, a C₁₋₄ alkanesulfonyloxy group which may be halogenated, or a benzenesulfonyloxy group which may be substituted;

with a compound represented by the following formula:

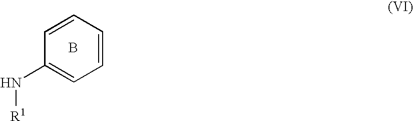

wherein the symbols respectively represent the same meaning as defined above;

to obtain a compound represented by the following formula:

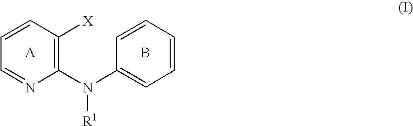

wherein the symbols respectively represent the same meaning as defined above; and at least one of ring A and ring B is substituted;

subsequently subjecting the compound represented by Formula (I) or a salt thereof to a ring closure reaction in the presence of a palladium catalyst, a ligand, and a base, to obtain a compound represented by the following formula:

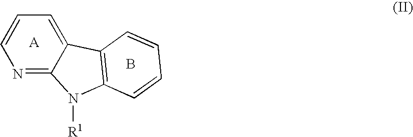

wherein the symbols respectively represent the same meaning as defined above; and at least one of ring A and ring B is substituted;

or a salt thereof, then subsequently subjecting the compound represented by Formula (II) or a salt thereof to a reaction for introducing a leaving group when necessary, to obtain a compound represented by the following formula:

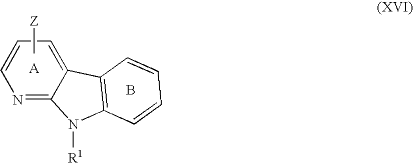

wherein Z represents a leaving group; other symbols respectively represent the same meaning as defined above; and at least one of ring A and ring B is substituted;

or a salt thereof, and subsequently subjecting the compound represented by Formula (II) or a salt thereof or the compound represented by Formula (XVI) or a salt thereof, to a coupling reaction.

[19] A method for preparation of a compound represented by the following formula:

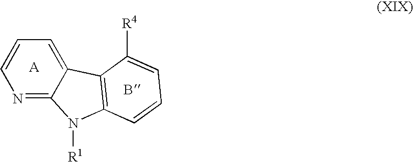

wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B″ represents a benzene ring which may be substituted; and at least one of ring A and ring B″ is substituted;

or a salt thereof, by reacting a compound represented by the following formula:

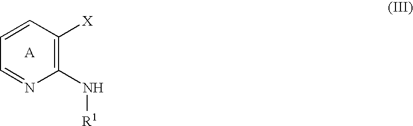

wherein ring A and R¹ respectively represent the same meaning as defined above; and X represents a leaving group;

with a compound represented by the following formula:

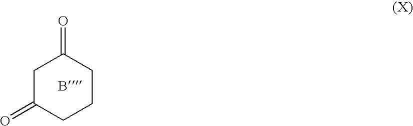

wherein ring B″″ represents a 1,3-cyclohexanedione ring which may be substituted;

to obtain a compound represented by the following formula:

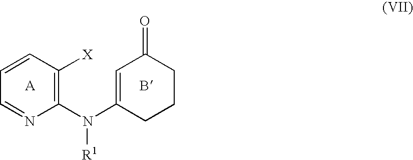

wherein ring A, X, and R¹ respectively represent the same meaning as defined above; and ring B′ represents a cyclohexenone ring which may be substituted; and at least one of ring A and ring B′ is substituted;

subsequently subjecting the compound represented by Formula (VII) to a ring closure reaction in the presence of a palladium catalyst, a ligand, and a base, to obtain a compound represented by the following formula:

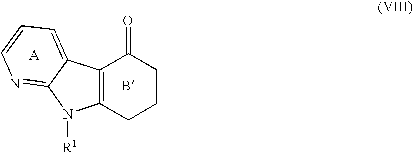

wherein the symbols respectively represent the same meaning as defined above; and at least one of ring A and ring B′ is substituted;

or a salt thereof, then subsequently subjecting ring B′ of the compound represented by Formula (VIII) or a salt thereof to an aromatization reaction to obtain a compound represented by the following formula:

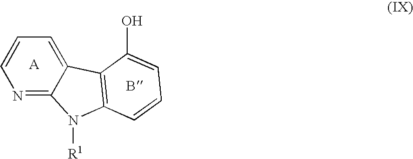

wherein the symbols respectively represent the same meaning as defined above; and at least one of ring A and ring B″ is substituted;

or a salt thereof, subsequently converting a hydroxyl group of the compound represented by Formula (IX) or a salt thereof to a leaving group to obtain a compound represented by the following formula:

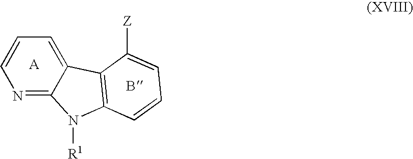

wherein Z represents a leaving group; other symbols respectively represent the same meaning as defined above; and at least one of ring A and ring B″ is substituted;

or a salt thereof, and subsequently subjecting the compound represented by Formula (XVIII) or a salt thereof to a coupling reaction.

[20] A method for preparation of a compound represented by the following formula:

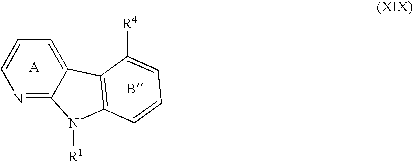

wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B″ represents a benzene ring which may be substituted; and at least one of ring A and ring B″ is substituted;

or a salt thereof, by reacting a compound represented by the following formula:

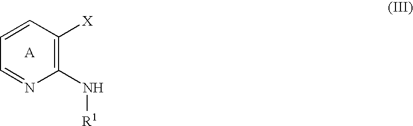

wherein ring A and R¹ respectively represent the same meaning as defined above; and X represents a leaving group;

with a compound represented by the following formula:

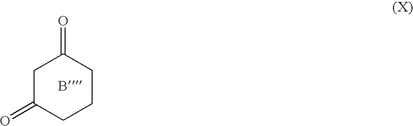

wherein ring B″″ represents a 1,3-cyclohexanedione ring which may be substituted;

to obtain a compound represented by the following formula:

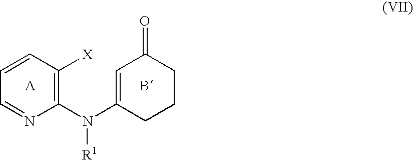

wherein ring A, X, and R¹ respectively represent the same meaning as defined above; and ring B′ represents a cyclohexenone ring which may be substituted; and at least one of ring A and ring B′ is substituted;

subsequently subjecting the compound represented by Formula (VII) to a ring closure reaction in the presence of a palladium catalyst, a ligand, and a base, to obtain a compound represented by the following formula:

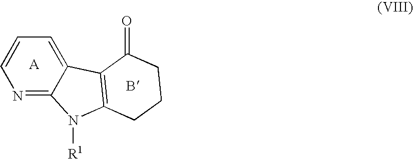

wherein the symbols respectively represent the same meaning as defined above; and at least one of ring A and ring B′ is substituted;

or a salt thereof, then subsequently subjecting ring B′ of the compound represented by Formula (VIII) or a salt thereof to an aromatization reaction to obtain a compound represented by the following formula:

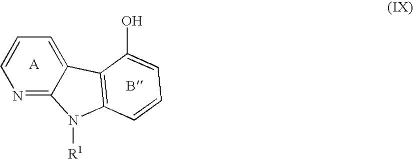

wherein the symbols respectively represent the same meaning as defined above; and at least one of ring A and ring B″ is substituted;

or a salt thereof, subsequently subjecting a hydroxyl group of the compound represented by Formula (IX) or a salt thereof to a coupling reaction.

[21] The method according to [8] above, wherein the substituent of ring A or ring B″ is a C₆₋₁₀ aryl group which may be substituted or a C₅₋₁₀ heteroaryl group which may be substituted.

[22] The compound according to [13] above, wherein the substituent of ring A or ring B′ is a C₆₋₁₀ aryl group which may be substituted or a C₅₋₁₀ heteroaryl group which may be substituted.

[23] The compound according to [14] above, wherein the substituent of ring A or ring B″ is a C₆₋₁₀ aryl group which may be substituted or a C₅₋₁₀ heteroaryl group which may be substituted.

[24] A compound represented by the following formula:

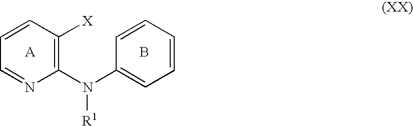

wherein ring A represents a pyridine ring which may be substituted; X represents a leaving group; R¹ represents a hydrogen atom, a C₁₋₁₀ alkyl group which may be substituted, or an acyl group; ring B represents a benzene ring which may be substituted or a pyridine ring which may be substituted; at least one of ring A and ring B is substituted; and the substituent(s) of ring A and/or ring B is (are) a substituent (substituents) selected from a halogen atom, an amino group which may be substituted, a C₁₋₁₀ alkoxy group which may be substituted, a C₁₋₁₀ alkoxy-carbonyl group which may be substituted, an aminocarbonyl group optionally having one or two substituent(s) on a nitrogen atom; a C₁₋₁₀ aryl group which may be substituted; and a C₅₋₁₀ heteroaryl group which may be substituted;

or a salt thereof.

[25] The compound according to [24] above, wherein R¹ is a hydrogen atom, at least one of ring A and ring B is substituted, and the substitutents of ring A and/or ring B are at least two kinds of substituents selected from a halogen atom, an amino group which may be substituted, a C₁₋₁₀ alkoxy group which may be substituted, a C₁₋₁₀ alkoxy-carbonyl group which may be substituted, an aminocarbonyl group optionally having one or two substituent(s) on a nitrogen atom, a C₆₋₁₀ aryl group which may be substituted, and a C₅₋₁₀ heteroaryl group which may be substituted.

[26] The compound according to [24] above, wherein R¹ is a hydrogen atom, at least one of ring A and ring B is substituted, and the substitutents of ring A and/or ring B are (i) at least one kind of a substituent selected from an amino group which may be substituted, a C₁₋₁₀ alkoxy group which may be substituted, a C₁₋₁₀ alkoxy-carbonyl group which may be substituted, and an aminocarbonyl group optionally having one or two substituent(s) on a nitrogen atom, and (ii) at least one kind of a substituent selected from an amino group which may be substituted, a C₆₋₁₀ aryl group which may be substituted, and a C₅₋₁₀ heteroaryl group which may be substituted.

[27] The compound according to [12] above, wherein R² is a halogen atom, provided that the following compounds are excluded;

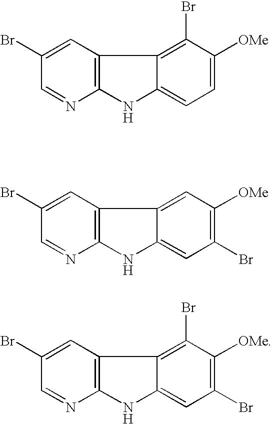

[28] The compound according to [12] above, wherein R³ is a halogen atom, provided that the following compounds are excluded;

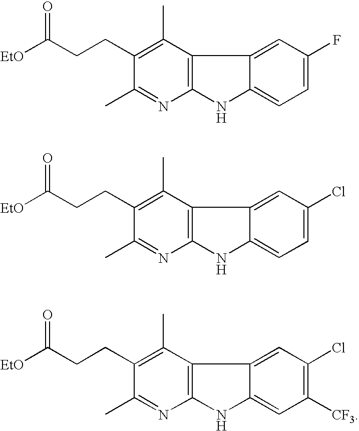

### Effect of the Invention

According to the methods for preparation of the invention, since expensive starting compounds or special reaction apparatuses such as those needed in conventional methods are not necessitated, α-carboline derivatives (II), (IX), (XV), (XVII), and (XIX) having a variety of substituents can be prepared in few steps conveniently as well as industrially advantageously. Furthermore, based on the development of the novel compounds using these methods, novel intermediates (XI), (XII), (XIII), and (XX) for establishing efficient methods for preparation of known pharmaceutical products can be provided.

Hereinafter, the definitions of the respective symbols in the formulas will be illustrated.

The “pyridine ring which may be substituted” represented by ring A, may have 1 to 3 substituents on the substitutable positions, and in the case of having a plurality of substituents, these substituents may be identical with or different from each other. Examples of these substituents include:

(1) a C₁₋₁₀ alkyl group which may be substituted with a halogen atom (for example, methyl, ethyl, n-propyl, isopropyl, n-butyl, isobutyl, sec-butyl, tert-butyl, n-pentyl, isopentyl, neopentyl, 1-methylpropyl, n-hexyl, isohexyl, 1,1-dimethylbutyl, 2,2-dimethylbutyl, 3,3-dimethylbutyl, 3,3-dimethylpropyl, 2-ethylbutyl, n-heptyl, 1-methylheptyl, 1-ethylhexyl, n-octyl, 1-methylheptyl, nonyl, cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, trifluoromethyl);

(2) a C₆₋₁₄ aryl group (for example, phenyl, naphthyl) which may be substituted with a substituent selected from a halogen atom, a cyano group, a nitro group, a hydroxyl group, an amino group, a C₁₋₁₀ alkyl group which may be substituted with a halogen atom, a C₂₋₁₀ alkenyl group, a C₂₋₁₀ alkynyl group, a C₁₋₁₀ alkoxy-carbonyl group which may be substituted with a halogen atom, a C₁₋₁₀ alkylcarbonyl group which may be substituted with a halogen atom, a C₁₋₁₀ alkylaminocarbonyl group which may be substituted with a halogen atom, a di-C₁₋₁₀ alkylaminocarbonyl group which may be substituted with a halogen atom, a C₁₋₁₀ alkylsulfonyl group which may be substituted with a halogen atom, a C₁₋₁₀ alkylsulfinyl group which may be substituted with a halogen atom, a C₁₋₁₀ alkylthio group which may be substituted with a halogen atom, a C₁₋₁₀ alkylsulfonylamino group which may be substituted with a halogen atom, a C₁₋₁₀ alkylamino group which may be substituted with a halogen atom, a di-C₁₋₁₀ alkylamino group which may be substituted with a halogen atom, a C₁₋₁₀ alkoxycarbonylamino group which may be substituted with a halogen atom, and a C₁₋₁₀ alkylcarbonylamino group which may be substituted with a halogen atom;

(3) a C₅₋₁₀ heteroaryl group (for example, a 5- to 6-membered aromatic monocyclic heterocyclic group such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl or the like; a 8- to 12-membered aromatic fused heterocyclic group such as benzofuranyl, isobenzofuranyl, benzo[b]thienyl, indolyl, isoindolyl, 1H-indazolyl, benzindazolyl, benzoxazolyl, 1,2-benzoisoxazolyl, benzothiazolyl, benzopyranyl, 1,2-benzoisothiazolyl, 1H-benzotriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, phenathridinyl, phenathrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl or the like) which may be substituted with a substituent selected from a halogen atom, a cyano group, a nitro group, a hydroxyl group, an amino group, a C₁₋₁₀ alkyl group which may be substituted with a halogen atom, C₂₋₁₀ alkenyl group, C₂₋₁₀ alkynyl group, a C₁₋₁₀ alkoxycarbonyl group which may be substituted with a halogen atom, a C₁₋₁₀ alkylcarbonyl group which may be substituted with a halogen atom, a C₁₋₁₀ alkylaminocarbonyl group which may be substituted with a halogen atom, a di-C₁₋₁₀ alkylaminocarbonyl group which may be substituted with a halogen atom, a C₁₋₁₀ alkylsulfonyl group which may be substituted with a halogen atom, a C₁₋₁₀ alkylsulfinyl group which may be substituted with a halogen atom, a C₁₋₁₀ alkylthio group which may be substituted with a halogen atom, a C₁₋₁₀ alkylsulfonylamino group which may be substituted with a halogen atom, a C₁₋₁₀ alkylamino group which may be substituted with a halogen atom, a di-C₁₋₁₀ alkylamino group which may be substituted with a halogen atom, a C₁₋₁₀ alkoxycarbonylamino group which may be substituted with a halogen atom, and a C₁₋₁₀ alkylcarbonylamino group which may be substituted with a halogen atom;

(4) a non-aromatic heterocyclic group (for example, tetrahydrofuryl, morpholino, thiomorpholino, piperidino, pyrrolidinyl, piperazinyl, oxodioxolyl, oxodioxolanyl, oxo-2-benzofuranyl, oxo-oxadiazolyl) which may be substituted with a C₁₋₁₀ alkyl group (for example, methyl, ethyl);

(5) an amino group which may be substituted {for example, an amino group which may be mono- or disubstituted with a substituent selected from a C₁₋₁₀ alkyl group (for example, methyl, ethyl), a C₁₋₁₀ alkyl-carbonyl group (for example, acetyl, isobutanoyl, isopentanoyl) and a C₁₋₁₀ alkoxy-carbonyl group (for example, methoxycarbonyl, ethoxycarbonyl, propoxycarbonyl, tert-butoxycarbonyl); a C₁₋₁₀ alkylsulfonylamino group (for example, methylsulfonylamino); a C₇₋₁₃ aralkylamino group (for example, benzylamino)};

(6) a cyclic imide group forming a fused ring together with ring A;

(7) an amidino group;

(8) a C₁₋₁₀ alkyl-carbonyl group (for example, acetyl, isobutanoyl, isopentanoyl);

(9) a C₁₋₁₀ alkoxy-carbonyl group (for example, methoxycarbonyl, ethoxycarbonyl, propoxycarbonyl, tert-butoxycarbonyl) which may be substituted with a halogen atom;

(10) a C₁₋₁₀ alkylsulfonyl group (for example, methylsulfonyl);

(11) an aminocarbonyl group which may have one or two substituents on the nitrogen atom {examples of the substituent: a C₁₋₁₀ alkyl group which may be substituted with a halogen atom such as described in (1) above, a C₆₋₁₄ aryl group which may be substituted such as described in (2) above, a C₅₋₁₀ heteroaryl group which may be substituted such as described in (3) above, a non-aromatic heterocyclic group which may be substituted with a C₁₋₁₀ alkyl group (for example, methyl, ethyl) such as described in (4) above, an amino group which may be substituted such as described in (5)};

(12) a thiocarbamoyl group which may have one or two substituents on the nitrogen atom {examples of the substituent: a C₁₋₁₀ alkyl group which may be substituted with a halogen atom such as described in (1) above, a C₆₋₁₄ aryl group which may be substituted such as described in (2) above, a C₅₋₁₀ heteroaryl group which may be substituted such as described in (3) above, a non-aromatic heterocyclic group which may be substituted with a C₁₋₁₀ alkyl group (for example, methyl, ethyl) such as described in (4) above, an amino group which may be substituted such as described in (5) above};

(13) a sulfamoyl group which may have one or two substituents on the nitrogen atom {examples of the substituent: a C₁₋₁₀ alkyl group which may be substituted with a halogen atom such as described in (1) above, a C₆₋₁₄ aryl group which may be substituted such as described in (2) above, a C₅₋₁₀ heteroaryl group which may be substituted such as described in (3) above, a non-aromatic heterocyclic group which may be substituted with a C₁₋₁₀ alkyl group (for example, methyl, ethyl) such as described in (4) above, an amino group which may be substituted such as described in (5) above};

(14) a carboxyl group;

(15) a hydroxyl group;

(16) a C₁₋₁₀ alkoxy group (for example, methoxy, ethoxy) which may be substituted with 1 to 3 halogen atoms (for example, fluorine, chlorine, bromine, iodine);

(17) a C₂₋₁₀ alkenyloxy group (for example, ethenyloxy) which may be substituted with 1 to 3 halogen atoms (for example, fluorine, chlorine, bromine, iodine);

(18) a C₃₋₁₀ cycloalkyloxy group (for example, cyclohexyloxy);

(19) a C₇₋₁₃ aralkyloxy group (for example, benzyloxy);

(20) a C₆₋₁₄ aryloxy group (for example, phenyloxy, naphthyloxy);

(21) a C₁₋₁₀ alkyl-carbonyloxy group (for example, acetyloxy, tert-butylcarbonyloxy);

(22) a thiol group;

(23) a C₁₋₁₀ alkylthio group (for example, methylthio, ethylthio) which may be substituted with 1 to 3 halogen atoms (for example, fluorine, chlorine, bromine, iodine);

(24) a C₇₋₁₃ aralkylthio group (for example, benzylthio);

(25) a C₆₋₁₄ arylthio group (for example, phenylthio, naphthylthio);

(26) a sulfo group;

(27) a cyano group;

(28) an azide group;

(29) a nitro group;

(30) a nitroso group;

(31) a halogen atom (for example, fluorine, chlorine, bromine, iodine);

(32) a C₁₋₁₀ alkylsulfinyl group (for example, methylsulfinyl);

(33) a non-aromatic heterocyclic (for example, morpholino)-carbonyl group;

(34) a C₆₋₁₄ arylcarbamoyl group; and the like.

Among these, a C₁₋₁₀ alkyl group which may be substituted with a halogen atom, a C₆₋₁₄ aryl group which may be substituted, a C₅₋₁₀ heteroaryl group which may be substituted, an amino group which may be substituted, a cyclic imide group forming a fused ring together with ring A, a C₁₋₁₀ alkoxy-carbonyl group which may be substituted with a halogen atom, a C₁₋₁₀ alkylsulfonyl group, an aminocarbonyl group which may have one or two substituents on the nitrogen atom, a thiocarbamoyl group which may have one or two substituents on the nitrogen atom, a carboxyl group, a hydroxyl group, a C₁₋₁₀ alkoxy group which may be substituted with 1 to 3 halogen atoms (for example, fluorine, chlorine, bromine, iodine), a cyano group, a nitro group, and a halogen atom are preferred, and a halogen atom, a hydroxyl group, an amino group which may be substituted, a C₁₋₁₀ alkoxycarbonyl group which may be substituted, an aminocarbonyl group which may have one or two substituents on the nitrogen atom, a C₆₋₁₀ aryl group which may be substituted, and a C₅₋₁₀ heteroaryl group which may be substituted are also preferred, while a halogen atom (for example, fluorine, chlorine, bromine, iodine), a hydroxyl group, an amino group which may be substituted, a C₁₋₁₀ alkoxy-carbonyl group which may be substituted with a halogen atom, an aminocarbonyl group which may have one or two substituents on the nitrogen atom, a C₆₋₁₀ aryl group which may be substituted, and a C₅₋₁₀ heteroaryl group which may be substituted are particularly preferred, and a C₆₋₁₀ aryl group which may be substituted and a C₅₋₁₀ heteroaryl group which may be substituted are also particularly preferred.

The “leaving group” represented by X and Z may be exemplified by a halogen atom (for example, fluorine, chlorine, bromine, iodine), a C₁₋₄ alkanesulfonyloxy group which may be halogenated (for example, methanesulfonyloxy, ethanesulfonyloxy, trifluoromethanesulfonyloxy), a benzenesulfonyloxy group which may be substituted, a halogenocarbonyl group (for example, chlorocarbonyl), a halogenosulfonyl group (for example, chlorosulfonyl), a C₁₋₄ alkylthio group (for example, methylthio, ethylthio) which may be substituted with a halogen atom (for example, fluorine, chlorine, bromine, iodine), a C₁₋₄ alkanesulfinyl group (for example, methanesulfinyl, ethanesulfinyl) which may be substituted with a halogen atom (for example, fluorine, chlorine, bromine, iodine), a C₁₋₄ alkanesulfonyl group (for example, methanesulfonyl, ethanesulfonyl) which may be substituted with a halogen atom (for example, fluorine, chlorine, bromine, iodine), an N,N-dialkylaminocarbonyloxy group, an N,N-dialkylaminocarbonylthio group or the like. Among these, a halogen atom, a C₁₋₄ alkanesulfonyloxy group which may be halogenated, and a benzenesulfonyloxy group which may be substituted are preferred.

The “benzenesulfonyloxy group which may be substituted” as a “leaving group” represented by X and Z may have one to the maximum allowed number of substituents on any of the substitutable positions, and in the case of being substituted with two or more substituents, the substituents may be identical with or different from each other. Examples of these substituents include the aforementioned substituents for ring A. Among them, a C₁₋₁₀ alkyl group which may be substituted with a halogen atom, a C₁₋₁₀ alkoxy group which may be substituted with a halogen atom, a nitro group, and a halogen atom are preferred.

The “C₁₋₁₀ alkyl group” of the “C₁₋₁₀ alkyl group which may be substituted” represented by R¹, R², and R⁴, may be exemplified by a methyl, ethyl, n-propyl, isopropyl, n-butyl, isobutyl, sec-butyl, tert-butyl, n-pentyl, isopentyl, neopentyl, 1-methylpropyl, n-hexyl, isohexyl, 1,1-dimethylbutyl, 2,2-dimethylbutyl, 3,3-dimethylbutyl, 3,3-dimethylpropyl, 2-ethylbutyl, n-heptyl, 1-methylheptyl, 1-ethylhexyl, n-octyl, 1-methylheptyl, or nonyl group. The “C₁₋₁₀ alkyl group which may be substituted” represented by R¹, R², and R⁴, may have one to the maximum allowed number of substituents on any of the substitutable positions, and in the case of being substituted with two or more substituents, the substituents may be identical with or different from each other. Examples of these substituents include the aforementioned substituents for ring A. Among them, a halogen atom (for example, fluorine, chlorine, bromine, iodine), a C₁₋₁₀ alkoxy group, and a mono- or di-C₁₋₁₀ alkylamino group are preferred, and in particular, fluorine is preferred.

The “acyl group” represented by R¹ and R⁴ may be exemplified by:

(1) a C₁₋₆ alkyl-carbonyl group (for example, acetyl, isobutanoyl, isopentanoyl);

(2) a C₁₋₆ alkoxy-carbonyl group (for example, methoxycarbonyl, ethoxycarbonyl, propoxycarbonyl, tert-butoxycarbonyl);

(3) a C₃₋₁₀ cycloalkyl-carbonyl group (for example, cyclopentylcarbonyl, cyclohexylcarbonyl);

(4) a C₆₋₁₄ aryl-carbonyl group (for example, benzoyl);

(5) a C₇₋₁₃ aralkyloxy-carbonyl group (for example, benzyloxycarbonyl);

(6) a carbamoyl group;

(7) a mono- or di-C₁₋₆ alkyl-carbamoyl group (for example, methylcarbamoyl, dimethylcarbamoyl);

(8) a mono- or di-C₆₋₁₄ aryl-carbamoyl group (for example, phenylcarbamoyl); or the like.

The “benzene ring which may be substituted or pyridine ring which may be substituted” represented by ring B may have 1 to 3 substituents on the substitutable positions, and in the case of having a plurality of substituents, these substituents may be identical with or different from each other. For these substituents, the same ones as the aforementioned substituents for ring A are used. Among them, a C₁₋₁₀ alkyl group which may be substituted with a halogen atom, a C₆₋₁₄ aryl group which may be substituted, a C₅₋₁₀ heteroaryl group which may be substituted, an amino group which may be substituted, a cyclic imide group forming a fused ring together with ring B, a C₁₋₁₀ alkoxy-carbonyl group which may be substituted with a halogen atom, a C₁₋₁₀ alkylsulfonyl group, an aminocarbonyl group which may have one or two substituents on the nitrogen atom, a thiocarbamoyl group which may have one or two substituents on the nitrogen atom, a carboxyl group, a hydroxyl group, a C₁₋₁₀ alkoxy group which may be substituted with 1 to 3 halogen atoms (for example, fluorine, chlorine, bromine, iodine), a cyano group, a nitro group, and a halogen atom are preferred, and a halogen atom, a hydroxyl group, an amino group which may be substituted, a C₁₋₁₀ alkoxycarbonyl group which may be substituted, an aminocarbonyl group which may have one or two substituents on the nitrogen atom, a C₆₋₁₀ aryl group which may be substituted, and a C₅₋₁₀ heteroaryl group which may be substituted are also preferred, while a halogen atom, a hydroxyl group, an amino group which may be substituted, a C₁₋₁₀ alkoxy-carbonyl group which may be substituted with a halogen atom, an aminocarbonyl group which may have one or two substituents on the nitrogen atom, a C₆₋₁₀ aryl group which may be substituted, and a C₅₋₁₀ heteroaryl group which may be substituted are particularly preferred.

The “cyclohexenone ring which may be substituted” represented by ring B′ may have 1 to 3 substituents on the substitutable positions, and in the case of having a plurality of substituents, these substituents may be identical with or different from each other. For these substituents, the same ones as the aforementioned substituents for ring A are used. Among them, a C₁₋₁₀ alkyl group which may be substituted with a halogen atom, a C₆₋₁₄ aryl group which may be substituted, a C₅₋₁₀ heteroaryl group which may be substituted, an amino group which may be substituted, a cyclic imide group forming a fused ring together with ring B′, a C₁₋₁₀ alkoxy-carbonyl group which may be substituted with a halogen atom, a C₁₋₁₀ alkylsulfonyl group, an aminocarbonyl group which may have one or two substituents on the nitrogen atom, a thiocarbamoyl group which may have one or two substituents on the nitrogen atom, a carboxyl group, a hydroxyl group, a C₁₋₁₀ alkoxy group which may be substituted with 1 to 3 halogen atoms (for example, fluorine, chlorine, bromine, iodine), a cyano group, a nitro group, and a halogen atom are preferred, while a halogen atom, a hydroxyl group, an amino group which may be substituted, a C₁₋₁₀ alkoxy-carbonyl group which may be substituted with a halogen atom, an aminocarbonyl group which may have one or two substituents on the nitrogen atom, a C₆₋₁₀ aryl group which may be substituted, and a C₅₋₁₀ heteroaryl group which may be substituted are particularly preferred, and a C₆₋₁₀ aryl group which may be substituted and a C₅₋₁₀ heteroaryl group which may be substituted are also particularly preferred.

The “benzene ring which may be substituted” represented by ring B″ may have 1 to 3 substituents on the substitutable positions, and in the case of having a plurality of substituents, these substituents may be identical with or different from each other. For these substituents, the same ones as the aforementioned substituents for ring A are used. Among them, a C₁₋₁₀ alkyl group which may be substituted with a halogen atom, a C₆₋₁₄ aryl group which may be substituted, a C₅₋₁₀ heteroaryl group which may be substituted, an amino group which may be substituted, a cyclic imide group forming a fused ring together with ring B″, a C₁₋₁₀ alkoxy-carbonyl group which may be substituted with a halogen atom, a C₁₋₁₀ alkylsulfonyl group, an aminocarbonyl group which may have one or two substituents on the nitrogen atom, a thiocarbamoyl group which may have one or two substituents on the nitrogen atom, a carboxyl group, a hydroxyl group, a C₁₋₁₀ alkoxy group which may be substituted with 1 to 3 halogen atoms (for example, fluorine, chlorine, bromine, iodine), a cyano group, a nitro group, and a halogen atom are preferred, while a halogen atom, a hydroxyl group, an amino group which may be substituted, a C₁₋₁₀ alkoxy-carbonyl group which may be substituted with a halogen atom, an aminocarbonyl group which may have one or two substituents on the nitrogen atom, a C₆₋₁₀ aryl group which may be substituted, and a C₅₋₁₀ heteroaryl group which may be substituted are particularly preferred, and a C₆₋₁₀ aryl group which may be substituted and a C₅₋₁₀ heteroaryl group which may be substituted are also particularly preferred.

The “benzene ring which may be further substituted in addition to R³“represented by ring B′″ may have 1 to 3 substituents on the substitutable positions, and in the case of having a plurality of substituents, these substituents may be identical with or different from each other. For these substituents, the same ones as the aforementioned substituents for ring A are used. Among them, a C₁₋₁₀ alkyl group which may be substituted with a halogen atom, a C₆₋₁₄ aryl group which may be substituted, a C₅₋₁₀ heteroaryl group which may be substituted, an amino group which may be substituted, a cyclic imide group forming a fused ring together with ring B′″, a C₁₋₁₀ alkoxycarbonyl group which may be substituted with a halogen atom, a C₁₋₁₀ alkylsulfonyl group, an aminocarbonyl group which may have one or two substituents on the nitrogen atom, a thiocarbamoyl group which may have one or two substituents on the nitrogen atom, a carboxyl group, a hydroxyl group, a C₁₋₁₀ alkoxy group which may be substituted with 1 to 3 halogen atoms (for example, fluorine, chlorine, bromine, iodine), a cyano group, a nitro group, and a halogen atom are preferred, while a halogen atom, a hydroxyl group, an amino group which may be substituted, a C₁₋₁₀ alkoxy-carbonyl group which may be substituted with a halogen atom, an aminocarbonyl group which may have one or two substituents on the nitrogen atom, a C₆₋₁₀ aryl group which may be substituted, and a C₅₋₁₀ heteroaryl group which may be substituted are particularly preferred.

The “1,3-cyclohexanedione ring which may be substituted” represented by ring B″″ may have 1 to 3 substituents on the substitutable positions, and in the case of having a plurality of substituents, these substituents may be identical with or different from each other. For these substituents, the same ones as the aforementioned substituents for ring A are used. Among them, a C₁₋₁₀ alkyl group which may be substituted with a halogen atom, a C₆₋₁₄ aryl group which may be substituted, a C₅₋₁₀ heteroaryl group which may be substituted, an amino group which may be substituted, a cyclic imide group forming a fused ring together with ring B″″, a C₁₋₁₀ alkoxy-carbonyl group which may be substituted with a halogen atom, a C₁₋₁₀ alkylsulfonyl group, an aminocarbonyl group which may have one or two substituents on the nitrogen atom, a thiocarbamoyl group which may have one or two substituents on the nitrogen atom, a carboxyl group, a hydroxyl group, a C₁₋₁₀ alkoxy group which may be substituted with 1 to 3 halogen atoms (for example, fluorine, chlorine, bromine, iodine), a cyano group, a nitro group, and a halogen atom are preferred, while a halogen atom, a hydroxyl group, an amino group which may be substituted, a C₁₋₁₀ alkoxy-carbonyl group which may be substituted with a halogen atom, an aminocarbonyl group which may have one or two substituents on the nitrogen atom, a C₆₋₁₀ aryl group which may be substituted, and a C₅₋₁₀ heteroaryl group which may be substituted are particularly preferred.

The “C₁₋₁₀ alkylthio group” of the “C₁₋₁₀ alkylthio group which may be substituted” represented by R², R³, and R⁴ may be exemplified by methylthio, ethylthio, n-propylthio, isopropylthio, n-butylthio, isobutylthio, sec-butylthio, tert-butylthio, n-pentylthio, isopentylthio, neopentylthio, 1-methylpropylthio, n-hexylthio, isohexylthio, 1,1-dimethylbutylthio, 2,2-dimethylbutylthio, 3,3-dimethylbutylthio, 3,3-dimethylpropylthio, 2-ethylbutylthio, n-heptylthio, 1-methylheptylthio, 1-ethylhexylthio, n-octylthio, 1-methylheptylthio, nonylthio or the like. The “C₁₋₁₀ alkylthio group which may be substituted” represented by R² and R³ may have one to the maximum allowed number of substituents on any of the substitutable positions, and in the case of having a plurality of substituents, these substituents may be identical with or different from each other. Examples of these substituents include the aforementioned substituents for ring A. Among them, a halogen atom (for example, fluorine, chlorine, bromine, iodine), a C₁₋₁₀ alkoxy group, a mono- or di-C₁₋₁₀ alkylamino group are preferred, and in particular, fluorine is preferred.

The “amino group which may be substituted” represented by R² and R³ and R⁴ may have one or two substituents (may be monosubstituted or disubstituted), and in the case of being substituted with two substituents, the substituents may be identical with or different from each other. Examples of these substituents include a C₁₋₁₀ alkyl group (for example, methyl, ethyl), a C₁₋₁₀ alkyl-carbonyl group (for example, acetyl, isobutanoyl, isopentanoyl), a C₁₋₁₀ alkoxy-carbonyl group (for example, methoxycarbonyl, ethoxycarbonyl, propoxycarbonyl, tert-butoxycarbonyl), a C₁₋₁₀ alkanesulfonyl group (for example, methanesulfonyl), a C₆₋₁₀ arylsulfonyl group (for example, benzene sulfonyl, p-tolylsulfonyl), a C₅₋₁₀ heteroarylsulfonyl group (for example, 2-thienylsulfonyl, 3-pyridylsulfonyl,), a C₇₋₁₃ aralkyl group (for example, benzyl) and the like.

The “C₁₋₁₀ alkoxy group” of the “C₁₋₁₀ alkoxy group which may be substituted” represented by R² and R³ and R⁴ may be exemplified by methoxy, ethoxy, n-propoxy, isopropoxy, n-butoxy, isobutoxy, sec-butoxy, tert-butoxy, n-pentyloxy, isopentyloxy, neopentyloxy, 1-methylpropoxy, n-hexyloxy, isohexyloxy, 1,1-dimethylbutoxy, 2, 2-dimethylbutoxy, 3,3-dimethylbutoxy, 3,3-dimethylpropoxy, 2-ethylbutoxy, n-heptyloxy, 1-methylheptyloxy, 1-ethylhexyloxy, n-octyloxy, 1-methylheptyloxy, nonyloxy, or the like. Furthermore, the “C₁₋₁₀ alkoxy group which may be substituted” represented by R³ may have one to the maximum allowed number of substituents on any of the substitutable positions, and in the case of being substituted with two or more substituents, these substituents may be identical with or different from each other. Examples of these substituents include the aforementioned substituents for ring A. Among them, a halogen atom (for example, fluorine, chlorine, bromine, iodine), a C₁₋₁₀ alkoxy group, a mono- or di-C₁₋₁₀ alkylamino group are preferred, and in particular, fluorine is preferred.

The “C₂₋₁₀ alkenyl group which may be substituted” represented by R⁴ may be exemplified by a C₂₋₁₀ alkenyl group such as ethynyl, 1-propenyl, isopropenyl, 2-methyl-1-propenyl, 1-butenyl, 2-butenyl, 3-butenyl, 2-ethyl-1-butenyl, 1-pentenyl, 2-pentenyl, 3-penteny, 4-pentenyl, 4-methyl-3-penteny, 1-hexenyl, 2-hexenyl, 3-hexenyl, 4-hexenyl, and 5-hexenyl. Examples of the substituents thereof include an ester group, an amide group, an alcohol group, an acetal group, a C₆₋₁₄ aryl group which may be substituted, a C₅₋₁₀ heteroaryl group which may be substituted, and the like.

The “C₂₋₁₀ alkynyl group which may be substituted” represented by R⁴ may be exemplified by a C₂₋₁₀ alkynyl group such as acetynyl group, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. Examples of the substituents include an ester group, an amide group, an alcohol group, an acetal group, a C₆₋₁₄ aryl group which may be substituted, a C₅₋₁₀ heteroaryl group which may be substituted, a silyl group, and the like.

The “C₆₋₁₄ aryl group which may be substituted” represented by R⁴ may be exemplified by phenyl or naphthyl which may be substituted with a substituent selected from a halogen atom, a cyano group, a nitro group, a hydroxyl group, an amino group, a C₁₋₁₀ alkyl group which may be substituted with a halogen atom, C₂₋₁₀ alkenyl group, C₂₋₁₀ alkynyl group, a C₁₋₁₀ alkoxy-carbonyl group which may be substituted with a halogen atom, a C₁₋₁₀ alkylcarbonyl group which may be substituted with a halogen atom, a C₁₋₁₀ alkylaminocarbonyl group which may be substituted with a halogen atom, a di-C₁₋₁₀ alkylaminocarbonyl group which may be substituted with a halogen atom, a C₁₋₁₀ alkylsulfonyl group which may be substituted with a halogen atom, a C₁₋₁₀ alkylsulfinyl group which may be substituted with a halogen atom, a C₁₋₁₀ alkylthio group which may be substituted with a halogen atom, a C₁₋₁₀ alkylsulfonylamino group which may be substituted with a halogen atom, a C₁₋₁₀ alkylamino group which may be substituted with a halogen atom, a di-C₁₋₁₀ alkylamino group which may be substituted with a halogen atom, a C₁₋₁₀ alkoxycarbonylamino group which may be substituted with a halogen atom, and a C₁₋₁₀ alkylcarbonylamino group which may be substituted with a halogen atom.

The “C₅₋₁₀ heteroaryl group which may be substituted” represented by R⁴ may be exemplified by a 5- to 6-membered aromatic monocyclic heterocyclic group such as furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl or the like; a 8- to 12-membered aromatic fused heterocyclic group such as benzofuranyl, isobenzofuranyl, benzo[b]thienyl, indolyl, isoindolyl, 1H-indazolyl, benzindazolyl, benzoxazolyl, 1,2-benzoisoxazolyl, benzothiazolyl, benzopyranyl, 1,2-benzoisothiazolyl, 1H-benzotriazolyl, quinolyl, isoquinolyl, cinnolinyl, quinazolinyl, quinoxalinyl, phthalazinyl, naphthyridinyl, purinyl, pteridinyl, carbazolyl, α-carbolinyl, β-carbolinyl, γ-carbolinyl, acridinyl, phenoxazinyl, phenothiazinyl, phenazinyl, phenoxathiinyl, thianthrenyl, phenathridinyl, phenathrolinyl, indolizinyl, pyrrolo[1,2-b]pyridazinyl, pyrazolo[1,5-a]pyridyl, imidazo[1,2-a]pyridyl, imidazo[1,5-a]pyridyl, imidazo[1,2-b]pyridazinyl, imidazo[1,2-a]pyrimidinyl, 1,2,4-triazolo[4,3-a]pyridyl, 1,2,4-triazolo[4,3-b]pyridazinyl or the like which may be substituted with a substituent selected from a halogen atom, a cyano group, a nitro group, a hydroxyl group, an amino group, a C₁₋₁₀ alkyl group which may be substituted with a halogen atom, C₂₋₁₀ alkenyl group, C₂₋₁₀ alkynyl group, a C₁₋₁₀ alkoxycarbonyl group which may be substituted with a halogen atom, a C₁₋₁₀ alkylcarbonyl group which may be substituted with a halogen atom, a C₁₋₁₀ alkylaminocarbonyl group which may be substituted with a halogen atom, a di-C₁₋₁₀ alkylaminocarbonyl group which may be substituted with a halogen atom, a C₁₋₁₀ alkylsulfonyl group which may be substituted with a halogen atom, a C₁₋₁₀ alkylsulfinyl group which may be substituted with a halogen atom, a C₁₋₁₀ alkylthio group which may be substituted with a halogen atom, a C₁₋₁₀ alkylsulfonylamino group which may be substituted with a halogen atom, a C₁₋₁₀ alkylamino group which may be substituted with a halogen atom, a di-C₁₋₁₀ alkylamino group which may be substituted with a halogen atom, a C₁₋₁₀ alkoxycarbonylamino group which may be substituted with a halogen atom, and a C₁₋₁₀ alkylcarbonylamino group which may be substituted with a halogen atom.

A C₇₋₁₃ aralkylthio group represented by R⁴ may be exemplified by benzylthio, or the like.

A C₆₋₁₄ arylthio group represented by R⁴ may be exemplified by phenylthio, naphthylthio, or the like.

A C₂₋₁₀ alkenyloxy group represented by R⁴ may be exemplified by ethenyloxy, or the like.

A C₃₋₁₀ cycloalkoxy represented by R⁴ may be exemplified by cyclohexyloxy, or the like.

A C₇₋₁₃ aralkyloxy group represented by R⁴ may be exemplified by benzyloxy, or the like.

A C₆₋₁₄ aryloxy group represented by R⁴ may be exemplified by phenyloxy, naphthyloxy, or the like.

A C₁₋₁₀ alkyl-carbonyloxy group represented by R⁴ may be exemplified by acetyloxy, tert-butylcarbonyloxy, or the like.

Hereinafter, the methods for preparation of the invention will be explained in detail.

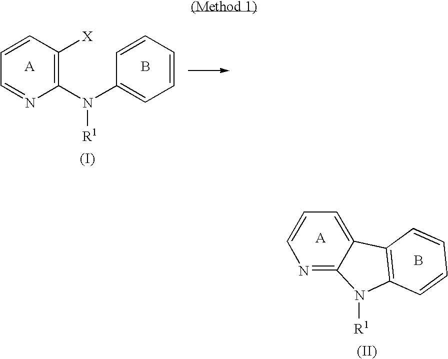

[In the formula, the symbols respectively represent the same meaning as defined above.]

An α-carboline derivative (II) can be obtained by subjecting an N-arylaminopyridine or N-heteroarylaminopyridine derivative (I) to a ring closure reaction in the presence of a palladium catalyst, a ligand and a base.

The N-arylaminopyridine or N-heteroarylaminopyridine derivative (I) may be a commercially available product, or may be synthesized according to (Method 2) or (Method 3) described below. Alternatively, the derivative may also be synthesized according to a method known per se, for example, the method described in Angew. Chem. Int. Ed., Vol. 42, p. 5400 (2003).

The present reaction can be performed in the absence or in the presence of a solvent. The solvent which may be used is not particularly limited as long as it does not affect the reaction, and examples thereof include aromatic hydrocarbons such as benzene, toluene, xylene and the like; aliphatic hydrocarbons such as hexane, pentane, heptane and the like; esters such as ethyl acetate, butyl acetate and the like; ethers such as diethyl ether, diisopropyl ether, t-butyl methyl ether, cyclopentyl methyl ether, tetrahydrofuran, 1,4-dioxane, anisole and the like; aliphatic halogenated hydrocarbons such as methylene chloride, chloroform, 1,2-dichloroethane and the like; alcohols such as methanol, ethanol, 1-propanol, 2-propanol, 1-butanol, 2-butanol, 2-methyl-1-propanol, t-butyl alcohol, and the like; amides such as N,N-dimethylformamide, N,N-dimethylacetamide, N-methylpiperidone and the like; dimethylsulfoxide, hexamethylphosphoric amide, dimethylimidazolidinone; nitrites such as acetonitrile, propionitrile and the like; ketones such as acetone, 2-butanone, water and the like. Among them, amides such as N,N-dimethylformamide, N,N-dimethylacetamide, N-methylpiperidone and the like are preferred, and in particular, N,N-dimethylacetamide is preferred. The amount of solvent to be used is preferably a 5- to 50-fold weight, more preferably a 5- to 30-fold weight, and particularly preferably a 5- to 20-fold weight, relative to the N-arylaminopyridine or N-heteroarylaminopyridine derivative (I).

The palladium catalyst used for the present reaction may be exemplified by a divalent palladium such as palladium acetate, palladium chloride, palladium bromide, palladium iodide, dichlorobis(benzonitrile)palladium (II), dichlorobis(acetonitrile)palladium (II) or the like; metallic palladium; palladium carbon; a zero-valent palladium such as bis(benzalacetone)palladium (0), tris(dibenzylideneacetone) dipalladium (0) or the like; a complex of a divalent to zero-valent palladium with a ligand to be described later (for example, tetrakis(triphenylphosphine)palladium, bis(tri-tert-butylphosphine)palladium, bis(triphenylphosphine)palladium dichloride, 1,1′-bis(diphenylphosphino)ferrocene palladium dichloride, 1,1′-bis(diphenylphosphino)ferrocene palladium dichloride-dichloromethane complex, 1,2-bis(diphenylphosphino)ethane palladium dichloride), or the like. Among them, palladium acetate, palladium chloride, tris(dibenzylideneacetone)dipalladium, 1,1′-bis(diphenylphosphino)ferrocene palladium dichloride, and 1,1′-bis(diphenylphosphino)ferrocene palladium dichloride-dichloromethane complex are preferred, and in particular, palladium acetate, and tris(dibenzylideneacetone)dipalladium are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the N-arylaminopyridine or N-heteroarylaminopyridine derivative (I).

The ligand to be used for the present reaction may be exemplified by an alkylphosphine ligand such as trimethylphosphine, triethylphosphine, tri-n-butylphosphine, di-tert-butylmethylphosphine, tri-tert-butylphosphine, tricyclohexylphosphine, butyl-di-1-adamantylphosphine, benzyl-di-1-adamantylphosphine, or the like; an alkylphosphonium ligand such as tri-n-butylphosphonium tetrafluoroborate, tri-tert-butylphosphonium tetrafluoroborate, di-tert-butylmethylphosphonium tetrafluoroborate, tricyclohexylphosphonium tetrafluoroborate, or the like; an arylphosphine ligand such as triphenylphosphine, tri-o-tolylphosphine, tri-p-tolylphosphine, tri(2-furyl)phosphine, tri(2-thienyl)phosphine, or the like; a bidentate phosphine ligand such as 1,2-bis(diphenylphosphino)ethane, 1,2-bis(diphenylphosphino)propane, 1,2-bis(diphenylphosphino)butane, αα′-bis(di-tert-butylphosphino)-o-xylene, or the like; a ferrocene type phosphine ligand such as 1,1′-bis(diphenylphosphino)ferrocene, 1,1′-bis(di-tert-butylphosphino)ferrocene, 1,1′-bis(diisopropylphosphino)ferrocene, 1,2,3,4,5-pentaphenyl-1′-(di-tert-butylphosphino)ferrocene, or the like; a biaryl type phosphine ligand such as 2,2′-bis(diphenylphosphino)-1,1′-binaphthyl, 2,2′-bis(di-p-tolylphosphino)-1,1′-binaphthyl, 2,2′-bis[di(3,5-xylyl)phosphino]-1,1′-binaphthyl, 2,2′-bis(diphenylphosphino)-1,1′-biphenyl, 2-di-tert-butylphosphino-1,1′-binaphthyl, 2-(di-tert-butylphosphino)-1,1′-biphenyl, 2-di-tert-butylphosphino-2′-(N,N-dimethylamino)biphenyl, 2-di-tert-butylphosphino-2′-methylbiphenyl, 2-(dicyclohexylphosphino)biphenyl, 2-(dicyclohexylphosphino)-2, 6′-dimethoxy-1,1′-biphenyl, 2-(dicyclohexylphosphino)-2′-(N,N-dimethylamino)biphenyl, 2-(dicyclohexylphosphino)-2′-methylbiphenyl, 2-(dicyclohexylphosphino)-2′,4′,6′-tri-isopropyl-1,1′-biphenyl, 2-(diphenylphosphino)-2′-(N,N-dimethylamino)biphenyl, or the like; a pyrrole type phosphine ligand such as N-phenyl-2-(di-tert-butylphosphino)pyrrole, N-phenyl-2-(dicyclohexylphosphino)pyrrole, or the like; a diphenyl ether type phosphine ligand such as 9,9-dimethyl-4,5-bis(diphenylphosphino)xanthene, bis(2-diphenylphosphinophenyl)ether, or the like; a carbene ligand such as 1,3-bis(2,6-diisopropylphenyl)-4,5-dihydroimidazolium tetrafluoroborate, 1,3-bis(2,6-diisopropylphenyl)-4,5-dihydroimidazolium chloride, 1,3-bis(2,4,6-trimethylphenyl)-4,5-dihydroimidazolium chloride, or the like; or the like. Among such ligands, an alkylphosphine ligand, an alkylphosphonium ligand, a ferrocene type phosphine ligand, and a biaryl type phosphine ligand are preferred, and di-tert-butylmethylphosphine, di-tert-butylmethylphosphonium tetrafluoroborate, tricyclohexylphosphine, tricyclohexylphosphonium tetrafluoroborate, 1,1′-bis(diphenylphosphino)ferrocene, 2-(dicyclohexylphosphino)biphenyl, and 2-(dicyclohexylphosphino)-2′-(N,N-dimethylamino)biphenyl are more preferred, with 1,1′-bis(diphenylphosphino)ferrocene, 2-(dicyclohexylphosphino)biphenyl, and 2-(dicyclohexylphosphino)-2′-(N,N-dimethylamino)biphenyl being even more preferred, and with 1,1′-bis(diphenylphosphino)ferrocene and 2-(dicyclohexylphosphino)biphenyl being particularly preferred. The amount of these ligands to be used is preferably 100 mol % or less, more preferably 0.001 mol % to 100 mol %, even more preferably 0.01 mol % to 50 mol %, and particularly preferably 1 mol % to 20 mol %, relative to the N-arylaminopyridine or N-heteroarylaminopyridine derivative (I).

The base to be used for the present reaction may be exemplified by an inorganic base such as cesium carbonate, potassium carbonate, sodium carbonate, lithium carbonate, potassium bicarbonate, sodium bicarbonate, cesium hydroxide, rubidium hydroxide, potassium hydroxide, sodium hydroxide, lithium hydroxide, cesium fluoride, potassium fluoride, sodium fluoride, tripotassium phosphate, or the like; an acetate such as cesium acetate, sodium acetate, potassium acetate, lithium acetate, or the like; a pivalate such as cesium pivalate, sodium pivalate, potassium pivalate, lithium pivalate, or the like; an alkali metal alkoxide such as potassium t-butoxide, sodium t-butoxide, sodium ethylate, potassium ethylate, sodium methylate, or the like; an alkali metal salt of hexamethyldisilazane such as lithium hexamethyldisilazide, sodium hexamethyldisilazide, potassium hexamethyldisilazide, or the like; a chain-like tertiary amine such as triethylamine, tributylamine, N,N-diisopropylethylamine, 1,8-bis(N,N-dimethylamino)naphthalene, or the like; a chain-like secondary amine such as diethylamine, dibutyl amine, or the like; a cyclic secondary amine such as piperidine, morpholine, pyrrolidine, or the like; a cyclic tertiary amine such as N-methylpiperidine, 1,5-diazabicyclo[4.3.0]non-5-ene (DBN), 1,8-diazabicyclo[5.4.0]undec-7-ene (DBU), 1,5-diazabicyclo[2.2.2]octane (DABCO), or the like; a heterocyclic aromatic amine such as pyridine, 4-(N,N-dimethylamino)pyridine, or the like; or the like. Among them, an organic base is preferred, while a cyclic tertiary amine such as 1,5-diazabicyclo[4.3.0]non-5-ene (DBN), 1,8-diazabicyclo[5.4.0]undec-7-ene (DBU), 1,5-diazabicyclo[2.2.2]octane (DABCO), or the like is more preferred, with 1,5-diazabicyclo[4.3.0]non-5-ene (DBN) and 1,8-diazabicyclo[5.4.0]undec-7-ene (DBU) being particularly preferred. The amount of these bases to be used is preferably 0.1- to 10-fold moles, more preferably 1- to 3-fold moles, and particularly preferably 1- to 2.5-fold moles, relative to the N-arylaminopyridine or N-heteroarylaminopyridine derivative (I).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

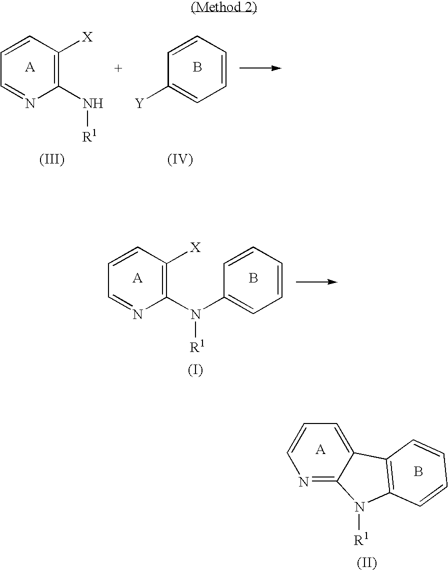

[In the Formula, the symbols respectively represent the same meaning as defined above.]

An α-carboline derivative (II) can be obtained by reacting an aminopyridine derivative (III) with a compound (IV) in the presence of a transition metal catalyst to obtain an N-arylaminopyridine or N-heteroarylaminopyridine derivative (I), and subsequently subjecting this N-arylaminopyridine or N-heteroarylaminopyridine derivative (I) to a ring closure reaction in the presence of a palladium catalyst, a ligand and a base.

(1) Reaction of Aminopyridine Derivative (III) and Compound (IV):

The aminopyridine derivative (III) may be a commercially available product, or may be synthesized according to a method known per se, for example the method described in Yamanaka, Hino, Nakagawa and Sakamoto, “Chemistry of Heterocyclic Compounds”, Kodansha, Ltd., 1988.

The compound (IV) may be a commercially available product, or may be synthesized by a method known per se, for example, the method described in the Chemical Society of Japan, “Lectures on Experimental Chemistry, 5^{th} Ed., Vol. 13, Synthesis of Organic Compounds I, Hydrocarbons and Halides”, Maruzen Co., Ltd., 2003.

The transition metal catalyst used in the present reaction may be exemplified by palladium, copper, or the like.

When the above mentioned transition metal catalyst is a palladium catalyst, the compound (I) can be synthesized by the following process.

The present reaction can be performed in the absence or in the presence of a solvent. For the solvent, the same solvents as those used in the (Method 1) described above can be used, but among them, aromatic hydrocarbons, alcohols, ethers and amides are preferred, with t-butanol, toluene, N,N-dimethylacetamide, and anisole being particularly preferred. The amount of the solvent to be used is preferably a 5- to 50-fold weight, more preferably a 5- to 30-fold weight, and particularly preferably a 5- to 20-fold weight, relative to the aminopyridine derivative (III).

The amount of the compound (IV) to be used is preferably 1 to 10 equivalents, and more preferably 1 to 5 equivalents, relative to the aminopyridine derivative (III).

For the palladium catalyst, the same catalysts as those used in the (Method 1) described above can be used, but among them, palladium chloride, palladium acetate and tris(dibenzylideneacetone)dipalladium are preferred, with palladium acetate being particularly preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the aminopyridine derivative (III).

For the present reaction, a ligand may be used together with the palladium catalyst. For such ligand, the same ligands as those used in the (Method 1) described above can be used, but among them, ferrocene type phosphine ligands and diphenyl ether type phosphine ligands are preferred, with 1,1′-bis(diphenylphosphino)ferrocene, 2,2′-bis(diphenylphosphino)-1,1′-binaphthyl, and 9,9-dimethyl-4,5-bis(diphenylphosphino)xanthene being particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the aminopyridine derivative (III).

For the present reaction, a base may also be used. For the base, the same bases as those used in the (Method 1) described above can be used, but among them, inorganic bases are preferred, with tripotassium phosphate, cesium carbonate and sodium t-butoxide being particularly preferred. The amount of these bases to be used is preferably 0.1- to 10-fold moles, more preferably 1- to 5-fold moles, and particularly preferably 1- to 2.5-fold moles, relative to the aminopyridine derivative (III).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

When the above mentioned transition metal catalyst is a copper catalyst, the compound (I) can be synthesized by the following process.

The present reaction can be performed in the absence or in the presence of a solvent. For the solvent, the same solvents as those used in the (Method 1) described above can be used, but among them, aromatic hydrocarbons, alcohols, ethers and amides are preferred, with t-butanol, toluene, anisole and N,N-dimethylacetamide, being particularly preferred. The amount of the solvent to be used is preferably a 5- to 50-fold weight, more preferably a 5- to 30-fold weight, and particularly preferably a 5- to 20-fold weight, relative to the aminopyridine derivative (III).

The amount of the compound (IV) to be used is preferably 1 to 10 equivalents, and more preferably 1 to 5 equivalents, relative to the aminopyridine derivative (III).

The copper catalyst used in the present reaction may be exemplified by copper, copper (I) iodide, copper (I) bromide, copper (II) bromide, copper (I) chloride, copper (II) chloride, copper (I) oxide, copper (II) oxide, copper (I) acetate, copper (II) acetate, copper (II) sulfate, copper (II) trifluorosulfonate, tetrakis(acetonitrile) copper (I) hexafluorophosphate, copper (II) acetylacetonate, bromotris(triphenylphosphine) copper (I), or the like. Among them, copper (I) iodide, copper (II) bromide, copper (I) chloride, and copper (I) acetate are preferred, and copper (I) iodide is particularly preferred. The amount of these copper catalysts to be used is preferably 100 mol % or less, more preferably 0.001 mol % to 100 mol %, even more preferably 0.01 mol % to 50 mol %, and particularly preferably 0.1 mol % to 20 mol %, relative to the aminopyridine derivative (III).

For the present reaction, a ligand may be used together with the copper catalyst. The ligand may be exemplified by diamines such as ethylenediamine, N,N-dimethylethylenediamine, N-methylethylenediamine, N,N′-dimethylethylenediamine, N,N-dimethylethylenediamine, N-butylethylenediamine, 1,2-diaminocyclohexane, N,N′-dimethylcyclohexane-1,2-diamine, N,N′-diethylcyclohexane-1,2-diamine, N,N′-diisopropylcyclohexane-1,2-diamine, N,N′-diacetylcyclohexane-1,2-diamine, and N,N,N″,N″-tetramethyl-1,2-cyclohexanediamine; diols such as ethylene glycol, propylene glycol, butylene glycol, 1,2-cyclohexanediol, pinacol, 2-methoxyethanol, diethylene glycol, and glycerol; amino acids such as L-proline, N-methylglycine, N,N-dimethylglycine: β-diketones such as 2-acetylcyclohexanone, dipivaloylmethane, 2-propionylcyclohexanone, and 2-isobutylcyclohexanone; 1,10-phenanthroline, neocuproine, ethanolamine, or the like. Among them, diamines and diols are preferred, with N,N′-dimethylethylenediamine, ethylene glycol, and ethanolamine, being particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the aminopyridine derivative (III).

For the present reaction, a base may also be used. For the base, the same bases as those used in the (Method 1) described above can be used, but among them, inorganic bases are preferred, while tripotassium phosphate, cesium carbonate, and potassium carbonate, are particularly preferred. The amount of these bases to be used is preferably 0.1- to 10-fold moles, more preferably 1- to 5-fold moles, and particularly preferably 1- to 2.5-fold moles, relative to the aminopyridine derivative (III).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

(2) Ring Closure Reaction:

The reaction can be performed in the same manner as in the (Method 1) described above.

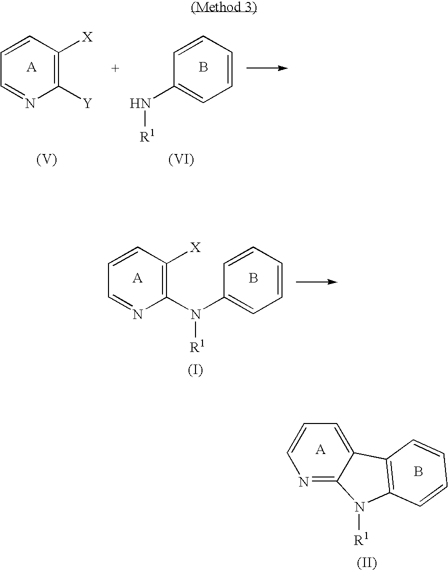

[In the Formula, the symbols respectively represent the same meaning as defined above.]

An α-carboline derivative (II) can be obtained by reacting a pyridine derivative (V) with an amine derivative (VI) to obtain an N-arylaminopyridine or N-heteroarylaminopyridine derivative (I), and subsequently subjecting this N-arylaminopyridine or N-heteroarylaminopyridine derivative (I) to a ring closure reaction in the presence of a palladium catalyst, a ligand and a base.

(1) Reaction of Pyridine Derivative (V) and Amine Derivative (VI):

The pyridine derivative (V) may be a commercially available product, or may be synthesized by a method known per se, for example, the method described in Yamanaka, Hino, Nakagawa and Sakamoto, “Chemistry of Heterocyclic Compounds”, Kodansha, Ltd., 1988.

The amine derivative (VI) may be a commercially available product, or may be synthesized by a method known per se, for example, the method described in the Chemical Society of Japan, “Lectures on Experimental Chemistry, 5^{th} Ed., Vol. 14, Synthesis of Organic Compounds II, Alcohols and Amines”, Maruzen Co., Ltd., 2003.

The present reaction can be performed in the absence or in the presence of a solvent. For the solvent, the same solvents as those used in the (Method 1) described above, lower aliphatic acids such as acetic acid, and the like can be used, but among them, aromatic hydrocarbons, alcohols, ethers, amides, and lower aliphatic acids are preferred, while t-butanol, toluene, xylene, cyclopentyl methyl ether, 1,4-dioxane, anisole, N,N-dimethylacetamide and acetic acid are particularly preferred. The amount of the solvent to be used is preferably a 5- to 50-fold weight, more preferably a 5- to 30-fold weight, and particularly preferably a 5- to 20-fold weight, relative to the pyridine derivative (V).

The amount of the amine derivative (VI) to be used is preferably 1 to 10 equivalents, and more preferably 1 to 5 equivalents, relative to the pyridine derivative (V).

For the present invention, a base may be used. For the base, the same bases as those used in the (Method 1) described above can be used, but among them, inorganic bases are preferred, while potassium acetate, tripotassium phosphate, cesium carbonate, and sodium t-butoxide are particularly preferred. The amount of these bases to be used is preferably 1- to 5-fold moles, more preferably 1- to 3-fold moles, and particularly preferably 1- to 2.5-fold moles, relative to the pyridine derivative (V).

The present reaction can be performed in the presence of a palladium catalyst. For such palladium catalyst, the same catalysts as those used in the (Method 1) described above can be used, but among them, palladium chloride, palladium acetate, and tris(dibenzylideneacetone)dipalladium are preferred, with palladium acetate being particularly preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the pyridine derivative (V).

For the present invention, a ligand may be used together with the palladium catalyst. For such ligand, the same ligands as those used in the (Method 1) described above can be used, but among them, ferrocene type phosphine ligands and diphenyl ether type phosphine ligands are preferred, with 1,1′-bis(diphenylphosphino)ferrocene, 2,2′-bis(diphenylphosphino)-1,1′-binaphthyl, and 9,9-dimethyl-4,5-bis(diphenylphosphino)xanthene being particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the pyridine derivative (V).

The reaction temperature is usually 0 to 200° C., preferably 10 to 150° C., and particularly preferably 25 to 150° C., while the reaction time is usually 1 to 100 hours, preferably 1 to 50 hours, and particularly preferably 1 to 25 hours.

(2) Ring Closure Reaction:

The reaction can be performed in the same manner as in the (Method 1) described above.

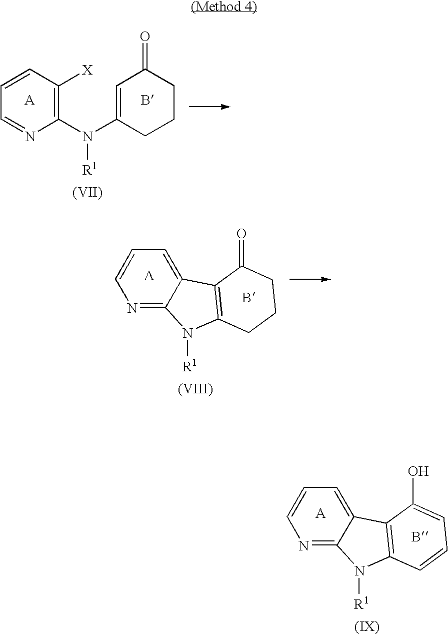

[In the formula, the symbols have the same meaning as defined above.]

An α-carboline derivative (IX) can be obtained by subjecting an N-pyridylenamine derivative (VII) to a ring closure reaction in the presence of a palladium catalyst, a ligand and a base to obtain a compound (VIII), and subsequently aromatizing the cyclohexenone ring (ring B′) of the compound (VIII).

(1) Ring Closure Reaction:

The N-pyridylenamine derivative (VII) may be synthesized according to (Method 5) to be described later, or may be synthesized according to a method known per se, for example, the method described in the Chemical Society of Japan, “Lectures on Experimental Chemistry, 5^{th} Ed., Vol. 14, Synthesis of Organic Compounds II, Alcohols and Amines”, Maruzen Co., Ltd., 2003.

The present reaction can be performed in the absence or in the presence of a solvent. For the solvent, the same solvents as those used in the (Method 1) described above can be used, but among them, aromatic hydrocarbons, alcohols, ethers and amides are preferred, while t-butanol, toluene, xylene, cyclopentyl methyl ether, and 1,4-dioxane are particularly preferred. The amount of the solvent to be used is preferably a 5- to 50-fold weight, more preferably a 5- to 30-fold weight, and particularly preferably a 5- to 20-fold weight, relative to the N-pyridylenamine derivative (VII).

For the palladium catalyst, the same catalysts as those used in the (Method 1) described above can be used, but among them, palladium chloride, palladium acetate, tris(dibenzylideneacetone)dipalladium, tetrakis(triphenylphosphine)palladium, bis(tri-tert-butylphosphine)palladium, bis(triphenylphosphine)palladium dichloride, 1,1′-bis(diphenylphosphino)ferrocene palladium dichloride, 1,1′-bis(diphenylphosphino)ferrocene palladium dichloride-dichloromethane complex, and 1,2-bis(diphenylphosphino)ethane palladium dichloride are preferred, while palladium acetate, tris(dibenzylideneacetone)dipalladium, tetrakis(triphenylphosphine)palladium, bis(tri-tert-butylphosphine)palladium, bis(triphenylphosphine)palladium dichloride, 1,1′-bis(diphenylphosphino)ferrocene palladium dichloride, and 1,1′-bis(diphenylphosphino)ferrocene palladium dichloride-dichloromethane complex are particularly preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the N-pyridylenamine derivative (VII).

For the ligand, the same ligands as those used in the (Method 1) described above can be used, but among them, arylphosphine ligands, alkylphosphine ligands, alkylphosphonium ligands, bidentate phosphine ligands, ferrocene type phosphine ligands and biaryl type phosphine ligands are preferred, while triphenylphosphine, 1,1′-bis(diphenylphosphino)ferrocene, tri-tert-butylphosphonium tetrafluoroborate, and tricyclohexylphosphonium tetrafluoroborate are particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the N-pyridylenamine derivative (VII).

For the base, the same bases as those used in the (Method 1) described above can be used, but among them, inorganic bases are preferred, with tripotassium phosphate and cesium carbonate being particularly preferred. 1,5-diazabicyclo[2.2.2]octane (DABCO) is also particularly preferred. The amount of these bases to be used is preferably 1- to 10-fold moles, more preferably 1- to 5-fold moles, further more preferably 1- to 3-fold moles and particularly preferably 1- to 2.5-fold moles, relative to the N-pyridylenamine derivative (VII).

The reaction temperature is usually 0 to 200° C., preferably 10 to 150° C., and particularly preferably 25 to 150° C., while the reaction time is usually 1 to 100 hours, preferably 1 to 50 hours, and particularly preferably 1 to 25 hours.

(2) Aromatization Reaction:

The aromatization reaction of the cyclohexenone ring of the compound (VIII) may be exemplified by a combination of halogenation of the ketone of the cyclohexenone ring at the α-position and a subsequent β-elimination reaction, a dehydrogenation reaction, a combination of an alkylidenation reaction of the ketone of the cyclohexenone ring at the α-position and a subsequent isomerization reaction of the double bond, or the like.

The halogenating agent that may be used for the halogenation of the ketone at the i-position may be exemplified by bromine, chlorine, iodine, N-bromosuccinimide, N-chlorosuccinimide, N-iodosuccinimide, sodium bromate, iodic acid, sodium iodate, 1,3-dibromo-5,5-dimethylhydantoin, tetra-n-butylammonium tribromide, pyridium hydrobromide perbromide, or the like. The β-elimination reaction can be performed by heating, and during the reaction, a base or a lithium salt (for example, lithium chloride, lithium bromide, lithium iodide) may be co-present. For the base, the same bases as those used in the (Method 1) described above can be used, but preferably lithium carbonate and lithium acetate can be used. In addition, the reaction conditions such as the types and the amount of use of the halogenating agent, base, lithium salt and solvent, as well as reaction temperature, reaction time and the like, may be appropriately determined in accordance with the type of the substituent of the compound (VIII) and the like, while referring to conventionally known halogenation reactions and β-elimination reactions.

The reagent that may be used for the dehydrogenation reaction may be exemplified by activated manganese dioxide, palladium carbon, Raney nickel, 2,3-dichloro-5,6-dicyanobenzoquinone, or the like. Furthermore, the reaction conditions such as the types and the amounts of use of the reagent and the solvent, as well as reaction temperature, reaction time and the like, may be appropriately determined in accordance with the type of the substituent of the compound (VIII) and the like, while referring to conventionally known dehydrogenation reactions.

The alkylidenation reaction of the ketone at the α-position can be performed by condensing the compound (VIII) with an aldehyde or a ketone. This reaction may also be performed in the presence of an appropriate base (for example, the bases used in the (Method 1) described above). The isomerization reaction of double bond can be performed by treating with an appropriate base (for example, 1,5-diazabicyclo[4.3.0]non-5-ene (DBN), 1,8-diazabicyclo[5.4.0]undec-7-ene (DBU), 1,5-diazabicyclo[2.2.2]octane (DABCO)). Furthermore, the reaction conditions such as the types and the amounts of use of the aldehyde, ketone, base and solvent, as well as reaction temperature, reaction time and the like, may be appropriately determined in accordance with the type of the substituent of the compound (VIII) and the like, while referring to conventionally known alkylidenation reactions and isomerization reactions.

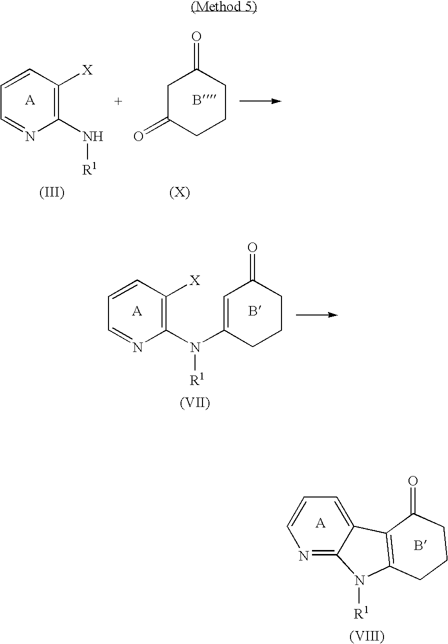

[In the Formula, the symbols respectively represent the same meaning as defined above.]

A compound (VIII) can be obtained by reacting an aminopyridine derivative (III) with a cyclohexanedione derivative (X) to obtain an N-pyridylenamine derivative (VII), and subsequently subjecting this N-pyridylenamine derivative (VII) to a ring closure reaction in the presence of a palladium catalyst, a ligand and a base.

(1) Reaction of Aminopyridine Derivative (III) and Cyclohexanedione Derivative (X):

The cyclohexanedione derivative (X) may be a commercially available product, or may be synthesized according to a method known per se, for example, the method described in J. Am. Chem. Soc., Vol. 78, p. 1645 (1950).

The present reaction can be performed in the absence or in the presence of a solvent. For the solvent, the same solvents as those used in the (Method 1) described above can be used, but among them, aromatic hydrocarbon solvents are preferred, with benzene, toluene and xylene being particularly preferred. The amount of the solvent to be used is preferably a 5- to 50-fold weight, more preferably a 5- to 30-fold weight, and particularly preferably a 5- to 20-fold weight, relative to the aminopyridine derivative (III).

The amount of the cyclohexanedione derivative (X) to be used is preferably 1 to 10 equivalents, and more preferably 1 to 5 equivalents, relative to the aminopyridine derivative (III).

The present reaction may be performed in the presence of an acid catalyst. Such acid catalyst may be exemplified by a mineral acid such as hydrochloric acid, sulfuric acid or the like; or an organic acid such as acetic acid, p-toluenesulfonic acid or the like. The amount of the acid catalyst to be used is preferably 100 mol % or less, more preferably 0.001 mol % to 100 mol %, even more preferably 0.01 mol % to 50 mol %, and particularly preferably 0.1 mol % to 20 mol %, relative to the aminopyridine derivative (III).

The reaction temperature is usually 0 to 200° C., preferably 10 to 150° C., and particularly preferably 25 to 150° C., while the reaction time is usually 1 to 100 hours, preferably 1 to 50 hours, and particularly preferably 1 to 25 hours.

(2) Ring Closure Reaction:

The reaction can be performed in the same manner as in the (Method 4) described above.

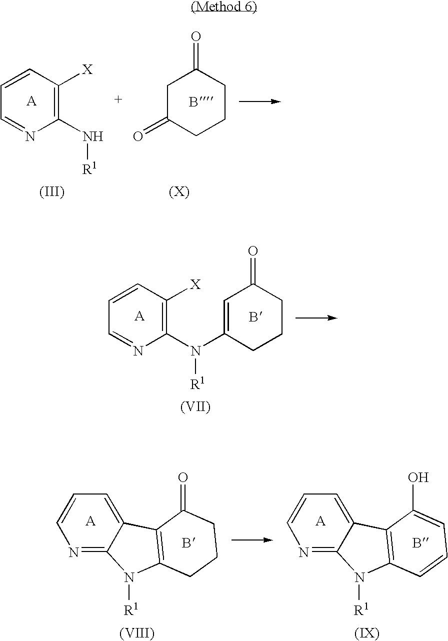

[In the Formula, the symbols respectively represent the same meaning as defined above.]

An α-carboline derivative (IX) can be obtained by reacting an aminopyridine derivative (III) with a cyclohexanedione derivative (X) to obtain an N-pyridylenamine derivative (VII), subsequently subjecting this N-pyridylenamine derivative (VII) to a ring closure reaction in the presence of a palladium catalyst, a ligand and a base to obtain a compound (VIII), and subsequently aromatizing the cyclohexenone ring (ring B′) of this compound (VIII).

(1) Reaction of Aminopyridine Derivative (III) and Cyclohexanedione Derivative (X):

The reaction can be performed in the same manner as in the (Method 5) described above.

(2) Ring Closure Reaction:

The reaction can be performed in the same manner as in the (Method 4) described above.

(3) Aromatization Reaction:

The reaction can be performed in the same manner as in the (Method 4) described above.

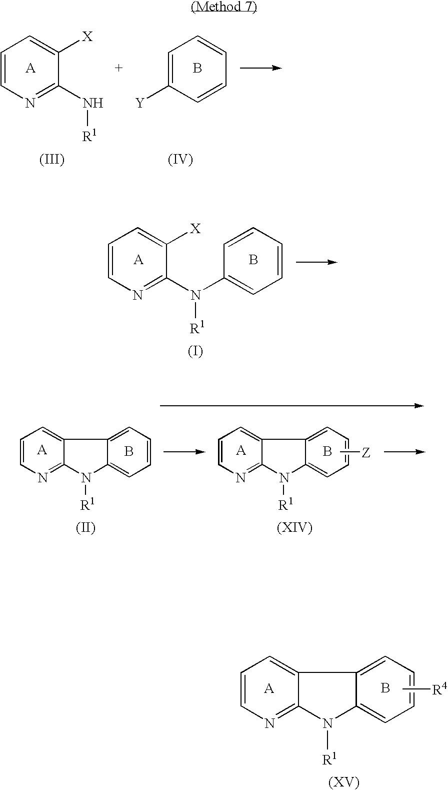

(1) Reaction of Aminopyridine Derivative (III) and Compound (IV):

The reaction can be performed in the same manner as in the (Method 2) described above.

(2) Ring Closure Reaction:

The reaction can be performed in the same manner as in the (Method 1) described above.

(3) Reaction for Introducing Leaving Group:

For the compound (II), when a leaving group does not exist on the ring B, a leaving group represented by Z such as a halogen atom, a C₁₋₄ alkanesulfonyloxy group which may be halogenated, a benzenesulfonyloxy group which may be substituted, and the like, can be introduced onto the ring B, by the reaction such as (1) a direct halogenation reaction of the ring B, (2) a reaction for converting an amino group which is a substituent on the ring B into halogen, (3) a reaction for converting a hydroxyl group which is a substituent on the ring B into a leaving group.

In addition, for the compound (II), when the compound (II) contains a substituent on the ring B, in which the substituent is a leaving group, a substituent R⁴ can be introduced onto the ring B, by being directly subjected to the coupling reaction to be described as follow, without being subjected to the reaction for introducing a leaving group.

Reaction for introducing leaving group 1 (direct halogenation reaction):

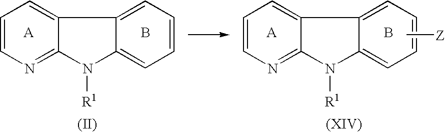

The leaving group Z (a halogen atom) can be introduced onto the ring B in the compound (II), by reacting the compound (II) (an α-carboline derivative) with a halogenating agent.

The halogenating agent may be exemplified by bromine, chlorine, iodine, N-bromosuccinimide, N-chlorosuccinimide, N-iodosuccinimide, sodium bromate, iodic acid, sodium iodate, 1,3-dibromo-5,5-dimethylhydantoin, tetra-n-butylammonium tribromide, pyridium hydrobromide perbromide, or the like. Among them, N-bromosuccinimide, N-iodosuccinimide, sodium bromate, sodium iodate, 1,3-dibromo-5,5-dimethylhydantoin, tetra-n-butylammonium tribromide, and pyridium hydrobromide perbromide are preferred. The amount of the halogenating agent to be used is preferably 1- to 15-fold moles, more preferably 1- to 10-fold moles, and particularly preferably 1- to 5-fold moles, relative to the compound (II).

The present reaction can be performed in the absence or in the presence of a solvent. For the solvent, the same solvents as those used in the (Method 1) described above can be used, but among them, aromatic hydrocarbons, ethers, and nitriles are preferred, while toluene, tetrahydrofuran, and acetonitrile are particularly preferred. The amount of the solvent to be used is preferably a 5- to 50-fold weight, more preferably a 5- to 30-fold weight, and particularly preferably a 5- to 20-fold weight, relative to the compound (II).

For the present reaction, acid may also used. The acid may be exemplified by methanesulfonic acid, p-toluenesulfonic acid, trifluoromethanesulfonic acid, trifluoroacetic acid, acetic acid, hydrochloric acid, sulfuric acid, nitric acid, or the like. Among them, methanesulfonic acid, p-toluenesulfonic acid, hydrochloric acid, and sulfuric acid are preferred, while methanesulfonic acid, and sulfuric acid are particularly preferred. The amount of these acids to be used is preferably 0.1- to 10-fold moles, more preferably 1- to 5-fold moles, and particularly preferably 1- to 2.5-fold moles, relative to the compound (II).

The reaction temperature is usually 0 to 200° C., preferably 0 to 150° C., and particularly preferably 0 to 100° C., while the reaction time is usually 1 to 100 hours, preferably 1 to 50 hours, and particularly preferably 1 to 25 hours.

Reaction for introducing leaving group 2 (reaction for converting an amino group into halogen):

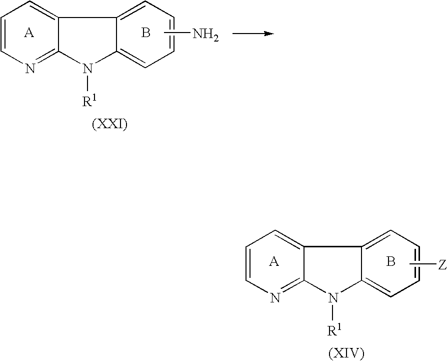

For the compound (XXI) containing an amino group as the substituent on the ring B of the compound (II) (α-carboline derivative), the amino group thereof can be transformed into a leaving group Z (a halogen atom) by reacting with nitrite salt in the presence of acid to obtain diazonium salt, subsequently decomposing the obtained diazonium salt in the presence of a halide salt.

The present reaction can be performed in the absence or in the presence of a solvent. For the solvent, the same solvents as those used in the (Method 1) described above can be used, but among them, aromatic hydrocarbons, ethers, nitrites, and water are preferred, while toluene, tetrahydrofuran, acetonitrile, and water are particularly preferred. The amount of the solvent to be used is preferably a 5- to 50-fold weight, more preferably a 5- to 30-fold weight, and particularly preferably a 5- to 20-fold weight, relative to aminocarboline (compound (XXI)).

The acid used in the present reaction may be exemplified by methanesulfonic acid, p-toluenesulfonic acid, trifluoromethanesulfonic acid, trifluoroacetic acid, acetic acid, hydrochloric acid, sulfuric acid, nitric acid, hydrobromic acid or the like. Among them, acetic acid, hydrochloric acid, sulfuric acid, and hydrobromic acid are preferred, while hydrochloric acid, sulfuric acid, and hydrobromic acid are particularly preferred. The amount of these acids to be used is preferably 0.1- to 20-fold moles, more preferably 1- to 10-fold moles, and particularly preferably 1- to 5-fold moles, relative to aminocarboline (compound (XXI)).

The nitrite salt used in the present reaction may be exemplified by sodium nitrite, potassium nitrite, silver nitrite, or the like. Among them, sodium nitrite is preferred. The amount of these nitrite salts to be used is preferably 0.1- to 10-fold moles, more preferably 1- to 5-fold moles, and particularly preferably 1- to 2.5-fold moles, relative to aminocarboline (compound (XXI)).

The halide salt used in the present reaction may be exemplified by copper halide such as copper (I) iodide, copper (I) bromide, copper (II) bromide, copper (I) chloride, and copper (II) chloride, sodium halide such as sodium iodide, potassium halide such as potassium iodide, or the like. Among them, copper (I) iodide, copper (I) bromide, copper (I) chloride, sodium iodide, and potassium iodide are particularly preferred. The amount of these halide ions to be used is preferably 0.1- to 10-fold moles, more preferably 1- to 5-fold moles, and particularly preferably 1- to 2.5-fold moles, relative to aminocarboline (compound (XXI)).

The reaction temperature is usually 0 to 200° C., preferably 0 to 150° C., and particularly preferably 0 to 100° C., while the reaction time is usually 1 to 100 hours, preferably 1 to 50 hours, and particularly preferably 1 to 25 hours.

Reaction for introducing leaving group 3 (reaction for converting a hydroxyl group into a leaving group):

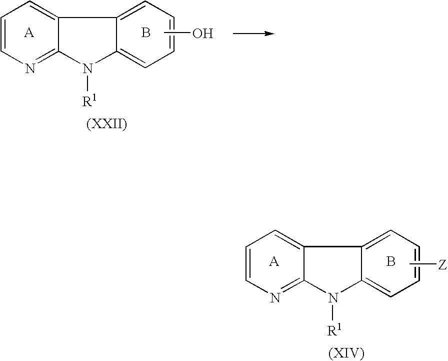

For the compound (XXII) containing a hydroxyl group as the substituent on the ring B of the compound (II) (α-carboline derivative), the hydroxyl group thereof can be converted to a leaving group Z such as a halogen atom, a C₁₋₄ alkanesulfonyloxy group which may be halogenated, a benzenesulfonyloxy group which may be substituted, a N,N-dialkylaminocarbonyloxy group, and an N,N-dialkylaminothiocarbonyloxy group.

For the compound (XXII), the hydroxyl group thereof can be converted to a halogen atom by reacting with a halogenating agent. The halogenating agent may be exemplified by phosphorous oxychloride, phosphorus pentachloride, phosphorus trichloride, phosphorus oxybromide, phosphorus tribromide, chlorine, bromine, or the like. Among them, phosphorous oxychloride and phosphorus pentachloride are particularly preferred. The amount of these halogenating agents to be used is preferably 0.1- to 10-fold moles, more preferably 1- to 5-fold moles, and particularly preferably 1- to 2.5-fold moles, relative to carboline (compound (XXII)) containing a hydroxyl group.

For the compound (XXII), the hydroxyl group thereof can be converted to a C₁₋₄ alkanesulfonyloxy group which may be halogenated or a benzenesulfonyloxy group which may be substituted, by reacting with a sulfonating agent. The sulfonating agent may be exemplified by a sulfonic anhydride such as trifluoromethanesulfonic anhydride, methanesulfonic anhydride, benzenesulfonic anhydride, a C₁₋₁₀ alkylsulfonyl chloride which may be substituted such as methanesulfonyl chloride, or C₆₋₁₄ aryl sulfonyl chloride which may be substituted such as p-toluenesulfonyl chloride. Among them, trifluoromethanesulfonic anhydride, methanesulfonic anhydride, p-toluenesulfonic anhydride, methanesulfonyl chloride, and p-toluenesulfonyl chloride are preferred. The amount of these sulfonating agents to be used is preferably 0.1- to 10-fold moles, more preferably 1- to 5-fold moles, and particularly preferably 1- to 2.5-fold moles, relative to carboline (compound (XXII)) containing a hydroxyl group.

For the compound (XXII), the hydroxyl group thereof can be converted to a N,N-dialkylaminocarbonyloxy group, by reacting with a N,N-dialkylaminocarbonylating agent. The N,N-dialkylaminocarbonylating agent may be exemplified by C₁₋₁₀ alkylcarbamoyl chloride such as dimethylcarbamoyl chloride and diethylcarbamoyl chloride. Among them, diethylcarbamoyl chloride is particularly preferred. The amount of these N,N-dialkylaminocarbonylating agents to be used is preferably 0.1- to 10-fold moles, more preferably 1- to 5-fold moles, and particularly preferably 1- to 2.5-fold moles, relative to carboline (compound (XXII)) containing a hydroxyl group.

For the compound (XXII), the hydroxyl group thereof can be converted to a N,N-dialkylaminothiocarbonyloxy group, by reacting with a N,N-dialkylaminothiocarbonylating agent. The N,N-dialkylaminothiocarbonylating agent may be exemplified by C₁₋₁₀ alkylthiocarbamoyl chloride such as dimethylthiocarbamoyl chloride and diethylthiocarbamoyl chloride. Among them, diethylthiocarbamoyl chloride is particularly preferred. The amount of these N,N-dialkylaminothiocarbonylating agents to be used is preferably 0.1- to 10-fold moles, more preferably 1- to 5-fold moles, and particularly preferably 1- to 2.5-fold moles, relative to carboline (compound (XXII)) containing a hydroxyl group.

The present reaction can be performed in the absence or in the presence of a solvent. For the solvent, the same solvents as those used in the (Method 1) described above can be used, but among them, aromatic hydrocarbons, aliphatic halogenated hydrocarbons, ethers, nitriles, and water are preferred, while toluene, pyridine, methylene chloride, tetrahydrofuran, acetonitrile, and water are particularly preferred. The amount of the solvent to be used is preferably a 5- to 50-fold weight, more preferably a 5- to 30-fold weight, and particularly preferably a 5- to 20-fold weight, relative to carboline (compound (XXII)) containing a hydroxyl group.

For the present reaction, a base may also be used. For the base, the same bases as those used in the (Method 1) described above can be used, but among them, an inorganic base, heterocyclic aromatic amine, and chain-like tertiary amine are preferred, while tripotassium phosphate, sodium carbonate, potassium carbonate, pyridine, triethylamine, and diisopropylethylamine are preferred. The amount of these bases to be used is preferably 0.1- to 10-fold moles, more preferably 1- to 5-fold moles, and particularly preferably 1- to 2.5-fold moles, relative to carboline (compound (XXII)) containing a hydroxyl group.

The reaction temperature is usually 0 to 200° C., preferably 0 to 150° C., and particularly preferably 0 to 100° C., while the reaction time is usually 1 to 100 hours, preferably 1 to 50 hours, and particularly preferably 1 to 25 hours.

(4) Coupling Reaction:

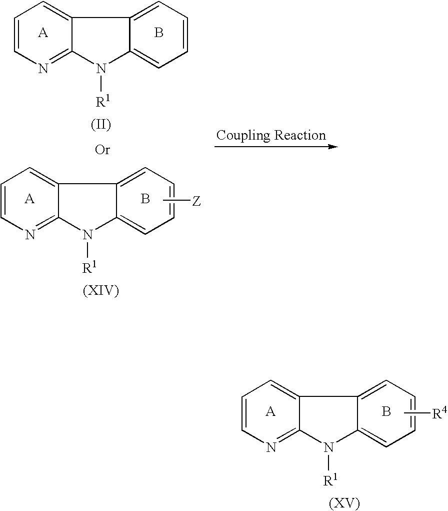

When the compound (XIV) (or the compound (II) in which the substituent on the ring B is a leaving group) is subjected to various cross-coupling reactions (for example, Suzuki reaction, Kumada reaction, Negishi reaction, Migita-Stille reaction, Mizoroki-Heck reaction, Sonogashira reaction, cyanation reaction, reaction for introducing hetero atom, carbon monoxide insertion reaction, and the like) in the presence of transition metal catalysts (for example, palladium catalyst, nickel catalyst), as described by F. Diederich and P. J. Stang, “Metal-catalyzed Cross-coupling Reactions”, Wiley-VCH, 1998, a substituent R⁴ such as an aromatic group, a heterocyclic aromatic group, an alkyl group, an alkenyl group, an alkynyl group, a carbonyl group, a cyano group or the like can be introduced onto ring B.

(4-1) When the above cross-coupling reaction is the Suzuki reaction, for the compound (XIV) obtained as described above (or the compound (II) in which the substituent on the ring B is a leaving group), a substituent R⁴ such as an aromatic group, a heterocyclic aromatic group, an alkyl group, an alkynyl group, and an alkenyl group can be introduced by reacting with an organoboron compound represented by the Formula:

(L)₂B—R⁴

in the presence of a transition metal catalyst.

In the Formula (L)₂B—R⁴, R⁴ may be exemplified by a substituent described above. A C₆₋₁₄ aryl group which may be substituted, a C₅₋₁₀ heteroaryl group which may be substituted, C₁₋₁₀ alkyl group which may be substituted, a C₂₋₁₀ alkynyl group which may be substituted, and a C₂₋₁₀ alkenyl group which may be substituted are particularly preferred.

L represents a hydroxyl group, a halogen atom, a C₁₋₁₀ alkyl group which may be substituted, or the like. Alternatively, an organoboron compound represented by the Formula (L)₂B—R⁴ includes the compounds represented by the following Formula:

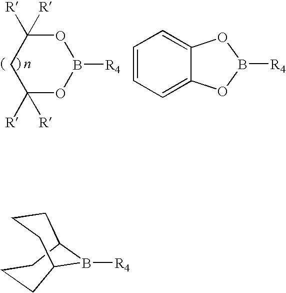

(in the Formula, R′ represents hydrogen, or a C₁₋₁₀ alkyl group which may be substituted, n is an integer of 1 to 5, and R⁴ is as described above.)

The amount of these organoboron compounds to be used is preferably 1- to 10-fold moles, more preferably 1- to 5-fold moles, and particularly preferably 1- to 2.5-fold moles, relative to the compound (XIV) (or the compound (II) in which the substituent on the ring B is a leaving group).

The present reaction can be performed in the absence or in the presence of a solvent. For the solvent, the same solvents as those used in the (Method 1) described above can be used, but among them, aromatic hydrocarbons, alcohols, ethers, amides, and water are preferred, with toluene, N,N-dimethylacetamide, tetrahydrofuran, 1,2-dimethoxyethane, and water being particularly preferred. The amount of the solvent to be used is preferably a 5- to 50-fold weight, more preferably a 5- to 30-fold weight, and particularly preferably a 5- to 20-fold weight, relative to the compound (XIV) (or the compound (II) in which the substituent on the ring B is a leaving group).

The transition metal catalyst used in the present reaction may be exemplified by palladium, nickel, or the like. For the palladium catalyst, the same catalysts as those used in the (Method 1) described above can be used, but among them, palladium acetate, palladium chloride, tris(dibenzylideneacetone)dipalladium (0), tetrakis(triphenylphosphine)palladium (0), bis(tri-tert-butylphosphine)palladium, bis(triphenylphosphine)palladium dichloride, 1,1′-bis(diphenylphosphino)ferrocene palladium dichloride, 1,1′-bis(diphenylphosphino)ferrocene palladium dichloride-dichloromethane complex, and palladium carbon are preferred, and in particular, palladium chloride, palladium acetate, tetrakis(triphenylphosphine)palladium(0), bis(triphenylphosphine)palladium dichloride, 1,1′-bis(diphenylphosphino)ferrocene palladium dichloride, and palladium carbon are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group). A nickel catalyst may be exemplified by nickel (II) acetylacetonate, tetrakis(triphenylphosphine)nickel(0), nickel chloride, bis(triphenylphosphine)nickel dichloride, bis(triphenylphosphine)nickel dibromide, bis(1,5-cyclooctadiene)nickel(0), 1,1′-bis(diphenylphosphino)ferrocene nickel dichloride, or 1,2-bis(diphenylphosphino)ethane nickel dichloride. Among them, bis(triphenylphosphine)nickel dichloride, nickel (II) acetylacetonate, and tetrakis(triphenylphosphine)nickel (0) are preferred. The amount of these nickel catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a ligand may be used together with the palladium catalyst or the nickel catalyst. For such ligand, the same ligands as those used in the (Method 1) described above can be used, but among them, alkyl phosphine ligands, aryl phosphine ligands, alkyl phosphonium ligands, ferrocene type phosphine ligands, and biaryl type phosphine ligands, are preferred, tri-tert-butylphosphine, triphenylphosphine, tri-o-tolylphosphine, di-tert-butylmethylphosphine, di-tert-butylmethylphosphonium tetrafluoroborate, tricyclohexylphosphine, tricyclohexylphosphonium tetrafluoroborate, 1,1′-bis(diphenylphosphino)ferrocene, 2-(dicyclohexylphosphino)biphenyl, 2-(dicyclohexylphosphino)-2′-(N,N-dimethylamino)biphenyl are more preferred, and tri-tert-butylphosphine, triphenylphosphine, tricyclohexylphosphine, 1,1′-bis(diphenylphosphino)ferrocene, 2-(dicyclohexylphosphino)biphenyl, 2-(dicyclohexylphosphino)-2′-(N,N-dimethylamino)biphenyl are particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a base may also be used. For the base, the same bases as those used in the (Method 1) described above can be used, but among them, inorganic bases are preferred, with tripotassium phosphate, cesium carbonate, sodium carbonate, and potassium carbonate being particularly preferred. The amount of these bases to be used is preferably 0.1- to 10-fold moles, more preferably 1- to 5-fold moles, and particularly preferably 1- to 2.5-fold moles, relative to the compound (XIV) (or the compound (II) in which the substituent on the ring B is a leaving group).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

(4-2) When the above cross-coupling reaction is the Kumada reaction, for the compound (XIV) obtained as described above (or the compound (II) in which the substituent on the ring B is a leaving group), a substituent R⁴ such as an aromatic group, a heterocyclic aromatic group, an alkyl group, an alkynyl group, and an alkenyl group can be introduced by reacting with a Grignard reagent represented by the Formula:

LMg—R⁴

in the presence of a transition metal catalyst.

In the Formula LMg—R⁴, R⁴ may be exemplified by a substituent described above. Among them, a C₆₋₁₄ aryl group which may be substituted, a C₅₋₁₀ heteroaryl group which may be substituted, C₁₋₁₀ alkyl group which may be substituted, a C₂₋₁₀ alkynyl group which may be substituted, and a C₂₋₁₀ alkenyl group which may be substituted are particularly preferred.

L represents a halogen atom (e.g., chlorine, bromine, and iodine).

The amount of these Grignard reagents to be used is preferably 1- to 10-fold moles, more preferably 1- to 5-fold moles, and particularly preferably 1- to 2.5-fold moles, relative to the compound (XIV) (or the compound (II) in which the substituent on the ring B is a leaving group).

The present reaction can be performed in the absence or in the presence of a solvent. For the solvent, the same solvents as those used in the (Method 1) described above can be used, but among them, aromatic hydrocarbons, ethers, and amides are preferred, with toluene, N,N-dimethylacetamide, tetrahydrofuran, and 1,2-dimethoxyethane being particularly preferred. The amount of the solvent to be used is preferably a 5- to 50-fold weight, more preferably a 5- to 30-fold weight, and particularly preferably a 5- to 20-fold weight, relative to the compound (XIV) (or the compound (II) in which the substituent on the ring B is a leaving group).

The transition metal catalyst used in the present reaction may be exemplified by palladium, nickel, or the like. For the palladium catalyst, the same catalysts as those used in the (Method 1) described above can be used, but among them, tetrakis(triphenylphosphine)palladium(0), bis(triphenylphosphine)palladium dichloride, and 1,1′-bis(diphenylphosphino)ferrocene palladium dichloride are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group). A nickel catalyst may be exemplified by nickel (II) acetylacetonate, tetrakis(triphenylphosphine)nickel(0), nickel chloride, bis(triphenylphosphine)nickel dichloride, bis(triphenylphosphine)nickel dibromide, bis(1,5-cyclooctadiene)nickel(0), 1,1′-bis(diphenylphosphino)ferrocene nickel dichloride, or 1,2-bis(diphenylphosphino)ethane nickel dichloride. Among them, bis(triphenylphosphine)nickel dichloride, nickel (II) acetylacetonate, and tetrakis(triphenylphosphine)nickel(0) are preferred. The amount of these nickel catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a ligand may be used together with the palladium catalyst or the nickel catalyst. For such ligand, the same ligands as those used in the (Method 1) described above can be used, but among them, ferrocene type phosphine ligands and alkyl phosphine ligands are preferred, with 1,1′-bis(diphenylphosphino)ferrocene, 1,1′-bis(di-tert-butylphosphino)ferrocene, 1,1′-bis(diisopropylphosphino)ferrocene and triphenylphosphine being particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

(4-3) When the above cross-coupling reaction is the Negishi reaction, for the compound (XIV) obtained as described above (or the compound (II) in which the substituent on the ring B is a leaving group), a substituent R⁴ such as an aromatic group, a heterocyclic aromatic group, an alkyl group, an alkynyl group, and an alkenyl group can be introduced by reacting with an organozinc reagent represented by the Formula:

LZn—R⁴

in the presence of a transition metal catalyst.

In the Formula LZn—R⁴, R⁴ may be exemplified by a substituent described above. Among them, a C₆₋₁₄ aryl group which may be substituted, a C₅₋₁₀ heteroaryl group which may be substituted, C₁₋₁₀ alkyl group which may be substituted, a C₂₋₁₀ alkynyl group which may be substituted, and a C₂₋₁₀ alkenyl group which may be substituted are particularly preferred.

L represents a halogen atom (e.g., chlorine, bromine, and iodine).

The amount of these organozinc reagents to be used is preferably 1- to 10-fold moles, more preferably 1- to 5-fold moles, and particularly preferably 1- to 2.5-fold moles, relative to the compound (XIV) (or the compound (II) in which the substituent on the ring B is a leaving group).

The present reaction can be performed in the absence or in the presence of a solvent. For the solvent, the same solvents as those used in the (Method 1) described above can be used, but among them, aromatic hydrocarbons, alcohols, ethers, and amides are preferred, with toluene, N,N-dimethylacetamide, tetrahydrofuran, and 1,2-dimethoxyethane being particularly preferred. The amount of the solvent to be used is preferably a 5- to 50-fold weight, more preferably a 5- to 30-fold weight, and particularly preferably a 5- to 20-fold weight, relative to the compound (XIV) (or the compound (II) in which the substituent on the ring B is a leaving group).

The transition metal catalyst used in the present reaction may be exemplified by palladium or the like. For the palladium catalyst, the same catalysts as those used in the (Method 1) described above can be used, but among them, palladium chloride, palladium acetate, tris(dibenzylideneacetone)dipalladium, tetrakis(triphenylphosphine)palladium (0), bis(triphenylphosphine)palladium dichloride, and 1,1′-bis(diphenylphosphino)ferrocene palladium dichloride are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a ligand may be used together with the palladium catalyst. For such ligand, the same ligands as those used in the (Method 1) described above can be used, but among them, ferrocene type phosphine ligands and aryl phosphine ligands are preferred, with 1,1′-bis(diphenylphosphino)ferrocene, 1,1′-bis(di-tert-butylphosphino)ferrocene, 1,1′-bis(diisopropylphosphino)ferrocene, triphenylphosphine tris(2-furyl)phosphine, and tri-o-tolylphosphine being particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

(4-4) When the above cross-coupling reaction is the Migita-Stille reaction, for the compound (XIV) obtained as described above (or the compound (II) in which the substituent on the ring B is a leaving group), a substituent R⁴ such as an aromatic group, a heterocyclic aromatic group, an alkyl group, an alkynyl group, and an alkenyl group can be introduced by reacting with an organotin reagent represented by the Formula:

(L)₃Sn—R⁴

in the presence of a transition metal catalyst.

In the Formula (L)₃Sn—R⁴, R⁴ may be exemplified by a substituent described above. Among them, a C₆₋₁₄ aryl group which may be substituted, a C₅₋₁₀ heteroaryl group which may be substituted, C₁₋₁₀ alkyl group which may be substituted, a C₂₋₁₀ alkynyl group which may be substituted, and a C₂₋₁₀ alkenyl group which may be substituted are particularly preferred.

L represents an alkyl group (e.g., methyl, ethyl, and butyl).

The amount of these organotin reagents to be used is preferably 1- to 10-fold moles, more preferably 1- to 5-fold moles, and particularly preferably 1- to 2.5-fold moles, relative to the compound (XIV) (or the compound (II) in which the substituent on the ring B is a leaving group).

The present reaction can be performed in the absence or in the presence of a solvent. For the solvent, the same solvents as those used in the (Method 1) described above can be used, but among them, aromatic hydrocarbons, ethers, and amides are preferred, with toluene, N,N-dimethylformamide, N,N-dimethylacetamide, tetrahydrofuran, and 1,2-dimethoxyethane being particularly preferred. The amount of the solvent to be used is preferably a 5- to 50-fold weight, more preferably a 5- to 30-fold weight, and particularly preferably a 5- to 20-fold weight, relative to the compound (XIV) (or the compound (II) in which the substituent on the ring B is a leaving group).

The transition metal catalyst used in the present reaction may be exemplified by palladium, nickel, or the like. For the palladium catalyst, the same catalysts as those used in the (Method 1) described above can be used, but among them, palladium chloride, palladium acetate, tetrakis(triphenylphosphine)palladium (0), tris(dibenzylideneacetone)dipalladium, bis(triphenylphosphine)palladium dichloride, and 1,1′-bis(diphenylphosphino)ferrocene palladium dichloride are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group). A nickel catalyst may be exemplified by nickel (II) acetylacetonate, tetrakis(triphenylphosphine)nickel (0), nickel chloride, bis(triphenylphosphine)nickel dichloride, bis(triphenylphosphine)nickel dibromide, bis(1,5-cyclooctadiene)nickel (0), 1,1′-bis(diphenylphosphino)ferrocene nickel dichloride, or 1,2-bis(diphenylphosphino)ethane nickel dichloride. Among them, nickel (II) acetylacetonate and tetrakis(triphenylphosphine)nickel (0) are preferred. The amount of these nickel catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a ligand may be used together with the palladium catalyst or the nickel catalyst. For such ligand, the same ligands as those used in the (Method 1) described above can be used, but among them, alkyl phosphine ligands, and aryl phosphine ligands are preferred, with triphenylphosphine being particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, an additive may also be used. The additive may be exemplified by an inorganic salt such as lithium chloride, potassium chloride, sodium bromide, and sodium iodide, or a phase-transfer catalyst such as tetrabutylammonium chloride, benzyltrimethylammonium chloride, and crown ethers. In particular, lithium chloride, tetrabutylammonium chloride, and benzyltrimethylammonium chloride are preferred. The amount of these additives to be used is preferably 0.1- to 10-fold moles, more preferably 0.1- to 5-fold moles, and particularly preferably 0.1- to 2-fold moles, relative to the compound (XIV) (or the compound (II) in which the substituent on the ring B is a leaving group).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

(4-5) For the compound (XIV) obtained as described above (or the compound (II) in which the substituent on the ring B is a leaving group), a substituent R⁴ such as an aromatic group and a heterocyclic aromatic group can be introduced by reacting with: (1) an alkene compound which may be substituted, in the presence of a transition metal catalyst, when the above cross-coupling reaction is the Mizoroki-Heck reaction; or (2) a C₆-14 arene compound which may be substituted or a C₅₋₁₀ heteroarene compound which may be substituted, when the above cross-coupling reaction is a direct arylation reaction for aromatics as similar to the Mizoroki-Heck reaction.

The alkene compound which may be substituted may be exemplified by C₂₋₁₀ alkene such as ethylene, 1-propene, isopropene, 2-methyl-1-propene, 1-butene, 2-butene, 3-butene, 2-ethyl-1-butene, 1-pentene, 2-pentene, 3-pentene, 4-pentene, 4-methyl-3-pentene, 1-hexene, 2-hexene, 3-hexene, 4-hexene, and 5-hexene. The substituent thereof may be exemplified by an ester group, an amide group, an alcohol group, an acetal group, a C₆₋₁₄ aryl group which may be substituted, a C₅₋₁₀ heteroaryl group which may be substituted, or the like.

A C₆₋₁₄ arene compound which may be substituted may be exemplified by benzene, naphthalene, or the like.

A C₅₋₁₀ heteroarene compound which may be substituted may be exemplified by furan, thiazole, thiophene, pyrrole, benzofuran, oxazole, indole, or the like.

Among these compounds, acrylic acid ester such as methyl acrylate, ethyl acrylate, and butyl acrylate, styrene, arylalcohol, acrolein diethyl acetal, thiazole, benzofuran, thiophene, indole, pyrrole, and the like are particularly preferred.

The amount of these compounds to be used is preferably 1- to 10-fold moles, more preferably 1- to 5-fold moles, and particularly preferably 1- to 2.5-fold moles, relative to the compound (XIV) (or the compound (II) in which the substituent on the ring B is a leaving group).

The present reaction can be performed in the absence or in the presence of a solvent. For the solvent, the same solvents as those used in the (Method 1) described above can be used, but among them, aromatic hydrocarbons, alcohols, amides, nitrites, and ethers are preferred, toluene, anisole, N,N-dimethylacetamide, N,N-dimethylformamide, N-methylpyrrolidone, dimethylsulfoxide, ethanol, acetonitrile, and tetrahydrofuran are more preferred, toluene, N,N-dimethylacetamide, and N,N-dimethylformamide are particularly preferred. The amount of the solvent to be used is preferably a 5- to 50-fold weight, more preferably a 5- to 30-fold weight, and particularly preferably a 5- to 20-fold weight, relative to the compound (XIV) (or the compound (II) in which the substituent on the ring B is a leaving group).

The transition metal catalyst used in the present reaction may be exemplified by palladium. For the palladium catalyst, the same catalysts as those used in the (Method 1) described above can be used, but among them, palladium acetate, tris(dibenzylideneacetone)dipalladium, palladium chloride, palladium acetate, tetrakis(triphenylphosphine)palladium(0), bis(triphenylphosphine)palladium dichloride, and 1,1′-bis(diphenylphosphino)ferrocene palladium dichloride, and tris(dibenzylideneacetone)dipalladium are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a ligand may be used together with the palladium catalyst. For such ligand, the same ligands as those used in the (Method 1) described above can be used, but among them, alkyl phosphine ligands, aryl phosphine ligands, biaryl type phosphine ligands, and ferrocene type phosphine ligands, are preferred, with triphenylphosphine, tri-o-tolylphosphine, 2,2′-bis(diphenylphosphino)-1,1′-binaphthyl, and 1,1′-bis(diphenylphosphino)ferrocene are particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a base may also be used. For the base, the same bases as those used in the (Method 1) described above can be used, but among them, an inorganic base and chain-like tertiary amine are preferred, while tripotassium phosphate, cesium carbonate, sodium carbonate, potassium carbonate, and triethylamine are preferred. The amount of these bases to be used is preferably 0.1- to 10-fold moles, more preferably 1- to 5-fold moles, and particularly preferably 1- to 2.5-fold moles, relative to the compound (XIV) (or the compound (II) in which the substituent on the ring B is a leaving group).

For the present reaction, an additive may also be used. The additive may be exemplified by an inorganic salt such as lithium chloride, potassium chloride, sodium bromide, and sodium iodide, a phase-transfer catalyst such as tetrabutylammonium chloride, benzyltrimethylammonium chloride, and crown ether, or a silver slat such as silver carbonate, and silver acetate. In particular, lithium chloride, tetrabutylammonium chloride, and benzyltrimethylammonium chloride are preferred. The amount of these additives to be used is preferably 0.1- to 10-fold moles, more preferably 0.1- to 5-fold moles, and particularly preferably 0.1- to 2-fold moles, relative to the compound (XIV) (or the compound (II) in which the substituent on the ring B is a leaving group).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

(4-6) For the compound (XIV) obtained as described above (or the compound (II) in which the substituent on the ring B is a leaving group), an alkynyl group (a substituent R⁴) can be introduced by reacting with an alkyne compound which may be substituted, in the presence of a transition metal catalyst, when the above cross-coupling reaction is the Sonogashira reaction.

The alkyne compound which may be substituted may be exemplified by C₂₋₁₀ alkynes such as acethylene, 1-propyne, 2-propyne, 1-butyne, 2-butyne, 3-butyne, 1-pentyne, 2-pentyne, 3-pentyne, 4-pentyne, 1-hexyne, 2-hexyne, 3-hexyne, 4-hexyne, and 5-hexyne. The substituent thereof may be exemplified by an ester group, an amide group, an amino group, an alcohol group, an acetal group, a C₆₋₁₄ aryl group which may be substituted, a C₅₋₁₀ heteroaryl group which may be substituted, a silyl group, or the like. Among these alkyne compounds, propargyl alcohol, propargyl amine, aryl acethylene, alkyl acethylene, and trimethylsilyl acethylene are particularly preferred.

The amount of these alkyne compounds to be used is preferably 1- to 10-fold moles, more preferably 1- to 5-fold moles, and particularly preferably 1- to 2.5-fold moles, relative to the compound (XIV) (or the compound (II) in which the substituent on the ring B is a leaving group).

The present reaction can be performed in the absence or in the presence of a solvent. For the solvent, the same solvents as those used in the (Method 1) described above can be used, but among them, aromatic hydrocarbons, alcohols, ethers, amides, nitriles, and water are preferred, with toluene, N,N-dimethylacetamide, tetrahydrofuran, 1,2-dimethoxyethane, acetonitrile, and water being particularly preferred. The amount of the solvent to be used is preferably a 5- to 50-fold weight, more preferably a 5- to 30-fold weight, and particularly preferably a 5- to 20-fold weight, relative to the compound (XIV) (or the compound (II) in which the substituent on the ring B is a leaving group).

The transition metal catalyst used in the present reaction may be exemplified by palladium. For the palladium catalyst, the same catalysts as those used in the (Method 1) described above can be used, but among them, palladium chloride, palladium acetate, tris(dibenzylideneacetone)dipalladium, tetrakis(triphenylphosphine)palladium(0), bis(triphenylphosphine)palladium dichloride, 1,1′-bis(diphenylphosphino)ferrocene palladium dichloride, and palladium carbon are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a ligand may be used together with the palladium catalyst. For such ligand, the same ligands as those used in the (Method 1) described above can be used, but among them, alkyl phosphine ligands, and aryl phosphine ligands, are preferred, with triphenylphosphine being particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a copper salt may also be used. For the copper salt, copper (I) iodide, copper (I) bromide, or the like may be exemplified. In particular, copper (I) iodide is preferred. The amount of these copper salts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a base may also be used. For the base, the same bases as those used in the (Method 1) described above can be used, but among them, an inorganic base and chain-like tertiary amine are preferred, while tripotassium phosphate, cesium carbonate, sodium carbonate, potassium carbonate, potassium acetate, triethylamine, and diisopropylethylamine are preferred. The amount of these bases to be used is preferably 0.1- to 10-fold moles, more preferably 1- to 5-fold moles, and particularly preferably 1- to 2.5-fold moles, relative to the compound (XIV) (or the compound (II) in which the substituent on the ring B is a leaving group).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

(4-7) For the compound (XIV) obtained as described above (or the compound (II) in which the substituent on the ring B is a leaving group), a cyano group (a substituent R⁴) can be introduced by reacting with a cyanide compound, in the presence of a transition metal catalyst, when the above cross-coupling reaction is a cyanation reaction.

For the cyanide compound, a metal cyanide compound such as zinc cyanide, copper cyanide, sodium cyanide, and potassium cyanide may be exemplified. Among them, zinc cyanide and sodium cyanide are particularly preferred. The amount of these cyanide compounds to be used is preferably 0.1- to 10-fold moles, more preferably 1- to 5-fold moles, and particularly preferably 1- to 2.5-fold moles, relative to the compound (XIV) (or the compound (II) in which the substituent on the ring B is a leaving group).

The present reaction can be performed in the absence or in the presence of a solvent. For the solvent, the same solvents as those used in the (Method 1) described above can be used, but among them, aromatic hydrocarbons, alcohols, ethers, amides, and nitriles are preferred, with toluene, N,N-dimethylacetamide, tetrahydrofuran, 1,2-dimethoxyethane, and acetonitrile, being particularly preferred. The amount of the solvent to be used is preferably a 5- to 50-fold weight, more preferably a 5- to 30-fold weight, and particularly preferably a 5- to 20-fold weight, relative to the compound (XIV) (or the compound (II) in which the substituent on the ring B is a leaving group).

The transition metal catalyst used in the present reaction may be exemplified by palladium. For the palladium catalyst, the same catalysts as those used in the (Method 1) described above can be used, but among them, palladium chloride, palladium acetate, tris(dibenzylideneacetone)dipalladium, tetrakis(triphenylphosphine)palladium (0), bis(triphenylphosphine)palladium dichloride, and 1,1′-bis(diphenylphosphino)ferrocene palladium dichloride are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a ligand may be used together with the palladium catalyst. For such ligand, the same ligands as those used in the (Method 1) described above can be used, but among them, alkyl phosphine ligands, aryl phosphine ligands, and biaryl type phosphine ligands, are preferred, with triphenylphosphine tricyclohexylphosphine, and 2,2′-bis(diphenylphoshino)-1,1′-binaphthyl are particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

For the compound (XIV) obtained as described above (or the compound (II) in which the substituent on the ring B is a leaving group), a cyano group (a substituent R⁴) can be also introduced by directly reacting with a metal cyanide compound (for example: copper cyanide), in the absence of a transition metal catalyst.

(4-8) For the compound (XIV) obtained as described above (or the compound (II) in which the substituent on the ring B is a leaving group), a hetero atom (for example, nitrogen, sulfur, and oxygen) can be introduced, that is, a substituent R⁴ such as a C₁₋₁₀ alkylamino group which may be substituted, a C₆₋₁₄ arylamino group which may be substituted, a C₇₋₁₃ aralkylamino group which may be substituted, a carboxylic amide group which may be substituted, a C₁₋₁₀ alkylthio group which may be substituted, a C₆₋₁₄ arylthio group which may be substituted, a C₁₋₁₀ alkoxy group which may be substituted, and a C₆₋₁₄ aryloxy group which may be substituted, can be introduced, by reacting with a C₁₋₁₀ alkylamine which may be substituted, a C₆₋₁₄ arylamine which may be substituted, a C₇₋₁₃ aralkylamine which may be substituted, a carboxylic amide which may be substituted, a C₁₋₁₀ alkylthiol which may be substituted, a C₆₋₁₄ arylthiol which may be substituted, a C₁₋₁₀ alcohol which may be substituted, a C₆₋₁₄ aryl alcohol which may be substituted, in the presence of a transition metal catalyst, when the above cross-coupling reaction is a reaction for introducing a hetero atom.

A C₁₋₁₀ alkylamine which may be substituted may be exemplified by methylamine or ethylamine. A C₆₋₁₄ arylamine which may be substituted may be exemplified by aniline. A C₇₋₁₃ aralkylamine which may be substituted may be exemplified by benzylamine. A carboxylic amide which may be substituted may be exemplified by formamide, acetamide, propionamide, benzamide, or amino acids. A C₁₋₁₀ alkylthiol which may be substituted may be exemplified by methanethiol, ethanethiol, or mercaptoacetic acid. A C₆₋₁₄ arylthiol which may be substituted may be exemplified by benzenethiol. A C₁₋₁₀ alcohol which may be substituted may be exemplified by methanol, ethanol, propanol, or butanol. A C₆₋₁₄ aryl alcohol which may be substituted may be exemplified by benzyl alcohol or phenol. Among them, benzylamine, aniline, amino acids, ethanethiol, benzenethiol, and butanol are particularly preferred.

The amount of these compounds to be used is preferably 1- to 10-fold moles, more preferably 1- to 5-fold moles, and particularly preferably 1- to 2.5-fold moles, relative to the compound (XIV) (or the compound (II) in which the substituent on the ring B is a leaving group).

The reaction can be performed in the same manner as in the (Method 2) described above.

(4-9) For the compound (XIV) obtained as described above (or the compound (II) in which the substituent on the ring B is a leaving group), a substituent R⁴ such as an ester group, an alkylaminocarbonyl group, and a dialkylaminocarbonyl group can be introduced by reacting with carbon monoxide and alcohols, or primary • secondary amine, in the presence of a transition metal catalyst, when the above cross-coupling reaction is a carbon monoxide insertion reaction.

The alcohols used in the present reaction may be exemplified by a C₁₋₁₀ alkyl alcohol which may be substituted, or a C₆₋₁₄ aryl alcohol which may be substituted. The primary • secondary amine may be exemplified by a C₁₋₁₀ alkylamine which may be substituted, a C₆₋₁₄ arylamine which may be substituted, or a C₅₋₁₀ heteroarylamine which may be substituted. Among them, methanol, ethanol, aniline, benzylamine are particularly preferred. The amount of these compounds to be used is preferably 0.1- to 10-fold moles, more preferably 1- to 5-fold moles, and particularly preferably 1- to 2.5-fold moles, relative to the compound (XIV) (or the compound (II) in which the substituent on the ring B is a leaving group).

The present reaction can be performed in the absence or in the presence of a solvent. For the solvent, the same solvents as those used in the (Method 1) described above can be used, but among them, aromatic hydrocarbons, alcohols, ethers, amides, and nitriles are preferred, with toluene, methanol, ethanol, N,N-dimethylacetamide, N,N-dimethylformamide, tetrahydrofuran, 1,2-dimethoxyethane, and acetonitrile being particularly preferred. The amount of the solvent to be used is preferably a 5- to 50-fold weight, more preferably a 5- to 30-fold weight, and particularly preferably a 5- to 20-fold weight, relative to the compound (XIV) (or the compound (II) in which the substituent on the ring B is a leaving group).

The transition metal catalyst used in the present reaction may be exemplified by palladium. For the palladium catalyst, the same catalysts as those used in the (Method 1) described above can be used, but among them, palladium chloride, palladium acetate, tris(dibenzylideneacetone)dipalladium, tetrakis(triphenylphosphine)palladium (0), and bis(triphenylphosphine)palladium dichloride, are preferred. The amount of these palladium catalysts to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a ligand may be used together with the palladium catalyst. For such ligand, the same ligands as those used in the (Method 1) described above can be used, but among them, alkyl phosphine ligands, aryl phosphine ligands, biaryl type phosphine ligands, and ferrocene type phosphine ligands are preferred, with triphenylphosphine, tricyclohexylphosphine, 2,2′-bis(diphenylphosphino)-1,1′-binaphthyl, 1,1′-bis(diphenylphosphino)ferrocene being particularly preferred. The amount of these ligands to be used is preferably 100 mol % or less, more preferably 0.001 mol % to 100 mol %, even more preferably 0.01 mol % to 50 mol %, and particularly preferably 0.1 mol % to 20 mol %, relative to the compound (XIV) (or the compound (II) in which the substituent on the ring B is a leaving group).

For the present reaction, a base may also be used. For the base, the same bases as those used in the (Method 1) described above can be used, but among them, an inorganic base and chain-like tertiary amine are preferred, while tripotassium phosphate, cesium carbonate, sodium carbonate, potassium carbonate, sodium acetate, triethylamine, and diisopropylethylamine are preferred. The amount of these bases to be used is preferably 0.1- to 10-fold moles, more preferably 1- to 5-fold moles, and particularly preferably 1- to 2.5-fold moles, relative to the compound (XIV) (or the compound (II) in which the substituent on the ring B is a leaving group).

The reaction temperature is usually 0 to 200° C., preferably 10 to 150° C., and particularly preferably 25 to 150° C., and the reaction time is usually 1 to 100 hours, preferably 1 to 50 hours, and particularly preferably 1 to 25 hours.

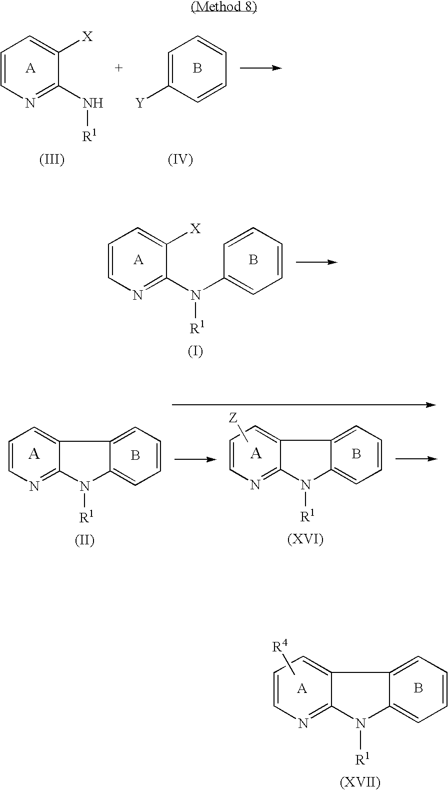

(1) Reaction of Aminopyridine Derivative (III) and Compound (IV):

The reaction can be performed in the same manner as in the (Method 2) described above.

(2) Ring Closure Reaction:

The reaction can be performed in the same manner as in the (Method 1) described above.

(3) Reaction for Introducing Leaving Group:

For the compound (II), when a leaving group does not exist on the ring A, a leaving group represented by Z such as a halogen atom, a C₁₋₄ alkanesulfonyloxy group which may be halogenated, a benzenesulfonyloxy group which may be substituted, and the like, can be introduced onto the ring A, by the reaction such as (1) a direct halogenation reaction of the ring A, (2) a reaction for converting an amino group which is a substituent on the ring A into halogen, (3) a reaction for converting a hydroxyl group which is a substituent on the ring A into a leaving group.

In addition, for the compound (II), when the compound (II) contains a substituent on the ring A, in which the substituent is a leaving group, a substituent R⁴ can be introduced onto the ring A, by being directly subjected to the coupling reaction to be described as follow, without being subjected to the reaction for introducing a leaving group.

The reaction can be performed in the same manner as in the (Method 7) described above.

(4) Coupling Reaction:

When the compound (XVI) (or the compound (II) in which the substituent on the ring A is a leaving group) is subjected to various cross-coupling reactions (for example, Suzuki reaction, Kumada reaction, Negishi reaction, Migita-Stille reaction, Mizoroki-Heck reaction, Sonogashira reaction, cyanation reaction, reaction for introducing hetero atom, carbon monoxide insertion reaction, and the like) in the presence of transition metal catalysts (for example, palladium catalyst, nickel catalyst), as described by F. Diederich and P. J. Stang, “Metal-catalyzed Cross-coupling Reactions”, Wiley-VCH, 1998, a substituent R⁴ such as an aromatic group, a heterocyclic aromatic group, an alkyl group, an alkenyl group, an alkynyl group, a carbonyl group, a cyano group or the like can be introduced onto ring A.

The reaction can be performed in the same manner as in the (Method 7) described above.

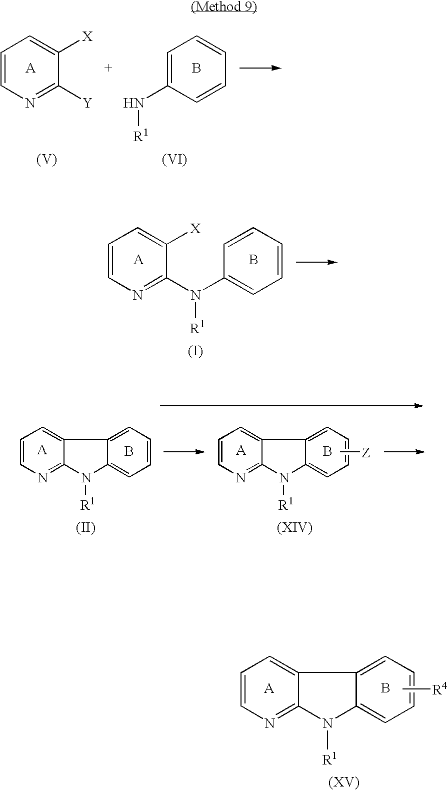

(1) Reaction of Pyridine Derivative (V) and Amine Derivative (VI):

The reaction can be performed in the same manner as in the (Method 3) described above.

(2) Ring Closure Reaction:

The reaction can be performed in the same manner as in the (Method 1) described above.

(3) Reaction for Introducing Leaving Group:

For the compound (II), when a leaving group does not exist on the ring B, a leaving group represented by Z such as a halogen atom, a C₁₋₄ alkanesulfonyloxy group which may be halogenated, a benzenesulfonyloxy group which may be substituted, and the like, can be introduced onto the ring B, by the reaction such as (1) a direct halogenation reaction of the ring B, (2) a reaction for converting an amino group which is a substituent on the ring B into halogen, (3) a reaction for converting a hydroxyl group which is a substituent on the ring B into a leaving group.

In addition, for the compound (II), when the compound (II) contains a substituent on the ring B, in which the substituent is a leaving group, a substituent R⁴ can be introduced onto the ring B, by being directly subjected to the coupling reaction to be described as follow, without being subjected to the reaction for introducing a leaving group.

The reaction can be performed in the same manner as in the (Method 7) described above.

(4) Coupling Reaction:

When the compound (XIV) (or the compound (II) in which the substituent on the ring B is a leaving group) is subjected to various cross-coupling reactions (for example, Suzuki reaction, Kumada reaction, Negishi reaction, Migita-Stille reaction, Mizoroki-Heck reaction, Sonogashira reaction, cyanation reaction, reaction for introducing hetero atom, carbon monoxide insertion reaction, and the like) in the presence of transition metal catalysts (for example, palladium catalyst, nickel catalyst), as described by F. Diederich and P. J. Stang, “Metal-catalyzed Cross-coupling Reactions”, Wiley-VCH, 1998, a substituent R⁴ such as an aromatic group, a heterocyclic aromatic group, an alkyl group, an alkenyl group, an alkynyl group, a carbonyl group, a cyano group or the like can be introduced onto ring B.

The reaction can be performed in the same manner as in the (Method 7) described above.

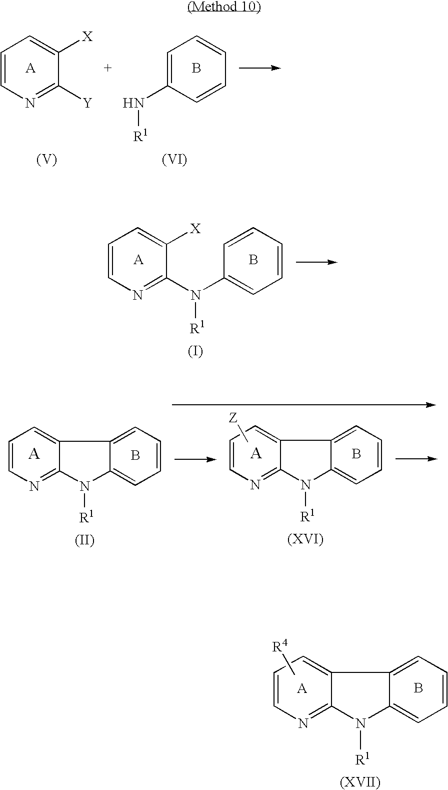

(1) Reaction of Pyridine Derivative (V) and Amine Derivative (VI):

The reaction can be performed in the same manner as in the (Method 3) described above.

(2) Ring Closure Reaction:

The reaction can be performed in the same manner as in the (Method 1) described above.

(3) Reaction for Introducing Leaving Group:

For the compound (II), when a leaving group does not exist on the ring A, a leaving group represented by Z such as a halogen atom, a C₁₋₄ alkanesulfonyloxy group which may be halogenated, a benzenesulfonyloxy group which may be substituted, and the like, can be introduced onto the ring A, by the reaction such as (1) a direct halogenation reaction of the ring A, (2) a reaction for converting an amino group which is a substituent on the ring A into halogen, (3) a reaction for converting a hydroxyl group which is a substituent on the ring A into a leaving group.

In addition, for the compound (II), when the compound (II) contains a substituent on the ring A, in which the substituent is a leaving group, a substituent R⁴ can be introduced onto the ring A, by being directly subjected to the coupling reaction to be described as follow, without being subjected to the reaction for introducing a leaving group.

The reaction can be performed in the same manner as in the (Method 7) described above.

(4) Coupling Reaction:

When the compound (XVI) (or the compound (II) in which the substituent on the ring A is a leaving group) is subjected to various cross-coupling reactions (for example, Suzuki reaction, Kumada reaction, Negishi reaction, Migita-Stille reaction, Mizoroki-Heck reaction, Sonogashira reaction, cyanation reaction, reaction for introducing hetero atom, carbon monoxide insertion reaction, and the like) in the presence of transition metal catalysts (for example, palladium catalyst, nickel catalyst), as described by F. Diederich and P. J. Stang, “Metal-catalyzed Cross-coupling Reactions”, Wiley-VCH, 1998, a substituent R⁴ such as an aromatic group, a heterocyclic aromatic group, an alkyl group, an alkenyl group, an alkynyl group, a carbonyl group, a cyano group or the like can be introduced onto ring A.

The reaction can be performed in the same manner as in the (Method 7) described above.

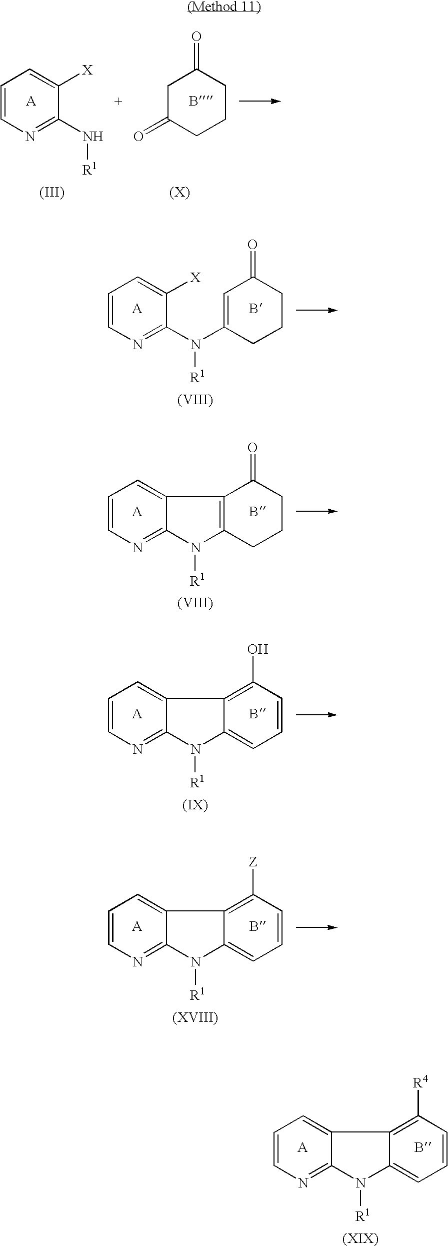

(1) Reaction of Aminopyridine Derivative (III) and Cyclohexanedione Derivative (X):

The reaction can be performed in the same manner as in the (Method 5) described above.

(2) Ring Closure Reaction:

The reaction can be performed in the same manner as in the (Method 4) described above. In addition, when the obtained compound (VIII) contains a leaving group on the ring A, the compound may be subjected to the cross-coupling reaction used in the (Method 7) described above, and subsequently the aromatizing reaction described as follow may be performed.

(3) Aromatization Reaction:

The reaction can be performed in the same manner as in the (Method 4) described above. In addition, when the obtained compound (IX) contains a leaving group on the ring A, the compound may be subjected to the cross-coupling reaction used in the (Method 7) described above, and subsequently the reaction for introducing leaving group described as follow may be performed.

(4) Reaction for Introducing Leaving Group:

For the compound (IX), a hydroxyl group on the ring B″ can be converted to a leaving group represented by Z such as a halogen atom, a C₁₋₄ alkanesulfonyloxy group which may be halogenated, and a benzenesulfonyloxy group which may be substituted.

The reaction can be performed in the same manner as in the (Method 7) described above.

(5) Coupling Reaction:

When the compound (XVIII) is subjected to various cross-coupling reactions (for example, Suzuki reaction, Kumada reaction, Negishi reaction, Migita-Stille reaction, Mizoroki-Heck reaction, Sonogashira reaction, cyanation reaction, reaction for introducing hetero atom, carbon monoxide insertion reaction, and the like) in the presence of transition metal catalysts (for example, palladium catalyst, nickel catalyst), as described by F. Diederich and P. J. Stang, “Metal-catalyzed Cross-coupling Reactions”, Wiley-VCH, 1998, a substituent R⁴ such as an aromatic group, a heterocyclic aromatic group, an alkyl group, an alkenyl group, an alkynyl group, a carbonyl group, a cyano group or the like can be introduced onto ring B″.

The reaction can be performed in the same manner as in the (Method 7) described above.

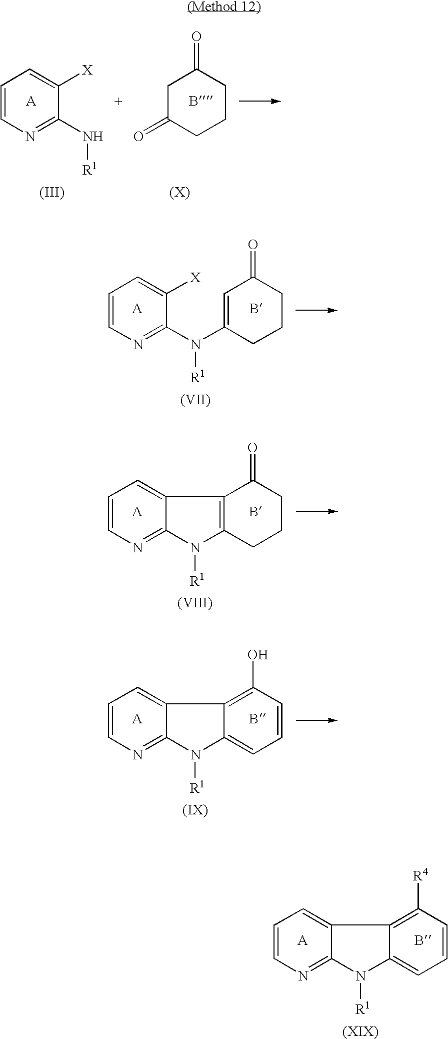

(1) Reaction of Aminopyridine Derivative (III) and Cyclohexanedione Derivative (X):

The reaction can be performed in the same manner as in the (Method 5) described above.

(2) Ring Closure Reaction:

The reaction can be performed in the same manner as in the (Method 4) described above.

(3) Aromatization Reaction:

The reaction can be performed in the same manner as in the (Method 4) described above.

(4) Coupling Reaction:

For the compound (IX), a substituent R⁴ represented as follow, can be introduced onto the ring B″, by subjecting a hydroxyl group on the ring B″ to a coupling reaction.

In the Formula (XIX), R⁴ may be exemplified by the substituent described above. In particular, a C₁₋₁₀ alkoxy group which may be substituted, a C₆₋₁₄ aryloxy group which may be substituted, a C₂₋₁₀ alkenyloxy group which may be substituted, and an acyl group may be preferred.

The reaction reagent used in the coupling reaction may be exemplified by alkyl halide, alkenyl halide, alkynyl halide or an equivalent thereof, alcohols, arylboronic acid, or acyl halide. Among them, C₁₋₁₀ alkyl halide which may be substituted, C₁₋₁₀ alkenyl halide which may be substituted, C₆₋₁₄ aryl halide which may be substituted, C₁₋₁₀ alkyl sulfonate which may be substituted, alcohols, acyl halide such as acid chloride are preferred. The amount of these reagents to be used is preferably 0.1- to 10-fold moles, more preferably 1- to 5-fold moles, and particularly preferably 1- to 2.5-fold moles, relative to the compound (IX).

The present reaction can be performed in the absence or in the presence of a solvent. For the solvent, the same solvents as those used in the (Method 1) described above can be used, but among them, aromatic hydrocarbons, aliphatic halogenated hydrocarbons, ethers, nitriles, and water are preferred, with toluene, pyridine, methylene chloride, tetrahydrofuran, acetonitrile, and water being particularly preferred. The amount of the solvent to be used is preferably a 5- to 50-fold weight, more preferably a 5- to 30-fold weight, and particularly preferably a 5- to 20-fold weight, relative to the compound (IX).

For the present reaction, a base may be used. For the base, the same bases as those used in the (Method 1) described above can be used, but among them, an inorganic base, heterocyclic aromatic amine, and chain-like tertiary amine are preferred, while tripotassium phosphate, sodium carbonate, potassium carbonate, pyridine, triethylamine, and diisopropylethylamine are preferred. The amount of these bases to be used is preferably 0.1- to 10-fold moles, more preferably 1- to 5-fold moles, and particularly preferably 1- to 2.5-fold moles, relative to the compound (IX).

For the present reaction, an activating agent and an additive may also be used. The activating agent may be exemplified by lewis acid such as aluminum chloride, tin chloride, and titanium chloride, azodicarboxylate ester such as diethyl azodicarboxylate, triphenylphosphine or the like. The additive may be exemplified by a phase-transfer catalyst such as tetrabutylammonium chloride, benzyltrimethylammonium chloride, and crown ether, or a metal salt such as copper (I) iodide and zinc chloride.

The reaction may be performed in the presence of a transition metal catalyst and a ligand. For the transition metal catalyst and the ligand, the copper catalyst and the ligand in the (Method 2) as described above, the palladium catalyst and the ligand in the (Method 1) as described above, or the nickel catalyst and the ligand in the (Method 7) as described above may be used.

The reaction temperature is usually 0 to 200° C., preferably 0 to 150° C., and particularly preferably 0 to 100° C., and the reaction time is usually 1 to 100 hours, preferably 1 to 50 hours, and particularly preferably 1 to 25 hours.

Each compound obtained in the above methods can be isolated and purified in accordance with a known means for separation and purification, for example, concentration, vacuum concentration, solvent extraction, crystallization, recrystallization, dissolution, and chromatography.

The salt of the compounds (II) and (IX) can be produced according to a method known per se, for example, by addition of an inorganic acid or organic acid to the compounds (II) and (IX), or the like.

When the stereoisomer would exist in the compounds (II) and (IX), any of each stereoisomer thereof and the mixture thereof is definitely included in the range of the present invention. These stereoisomers can also be produced independently, if desired.

In addition, the compounds (II) and (IX), or a salt thereof may be hydrate, and any of hydrate and nonhydrate is included in the range of the present invention.

Are novel compounds, of the compound (II), the compounds represented by the Formula,

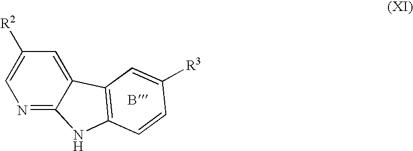

[in the Formula, the symbols respectively represent the same meaning as defined above], and the Formula,

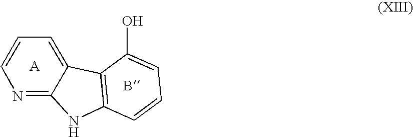

[in the Formula, the symbols respectively represent the same meaning as defined above], or a salt thereof; of the compound (IX), the compound represented by the Formula,

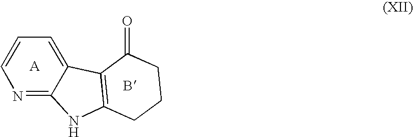

[in the Formula, the symbols respectively represent the same meaning as defined above], or a salt thereof, (except the following compounds):

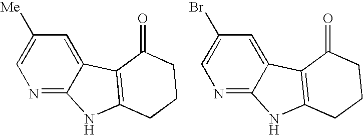

and of the compound (I), the compound represented by the Formula,

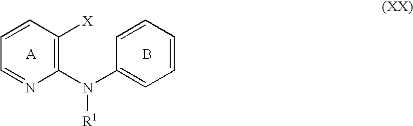

[in the Formula, the symbols respectively represents the same meaning as defined above, at least one of ring A and ring B is substituted, and the substituent(s) of the ring A and/or the ring B is (are) a substituent (substituents) selected from a halogen atom, an amino group which may be substituted, a C₁₋₁₀ alkoxy group which may be substituted, a C₁₋₁₀ alkoxy-carbonyl group which may be Substituted, an aminocarbonyl group which may have one or two substituents on the nitrogen atom, a C₆₋₁₀ aryl group which may be substituted, and a C₅₋₁₀ heteroaryl group which may be substituted], or a salt thereof.

Among the compound represented by the above-mentioned Formula (XI), the compound wherein R² is a halogen atom (for example, fluorine, chlorine, bromine, iodine), or a salt thereof, (except the following compounds):

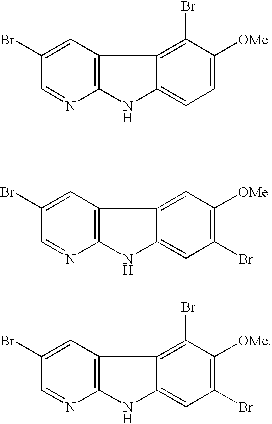

is preferred.

Among the compound represented by the above-mentioned Formula (XI), the compound wherein R³ is a halogen atom (for example, fluorine, chlorine, bromine, iodine), or a salt thereof, (except the following compounds):

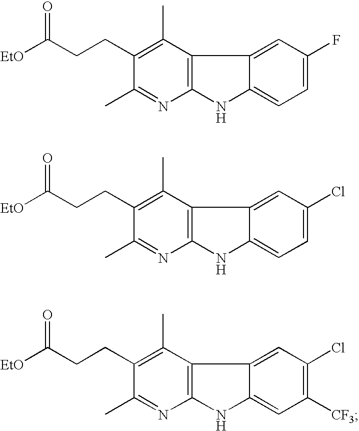

is also preferred.

Among the compound represented by the above-mentioned Formula (XX), the compound wherein R¹ is a hydrogen atom, at least one of ring A and ring B is substituted, and the substituents of ring A and/or ring B are at least two kinds of substituents selected from a halogen atom, an amino group which may be substituted, a C₁₋₁₀ alkoxy group which may be substituted, a C₁₋₁₀ alkoxy-carbonyl group which may be substituted, an aminocarbonyl group optionally having one or two substituent(s) on a nitrogen atom, a C₆₋₁₀ aryl group which may be substituted, and a C₅₋₁₀ heteroaryl group which may be substituted is preferred.

In such a preferred embodiment, the ring A may have at least two kinds of substituents selected from the above-mentioned substituent group, the ring B may have at least two kinds of substituents selected from the above-mentioned substituent group, or the ring A may have one kind of a substituent selected from the above-mentioned substituent group and the ring B may have other one kind of a substituent selected from the above-mentioned substituent group.

Among the compound represented by the above-mentioned Formula (XX), the compound wherein R¹ is a hydrogen atom, at least one of ring A and ring B is substituted, and the substituents of ring A and/or ring B are (i) at least one kind of a substituent selected from an amino group which may be substituted, a C₁₋₁₀ alkoxy group which may be substituted, a C₁₋₁₀ alkoxy-carbonyl group which may be substituted, and an aminocarbonyl group optionally having one or two substituent(s) on a nitrogen atom, and (ii) at least one kind of a substituent selected from an amino group which may be substituted, a C₆₋₁₀ aryl group which may be substituted, and a C₅₋₁₀ heteroaryl group which may be substituted, is also preferred.

In such a preferred embodiment, the ring A may have at least one kind of a substituent selected from the above-mentioned group (i) and at least one kind of a substituent selected from the above-mentioned group (ii), the ring B may have at least one kind of a substituent selected from the above-mentioned group (i) and at least one kind of a substituent selected from the above-mentioned group (ii), the ring A may have at least one kind of a substituent selected from the above-mentioned group (i) and the ring B may have at least one kind of a substituent selected from the above-mentioned group (ii), or the ring A may have at least one kind of a substituent selected from the above-mentioned group (ii) and the ring B may have at least one kind of a substituent selected from the above-mentioned group (i).

α-carboline derivatives obtained by the producing method of the present invention are useful for, for example, pharmaceutical products, agrochemicals, food products, cosmetic products, and chemical products, or as intermediates thereof.

For example, of the compound obtained by the producing method of the present invention (Method 1, Method 7, and Method 11), the compound in which R³ or R⁴ is an alkoxycarbonyl group, an alkylaminocarbonyl group, or dialkylaminocarbonyl group, is the α-carboline derivative having a CDK1/CDK5 (Cyclin-Dependent Kinase) inhibitory action and a GSK-3 (Glycogen Synthase Kinase) inhibitory action described in the Patent Document 1. The novel compounds (XI), (XII), (XIII), and (XX), are novel intermediates of the α-carboline derivative described in the Patent Document 1.

For example, of the compound obtained by the producing method of the present invention (Method 1, Method 7, and Method 11), the compound in which R³ or R⁴ is a cyano group, can be derivatized to the α-carboline derivative having a CDK1/CDK5 (Cyclin-Dependent Kinase) inhibitory action and a GSK-3 (Glycogen Synthase Kinase) inhibitory action described in the Patent Document 1, by converting the cyano group into an alkoxycarbonyl group. The novel compounds (XI), (XII), (XIII), and (XX), are novel intermediates of the α-carboline derivative described in the Patent Document 1.

The compound obtained by the producing method of the present invention can be derivatized to the α-carboline derivative having a CDK1 inhibitory action described in the Patent Document 6, by performing the Suzuki reaction described in the producing method of the present invention (Method 8 and Method 10) for the ring A. In addition, the compound obtained by the producing method of the present invention (Method 1) can be derivatized to the α-carboline derivative having a CDK1 inhibitory action described in the Patent Document 6, when the substituent of the ring A or ring B is an amino group which may be substituted or a C₆₋₁₀ aryl group which may be substituted. The novel compounds (XI), (XII), (XIII), and (XX), are novel intermediates of the α-carboline derivative described in the Patent Document 6.

The compound obtained by the producing method of the present invention can be derivatized to the carboline derivative having an antibacterial activity described in the Patent Document 5, by performing the Mizoroki-Heck reaction described in the producing method of the present invention (Method 7), for the ring B or the ring B″.

The compound obtained by the producing method of the present invention can be derivatized to the carboline derivative having a β-3 agonist activity described in the Non-Patent Document 4, by performing the coupling reaction described in the producing method of the present invention (Method 12), for the ring B or the ring B″.

### EXAMPLES

Hereinafter, the invention will be explained in more detail with reference to Examples, but the invention is not intended to be limited to such Examples.

### Example 1

### (1) 3-Bromo-5-methyl-N-phenylpyridine-2-amine

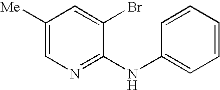

Process using Pd Catalyst

Under a nitrogen atmosphere, palladium acetate (336.8 mg, 1.5 mmol), 4,5-bis(diphenylphosphino)-9,9-dimethylxanthene (Xantphos) (867.9 mg, 1.5 mmol), sodium tert-butoxide (6.73 g, 70 mmol), and tert-butanol (100 ml) were mixed, and to this solution, 2-amino-3-bromo-5-methylpyridine (9.35 g, 50 mmol)and a solution of iodobenzene (10.2 g, 50 mmol) in tert-butanol (100 ml) were added at room temperature. The mixture was heated to reflux for 1 hour. After completion of the reaction, the reaction solution was cooled to room temperature, and ethanol (200 ml) was added thereto. The insoluble was filtered off through celite, and was washed twice with ethanol (20 ml). The filtrate was concentrated under reduced pressure. Ethyl acetate (200 ml) was added to the concentrate, and the mixture was washed twice with 10% brine (200 ml). The organic layer was dried over magnesium sulfate, and the filtrate was concentrated under reduced pressure. Ethanol (140 ml) was added to the concentrate, the mixture was heated to 50° C., and water (210 ml) was added dropwise thereto. The mixture was cooled to room temperature and stirred for 1 hour. The crystals were collected by filtration, washed with ethanol/water (2/3, 21 ml), and dried under reduced pressure at 50° C., to yield the title compound (11.5 g) (yield 87.0%).

¹H-NMR (CDCl₃, TMS, 300 MHz) δ (ppm): 2.24 (3H, s), 6.89 (1H, brs), 7.01-7.06 (1H, m), 7.31-7.36 (2H, m), 7.58-7.61 (3H, m), 7.80 (1H, s).

¹³C-NMR (CDCl₃, TMS, 300 MHz) δ (ppm): 17.1, 106.1, 119.4, 122.3, 125.1, 128.9, 140.2, 140.9, 146.1, 149.8.

High resolution mass spectrometry (C₁₂H₁₁BrN₂)

Theoretical value: 261.0027 [M]

Measured value: 261.0027 [M−H]⁺

Melting point: 63.2° C.

### (2) 3-Methyl-9H-pyrido[2,3-b]indole

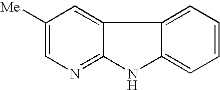

Under a nitrogen atmosphere, palladium acetate (269.4 mg, 1.2 mmol), 2-(dicyclohexylphosphino)biphenyl (841.2 mg, 2.4 mmol) and degassed N,N-dimethylacetamide (25 ml) were mixed, and the mixture was stirred at room temperature for 30 minutes. To this reaction solution, 3-bromo-5-methyl-N-phenylpyridine-2-amine (10.5 g, 40 mmol) and a solution of 1,8-diazabicyclo[5.4.0]-7-undecene (12.0 g, 80 mmol) in N,N-dimethylacetamide (10 ml) were added. The mixture was stirred at 130° C. for 1 hour. After completion of the reaction, the reaction solution was cooled to room temperature, and water (70 ml) was added thereto. The crystals were collected by filtration and washed with water (6 ml). Water (35 ml) was added to the obtained crude crystals, and the mixture was stirred at room temperature for 30 minutes. The crystals were collected by filtration, washed with water (20 ml), and dried under reduced pressure at 60° C., to yield the title compound (7.7 g) (yield 100%).

¹H-NMR (DMSO-d₆, TMS, 300 MHz) δ (ppm): 2.45 (3H, s), 7.16-7.21 (1H, m), 7.39-7.48 (2H, m), 8.11 (1H, d, J=7.8 Hz), 8.26 (1H, d, J=1.5 Hz), 8.30 (1H, s), 11.59 (1H, brs).

¹³C-NMR (DMSO-d₆, TMS, 300 MHz) δ (ppm): 18.2, 111.3, 115.1, 119.3, 120.3, 121.2, 123.6, 126.5, 128.6, 139.3, 146.7, 150.7.

Melting point: 270.4° C.

### (3) 3-Bromo-5-methyl-N-phenylpyridine-2-amine

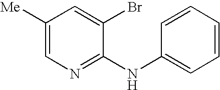

Process using Cu Catalyst

Under a nitrogen atmosphere, 2-amino-3-bromo-5-methylpyridine (2.00 g, 10.69 mmol), iodobenzene (2.18 g, 10.69 mmol), copper (I) iodide (204 mg, 0.11 mmol), ethanolamine (131 mg, 0.22 mmol), potassium carbonate (2.22 g, 16.04 mmol), and anisole (30 ml) were mixed, and the mixture was stirred at room temperature for 10 minutes. The mixture was heated and stirred at 130° C. for 8 hours. The reaction solution was cooled to room temperature, and water (30 ml) was added thereto. The mixture was concentrated under reduced pressure. To the residue, ethyl acetate (40 ml) and 25% aqueous ammonia (16 ml) were added. The organic layer was separated and washed sequentially, twice with 25% aqueous ammonia (10 ml), twice with 1N hydrochloric acid (10 ml), and once with saturated brine (10 ml). The organic layer was concentrated. The concentrate was subjected to silica gel column chromatography (silica gel 20 g, eluent: ethyl acetate:n-hexane=1:20), and the effective fraction was concentrated. Methanol (1 ml) and water (0.5 ml) were added to the residue, and the mixture was stirred at room temperature for 30 minutes. Next, the crystals were collected by filtration, and dried at 40° C. under reduced pressure, to yield the title compound (700 mg). (HPLC area%: 89.3% (3-iodo-5-methyl-N-phenylpyridine-2-amine: 9.7%).

The spectral data was confirmed to be the same as for the title compound obtained in the above (1).

### (4) 3-Methyl-9H-pyrido[2,3-b]indole

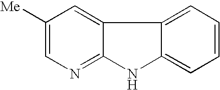

Under a nitrogen atmosphere, palladium acetate (13 mg, 0.06 mmol), 2-(dicyclohexylphosphino)biphenyl (40 mg, 0.11 mmol) and N,N-dimethylacetamide (1.5 ml) were mixed, and the mixture was stirred at room temperature for 10 minutes. To the reaction solution, 3-bromo-5-methyl-N-phenylpyridine-2-amine (500 mg, 1.90 mmol) and 1,8-diazabicyclo[5.4.0]-7-undecene (579 g, 3.80 mmol) were added. The mixture was stirred at 130° C. for 20 hours. The reaction solution was cooled to room temperature, and water (3 ml) was added thereto. The crystals were collected by filtration, and washed twice with methanol/water (1/1, 1 ml) and once with water (1 ml). The crystals were dried under reduced pressure at 50° C., to yield the title compound (331 mg) (yield after two processes starting from 2-amino-3-bromo-5-methylpyridine 23.8%).

The spectral data was confirmed to be the same as for the title compound obtained in the above (2).

### (5) 6-Bromo-3-methyl-9H-pyrido[2,3-b]indole

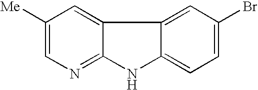

N-bromosuccinimide (2.67 g, 15 mmol) was added in portions to a solution of 3-methyl-9H-pyrido[2,3-b]indole (911.0 mg, 5 mmol) in tetrahydrofuran (300 ml), while maintaining the internal temperature at 10° C. or lower. The reaction solution was stirred for 4 hours at an internal temperature of 0 to 10° C. To this reaction solution, a 10% aqueous sodium sulfite solution (200 ml) was added. The organic layer was collected by phase separation, and then washed sequentially, twice with a 20% aqueous sodium carbonate solution (200 ml) and once with saturated brine (200 ml). The organic layer was dried over magnesium sulfate, and the filtrate was concentrated under reduced pressure. Acetone (10 ml) was added to the concentrate, and the mixture was stirred for 30 minutes while heating under reflux. The crystals were collected by filtration, washed twice with acetone (2 ml), and dried under reduced pressure at 50° C., to yield the title compound (901.7 mg) (yield 69.1%).

¹H-NMR (DMSO-d₆, TMS, 300 MHz) δ (ppm): 2.44 (3H, s), 7.43 (1H, d, J=8.6 Hz), 7.55 (1H, dd, J=2.0 Hz, 8.6 Hz), 8.31 (1H, d, J=1.5 Hz), 8.36 (2H, m).

¹³C-NMR (DMSO-d_{6,} TMS, 300 MHz) δ (ppm): 18.2, 111.3, 113.3, 114.2, 122.2, 123.8, 124.2, 128.9, 129.3, 138.0, 147.6, 150.8.

High resolution mass spectrometry (C₁₂H₉BrN₂)

Theoretical value: 259.9949 [M⁺]

Measured value: 259.9950 [M⁺]

Melting point: 286.8° C.

### Example 2

### (1) Ethyl 3-[(3-bromo-5-methylpyridin-2-yl)amino]benzoate

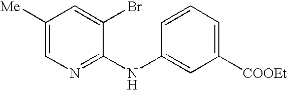

Under a nitrogen atmosphere, palladium acetate (360 mg, 1.6 mmol), 4,5-bis(diphenylphosphino)-9,9-dimethylxanthene (Xantphos) (928 mg, 1.6 mmol), and toluene (100 ml) were mixed, and the mixture was stirred at room temperature for 15 minutes. To this solution, 2-amino-3-bromo-5-methylpyridine (10.00 g, 53.46 mmol), ethyl m-iodobenzoate (14.76 g, 53.46 mmol), and cesium carbonate (24.39 g, 74.84 mmol) were added. The mixture was stirred at an internal temperature of 100 to 105° C. for 4 hours. The reaction solution was cooled to room temperature, and water (40 ml) was added thereto. Activated carbon Shirasagi A (500 mg) was added to the mixture, which was then filtered. The organic layer was separated and washed sequentially, twice with water (40 ml) and once with 5% brine (40 ml). The organic layer was concentrated under reduced pressure, to yield the title compound (19.89 g).

¹H-NMR (CDCl₃, TMS, 300 MHz) δ (ppm): 1.41 (3H, t, J=7.1 Hz), 2.24 (3H, s), 4.40 (2H, q, J=7.1 Hz), 6.99 (1H, brs), 7.37-7.43 (1H, m), 7.61-7.64 (1H, m), 7.69-7.79 (1H, m), 7.96 (1H, d, J=2.2 Hz), 7.99-8.01 (1H, m), 8.13-8.14 (1H, m).

Mass analysis (C₁₅H₁₅BrN₂O₂)

Theoretical value: 334

Measured value: 335 [M+H]⁺

Elemental analysis (C₁₅H₁₅BrN₂O₂)

Theoretical value: C, 53.75; H, 4.51; N, 8.36; Br, 23.84; O, 9.55

Measured value: C, 53.88; H, 4.43; N, 8.18; Br, 23.49

Melting point: 50.8 to 52.8° C.

### (2) Ethyl 3-methyl-9H-pyrido[2,3-b]indole-7-carboxylate

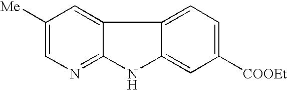

Under a nitrogen atmosphere, palladium acetate (480 mg, 2.14 mmol), 2-(dicyclohexylphosphino)biphenyl (1.50 g, 4.28 mmol) and degassed N,N-dimethylacetamide (20 ml) were mixed, and the mixture was stirred at room temperature for 30 minutes. To this reaction solution, ethyl 3-[(3-bromo-5-methylpyridin-2-yl)amino]benzoate obtained in (1) above, 1,8-diazabicyclo[5.4.0]-7-undecene (16.28 g, 106.92 mmol), and N,N-dimethylacetamide (20 ml) were added. The mixture was stirred at 150° C. for 5 hours. After completion of the reaction, the reaction solution was cooled to room temperature, and water (80 ml) was added. The mixture was stirred at room temperature for 30 minutes, and the crystals were collected by filtration and washed twice with ethanol/water (1/2, 50 ml) and once with water (50 ml). The crystals were dried under reduced pressure at 50° C., to yield the title compound (6.36 g) (yield after two processes starting from 2-amino-3-bromo-5-methylpyridine 46.8%).

¹H-NMR (DMSO-d₆, TMS, 300 MHz) δ (ppm): 1.38 (3H, t, J=7.1 Hz), 2.48 (3H, s), 4.37 (2H, q, J=7.1 Hz), 7.82 (1H, dd, J=1.4 Hz, 8.2 Hz), 8.11 (1H, s), 8.24 (1H, d, J=8.2 Hz), 8.38 (1H, d, J=1.7 Hz), 8.42 (1H, s), 11.91 (1H, s).

High resolution mass spectrometry (C₁₅H₁₄N₂O₂)

Theoretical value: 254.1055 [M⁺]

Measured value: 254.1054 [M⁺]

Melting point: 305° C.

### (3) Ethyl 6-bromo-3-methyl-9H-pyrido[2,3-b]indole-7-carboxylate

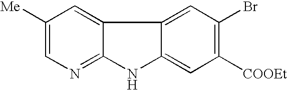

Tetrabutylammonium bromide (253 mg, 0.79 mmol) and N-bromosuccinimide (2.80 g, 15.74 mmol) were added to a solution of ethyl 3-methyl-9H-pyrido[2,3-b]indole-7-carboxylate (2.00 g, 7.87 mmol) in tetrahydrofuran (80 ml), and the mixture was stirred at an internal temperature of 40° C. for 1 hour. The reaction solution was cooled to room temperature, and a 10% aqueous sodium sulfite solution (20 ml) was added thereto. Ethyl acetate (50 ml) was added to the mixture, and the organic layer was separated and washed sequentially, three times with a saturated aqueous sodium bicarbonate solution (20 ml) and twice with saturated brine (20 ml). The organic layer was concentrated under reduced pressure. Tetrahydrofuran (4 ml) was added to the concentrate, and the mixture was stirred at room temperature for 30 minutes. The crystals were collected by filtration. Tetrahydrofuran (2 ml) and ethyl acetate (2 ml) were added to the obtained crude crystals, and the mixture was stirred for 30 minutes at 50° C., and for another 30 minutes at room temperature. The crystals were collected by filtration, washed with tetrahydrofuran/ethyl acetate (1/2, 1 ml), and dried under reduced pressure at 50° C., to yield the title compound (730 mg) (yield 27.9%).

¹H-NMR (DMSO-d₆, TMS, 300 MHz) δ (ppm): 1.38 (3H, t, J=7.1 Hz), 2.47 (3H, s), 4.39 (2H, q, J=7.1 Hz), 7.89 (1H, s), 8.39 (1H, d, J=1.8 Hz), 8.44 (1H, s), 8.56 (1H, s), 12.03 (1H, brs).

High resolution mass spectrometry (C₁₅H₁₃BrN₂O₂)

Theoretical value: 332.0160 [M⁺]

Measured value: 332.0150 [M⁺]

Melting point: 228.2 to 230.1° C.

### Example 3

### (1) 3-bromo-5-methyl-N-(2-methylphenyl)pyridine-2-amine

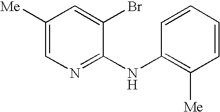

Under a nitrogen atmosphere, palladium acetate (252 mg, 1.12 mmol), 4,5-bis(diphenylphosphino)-9,9-dimethylxanthene (Xantphos) (650 mg, 1.12 mmol), and toluene (70 ml) were mixed, and the mixture was stirred at room temperature for 15 minutes. To this solution, 2-amino-3-bromo-5-methylpyridine (7.00 g, 37.43 mmol), o-iodotoluene (8.16 g, 37.43 mmol), and cesium carbonate (17.07 g, 52.40 mmol) were added. The mixture was stirred at an internal temperature of 100 to 105° C. for 4 hours. The reaction solution was cooled to room temperature, and water (56 ml) and toluene (70 ml) were added thereto. The organic layer was separated and washed sequentially with water (56 ml) and 5% brine (56 ml). Activated carbon Shirasagi A (350 mg) was added to the organic layer, which was then filtered, and the filtrate was concentrated under reduced pressure. The concentrate was subjected to silica gel column chromatography (silica gel 25 g, eluent; ethyl acetate:hexane=1:8), and the effective fraction was concentrated under reduced pressure, to yield the title compound (6.50 g).

¹H-NMR (CDCl₃, TMS, 300 MHz) δ (ppm): 2.23 (3H, s), 2.33 (3H, s), 6.73 (1H, brs), 6.99-7.04 (1H, m), 7.22 (2H, d, J=7.7 Hz), 7.60 (1H, d, J=1.5 Hz), 7.97 (1H, d, J=1.1 Hz), 8.01 (1H, d, J=8.0 Hz)

High resolution mass spectrometry (C₁₃H₁₃BrN₂)

Theoretical value: 276.0262 [M⁺]

Measured value: 276.0259 [M⁺]

### (2) 3,8-Dimethyl-9H-pyrido[2,3-b]indole

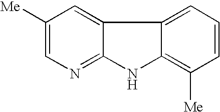

Under a nitrogen atmosphere, palladium acetate (158 mg, 1.12 mmol), 2-(dicyclohexylphosphino)biphenyl (493 mg, 2.25 mmol), and degassed N,N-dimethylacetamide (13 ml) were mixed, and the mixture was stirred at room temperature for 30 minutes. 3-Bromo-5-methyl-N-(2-methylphenyl)pyridine-2-amine obtained in (1) above, 1,8-diazabicyclo[5.4.0]-7-undecene (7.14 g, 74.86 mmol), and N,N-dimethylacetamide (6.5 ml) were added to the reaction solution. The mixture was stirred at 150° C. for 5 hours. Palladium acetate (526 mg, 3.74 mmol) and 2-(dicyclohexylphosphino)biphenyl (1.64 g, 7.48 mmol) were added thereto, and the mixture was further stirred for 4 hours. After completion of the reaction, the reaction solution was cooled to room temperature, and water (35 ml) was added thereto. The mixture was stirred at room temperature for 30 minutes, and the crystals were collected by filtration, and washed with ethanol/water (1/2, 12 ml) and water (12 ml). Ethyl acetate (35 ml) was added to the obtained crude crystals, the mixture was stirred for 30 minutes at 50° C. and for another 30 minutes at room temperature, and then the crystals were collected by filtration. The crystals were washed with ethyl acetate (5 ml) and dried under reduced pressure at 50° C., to yield the title compound (3.24 g) (yield after two processes starting from 2-amino-3-bromo-5-methylpyridine: 44.1%).

¹H-NMR (DMSO-d₆, TMS, 300 MHz) δ (ppm): 2.46 (3H, s), 2.55 (3H, s), 7.11 (1H, t, J=7.6 Hz), 7.24 (1H, d, J=7.1 Hz), 7.94 (1H, d, J=7.7 Hz), 8.29 (2H, s), 11.60 (1H, s).

High resolution mass spectrometry (C₁₃H₁₂N₂)

Theoretical value: 196.1000 [M⁺]

Measured value: 196.0999 [M⁺]

Melting point: 264.6 to 266.2° C.

### (3) 6-Bromo-3,8-dimethyl-9H-pyrido[2,3-b]indole

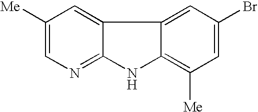

N-bromosuccinimide (4.35 g, 24.47 mmol) was added to a solution of 3,8-dimethyl-9H-pyrido[2,3-b]indole (3.20 g, 16.31 mmol) in tetrahydrofuran (150 ml), and the mixture was stirred at room temperature for 15 minutes. N-bromosuccinimide (1.45 g, 8.16 mmol) was further added thereto, and the mixture was stirred at room temperature for 30 minutes. A 15% aqueous sodium sulfite solution (50 ml) was added to the reaction solution, and the mixture was stirred at room temperature for 30 minutes. Ethyl acetate (50 ml) was added to the mixture. The organic layer was then separated and washed sequentially, three times with a saturated aqueous sodium bicarbonate solution (50 ml) and twice with saturated brine (30 ml). The organic layer was concentrated under reduced pressure. Ethyl acetate (24 ml) was added to the concentrate, and the mixture was stirred for 30 minutes at 50° C. and for another 30 minutes at room temperature. The crystals were collected by filtration and washed twice with ethyl acetate (5 ml). Ethanol (15 ml) and water (3 ml) were added to the obtained crude crystals, and the mixture was stirred for 30 minutes at 50° C. and for another 30 minutes at room temperature. The crystals were collected by filtration, and washed once with ethanol (10 ml) and twice with water (10 ml). The crystals were dried under reduced pressure at 50° C., to yield the title compound (1.52 g) (yield 33.9%).

¹H-NMR (DMSO-d₆, TMS, 300 MHz) δ (ppm): 2.45 (3H, s), 2.54 (3H, s), 7.40 (1H, d, J=0.8 Hz), 8.18 (1H, d, J=1.2 Hz), 8.32 (1H, d, J=1.9 Hz), 8.35 (1H, s), 11.81 (1H, s).

High resolution mass spectrometry (C₁₃H₁₁BrN₂)

Theoretical value: 274.0106[M⁺]

Measured value: 274.0097[M⁺]

Melting point: 284.9 to 286.9° C.

### Example 4

### 3,8-Dimethyl-9H-pyrido[2,3-b]indole-6-carbonitrile

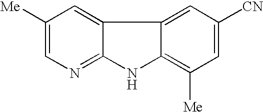

Under a nitrogen atmosphere, 6-bromo-3,8-dimethyl-9H-pyrido[2,3-b]indole (100 mg, 0.363 mmol), zinc cyanide (23 mg, 0.200 mmol), tetrakis(triphenylphosphine)palladium(0) (42 mg, 0.036 mmol), and 1-methyl-2-pyrrolidone (0.7 ml) were mixed. The mixture was heated and stirred at an internal temperature of 100° C. for 2 hours. The mixture was cooled to room temperature, and 12.5% aqueous ammonia (2 ml), ethyl acetate (5 ml), and 2-butanone (5 ml) were added to the mixture. The organic layer was separated and washed with saturated brine (3 ml). The organic layer was concentrated, and acetonitrile (0.3 ml) and ethyl acetate (0.5 ml) were added to the residue. The crystals were collected by filtration. Acetonitrile (0.2 ml) and ethyl acetate (0.3 ml) were added to the obtained crude crystals, and the crystals were collected by filtration. The crystals were dried under reduced pressure at 50° C., to yield the title compound (40 mg, yield 50%).

¹H-NMR (DMSO-d₆, TMS, 300 MHz) δ (ppm): 2.48 (3H, s), 2.59 (3H, s), 7.63 (1H, s), 8.39 (1H, d, J=2.1 Hz), 8.42 (1H, s), 8.52 (1H, s), 12.38 (1H, br).

Mass analysis (C₁₄H₁₁N₃)

Theoretical value: 221.0953[M⁺]

Measured value: 221.0950[M⁺]

Melting point: 301 to 304° C.

### Example 5

### Ethyl (2E)-3-(3,8-dimethyl-9H-pyrido[2,3-b]indol-6-yl)acrylate

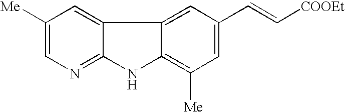

Under a nitrogen atmosphere, palladium acetate (8 mg, 0.036 mmol), triphenylphosphine (19 mg, 0.073 mmol) and 1-methyl-2-pyrrolidone (0.5 ml) were mixed. The mixture was stirred at room temperature for 30 minutes. 6-Bromo-3,8-dimethyl-9H-pyrido[2,3-b]indole (100 mg, 0.363 mmol), ethyl acrylate (146 mg, 1.452 mmol), potassium acetate (71 mg, 0.726 mmol), benzyltriethylammonium chloride (83 mg, 0.363 mmol) and 1-methyl-2-pyrrolidone (0.3 ml) were added thereto, and the mixture was stirred at room temperature for 15 minutes. The mixture was heated and stirred at an internal temperature of 90° C. for 8 hours. The mixture was cooled to room temperature, and ethyl acetate (10 ml), water (3 ml), activated carbon Shirasagi A were added to the mixture, which was then filtered. The organic layer was separated and washed with saturated brine (3 ml). The organic layer was concentrated, ethyl acetate (0.5 ml) was added to the residue, and the crystals were collected by filtration. Ethyl acetate (0.3 ml) was added to the obtained crude crystals, and the crystals were collected by filtration. The crystals were dried under reduced pressure at 50° C., to yield the title compound (30 mg) (yield 28.0%).

¹H-NMR (DMSO-d₆, TMS, 300 MHz) δ (ppm): 1.29 (3H, t, J=7.1 Hz), 2.48 (3H, s), 2.57 (3H, s), 4.21 (2H, q, J=7.1 Hz), 6.60 (1H, d, J=15.9 Hz), 7.66 (1H, s), 7.78 (1H, d, J=15.9 Hz), 8.33 (2H, s), 8.36 (1H, s), 11.92 (1H, s).

Mass analysis (C₁₈H₁₈N₂O₂)

Theoretical value: 294.1368 [M⁺]

Measured value: 294.1365 [M⁺]

Melting point: 253 to 256° C.

### Example 6

### 3,8-Dimethyl-6-phenyl-9H-pyrido[2,3-b]indole

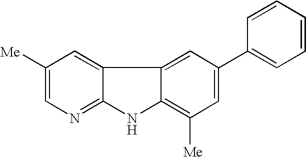

Under a nitrogen atmosphere, 6-bromo-3,8-dimethyl-9H-pyrido[2,3-b]indole (150 mg, 0.55 mmol), sodium carbonate (116 mg, 1.09 mmol), phenylboronic acid (80 mg, 0.65 mmol), N,N-dimethylacetamide (1.5 ml) and water (0.2 ml) were mixed. Tetrakis(triphenylphosphine)palladium(0) (63 mg, 0.06 mmol) was added to the mixture. The resulting mixture was heated and stirred at 100° C. for 3 hours. After completion of the reaction, the reaction solution was cooled to room temperature, and water (2 ml) was added thereto. The mixture was stirred at room temperature for 30 minutes, the crystals were collected by filtration, and the crystals were washed with methanol/water (1/2, 1 ml). The obtained crude crystals were suspended in methanol (3 ml) at room temperature, and the crystals were collected by filtration. The crystals were dried under reduced pressure at 50° C., to yield the title compound (15 mg) (yield 10.1%).

¹H-NMR (DMSO-d₆, TMS, 300 MHz) δ (ppm): 2.48 (3H, s), 2.62 (3H, s), 7.34 (1H, t, J=7.6 Hz), 7.49 (2H, t, J=7.6 Hz), 7.58 (1H, s), 7.75 (2H, d, J=7.5 Hz), 8.29 (2H, d, J=7.1 Hz), 8.39 (1H, s), 11.69 (1H, s).

High resolution mass spectrometry (C₁₉H₁₆N₂)

Theoretical value: 272.1313 [M⁺]

Measured value: 272.1311 [M⁺]

Melting point: 274 to 277° C.

### Example 7

### (1) Methyl 3-iodo-2-methylbenzoate

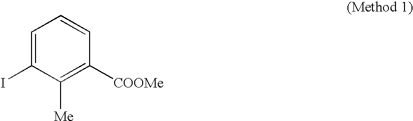

3-Iodo-2-methylbenzoic acid (10.00 g, 38.16 mmol), methanol (50 ml) and concentrated sulfuric acid (0.6 ml, 11.45 mmol) were mixed, and the mixture was stirred for 5 hours while heating to reflux. The reaction solution was concentrated, ethyl acetate (80 ml) and water (30 ml) were added to the residue. The organic layer was separated and washed three times with a saturated aqueous sodium bicarbonate solution (30 ml) and once with 10% brine (30 ml). The organic layer was concentrated, to yield the title compound (9.22 g) (yield 87.5%).

¹H-NMR (CDCl₃, TMS, 300 MHz) δ (ppm): 2.67 (3H, s), 3.90 (3H, s), 6.92 (1H, t, J=7.8 Hz), 7.74 (1H, dd, J=1.2 Hz, 7.8 Hz), 7.98 (1H, dd, J=1.2 Hz, 7.9 Hz).

(Method 2)

3-Iodo-2-methylbenzoic acid (38.00 g, 145.01 mmol), tetrahydrofuran (114 ml) and N,N-dimethylformamide (0.56 ml, 7.25 mmol) were mixed, and oxalyl chloride (27.61 g, 217.52 mmol) was added dropwise to the mixture with ice cooling over about 15 minutes. The mixture was stirred for 1 hour with ice cooling, and the reaction solution was concentrated under reduced pressure. Separately, triethylamine (24.95 g, 246.52 mmol) and methanol (76 ml) were mixed, and a solution of the acid chloride prepared above in tetrahydrofuran (76 ml) was added dropwise thereto under ice cooling over about 20 minutes. After completion of the dropwise addition, the mixture was stirred at room temperature for 30 minutes. Ethyl acetate (570 ml) and water (190 ml) were added to the reaction solution. The organic layer was separated and washed twice with 10% brine (38 ml). The organic layer was concentrated, to yield the title compound (40.58 g).

### (2) Methyl 3-[(3-bromo-5-methylpyridin-2-yl)amino]-2-methylbenzoate

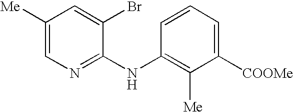

Process using Pd Catalyst

Under a nitrogen atmosphere, palladium acetate (223 mg, 0.99 mmol), 4,5-bis(diphenylphosphino)-9,9-dimethylxanthene (Xantphos) (575 mg, 0.99 mmol) and toluene (62 ml) were mixed, and the mixture was stirred at room temperature for 15 minutes. To this solution, 2-amino-3-bromo-5-methylpyridine (6.20 g, 33.15 mmol), methyl 3-iodo-2-methylbenzoate (9.15 g, 33.15 mmol) and cesium carbonate (15.12 g, 46.41 mmol) were added. The mixture was stirred at an internal temperature of 100 to 105° C. for 7 hours. The reaction solution was cooled to room temperature, and water (50 ml) and toluene (50 ml) were added thereto. The organic layer was separated and washed sequentially with water (40 ml) and 10% brine (40 ml). Silica gel (6 g) was added to the organic layer, the mixture was filtered, and the filtrate was concentrated under reduced pressure. Ethyl acetate (0.5 ml) and n-hexane (6 ml) were added to the concentrate, and the mixture was stirred at room temperature for 30 minutes. The crystals were collected by filtration, washed with ethyl acetate/n-hexane (1/12, 5 ml), and dried under reduced pressure at 40° C., to yield the title compound (4.81 g) (yield 43.3%).

¹H-NMR (CDCl₃, TMS, 300 MHz) δ (ppm): 2.23 (3H, s), 2.52 (3H, s), 3.91 (3H, s), 6.78 (1H, brs), 7.27 (1H, t, J=8.0 Hz), 7.57-7.62 (2H, m), 7.95 (1H, d, J=1.1 Hz), 8.10 (1H, dd, J=1.0 Hz, 8.1 Hz).

High resolution mass spectrometry (C₁₅H₁₅BrN₂O₂) Theoretical value: 334.0317 [M⁺]

Measured value: 334.0313 [M⁺]

Melting point: 63.9 to 64.7° C.

### (3) Methyl 3,8-dimethyl-9H-pyrido[2,3-b]indole-7-carboxylate

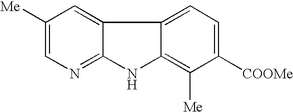

Under a nitrogen atmosphere, palladium acetate (96 mg, 0.43 mmol), 2-(dicyclohexylphosphino)biphenyl (301 mg, 0.86 mmol) and degassed N,N-dimethylacetamide (9.6 ml) were mixed, and the mixture was stirred at room temperature for 30 minutes. Methyl 3-[(3-bromo-5-methylpyridin-2-yl)amino]-2-methylbenzoate (4.80 g, 14.32 mmol), 1,8-diazabicyclo[5.4.0]-7-undecene (4.36 g, 28.64 mmol) and N,N-dimethylacetamide (4.8 ml) were added to the mixture. The mixture was stirred at 150° C. for 2 hours. After completion of the reaction, the reaction solution was cooled to room temperature, and water (14.4 ml) was added thereto. The mixture was stirred at room temperature for 30 minutes, and the crystals were collected by filtration and washed sequentially once with ethanol/water (1/2, 10 ml) and twice with water (10 ml). The crystals were dried under reduced pressure at 50° C., to yield the title compound (2.14 g) (yield 58.8%).

¹H-NMR (DMSO-d₆, TMS, 300 MHz) δ (ppm): 2.47 (3H, s), 2.78 (3H, s), 3.88 (3H, s), 7.68 (1H, d, J=8.3 Hz), 8.04 (1H, d, J=8.3 Hz), 8.38-8.39 (2H, m), 11.91 (1H, s).

High resolution mass spectrometry (C₁₅H₁₄N₂O₂)

Theoretical value: 254.1055 [M⁺]

Measured value: 254.1056 [M⁺]

Melting point: 296.6 to 298.7° C.

### (4) Methyl 3-[(3-bromo-5-methylpyridin-2-yl)amino]-2-methylbenzoate

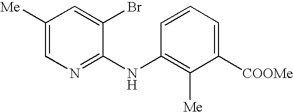

Process using Cu Catalyst

Under a nitrogen atmosphere, 2-amino-3-bromo-5-methylpyridine (2.00 g, 10.69 mmol), methyl 3-iodo-2-methylbenzoate (2.95 g, 10.69 mmol), copper (I) iodide (204 mg, 0.11 mmol), ethanol amine (131 mg, 0.22 mmol), potassium carbonate (2.22 g, 16.04 mmol), and anisole (30 ml) were mixed, and the mixture was stirred at room temperature for 10 minutes. The mixture was heated and stirred at 130° C. for 8 hours. The reaction solution was cooled to room temperature, and water (30 ml) was added thereto. The mixture was concentrated under reduced pressure. The concentrate was dissolved in ethyl acetate (50 ml), activated carbon Shirasagi A was added, and the mixture was filtered. Water (20 ml) was added to the filtrate. The organic layer was separated and washed with water (20 ml). The organic layer was concentrated. The concentrate was subjected to silica gel column chromatography (silica gel 20 g, eluent: ethyl acetate:n-hexane=1:20), and the effective fraction was concentrated. Ethyl acetate (0.2 ml) and n-hexane (5 ml) were added to the residue, and the mixture was stirred at room temperature for 30 minutes. Next, the crystals were collected by filtration, and dried at 50° C. under reduced pressure, to yield the title compound (1.13 g). (HPLC area %: 83.4% (methyl 3-[(3-iodo-5-methylpyridin-2-yl)amino]-2-methylbenzoate: 16.1%)).

The spectral data was confirmed to be the same as for the title compound obtained in the above (2).

### (5) Methyl 3,8-dimethyl-9H-pyrido[2,3-b]indole-7-carboxylate

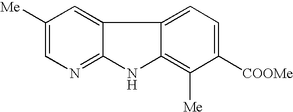

Under a nitrogen atmosphere, palladium acetate (10 mg, 0.04 mmol), 2-(dicyclohexylphosphino)biphenyl (31 mg, 0.09 mmol) and N,N-dimethylacetamide (1.5 ml) were mixed, and the mixture was stirred at room temperature for 10 minutes. Methyl 3-[(3-bromo-5-methylpyridin-2-yl)amino]-2-methylbenzoate (500 mg, 1.49 mmol), and 1,8-diazabicyclo[5.4.0]-7-undecene (454 mg, 2.98 mmol) were added. The mixture was stirred at 130° C. for 3 hours. After completion of the reaction, the reaction solution was cooled to room temperature, and water (3 ml) was added. The mixture was stirred at room temperature for 30 minutes, and the crystals were collected by filtration and washed twice with methanol/water (1/1, 1 ml) and once with water (1 ml). The crystals were dried under reduced pressure at 50° C., to yield the title compound (yield after two processes starting from 2-amino-3-bromo-5-methylpyridine 23.2%).

The spectral data was confirmed to be the same as for the title compound obtained in the above (3).

### (6) Methyl 6-bromo-3,8-dimethyl-9H-pyrido[2,3-b]indole-7-carboxylate

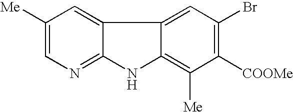

Methyl 3,8-dimethyl-9H-pyrido[2,3-b]indole-7-carboxylate (1.00 g, 3.93 mmol), tetrabutylammonium bromide (127 mg, 0.39 mmol) and tetrahydrofuran (100 ml) were mixed, and N-bromosuccinimide (1.05 g, 5.90 mmol) was added to the mixture, which was then stirred at an internal temperature of 60° C. for 1 hour. N-bromosuccinimide (1.40 g, 7.86 mmol) was further added thereto, and the resulting mixture was stirred for 30 minutes. The reaction solution was cooled, 10% aqueous sodium sulfite solution (30 ml) was added thereto, and the mixture was stirred at room temperature for 30 minutes. After addition of ethyl acetate (50 ml), the organic layer was separated and washed sequentially, three times with a saturated aqueous sodium bicarbonate solution (30 ml) and twice with saturated brine (30 ml). The organic layer was concentrated under reduced pressure. Ethyl acetate (1.5 ml) and n-hexane (3 ml) were added to the concentrate, and the mixture was stirred for 30 minutes at 50° C. and for another 30 minutes at room temperature. The crystals were collected by filtration, and washed with ethyl acetate/n-hexane (1/1, 2 ml). The crystals were dried under reduced pressure at 50° C., to yield the title compound (975 mg) (yield 74.4%).

¹H-NMR (DMSO-d₆, TMS, 300 MHz) δ (ppm): 2.47 (3H, s), 2.52 (3H, s), 3.93 (3H, s), 8.36 (1H, s), 8.38 (1H, d, J=1.7 Hz), 8.42 (1H, s), 12.03 (1H, s).

High resolution mass spectrometry (C₁₅H₁₃BrN₂O₂)

Theoretical value: 332.0160 [M⁺]

Measured value: 332.0153 [M⁺]

Melting point: 286.5 to 287.3° C.

### (7) Methyl 6-iodo-3,8-dimethyl-9H-pyrido[2,3-b]indole-7-carboxylate

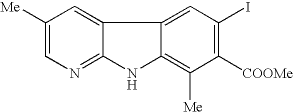

Methyl 3,8-dimethyl-9H-pyrido[2,3-b]indole-7-carboxylate (100 mg, 0.393 mmol), iodine (40 mg, 0.157 mmol), iodic acid (40 mg, 0.08 mmol), acetic acid (1 ml) and 64% sulfuric acid (0.1 ml) were mixed, and the mixture was stirred at an internal temperature of 80° C. for 1.5 hours. The reaction solution was ice-cooled, and the pH was adjusted to 4 to 5 using a 2N aqueous sodium hydroxide solution. Then, methanol (0.5 ml) was added thereto, and the mixture was stirred. The crystals were collected by filtration, and washed with methanol/water (1/2, 0.5 ml) and water (0.5 ml). Tetrahydrofuran (1.5 ml) was added to the crude crystals, the mixture was stirred at room temperature, and then the crystals were collected by filtration. The crystals were dried under reduced pressure at 40° C., to yield the title compound (45 mg) (yield 30.1%).

¹H-NMR (DMSO-d₆, TMS, 300 MHz) δ (ppm): 2.45 (3H, s), 2.51 (3H, s), 3.91 (3H, s), 8.35 (1H, d, J=1.3 Hz), 8.39 (1H, s), 8.52 (1H, s), 11.97 (1H, s).

High resolution mass spectrometry (C₁₅H₁₃IN₂O₂)

Theoretical value: 380.0022 [M⁺]

Measured value: 380.0030 [M⁺]

### (8) Methyl 5,6-dibromo-3,8-dimethyl-9H-pyrido[2,3-b]indole-7-carboxylate

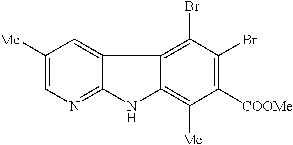

Methyl 3,8-dimethyl-9H-pyrido[2,3-b]indole-7-carboxylate (100 mg, 0.393 mmol) and trifluoroacetic acid (0.5 ml) were mixed. At an internal temperature of 80° C., 1,3-dibromo-5,5-dimethylhydantoin (22 mg, 0.08 mmol) was added in six portions at an interval of 10 minutes. After completion of the addition, the mixture was stirred for 1.5 hours. The reaction solution was cooled, water (1 ml) was added with ice cooling, and the mixture was adjusted to pH 7 to 8 using an 8N aqueous sodium hydroxide solution. The mixture was stirred for 30 minutes with ice cooling, and the crystals were collected by filtration. The crystals were washed twice with methanol/water (1/1, 0.5 ml) and once with water (1 ml). Methanol (0.5 ml) was added to the obtained crude crystals, the mixture was stirred at room temperature for 30 minutes, and the crystals were collected by filtration. The crystals were washed with methanol (0.5 ml), and dried under reduced pressure at 40° C., to yield the title compound (80 mg) (yield 49.4%).

¹H-NMR (DMSO-d₆, TMS, 300 MHz) δ (ppm): 2.49 (3H, s), 2.50 (3H, s), 3.94 (3H, s), 8.45 (1H, d, J=1.6 Hz), 8.73 (1H, s), 12.39 (1H, s).

High resolution mass spectrometry (C₁₅H₁₂Br₂N₂O₂)

Theoretical value: 409.9266 [M⁺]

Measured value: 409.9265 [M⁺]

Melting point: 292.9 to 295.2° C. (dec.)

### (9) Methyl 9-acetyl-3,8-dimethyl-9H-pyrido[2,3-b]indole-7-carboxylate

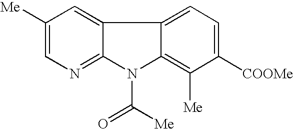

Methyl-3,8-dimethyl-9H-pyrido[2,3-b]indole-7-carboxylate (4.50 g, 17.70 mmol) and acetic anhydride (45 ml) were mixed, and the mixture was stirred for 8 hours while heating to reflux. The reaction solution was cooled to room temperature, water (90 ml) was added thereto, and the mixture was concentrated under reduced pressure. Ethyl acetate (450 ml) and water (30 ml) were added to the concentrate. The organic layer was separated and washed three times with 1N hydrochloric acid (30 ml). The organic layer was concentrated under reduced pressure, methanol (40 ml) was added to the residue, and the mixture was stirred at room temperature for 1 hour. The crystals were collected by filtration, and washed with methanol (9 ml). The crystals were dried under reduced pressure at 50° C., to yield the title compound (3.40 g) (yield 64.8%).

¹H-NMR (DMSO-d₆, TMS, 300 MHz) δ (ppm): 2.39 (3H, s), 2.48 (3H, s), 3.11 (3H, s), 3.90 (3H, s), 7.85 (1H, d, J=8.1 Hz), 8.08 (1H, d, J=8.0 Hz), 8.43 (1H, s), 8.45 (1H, s).

High resolution mass spectrometry (C₁₇H₁₆N₂O₃)

Theoretical value: 296.1161 [M⁺]

Measured value: 296.1161 [M⁺]

Melting point: 141.4 to 142.7° C.

### (10) Methyl 9-acetyl-5-bromo-3,8-dimethyl-9H-pyrido[2,3-b]indole-7-carboxylate

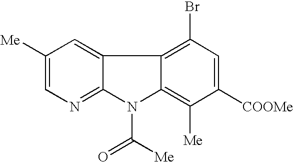

Methyl 9-acetyl-3,8-dimethyl-9H-pyrido[2,3-b]indole-7-carboxylate (1.00 g, 3.375 mmol), potassium sulfate (29 mg, 0.17 mmol) and 64% sulfuric acid (5 ml) were mixed. Under ice cooling, sodium bromate (509 mg, 3.375 mmol) was added in small portions over about 30 minutes. After completion of the addition, the mixture was stirred for 1 hour with ice cooling. Under ice cooling, the mixture was adjusted to near pH 8 by adding 4N aqueous sodium hydroxide solution. Ethyl acetate (50 ml) was added thereto, and the organic layer was separated. Ethyl acetate (20 ml) and tetrahydrofuran (10 ml) were added to the aqueous layer, and the organic layer was separated. The organic layers were combined, and washed sequentially with a 10% aqueous sodium sulfite solution (10 ml) and saturated brine (10 ml). The organic layer was concentrated under reduced pressure, and tetrahydrofuran (1 ml) and ethyl acetate (4 ml) were added to the concentrate. The mixture was stirred at room temperature for 30 minutes, and then the crystals were collected by filtration. The crystals were washed with ethyl acetate (1 ml). Tetrahydrofuran (0.5 ml) and ethyl acetate (4 ml) were added to the obtained crude crystals, the mixture was stirred at room temperature for 30 minutes, and then the crystals were collected by filtration. The crystals were washed with ethyl acetate (1 ml), and dried under reduced pressure at 50° C., to yield the title compound (345 mg) (yield 27.3%).

¹H-NMR (DMSO-d₆, TMS, 300 MHz) δ (ppm): 2.34 (3H, s), 2.47 (3H, s), 3.12 (3H, s), 3.90 (3H, s), 7.99 (1H, s), 8.49 (1H, s), 8.76 (1H, s).

High resolution mass spectrometry (C₁₇H₁₅BrN₂O₃)

Theoretical value: 374.0266 [M⁺]

Measured value: 374.0266 [M⁺]

Melting point: 171.9 to 173.6° C.

### Example 8

### 5-[3-(ethylsulfonyl)phenyl]-3,8-dimethyl-9H-pyrido[2,3-b]indole-7-carboxylic acid

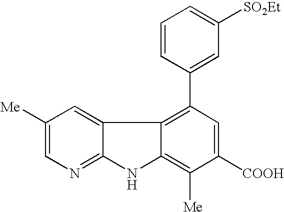

Under a nitrogen atmosphere, methyl 9-acetyl-5-bromo-3,8-dimethyl-9H-pyrido[2,3-b]indole-7-carboxylate (130 mg, 0.35 mmol), sodium carbonate (110 mg, 1.04 mmol), [3-(ethylsulfonyl)phenyl]boronic acid (148 mg, 0.69 mmol), N,N-dimethylacetamide (1.1 ml) and water (0.3 ml) were mixed. Tetrakis(triphenylphosphine)palladium (0) (20 mg, 0.02 mmol) was added to the mixture. The resulting mixture was heated and stirred at 100° C. for 2 hours. After completion of the reaction, the reaction solution was cooled to 60° C., and a 4N aqueous sodium hydroxide solution (0.2 ml) was added thereto. The mixture was stirred at 60° C. for 1 hour, cooled to room temperature, and the pH was adjusted to 5 to 6 using 2N hydrochloric acid. The mixture was stirred at room temperature for 30 minutes. The crystals were collected by filtration, and washed twice with methanol/water (1/2, 1 ml) and twice with water (1 ml). The crystals were dried under reduced pressure at 50° C., to yield the title compound (130 mg) (yield 91.9%).

¹H-NMR (DMSO-d₆, TMS, 300 MHz) δ (ppm): 1.19 (3H, t, J=7.3 Hz), 2.28 (3H, s), 2.80 (3H, s), 3.43 (2H, q, J=7.3 Hz), 7.51 (1H, s), 7.59 (1H, s), 7.89 (1H, dd, J=7.6, 7.8 Hz), 7.99-8.05 (2H, m), 8.10 (1H, s), 8.36 (1H, s), 11.90 (1H, br).

### Example 9

### (1) 3-Iodo-2-methyl-N-(1-methylpiperidin-4-yl)benzamide

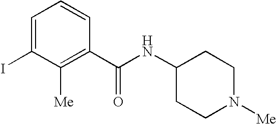

3-Iodo-2-methylbenzoic acid (1.00 g, 3.82 mmol), tetrahydrofuran (3 ml) and N,N-dimethylformamide (3 drops) were mixed, and oxalyl chloride (533 mg, 4.20 mmol) was added dropwise thereto with ice cooling. The mixture was stirred for 1 hour with ice cooling, and the reaction solution was concentrated under reduced pressure. Separately, 4-amino-1-methylpiperidine (479 mg, 4.20 mmol), triethylamine (386 mg, 3.82 mmol) and tetrahydrofuran (4 ml) were mixed, and a solution of the acid chloride prepared above in tetrahydrofuran (4 ml) was added dropwise to the mixture under ice cooling over about 20 minutes. After completion of the dropwise addition, the mixture was stirred at room temperature for 1 hour. 2-Butanone (50 ml) and water (20 ml) were added to the reaction solution. The organic layer was separated and washed with saturated brine (10 ml). The organic layer was concentrated under reduced pressure, ethyl acetate (2 ml) and n-hexane (2 ml) were added to the residue, and the mixture was stirred at room temperature for 30 minutes. The crystals were collected by filtration, and washed with ethyl acetate/n-hexane (1/1, 3 ml). The crystals were dried under reduced pressure at 50° C., to yield the title compound (756 mg) (yield 55.3%).

¹H-NMR (CDCl₃, TMS, 300 MHz) δ (ppm): 1.52-1.62 (2H, m), 2.01-2.06 (2H, m), 2.11-2.20 (2H, m), 2.30 (3H, s), 2.49 (3H, s), 2.79-2.83 (2H, m), 3.93-4.02 (1H, m), 5.65 (1H, d, J=6.9 Hz), 6.89 (1H, t, J=7.5 Hz), 7.25-7.27 (1H, m), 7.87 (1H, dd, J=1.1 Hz, 7.9 Hz).

High resolution mass spectrometry (C₁₄H₁₉IN₂O)

Theoretical value: 358.0542 [M⁺]

Measured value: 358.0533 [M⁺]

Melting point: 205.2 to 206.8° C.

### (2) 3-[(3-Bromo-5-methylpyridin-2-yl)amino]-2-methyl-N-(1-methylpiperidin-4-yl)benzamide

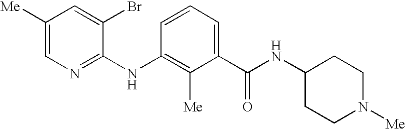

Under a nitrogen atmosphere, palladium acetate (42 mg, 0.19 mmol), 4,5-bis(diphenylphosphino)-9,9-dimethylxanthene (Xantphos) (108 mg, 0.37 mmol) and toluene (7 ml) were mixed, and the mixture was stirred at room temperature for 15 minutes. To this solution, 2-amino-3-bromo-5-methylpyridine (350 mg, 1.87 mmol), 3-iodo-2-methyl-N-(1-methylpiperidin-4-yl)benzamide (670 mg, 1.87 mmol) and cesium carbonate (1.46 g, 4.49 mmol) were added. The mixture was stirred at an internal temperature of 100 to 105° C. for 5 hours. The reaction solution was cooled to room temperature, and water (20 ml) and 2-butanone (50 ml) were added thereto. The organic layer was separated and washed with saturated brine (10 ml). The organic layer was concentrated under reduced pressure, ethyl acetate (7 ml) was added to the concentrate, and the mixture was stirred at room temperature for 30 minutes. The crystals were collected by filtration, and washed with ethyl acetate (2 ml). Methanol (0.5 ml) and ethyl acetate (5 ml) were added to the obtained crude crystals, and the mixture was stirred at room temperature for 30 minutes. The crystals were collected by filtration, and washed with ethyl acetate (2 ml). The crystals were dried under reduced pressure at 50° C., to yield the title compound (295 mg) (yield 37.8%).

¹H-NMR (CDCl₃, TMS, 300 MHz) δ (ppm): 1.49-1.62 (2H, m), 2.05-2.12 (2H, m), 2.16-2.23 (2H, m), 2.23 (3H, s), 2.30 (3H, s), 2.36 (3H, s), 2.78-2.82 (2H, m), 3.95-4.05 (1H, m), 5.69 (1H, d, J=8.0 Hz), 6.75 (1H, s), 7.08 (1H, d, J=6.7 Hz), 7.24 (1H, t, J=7.9 Hz), 7.61 (1H, d, J=1.9 Hz), 7.94 (1H, d, J=1.0 Hz), 8.03 (1H, d, J=7.5 Hz).

High resolution mass spectrometry (C₂₀H₂₅BrN₄O)

Theoretical value: 416.1212 [M⁺]

Measured value: 416.1202 [M⁺]

Melting point: 262.1 to 266.5° C.

### (3) 3,8-dimethyl-N-(1-methylpiperidin-4-yl)-9H-pyrido[2,3-b]indole-7-carboxamide

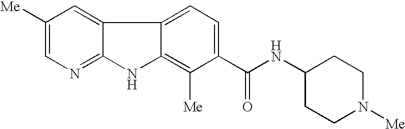

Under a nitrogen atmosphere, palladium acetate (13 mg, 0.06 mmol), 2-(dicyclohexylphosphino)biphenyl (42 mg, 0.12 mmol) and degassed N,N-dimethylacetamide (1 ml) were mixed, and the mixture was stirred at room temperature for 30 minutes. 3-[(3-Bromo-5-methylpyridin-2-yl)amino]-2-methyl-N-(1-methylpiperidin-4-yl)benzamide (250 mg, 0.60 mmol), 1,8-diazabicyclo[5.4.0]-7-undecene (182 mg, 1.20 mmol) and N,N-dimethylacetamide (0.75 ml) were added thereto. The mixture was stirred at 150° C. for 2 hours. After completion of the reaction, the reaction solution was cooled to room temperature, and water (3.5 ml) was added thereto. The mixture was stirred at room temperature for 30 minutes, and the crystals were collected by filtration, and washed once with ethanol/water (1/2, 0.5 ml) and twice with water (1 ml). The crystals were dried under reduced pressure at 50° C., to yield the title compound (130 mg) (yield 64.5%).

¹H-NMR (DMSO-d₆, TMS, 300 MHz) δ (ppm): 1.55-1.63 (2H, m), 1.81-1.85 (2H, m), 1.96-2.03 (2H, m), 2.19 (3H, s), 2.48 (3H, s), 2.57 (3H, s), 2.76-2.80 (2H, m), 3.73-3.82 (1H, m), 7.16 (1H, d, J=8.3 Hz), 7.98 (1H, d, J=8.3 Hz), 8.17 (1H, d, J=7.0 Hz), 8.30 (1H, d, J=6.9 Hz), 11.72 (1H, s).

High resolution mass spectrometry (C₂₀H₂₄N₄O)

Theoretical value: 336.1950 [M⁺]

Measured value: 336.1954 [M⁺]

Melting point: 335.8 to 336.8° C.

### Example 10

### (1) 3-Bromo-N-(5-chloro-2-methoxyphenyl)-5-methylpyridine-2-amine

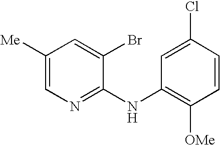

Under a nitrogen atmosphere, tris(dibenzylideneacetone)dipalladium (2.29 g, 2.5 mmol), 1,1′-bis(diphenylphosphino)ferrocene (2.77 g, 5 mmol), sodium tert-butoxide (6.73 g, 70 mmol) and toluene (150 ml) were mixed. To this solution, 2-amino-3-bromo-5-methylpyridine (9.82 g, 52.5 mmol), 4-chloro-2-iodo-1-methoxybenzene (13.42 g, 50 mmol) and toluene (100 ml) were added. The mixture was stirred at an internal temperature of 90° C. for 2 hours. The reaction solution was cooled to room temperature, and 1N hydrochloric acid (150 ml) was added thereto. The insoluble was filtered off and washed twice with toluene (50 ml). The organic layer was separated and washed sequentially, once with 5N aqueous sodium hydroxide solution (50 ml) and twice with water (50 ml). The organic layer was concentrated under reduced pressure, ethanol/acetone (4/1, 30 ml) was added to the concentrate, and the crystals were collected by filtration. The crystals were washed three times with ethanol/acetone (4/1, 15 ml), and dried under reduced pressure at 50° C., to yield the title compound (12.88 g) (yield 78.6%).

¹H-NMR (CDCl₃, TMS, 300 MHz) δ (ppm): 2.23 (3H, s), 3.92 (3H, s), 6.77 (1H, d, J=8.6 Hz), 6.87 (1H, dd, J=2.5 Hz, 8.6 Hz), 7.59 (1H, d, J=1.5 Hz), 7.75 (1H, brs), 8.05 (1H, d, J=1.0 Hz), 8.69 (1H, d, J=2.5 Hz).

¹³C-NMR (CDCl₃, TMS, 75 MHz) δ (ppm): 17.1, 56.2, 106.8, 110.4, 117.1, 120.1, 125.4, 126.0, 131.2, 140.9, 146.0, 146.3, 149.3.

Mass analysis (C₁₃H₁₂N₂OBrCl)

Theoretical value: 326

Measured value: 327 [M+H]⁺

Elemental analysis (C₁₃H₁₂N₂OBrCl)

Theoretical value: C, 47.66; H, 3.69; N, 8.55; Br, 24.39; Cl, 10.82

Measured value: C, 47.94; H, 3.62; N, 8.68; Br, 24.36; Cl, 10.86

Melting point: 140.2° C.

### (2) 5-Chloro-8-methoxy-3-methyl-9H-pyrido[2,3-b]indole

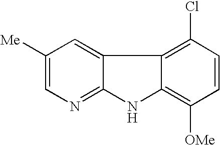

Under a nitrogen atmosphere, palladium acetate (62 mg, 0.27 mmol), 2-(dicyclohexylphosphino)biphenyl (193 mg, 0.55 mmol) and degassed N,N-dimethylacetamide (9 ml) were mixed, and the mixture was stirred at room temperature for 30 minutes. 3-Bromo-N-(5-chloro-2-methoxy phenyl)-5-methylpyridine-2-amine (3.00 g, 9.16 mmol), 1,8-diazabicyclo[5.4.0]-7-undecene (2.79 g, 18.32 mmol) and N,N-dimethylacetamide (3 ml) were added thereto. The mixture was stirred at 150° C. for 3 hours. After completion of the reaction, the reaction solution was cooled to room temperature, and water (18 ml) was added thereto. The mixture was stirred at room temperature for 30 minutes, and the crystals were collected by filtration, and washed twice with ethanol/water (1/1, 6 ml) and twice with water (6 ml). Ethyl acetate (10 ml) was added to the obtained crude crystals, the mixture was stirred for 30 minutes at 50° C. and for another 30 minutes at room temperature, and the crystals were collected by filtration. The crystals were washed twice with ethyl acetate (4 ml), and dried under reduced pressure at 50° C., to yield the title compound (1.25 g) (yield 55.3%).

¹H-NMR (DMSO-d₆, TMS, 300 MHz) δ (ppm): 2.48 (3H, s), 3.98 (3H, s), 7.05 (1H, d, J=8.5 Hz), 7.17 (1H, d, J=8.5 Hz), 8.35 (1H, d, J=1.7 Hz), 8.50 (1H, d, J=1.7 Hz), 12.11 (1H, brs).

¹³C-NMR (DMSO-d₆, TMS, 75 MHz) δ (ppm): 18.2, 56.1, 108.1, 114.1, 117.9, 119.2, 119.6, 124.3, 129.9, 130.2, 144.9, 147.5, 150.1.

Mass analysis (C₁₃H₁₁N₂OCl)

Theoretical value: 246

Measured value: 247 [M+H]⁺

Elemental analysis (C₁₃H₁₁N₂OCl)

Theoretical value: C, 63.29; H, 4.49; N, 11.36; Cl, 14.37

Measured value: C, 63.21; H, 4.26; N, 11.44; Cl, 14.37

Melting point: 300.5° C.

### Example 11

### (1) 3-bromo-N-(2-bromophenyl)-5-methylpyridine-2-amine

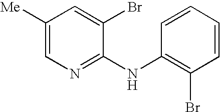

Under a nitrogen atmosphere, palladium acetate (120 mg, 0.53 mmol), 4,5-bis(diphenylphosphino)-9,9-dimethylxanthene (Xantphos) (309 mg, 0.53 mmol) and anisole (30 ml) were mixed, and the mixture was stirred at room temperature for 10 minutes. To this solution, 2-amino-3-bromo-5-methylpyridine (2.00 g, 10.69 mmol), 1-bromo-2-iodobenzene (3.03 g, 10.69 mmol) and cesium carbonate (4.88 g, 14.97 mmol) were added, and the mixture was stirred at room temperature for 15 minutes. The mixture was heated and stirred at 130° C. for 6 hours. After completion of the reaction, the reaction solution was cooled to room temperature, and water (40 ml) was added thereto. The mixture was concentrated under reduced pressure. Ethyl acetate (100 ml) and tetrahydrofuran (20 ml) were added to the concentrate. The organic layer was separated and washed sequentially, once with water (20 ml), twice with 1N hydrochloric acid (50 ml), and once with saturated brine (20 ml). The organic layer was concentrated under reduced pressure. The concentrate was dissolved in tetrahydrofuran (50 ml), silica gel (5 g) was added and the mixture was filtered. The filtrate was concentrated under reduced pressure. Methanol (5 ml) and water (0.5 ml) were added to the residue, and the mixture was stirred at room temperature for 1 hour. Subsequently, the crystals were collected by filtration, and dried under reduced pressure at room temperature, to yield the title compound (2.45 g) (yield 67%).

¹H-NMR (DMSO-d₆, TMS, 300 MHz) δ (ppm): 2.22 (3H, s), 7.00 (1H, td, J=1.6, 7.4 Hz), 7.38 (1H, td, J=1.4, 7.1 Hz), 7.66 (1H, dd, J=1.4, 8.0 Hz), 7.73 (1H, s), 7.90 (1H, s), 8.03 (1H, d, J=1.0 Hz), 8.29 (1H, dd, J=1.4, 8.2 Hz).

High resolution mass spectrometry (C₁₂H₁₀Br₂N₂)

Theoretical value: 339.9211 [M⁺]

Measured value: 339.9211 [M⁺]

Melting point: 54.7 to 56.5° C.

### (2) 8-Bromo-3-methyl-9H-pyrido[2,3-b]indole

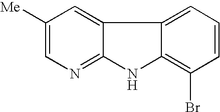

Under a nitrogen atmosphere, palladium acetate (16 mg, 0.07 mmol), 2-(dicyclohexylphosphino)biphenyl (49 mg, 0.14 mmol) and N,N-dimethylacetamide (2.4 ml) were mixed, and the mixture was stirred at room temperature for 10 minutes. 3-Bromo-N-(2-bromophenyl)-5-methylpyridine-2-amine (800 mg, 2.34 mmol) and 1,8-diazabicyclo[5.4.0]-7-undecene (712 mg, 4.45 mmol) were added. The mixture was stirred at 130° C. for 8 hours. Palladium acetate (32 mg, 0.14 mmol) and 2-(dicyclohexylphosphino)biphenyl (98 mg, 0.28 mmol) were added thereto, and the mixture was further stirred at 130° C. for 5 hours. Palladium acetate (53 mg, 0.23 mmol), 2-(dicyclohexylphosphino)biphenyl (164 mg, 0.46 mmol) and 1,8-diazabicyclo[5.4.0]-7-undecene (356 mg, 2.34 mmol) were added, and the mixture was further stirred at 130° C. for 9 hours. After completion of the reaction, the reaction solution was cooled to room temperature. Water (4 ml), ethyl acetate (16 ml) and tetrahydrofuran (4 ml) were added to the reaction solution. The organic layer was separated, washed with water (4 ml) and concentrated. Ethyl acetate (4 ml) was added to the residue. The mixture was stirred at room temperature for 30 minutes. The crystals were collected by filtration. The crystals were dried under reduced pressure at 50° C., to yield the title compound (90 mg) (yield 14.7%).

¹H-NMR (DMSO-d₆, TMS, 300 MHz) δ (ppm): 2.48 (3H, s), 7.17 (1H, t, J=7.8 Hz), 7.67 (1H, dd, J=0.7, 7.8 Hz), 8.17 (1H, dd, J=0.7, 7.7 Hz), 8.37 (1H, s), 8.38 (1H, s), 11.93 (1H, br).

High resolution mass spectrometry (C₁₂H₉BrN₂)

Theoretical value: 259.9949 [M⁺]

Measured value: 259.9940 [M⁺]

Melting point: 389.0° C. (dec.)

### Example 12

### (1) 3-Bromo-5-methyl-N-1-naphthylpyridine-2-amine

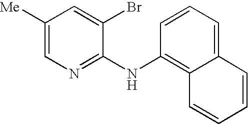

Under a nitrogen atmosphere, palladium acetate (120 mg, 0.53 mmol), 4,5-bis(diphenylphosphino)-9,9-dimethylxanthene (Xantphos) (309 mg, 0.53 mmol) and anisole (30 ml) were mixed, and the mixture was stirred at room temperature for 10 minutes. To this solution, 2-amino-3-bromo-5-methylpyridine (2.00 g, 10.69 mmol), 1-iodonaphthalene (2.72 g, 10.69 mmol) and cesium carbonate (4.88 g, 14.97 mmol) were added. The mixture was stirred at room temperature for 15 minutes. The mixture was heated and stirred at 130° C. for 8 hours. After completion of the reaction, the reaction solution was cooled to room temperature, and water (40 ml) was added thereto. The mixture was concentrated under reduced pressure. Ethyl acetate (100 ml) was added to the concentrate. The organic layer was separated and washed with water (20 ml). The organic layer was concentrated under reduced pressure. Methanol (20 ml) was added to the residue, and the mixture was stirred at room temperature for 30 minutes. The crystals were collected by filtration, and washed with methanol (2 ml). The crystals were dried under reduced pressure, to yield the title compound (2.10 g) (yield 63%).

¹H-NMR (DMSO-d₆, TMS, 300 MHz) δ (ppm): 2.16 (3H, s), 7.46-7.53 (3H, m), 7.58-7.60 (1H, m), 7.73 (1H, s), 7.76-7.78 (1H, m), 7.83-7.86 (2H, m), 7.92-7.96 (1H, m), 8.16 (1H, s).

High resolution mass spectrometry (C₁₆H₁₃BrN₂)

Theoretical value: 311.0184 [M−H]⁺

Measured value: 311.0179 [M−H]⁺

Melting point: 127.7 to 132.6° C.

### (2) 8-Methyl-11H-benzo[g]pyrido[2,3-b]indole

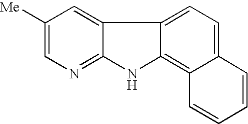

Under a nitrogen atmosphere, palladium acetate (11 mg, 0.05 mmol), 2-(dicyclohexylphosphino)biphenyl (34 mg, 0.10 mmol) and N,N-dimethylacetamide (1.5 ml) were mixed, and the mixture was stirred at room temperature for 10 minutes. 3-Bromo-5-methyl-N-1-naphthylpyridine-2-amine (500 mg, 1.60 mmol) and 1,8-diazabicyclo[5.4.0]-7-undecene (487 mg, 3.19 mmol) were added thereto. The mixture was stirred at 130° C. for 3.5 hours. After completion of the reaction, the reaction solution was cooled to room temperature, and water (3 ml) was added thereto. The mixture was stirred at room temperature for 30 minutes, and the crystals were collected by filtration and washed twice with methanol/water (1/1, 1 ml) and once with water (1 ml). The crystals were dried under reduced pressure at 50° C., to yield the title compound (322 mg) (yield 86.8%).

¹H-NMR (DMSO-d₆, TMS, 300 MHz) δ (ppm): 2.52 (3H, s), 7.57-7.71 (3H, m), 8.06 (1H, d, J=7.6 Hz), 8.21 (1H, d, J=8.6 Hz), 8.35 (1H, d, J=2.0 Hz), 8.40 (1H, d, J=1.3 Hz), 8.61 (1H, d, J=8.1 Hz), 12.64 (1H, br).

High resolution mass spectrometry (C₁₆H₁₂N₂)

Theoretical value: 232.1000 [M⁺]

Measured value: 232.0996 [M⁺]

Melting point: 301.1 to 303.5° C.

### Example 13

### (1) 3-Bromo-5-methyl-N-[4-(methylthio)phenyl]pyridine-2-amine

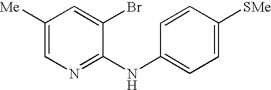

Under a nitrogen atmosphere, palladium acetate (90 mg, 0.40 mmol), 4,5-bis(diphenylphosphino)-9,9-dimethylxanthene (Xantphos) (348 mg, 0.60 mmol), and anisole (25 ml) were mixed, and the mixture was stirred at room temperature for 10 minutes. To this solution, 2-amino-3-bromo-5-methylpyridine (1.50 g, 8.02 mmol), 4-iodothioanisole (2.01 g, 8.02 mmol) and cesium carbonate (3.66 g, 11.22 mmol) were added and the mixture was stirred at room temperature for 10 minutes. The mixture was heated and stirred at 130° C. for 4 hours. Palladium acetate (90 mg, 0.40 mmol), 4,5-bis(diphenylphosphino)-9,9-dimethylxanthene (Xantphos) (348 mg, 0.60 mmol) and cesium carbonate (2.61 g, 8.02 mmol) were added, and the mixture was further stirred at 130° C. for 6 hours. The reaction solution was cooled to room temperature, and water (45 ml) was added thereto, and the mixture was concentrated under reduced pressure. Ethyl acetate (70 ml) and tetrahydrofuran (15 ml) were added to the concentrate. The organic layer was separated and washed sequentially, with 1N hydrochloric acid (15 ml), with a saturated aqueous sodium hydrogencarbonate solution (15 ml) and with saturated brine (15 ml). The organic layer was concentrated under reduced pressure. The concentrate was subjected to silica gel column chromatography (silica gel 25 g, eluent: ethyl acetate:n-hexane=1:20), and the effective fraction was concentrated. Methanol (1.5 ml) was added to the residue, and the crystals were collected by filtration. The crystals were dried under reduced pressure at room temperature, to yield the title compound (500 mg) (yield 20%).

¹H-NMR (DMSO-d₆, TMS, 300 MHz) δ (ppm): 2.20 (3H, s), 2.45 (3H, s), 7.20 (1H, s), 7.24 (1H, d, J=2.7 Hz), 7.57 (1H, s), 7.60 (1H, d, J=1.5 Hz), 7.81 (1H, s), 7.97-8.00 (2H, s).

High resolution mass spectrometry (C₁₃H₁₃BrN₂S)

Theoretical value: 306.9905 [M−H]⁺

Measured value: 306.9904 [M−H]⁺

Melting point: 54.5 to 57.3° C.

### (2) 3-Methyl-6-(methylthio)-9H-pyrido[2,3-b]indole

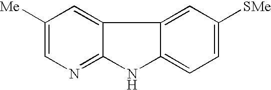

Under a nitrogen atmosphere, palladium acetate (9 mg, 0.04 mmol), 2-(dicyclohexylphosphino)biphenyl (27 mg, 0.08 mmol) and N,N-dimethylacetamide (1.2 ml) were mixed, and the mixture was stirred at room temperature for 10 minutes. 3-Bromo-5-methyl-N-[4-(methylthio)phenyl]pyridine-2-amine (400 mg, 1.29 mmol) and 1,8-diazabicyclo[5.4.0]-7-undecene (394 mg, 2.59 mmol) were added thereto. The mixture was stirred at 130° C. for 6 hours. After completion of the reaction, the reaction solution was cooled to room temperature, and water (2.4 ml) was added thereto. The mixture was stirred at room temperature for 1 hour, the crystals were collected by filtration, and washed with methanol (1 ml) and twice with water (1 ml). The crystals were dried under reduced pressure at 50° C., to yield the title compound (253 mg) (yield 85.5%).

¹H-NMR (DMSO-d₆, TMS, 300 MHz) δ (ppm): 2.46 (3H, s), 2.55 (3H, s), 7.44-7.45 (2H, m), 8.15 (1H, s), 8.30 (1H, s), 8.45 (1H, d, J=1.2 Hz), 11.83 (1H, s).

High resolution mass spectrometry (C₁₃H₁₂N₂S)

Theoretical value: 288.0721 [M⁺]

Measured value: 288.0711 [M⁺]

Melting point: 155° C. (dec.)

### Example 14

### (1) 4-[(3-bromo-5-methylpyridin-2-yl)amino]benzaldehyde

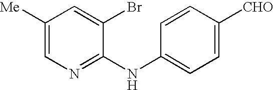

Under a nitrogen atmosphere, palladium acetate (48 mg, 0.21 mmol), 4,5-bis(diphenylphosphino)-9,9-dimethylxanthene (Xantphos) (186 mg, 0.32 mmol) and anisole (12 ml) were mixed, and the mixture was stirred at room temperature for 10 minutes. To this solution, 2-amino-3-bromo-5-methylpyridine (800 mg, 4.28 mmol), p-iodobenzaldehyde (992 mg, 4.28 mmol), and cesium carbonate (1.95 g, 5.99 mmol) were added. The mixture was stirred at room temperature for 10 minutes. The mixture was heated and stirred at 130° C. for 7 hours. After completion of the reaction, the reaction solution was cooled to room temperature, and water (15 ml) was added thereto. The mixture was concentrated under reduced pressure. Ethyl acetate (80 ml) and water (20 ml) were added to the concentrate. The organic layer was separated and washed with saturated brine (15 ml). Activated carbon Shirasagi A was added to the organic layer, which was then filtered. The filtrate was concentrated under reduced pressure. The concentrate was subjected to plate silica gel chromatography (eluent:ethyl acetate/n-hexane=1/3), and the effective fraction was concentrated. Methanol (0.5 ml) was added to the residue. The crystals were collected by filtration and dried under reduced pressure at room temperature, to yield the title compound (280 mg) (yield 23%).

¹H-NMR (DMSO-d₆, TMS, 300 MHz) δ (ppm): 2.26 (3H, s), 7.80 (4H, s), 7.95 (1H, d, J=1.5 Hz), 8.14 (1H, s), 8.68 (1H, s), 9.83 (1H, s).

High resolution mass spectrometry (C₁₃H₁₁Br₂N₂O)

Theoretical value: 288.9977 [M−H]⁺

Measured value: 288.9977 [M−H]⁺

Melting point: 94.7 to 96.2° C.

### (2) 3-Methyl-9H-pyrido[2,3-b]indole-6-carbaldehyde

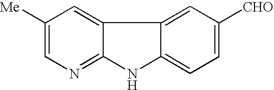

Under a nitrogen atmosphere, palladium acetate (4 mg, 0.02 mmol), 2-(dicyclohexylphosphino)biphenyl (11 mg, 0.03 mmol) and N,N-dimethylacetamide (0.45 ml) were mixed, and the mixture was stirred at room temperature for 10 minutes. 4-[(3-Bromo-5-methylpyridin-2-yl)amino]benzaldehyde (150 mg, 0.52 mmol), 1,8-diazabicyclo[5.4.0]-7-undecene (157 mg, 1.03 mmol), and N,N-dimethylacetamide (0.3 ml) were added. The mixture was stirred at 130° C. for 4 hours. After completion of the reaction, the reaction solution was cooled to room temperature, and water (2 ml) and methanol (1.5 ml) were added. The mixture was stirred at room temperature for 30 minutes, and the crystals were collected by filtration and washed twice with methanol/water (1/1, 0.5 ml) and once with water (1 ml). The obtained crude crystals were suspended in methanol (0.3 ml), and the crystals were collected by filtration. The crystals were dried under reduced pressure at 50° C., to yield the title compound (12 mg) (yield 11.1%).

¹H-NMR (DMSO-d₆, TMS, 300 MHz) δ (ppm): 2.48 (3H, s), 7.62 (1H, d, J=8.4 Hz), 7.98 (1H, dd, J=1.5, 8.5 Hz), 8.36 (1H, d, J=1.6 Hz), 8.47 (1H, d, J=1.2 Hz), 8.73 (1H, d, J=1.0 Hz), 10.05 (1H, s), 12.22 (1H, s).

High resolution mass spectrometry (C₁₃H₁₀N₂O)

Theoretical value: 210.0793 [M⁺]

Measured value: 210.0780 [M⁺]

Melting point: 279.2 to 282.2° C.

### Example 15

### (1) 1-{4-[(3-Bromo-5-methylpyridin-2-yl)amino]phenyl}ethanone

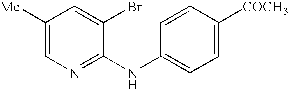

Process using Pd Catalyst

Under a nitrogen atmosphere, palladium acetate (120 mg, 0.53 mmol), 4,5-bis(diphenylphosphino)-9,9-dimethylxanthene (Xantphos) (464 mg, 0.80 mmol), and anisole (30 ml) were mixed, and the mixture was stirred at room temperature for 10 minutes. To this solution, 2-amino-3-bromo-5-methylpyridine (2.00 g, 10.69 mmol), 4′-iodoacetophenone (2.63 g, 10.69 mmol) and cesium carbonate (4.88 g, 14.97 mmol) were added, and the mixture was stirred at room temperature for 10 minutes. The mixture was heated and stirred at 130° C. for 6 hours. After completion of the reaction, the reaction solution was cooled to room temperature, and water (35 ml) was added thereto. The mixture was concentrated under reduced pressure. Ethyl acetate (100 ml) was added to the concentrate. The organic layer was separated and washed sequentially, with 1N hydrochloric acid (15 ml), with a saturated aqueous sodium hydrogencarbonate solution (15 ml), and with saturated brine (15 ml). The organic layer was concentrated under reduced pressure. The residue was subjected to silica gel column chromatography (silica gel 20 g, eluent: ethyl acetate/n-hexane=1/15-1/8), and the effective fraction was concentrated. Methanol (6 ml) was added to the residue, and the crystals were collected by filtration. The crystals were dried at room temperature under reduced pressure, to yield the title compound (1.46 g) (yield 45%).

¹H-NMR (DMSO-d₆, TMS, 300 MHz) δ (ppm): 2.23 (3H, s), 2.51 (3H, s), 7.71 (1H, s), 7.74 (1H, s), 7.86 (1H, s), 7.89-7.90 (2H, m), 8.09 (1H, s), 8.50 (1H, s).

High resolution mass spectrometry (C₁₄H₁₃BrN₂O)

Theoretical value: 303.0133 [M−H]⁺

Measured value: 303.0132 [M−H]⁺

Melting point: 91.9 to 93.0° C.

### (2) 1-(3-methyl-9H-pyrido[2,3-b]indol-6-yl)ethanone

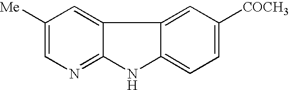

Under a nitrogen atmosphere, palladium acetate (22 mg, 0.10 mmol), 2-(dicyclohexylphosphino)biphenyl (69 mg, 0.20 mmol) and N,N-dimethylacetamide (3 ml) were mixed, and the mixture was stirred at room temperature for 10 minutes. 1-{4-[(3-Bromo-5-methylpyridin-2-yl)amino]phenyl}ethanone (1.00 g, 3.28 mmol) and 1,8-diazabicyclo[5.4.0]-7-undecene (998 mg, 6.55 mmol) were added thereto. The mixture was stirred at 130° C. for 3 hours. After completion of the reaction, the reaction solution was cooled to room temperature, and water (6 ml) was added thereto. The mixture was stirred at room temperature for 30 minutes, and the crystals were collected by filtration and washed once with methanol/water (1/2, 2 ml) and twice with water (2 ml). The obtained crude crystals were suspended in methanol (8 ml), and the crystals were collected by filtration and dried under reduced pressure at 50° C., to yield the title compound (620 mg) (yield 84.4%).

¹H-NMR (DMSO-d₆, TMS, 300 MHz) δ (ppm): 2.49 (3H, s), 2.68 (3H, s), 7.56 (1H, d, J=8.6 Hz), 8.08 (1H, dd, J=1.7, 8.6 Hz), 8.35 (1H, d, J=1.6 Hz), 8.52 (1H, d, J=1.4 Hz), 8.87 (1H, d, J=1.4 Hz), 12.14 (1H, s).

High resolution mass spectrometry (C₁₄H₁₂N₂O)

Theoretical value: 224.0950 [M⁺]

Measured value: 224.0948 [M⁺]

Melting point: 287.6 to 290.6° C.

### (3) 1-{4-[(3-Bromo-5-methylpyridin-2-yl)amino]phenyl}ethanone

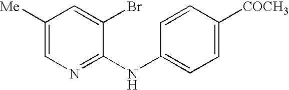

Process using Cu Catalyst

Under a nitrogen atmosphere, 2-amino-3-bromo-5-methylpyridine (1.50 g, 8.02 mmol), 4′-iodoacetophenone (1.97 g, 8.02 mmol), copper (I) iodide (153 mg, 0.80 mmol), ethanol amine (98 mg, 1.60 mmol), potassium carbonate (1.66 g, 12.03 mmol) and anisole (22.5 ml) were mixed, and the mixture was stirred at room temperature for 10 minutes. The mixture was heated and stirred at 130° C. for 8 hours. The reaction solution was cooled to room temperature, and water (22.5 ml) was added thereto. The mixture was concentrated under reduced pressure. To the residue, ethyl acetate (30 ml), water (10 ml) and activated carbon Shirasagi A were added, and the mixture was filtered. The organic layer was separated and washed with water (10 ml). The organic layer was concentrated. The concentrate was subjected to silica gel column chromatography (silica gel 20 g, eluent: ethyl acetate/n-hexane=1/20), and the effective fraction was concentrated. Methanol (2 ml) was added to the residue, and the mixture was stirred at room temperature for 30 minutes. Next, the crystals were collected by filtration, and dried at 40° C. under reduced pressure, to yield the title compound (525 mg). (HPLC area %: title compound: 82.1%, 1-{4-[(3-iodo-5-methylpyridin-2-yl)amino]phenyl}ethanone: 17.2%).

The spectral data was confirmed to be the same as for the title compound obtained in the above (1).

### (4) 1-(3-Methyl-9H-pyrido[2,3-b]indol-6-yl)ethanone

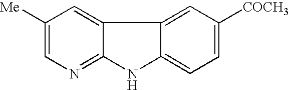

Under a nitrogen atmosphere, palladium acetate (9 mg, 0.04 mmol), 2-(dicyclohexylphosphino)biphenyl (28 mg, 0.08 mmol) and N,N-dimethylacetamide (1.2 ml) were mixed, and the mixture was stirred at room temperature for 10 minutes. 1-{4-[(3-Bromo-5-methylpyridin-2-yl)amino]phenyl}ethanone (400 mg, 1.31 mmol) and 1,8-diazabicyclo[5.4.0]-7-undecene (399 mg, 2.62 mmol) were added to the mixture. The mixture was stirred at 130° C. for 3 hours. After completion of the reaction, the reaction solution was cooled to room temperature, and water (2.4 ml) was added thereto. The mixture was stirred at room temperature for 30 minutes, and the crystals were collected by filtration, and washed twice with methanol/water (1/1, 1 ml) and once with water (1 ml). The crystals were dried under reduced pressure at 50° C., to yield the title compound (201 mg) (yield after two processes starting from 2-amino-3-bromo-5-methylpyridine: 14.7%).

The spectral data was confirmed to be the same as for the title compound obtained in the above (2).

### Example 16

### (1) 4-[(3-Bromo-5-methylpyridin-2-yl)amino]benzonitrile

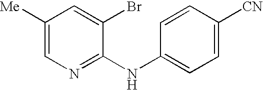

Under a nitrogen atmosphere, palladium acetate (120 mg, 0.53 mmol), 4,5-bis(diphenylphosphino)-9,9-dimethylxanthene (Xantphos) (464 mg, 0.80 mmol) and anisole (30 ml) were mixed, and the mixture was stirred at room temperature for 10 minutes. To this solution, 2-amino-3-bromo-5-methylpyridine (2.00 g, 10.69 mmol), 4-iodobenzonitrile (2.45 g, 10.69 mmol) and cesium carbonate (4.88 g, 14.97 mmol) were added. The mixture was stirred at room temperature for 10 minutes. The mixture was heated and stirred at 130° C. for 13 hours. After completion of the reaction, the reaction solution was cooled to room temperature, water (50 ml) was added thereto, and the mixture was concentrated under reduced pressure. Methanol (40 ml) was added to the concentrate, and the mixture was stirred at room temperature for 30 minutes. The crystals were collected by filtration. The obtained crude crystals were suspended in ethyl acetate (3 ml) at room temperature for 30 minutes, and the crystals were collected by filtration. The crystals were dried under reduced pressure at room temperature, to yield the title compound (2.10 g) (yield 68%).

¹H-NMR (DMSO-d₆, TMS, 300 MHz) δ (ppm): 2.24 (3H, s), 7.67 (2H, dd, J=1.9, 7.0 Hz), 7.78 (2H, dd, J=1.9, 7.0 Hz), 7.92 (1H, d, J=1.5 Hz), 8.10 (1H, d, J=1.2 Hz), 8.64 (1H, br).

High resolution mass spectrometry (C₁₃H₁₀BrN₃)

Theoretical value: 285.9980 [M−H]⁺

Measured value: 285.9972 [M−H]⁺

Melting point: 163.4 to 165.3° C.

### (2) 3-Methyl-9H-pyrido[2,3-b]indole-6-carbonitrile

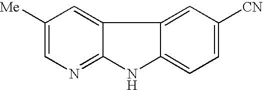

Under a nitrogen atmosphere, palladium acetate (23 mg, 0.10 mmol), 2-(dicyclohexylphosphino)biphenyl (73 mg, 0.21 mmol) and N,N-dimethylacetamide (3 ml) were mixed, and the mixture was stirred at room temperature for 10 minutes. 4-[(3-Bromo-5-methylpyridin-2-yl)amino]benzonitrile (1.00 g, 3.47 mmol) and 1,8-diazabicyclo[5.4.0]-7-undecene (1.06 g, 6.94 mmol) were added thereto. The mixture was stirred at 130° C. for 5 hours. After completion of the reaction, the reaction solution was cooled to room temperature, and water (6 ml) was added thereto. The mixture was stirred at room temperature for 30 minutes, and the crystals were collected by filtration, and washed twice with methanol/water (1/2, 2 ml) and once with water (2 ml). The crystals were dried under reduced pressure at 50° C., to yield the title compound (710 mg) (yield 98.7%).

¹H-NMR (DMSO-d₆, TMS, 300 MHz) δ (ppm): 2.49 (3H, s), 7.64 (1H, d, J=8.5 Hz), 7.82 (1H, dd, J=1.5, 8.5 Hz), 8.40 (1H, s), 8.46 (1H, s), 8.71 (1H, s), 12.27 (1H, s).

High resolution mass spectrometry (C₁₃H₉N₃)

Theoretical value: 207.0796 [M⁺]

Measured value: 207.0789 [M⁺]

Melting point: 285.6 to 288.6° C.

### Example 17

### (1) N-biphenyl-4-yl-3-bromo-5-methylpyridine-2-amine

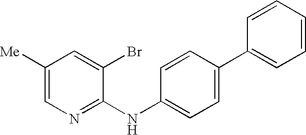

Under a nitrogen atmosphere, palladium acetate (120 mg, 0.53 mmol), 4,5-bis(diphenylphosphino)-9,9-dimethylxanthene (Xantphos) (464 mg, 0.80 mmol) and anisole (30 ml) were mixed, and the mixture was stirred at room temperature for 10 minutes. To this solution, 2-amino-3-bromo-5-methylpyridine (2.00 g, 10.69 mmol), 4-iodobiphenyl (3.00 g, 10.69 mmol) and cesium carbonate (4.88 g, 14.97 mmol) were added. The mixture was stirred at room temperature for 10 minutes. The mixture was heated and stirred at 130° C. for 9 hours. After completion of the reaction, the reaction solution was cooled to room temperature, and water (30 ml) was added thereto. The mixture was concentrated under reduced pressure. Ethyl acetate (100 ml) and water (30 ml) were added to the concentrate. The organic layer was separated and washed with saturated brine (15 ml). The organic layer was concentrated under reduced pressure. The residue was subjected to silica gel column chromatography (silica gel 20 g, eluent: ethyl acetate/n-hexane=1/15), and the effective fraction was concentrated. The concentrate was subjected to the same type of column chromatography again, and the effective fraction was concentrated. Methanol (4 ml) was added to the residue, and the crystals were collected by filtration, and dried at room temperature under reduced pressure, to yield the title compound (860 mg) (yield 24%).

¹H-NMR (DMSO-d₆, TMS, 300 MHz) δ (ppm): 2.22 (3H, s), 7.29-7.34 (1H, m), 7.42 (1H, s), 7.45 (1H, d, J=6.5 Hz), 7.58 (1H, s), 7.61 (1H, s), 7.64 (1H, s), 7.67 (1H, s), 7.71 (1H, s), 7.74 (1H, s), 7.85 (1H, d, J=1.4 Hz), 8.02 (1H, s), 8.10 (1H, s).

High resolution mass spectrometry (C₁₈H₁₅BrN₂)

Theoretical value: 337.0340 [M−H]⁺

Measured value: 337.0341 [M−H]⁺

Melting point: 90.0 to 91.3° C.

### (2) 3-Methyl-6-phenyl-9H-pyrido[2,3-b]indole

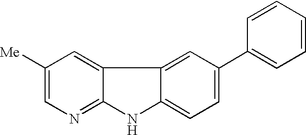

Under a nitrogen atmosphere, palladium acetate (10 mg, 0.04 mmol), 2-(dicyclohexylphosphino)biphenyl (31 mg, 0.09 mmol) and N,N-dimethylacetamide (1.5 ml) were mixed, and the mixture was stirred at room temperature for 10 minutes. N-biphenyl-4-yl-3-bromo-5-methylpyridine-2-amine (500 mg, 1.47 mmol) and 1,8-diazabicyclo[5.4.0]-7-undecene (449 mg, 2.95 mmol) were added to the mixture. The mixture was stirred at 130° C. for 15 hours. After completion of the reaction, the reaction solution was cooled to room temperature, and water (3 ml) was added thereto. The mixture was stirred at room temperature for 30 minutes, and the crystals were collected by filtration, and washed twice with methanol/water (1/1, 1 ml) and once with water (1 ml). The crystals were dried under reduced pressure at 50° C., to yield the title compound (375 mg) (yield 98.5%).

¹H-NMR (DMSO-d₆, TMS, 300 MHz) δ (ppm): 2.49 (3H, s), 7.36 (1H, t, J=7.4 Hz), 7.48-7.53 (2H, m), 7.58 (1H, d, J=8.5 Hz), 7.76-7.80 (3H, m), 8.32 (1H, s), 8.47-8.49 (2H, m), 11.79 (1H, s).

High resolution mass spectrometry (C₁₈H₁₄N₂)

Theoretical value: 258.1157 [M⁺]

Measured value: 258.1157 [M⁺]

Melting point: 300.1 to 302.8° C.

### Example 18

### (1) 3-Bromo-N-(2,4-dimethoxyphenyl)-5-methylpyridine-2-amine

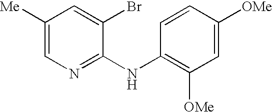

Under a nitrogen atmosphere, 2-amino-3-bromo-5-methylpyridine (2.00 g, 10.69 mmol), palladium chloride (19 mg, 0.11 mmol), 4,5-bis(diphenylphosphino)-9,9-dimethylxanthene (Xantphos) (93 mg, 0.16 mmol) and toluene (30 ml) were mixed, and the mixture was stirred at room temperature for 15 minutes. To this mixture, 2,4-dimethoxyiodobenzene (2.82 g, 10.69 mmol) and sodium tert-butoxide (1.44 g, 14.97 mmol) were added. The mixture was stirred at room temperature for 10 minutes. The mixture was heated and stirred at 100° C. for 7 hours. After completion of the reaction, the reaction solution was cooled to 60° C., water (10 ml) was added thereto. The organic layer was separated and washed with water (10 ml). The organic layer was concentrated under reduced pressure. Methanol (6 ml) and water (2 ml) were added to the residue, and the mixture was stirred at room temperature for 30 minutes. The crystals were collected by filtration, and dried under reduced pressure at 40° C., to yield the title compound (2.75 g) (yield 79.6%).

¹H-NMR (DMSO-d₆, TMS, 300 MHz) δ (ppm): 2.18 (3H, s), 3.75 (3H, s), 3.86 (3H, s), 6.52 (1H, dd, J=2.6, 8.8 Hz), 6.66 (1H, d, J=2.6 Hz), 7.42 (1H, s), 7.79 (1H, d, J=1.5 Hz), 7.96 (1H, s), 8.13 (1H, d, J=8.8 Hz).

High resolution mass spectrometry (C₁₄H₁₅BrN₂O₂)

Theoretical value: 322.0317 [M⁺]

Measured value: 322.0319 [M⁺]

Melting point: 77.6 to 80.6° C.

### (2) 6,7-Dimethoxy-3-methyl-9H-pyrido[2,3-b]indole

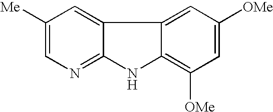

Under a nitrogen atmosphere, palladium acetate (21 mg, 0.09 mmol), 2-(dicyclohexylphosphino)biphenyl (65 mg, 0.19 mmol) and N,N-dimethylacetamide (3 ml) were mixed, and the mixture was stirred at room temperature for 10 minutes. 3-Bromo-N-(2,4-dimethoxyphenyl)-5-methylpyridine-2-amine (1.00 g, 3.09 mmol) and 1,8-diazabicyclo[5.4.0]-7-undecene (942 mg, 6.19 mmol) were added to the reaction solution. The mixture was stirred at 130° C. for 9 hours. Palladium acetate (63 mg, 0.28 mmol) and 2-(dicyclohexylphosphino)biphenyl (195 mg, 0.56 mmol) were added thereto, and the mixture was further stirred at 130° C. for 7 hours. The reaction solution was cooled to room temperature, and water (6 ml) was added thereto. The mixture was stirred at room temperature for 30 minutes, and the crystals were collected by filtration, and washed twice with methanol (2 ml) and once with water (2 ml). The crystals were dried under reduced pressure at 50° C., to yield the title compound (418 mg) (yield 55.8%).

¹H-NMR (DMSO-d₆, TMS, 300 MHz) δ (ppm): 2.45 (3H, s), 3.85 (3H, s), 3.96 (3H, s), 6.71 (1H, d, J=1.7 Hz), 7.28 (1H, d, J=1.7 Hz), 8.26 (1H, s), 8.32 (1H, s), 11.67 (1H, s).

High resolution mass spectrometry (C₁₄H₁₄N₂O₂)

Theoretical value: 242.1055 [M⁺]

Measured value: 242.1053 [M⁺]

Melting point: 214.0 to 217.0° C.

### Example 19

### (1) N-biphenyl-2-yl-3-bromo-5-methylpyridine-2-amine

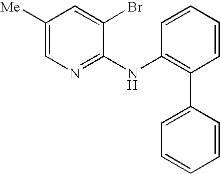

Under a nitrogen atmosphere, 2-amino-3-bromo-5-methylpyridine (2.00 g, 10.69 mmol), palladium chloride (19 mg, 0.11 mmol), 4,5-bis(diphenylphosphino)-9,9-dimethylxanthene (Xantphos) (93 mg, 0.16 mmol) and toluene (30 ml) were mixed, and the mixture was stirred at room temperature for 15 minutes. To this mixture, 2-iodobiphenyl (3.00 g, 10.69 mmol) and sodium tert-butoxide (1.44 g, 14.97 mmol) were added. The mixture was stirred at room temperature for 10 minutes. The mixture was heated and stirred at 100° C. for 7.5 hours. After completion of the reaction, the reaction solution was cooled to 60° C., and water (10 ml) was added thereto. The organic layer was separated and washed with water (10 ml). The organic layer was concentrated under reduced pressure. Methanol (4 ml) was added to the residue, and the mixture was stirred at room temperature for 30 minutes. The crystals were collected by filtration, and dried under reduced pressure at 40° C., to yield the title compound (2.93 g) (yield 81%).

¹H-NMR (DMSO-d₆, TMS, 300 MHz) δ (ppm): 2.18 (3H, s), 7.15 (1H, td, J=1.0, 14.8 Hz), 7.27 (1H, dd, J=1.6, 7.6 Hz), 7.37-7.50 (7H, m), 7.72-7.73 (1H, m), 7.92 (1H, s), 8.15 (1H, d, J=8.1 Hz).

High resolution mass spectrometry (C₁₈H₁₅BrN₂)

Theoretical value: 337.0340 [M−H]⁺

Measured value: 337.0337 [M−H]⁺

Melting point: 97.0 to 99.1° C.

### (2) 3-Methyl-8-phyenyl-9H-pyrido[2,3-b]indole

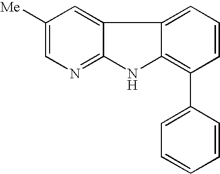

Under a nitrogen atmosphere, palladium acetate (10 mg, 0.04 mmol), 2-(dicyclohexylphosphino)biphenyl (31 mg, 0.09 mmol) and N,N-dimethylacetamide (1.5 ml) were mixed, and the mixture was stirred at room temperature for 10 minutes. N-biphenyl-2-yl-3-bromo-5-methylpyridine-2-amine (500 mg, 1.47 mmol) and 1,8-diazabicyclo[5.4.0]-7-undecene (449 mg, 2.95 mmol) were added thereto. The mixture was stirred at 130° C. for 2 hours. After completion of the reaction, the reaction solution was cooled to room temperature, and water (3 ml) was added thereto. The mixture was stirred at room temperature for 30 minutes. The crystals were collected by filtration, washed twice with methanol/water (1/1, 1 ml) and once with water (1 ml), and dried under reduced pressure at 50° C., to yield the title compound (370 mg) (yield 97.2%).

¹H-NMR (DMSO-d₆, TMS, 300 MHz) δ (ppm): 2.49 (3H, s), 7.32 (1H, t, J=7.6 Hz), 7.45-7.47 (2H, m), 7.53-7.58 (2H, m), 7.72 (1H, s), 7.73 (1H, d, J=1.3 Hz), 8.15 (1H, d, J=7.6 Hz), 8.29 (1H, d, J=1.6 Hz), 8.38 (1H, s), 11.52 (1H, s).

High resolution mass spectrometry (C₁₈H₁₄N₂)

Theoretical value: 258.1157 [M⁺]

Measured value: 258.1154 [M⁺]

Melting point: 229.2 to 231.2° C.

### Example 20

### (1) 3-Bromo-N-(2-methoxyphenyl)-5-methylpyridine-2-amine

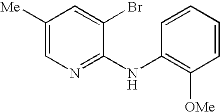

Process using Pd Catalyst

Under a nitrogen atmosphere, 2-amino-3-bromo-5-methylpyridine (10.00 g, 53.46 mmol), palladium chloride (47 mg, 0.27 mmol), 4,5-bis(diphenylphosphino)-9,9-dimethylxanthene (Xantphos) (232 mg, 0.40 mmol) and toluene (150 ml) were mixed, and the mixture was stirred at room temperature for 15 minutes. To this solution, 2-iodoanisole (12.51 g, 53.46 mmol) and sodium tert-butoxide (7.19 g, 74.84 mmol) were added. The mixture was stirred at room temperature for 10 minutes. The mixture was heated and stirred at 100° C. for 9 hours. After completion of the reaction, the reaction solution was cooled to 60° C., and water (50 ml) was added thereto. The mixture was stirred for 10 minutes. The organic layer was collected by separation, and washed with water (50 ml). The organic layer was concentrated under reduced pressure. Methanol (30 ml) and water (15 ml) were added to the concentrate, and the mixture was stirred at room temperature for 1 hour. The crystals were collected by filtration, and dried under reduced pressure at 50° C., to yield the title compound (14.28 g) (yield 91.1%).

¹H-NMR (DMSO-d₆, TMS, 300 MHz) δ (ppm): 2.21 (3H, s), 3.92 (3H, s), 6.92-7.00 (2H,m), 7.04-7.08 (1H, m), 7.72 (1H, s), 7.86 (1H, d, J=1.5 Hz), 8.05 (1H, d, J=1.0 Hz), 8.42-8.46 (1H, m).

High resolution mass spectrometry (C₁₃H₁₃BrN₂O)

Theoretical value: 292.0211 [M⁺]

Measured value: 292.0205 [M⁺]

Melting point: 111.8 to 114.4° C.

### (2) 8-Methoxy-3-methyl-9H-pyrido[2,3-b]indole

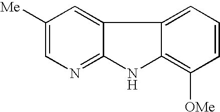

Under a nitrogen atmosphere, palladium acetate (735 mg, 3.27 mmol), 2-(dicyclohexylphosphino)biphenyl (2.30 g, 6.55 mmol) and N,N-dimethylacetamide (160 ml) were mixed, and the mixture was stirred at room temperature for 10 minutes. 3-Bromo-N-(2-methoxyphenyl)-5-methylpyridine-2-amine (32.00 g, 109.16 mmol) and 1,8-diazabicyclo[5.4.0]-7-undecene (33.24 g, 218.31 mmol) were added to the reaction solution. The mixture was stirred at 130° C. for 2 hours. After completion of the reaction, the reaction solution was cooled to 40° C., and water (320 ml) was added thereto. The mixture was stirred at room temperature for 1 hour, and the crystals were collected by filtration, and washed twice with methanol/water (1/2, 48 ml) and once with water (48 ml). The crystals were dried under reduced pressure at 50° C., to yield the title compound (22.08 g) (yield 95.3%).

¹H-NMR (DMSO-d₆, TMS, 300 MHz) δ (ppm): 2.46 (3H, s), 3.98 (3H, s), 7.04 (1H, d, J=7.5 Hz), 7.14 (1H, t, J=7.8 Hz), 7.70 (1H, d, J=7.7 Hz), 8.28 (2H, s), 11.72 (1H, s).

High resolution mass spectrometry (C₁₃H₁₂N₂O)

Theoretical value: 212.0950 [M⁺]

Measured value: 212.0946 [M⁺]

Melting point: 215.9 to 220.2° C.

### (3) 3-Bromo-N-(2-methoxyphenyl)-5-methylpyridine-2-amine

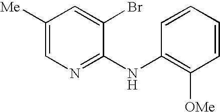

Process using Cu Catalyst

Under a nitrogen atmosphere, 2-amino-3-bromo-5-methylpyridine (1.00 g, 5.35 mmol), 2-iodoanisole (1.25 g, 5.35 mmol), copper (I) iodide (102 mg, 0.54 mmol), ethanol amine (65 mg, 1.07 mmol), potassium carbonate (1.11 g, 8.02 mmol) and anisole (15 ml) were mixed, and the mixture was stirred at room temperature for 10 minutes. The mixture was heated and stirred at 130° C. for 18 hours. The reaction solution was cooled to room temperature, and water (15 ml) was added thereto. The mixture was concentrated under reduced pressure. To the concentrate, methanol (14 ml) and 25% aqueous ammonia (7 ml) were added. The mixture was stirred at room temperature for 30 minutes. The crystals were collected by filtration, and dried at room temperature under reduced pressure, to yield the title compound (720 mg). (HPLC area %: 79.2% (3-iodo-N-(2-methoxyphenyl)-5-methylpyridine-2-amine: 17.1%)).

The spectral data was confirmed to be the same as for the title compound obtained in the above (1).

### (4) 8-Methoxy-3-methyl-9H-pyrido[2,3-b]indole

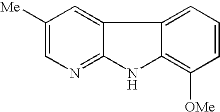

Under a nitrogen atmosphere, palladium acetate (12 mg, 0.05 mmol), 2-(dicyclohexylphosphino)biphenyl (36 mg, 0.10 mmol) and N,N-dimethylacetamide (0.9 ml) were mixed, and the mixture was stirred at room temperature for 10 minutes. 3-Bromo-N-(2-methoxyphenyl)-5-methylpyridine-2-amine (300 mg, 1.02 mmol), 1,8-diazabicyclo[5.4.0]-7-undecene (312 mg, 2.04 mmol) and N,N-dimethylacetamide (0.6 ml) were added to the reaction solution. The mixture was stirred at 130° C. for 3 hours. The reaction solution was cooled to room temperature, and water (3 ml) was added thereto. The mixture was stirred at room temperature for 30 minutes, and the crystals were collected by filtration, and washed twice with methanol/water (1/1, 1 ml) and once with water (1 ml). The crystals were dried under reduced pressure at 50° C., to yield the title compound (190 mg) (yield after two processes starting from 2-amino-3-bromo-5-methylpyridine: 40.2%).

The spectral data was confirmed to be the same as for the title compound obtained in the above (2).

### (5) 5-Iodo-8-methoxy-3-methyl-9H-pyrido[2,3-b]indole

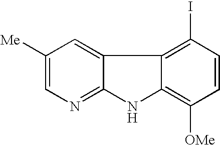

8-Methoxy-3-methyl-9H-pyrido[2,3-b]indole (10.00 g, 47.11 mmol) was suspended in acetonitrile (100 ml), and methanesulfonic acid (22.64 g, 235.57 mmol) was added dropwise to the suspension at an internal temperature of 20 to 30° C. N-iodosuccinimide (11.13 g, 49.47 mmol) was added thereto, and the mixture was stirred at room temperature for 2 to 4 hours. Methanol (30 ml) and sodium sulfite (7.13 g)/water (30 ml) were added to the reaction solution. The pH was adjusted to 8 to 9 using 4N aqueous sodium hydroxide solution. The mixture was stirred at room temperature for 1 hour. The crystals were collected by filtration, and washed once with methanol/water (1/1, 30 ml) and twice with water (30 ml). The crystals were dried under reduced pressure at 50° C., to yield the title compound (15.78 g) (yield 99.1%).

¹H-NMR (DMSO-d₆, TMS, 300 MHz) δ (ppm): 2.51 (3H, s), 3.99 (3H, s), 6.93 (1H, d, J=8.4 Hz), 7.59 (1H, d, J=8.3 Hz), 8.39 (1H, d, J=1.4 Hz), 8.92 (1H, s), 12.29 (1H, s).

High resolution mass spectrometry (C₁₃H₁₁IN₂O)

Theoretical value: 337.9916 [M⁺]

Measured value: 337.9915 [M⁺]

Melting point: 260.8 to 264.3° C.

### (6) 5-Bromo-8-methoxy-3-methyl-9H-pyrido[2,3-b]indole

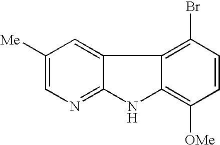

8-Methoxy-3-methyl-9H-pyrido[2,3-b]indole (7.00 g, 32.98 mmol) was suspended in acetonitrile (70 ml), and methanesulfonic acid (6.34 g, 65.96 mmol) was added dropwise to the suspension at an internal temperature of 20 to 30° C. N-bromosuccinimide (5.58 g, 31.33 mmol) was added thereto, and the mixture was stirred at room temperature for 30 minutes. Methanol (21 ml) and sodium sulfite (4.57 g)/water (21 ml) were added to the reaction solution. The pH was adjusted to 8 to 9 using a 2N aqueous sodium hydroxide solution. The mixture was stirred at room temperature for 1 hour. The crystals were collected by filtration, and washed once with methanol/water (1/1, 21 ml) and twice with water (21 ml). The crystals were dried under reduced pressure at 50° C., to yield the title compound (9.27 g) (yield 96.5%).

¹H-NMR (DMSO-d₆, TMS, 300 MHz) δ (ppm): 2.48 (3H, s), 3.97 (3H, s), 7.00 (1H, d, J=8.5 Hz), 7.32 (1H, d, J=8.4 Hz), 8.36 (1H, d, J=1.7 Hz), 8.63 (1H, d, J=1.3 Hz), 12.12 (1H, s).

High resolution mass spectrometry (C₁₃H₁₁BrN₂O)

Theoretical value: 290.0055 [M⁺]

Measured value: 290.0054 [M⁺]

Melting point: 310.4 to 312.5° C.

### (7) 3-Methyl-9H-pyrido[2,3-b]indol-8-ol

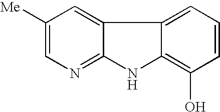

8-Methoxy-3-methyl-9H-pyrido[2,3-b]indole (5.00 g, 23.56 mmol) was suspended in 47% hydrobromic acid (90 ml), and the suspension was stirred for 30 hours at an internal temperature of 90 to 100° C. The reaction solution was cooled to an internal temperature of 5° C. The pH was adjusted to 7 to 8 using 25% aqueous ammonia. The mixture was stirred at room temperature for 1 hour. The crystals were collected by filtration, and washed once with methanol/water (1/1, 151 ml) and twice with water (15 ml). The crystals were dried under reduced pressure at 50° C., to yield the title compound (4.00 g) (yield 85.7%).

¹H-NMR (DMSO-d₆, TMS, 300 MHz) δ (ppm): 2.46 (3H, s), 6.87-6.90 (1H, m), 7.01 (1H, t, J=7.7 Hz), 7.56 (1H, d, J=7.6 Hz), 8.24-8.28 (2H, m), 9.74 (1H, s), 11.38 (1H, s).

High resolution mass spectrometry (C₁₂H₁₀N₂O)

Theoretical value: 198.0793 [M⁺]

Measured value: 198.0789 [M⁺]

Melting point: 266.9 to 271.0° C.

### (8) 5-Iodo-3-methyl-9H-pyrido[2,3-b]indol-8-ol

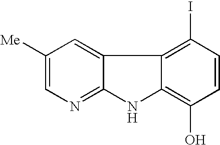

3-Methyl-9H-pyrido[2,3-b]indol-8-ol (800 mg, 4.04 mmol) was suspended in acetonitrile (8 ml), and methanesulfonic acid (1.94 g, 20.18 mmol) was added dropwise to the suspension at an internal temperature of 20 to 30° C. N-iodosuccinimide (999 mg, 4.44 mmol) was added thereto, and the mixture was stirred at room temperature for 30 minutes. Methanol (2.4 ml) and sodium sulfite (610 mg)/water (2.4 ml) were added to the reaction solution. The pH was adjusted to 6 to 7 using a 4N aqueous sodium hydroxide solution. The mixture was stirred at room temperature for 1 hour. The crystals were collected by filtration, and washed with methanol/water (1/1, 2.4 ml) and with water (2.4 ml). The crystals were dried under reduced pressure at 50° C., to yield the title compound (1.18 g) (yield 90.0%).

¹H-NMR (DMSO-d₆, TMS, 300 MHz) δ (ppm): 2.48 (3H, s), 6.72 (1H, d, J=8.2 Hz), 7.42 (1H, d, J=8.2 Hz), 8.34 (1H, d, J=1.9 Hz), 8.83 (1H, d, J=1.5 Hz), 10.10 (1H, br), 11.77 (1H, s).

High resolution mass spectrometry (C₁₂H₉IN₂O)

Theoretical value: 323.9760 [M⁺]

Measured value: 323.9755 [M⁺]

Melting point: 214.8 to 219.4° C.

### (9) 3-Methyl-9H-pyrido[2,3-b]indol-8-yl trifluoromethanesulfonate

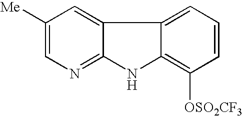

3-Methyl-9H-pyrido[2,3-b]indol-8-ol (5.00 g, 25.22 mmol) was suspended in pyridine (25 ml). Trifluoromethanesulfonic anhydride (8.54 g, 30.27 mmol) was added dropwise to the suspension with ice cooling. The mixture was stirred for 2 hours with ice cooling. Acetonitrile (50 ml) was added to the reaction solution, and the mixture was concentrated under reduced pressure. Acetonitrile (10 ml) and water (30 ml) were added to the residue, and the mixture was stirred at room temperature for 30 minutes. The crystals were collected by filtration. The crystals were washed with acetonitrile/water (1/2, 10 ml) and with acetonitrile (5 ml). The obtained crude crystals were suspended in ethyl acetate (40 ml), and the crystals were collected by filtration. The crystals were further suspended in acetonitrile (10 ml), collected by filtration, and dried under reduced pressure at room temperature, to yield the title compound (3.65 g) (yield 44%).

¹H-NMR (DMSO-d₆, TMS, 300 MHz) δ (ppm): 2.45 (3H, s), 7.33 (1H, t, J=8.0 Hz), 7.56 (1H, d, J=8.1 Hz), 8.27 (1H, d, J=7.7 Hz), 8.42 (1H, s), 8.45 (1H, s), 12.55 (1H, s).

High resolution mass spectrometry (C₁₃H₉F₃N₂O₃S)

Theoretical value: 330.0286 [M⁺]

Measured value: 330.0289 [M⁺]

Melting point: 220.8 to 222.0° C.

### Example 21

### Ethyl (2E)-3-(3-methyl-9H-pyrido[2,3-b]indol-8-yl)acrylate

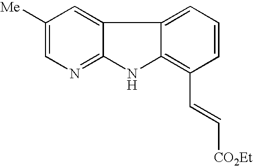

Under a nitrogen atmosphere, palladium chloride (27 mg, 0.15 mmol), lithium chloride (257 mg, 6.06 mmol)and N,N-dimethylacetamide (3 ml) were mixed, and the mixture was stirred at room temperature for 10 minutes. 3-Methyl-9H-pyrido[2,3-b]indol-8-yl trifluoromethanesulfonate (1.00 g, 3.03 mmol), ethyl acrylate (606 mg, 6.06 mmol), triethylamine (613 mg, 6.06 mmol) and N,N-dimethylacetamide (2 ml) were added thereto. The mixture was stirred at 100° C. for 6.5 hours. The reaction solution was cooled to room temperature, and water (9 ml) was added thereto. The mixture was stirred at room temperature for 30 minutes, and the crystals were collected by filtration and washed twice with methanol/water (1/1, 2 ml) and twice with water (2 ml). The obtained crude crystals were suspended in ethyl acetate (8 ml), and the crystals were collected by filtration. The crystals were washed with ethyl acetate (3 ml) and dried under reduced pressure at 50° C., to yield the title compound (420 mg) (yield 49.5%).

¹H-NMR (DMSO-d₆, TMS, 300 MHz) δ (ppm): 1.33 (3H, t, J=7.0 Hz), 2.48 (3H, s), 4.26 (2H, q, J=7.0 Hz), 6.78 (1H, d, J=15.8 Hz), 7.26 (1H, t, J=7.6 Hz), 7.93 (1H, d, J=7.4 Hz), 8.21 (1H, d, J=7.5 Hz), 8.34-8.36 (2H, m), 8.39 (1H, d, J=15.6 Hz), 12.23 (1H, s).

High resolution mass spectrometry (C₁₇H₁₆N₂O₂)

Theoretical value: 280.1212 [M⁺]

Measured value: 280.1202 [M⁺]

Melting point: 259.1 to 262.9° C.

### Example 22

### 3-Methyl-8-phenyl-9H-pyrido[2,3-b]indole

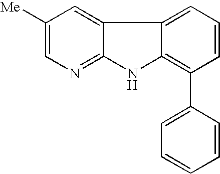

Under a nitrogen atmosphere, 3-methyl-9H-pyrido[2,3-b]indol-8-yl trifluoromethanesulfonate (500 mg, 1.51 mmol), sodium carbonate (321 mg, 3.03 mmol), phenylboronic acid (222 mg, 1.82 mmol), N,N-dimethylacetamide (3 ml) and water (0.5 ml) were mixed. To this mixture, tetrakis(triphenylphosphine)palladium (0) (175 mg, 0.15 mmol) was added. The mixture was heated and stirred at 100° C. for 2.5 hours. After completion of the reaction, the reaction solution was cooled to room temperature, and water (4 ml) was added thereto. The mixture was stirred at room temperature for 30 minutes, the crystals were collected by filtration and washed twice with methanol/water (1/2, 2 ml). The obtained crude crystals were suspended in tetrahydrofuran (2 ml) at room temperature, and the crystals were collected by filtration. The crystals were dried under reduced pressure at 40° C., to yield the title compound (230 mg) (yield 58.8%).

¹H-NMR (DMSO-d₆, TMS, 300 MHz) δ (ppm): 2.49 (3H, s), 7.32 (1H, t, J=7.6 Hz), 7.45-7.47 (2H, m), 7.53-7.58 (2H, m), 7.72 (1H, s), 7.73 (1H, d, J=1.3 Hz), 8.15 (1H, d, J=7.6 Hz), 8.29 (1H, d, J=1.6 Hz), 8.38 (1H, s), 11.52 (1H, s).

High resolution mass spectrometry (C₁₈H₁₄N₂)

Theoretical value: 258.1157 [M⁺]

Measured value: 258.1154 [M⁺]

Melting point: 229.2 to 231.2° C.

### Example 23

### (1) {2-[(3-Bromo-5-methylpyridin-2-yl)amine]phenyl}methanol

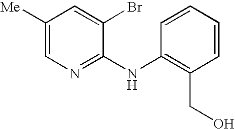

Under a nitrogen atmosphere, 2-amino-3-bromo-5-methylpyridine (1.50 g, 8.02 mmol), 2-iodobenzyl alcohol (1.88 g, 8.02 mmol), copper (I) iodide (153 mg, 0.80 mmol), ethanol amine (98 mg, 1.60 mmol), potassium carbonate (2.22 g, 16.04 mmol) and anisole (22.5 ml) were mixed, and the mixture was stirred at room temperature for 10 minutes. The mixture was heated and stirred at 130° C. for 7 hours. The reaction solution was cooled to room temperature, and water (22.5 ml) was added thereto. The mixture was concentrated under reduced pressure. To the concentrate, ethyl acetate (30 ml), water (10 ml) and activated carbon Shirasagi A were added, and the mixture was filtered. The organic layer was separated, washed with water (10 ml) and concentrated under reduced pressure. Methanol (1 ml) was added to the residue, and the mixture was stirred at room temperature for 30 minutes. The crystals were collected by filtration, and dried at room temperature under reduced pressure, to yield the title compound (470 mg) (yield 20.0%).

¹H-NMR (DMSO-d₆, TMS, 300 MHz) δ (ppm): 2.24 (3H, s), 2.49 (1H, br), 4.72 (2H, d, J=5.3 Hz), 7.06 (1H, t, J=7.4 Hz), 7.30-7.37 (2H, m), 7.63 (1H, d, J=1.7 Hz), 7.82-8.00 (3H, m).

High resolution mass spectrometry (C₁₃H₁₃BrN₂O)

Theoretical value: 292.0211 [M⁺]

Measured value: 292.0208 [M⁺]

Melting point: 129.3 to 131.8° C.

### (2) (3-Methyl-9H-pyrido[2,3-b]indol-8-yl)methanol

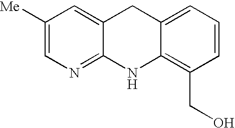

Under a nitrogen atmosphere, palladium acetate (7 mg, 0.03 mmol), 2-(dicyclohexylphosphino)biphenyl (22 mg, 0.06 mmol) and N,N-dimethylacetamide (0.9 ml) were mixed, and the mixture was stirred at room temperature for 10 minutes. {2-[(3-Bromo-5-methylpyridin-2-yl)amine]phenyl}methanol (300 mg, 1.02 mmol) and 1,8-diazabicyclo[5.4.0]-7-undecene (312 mg, 2.05 mmol) were added thereto. The mixture was stirred at 130° C. for 2 hours. After completion of the reaction, the reaction solution was cooled to room temperature, and water (1.8 ml) was added thereto. The mixture was stirred at room temperature for 30 minutes, and the crystals were collected by filtration and washed twice with methanol/water (1/1, 0.6 ml) and once with water (0.6 ml). The crystals were dried under reduced pressure at 50° C., to yield the title compound (192 mg) (yield 88.4%).

¹H-NMR (DMSO-d₆, TMS, 300 MHz) δ (ppm): 2.46 (3H, s), 4.87 (2H, d, J=5.7 Hz), 5.23 (1H, t, J=5.7 Hz), 7.19 (1H, d, J=7.6 Hz), 7.47 (1H, d, J=7.3 Hz), 8.01 (1H, d, J=7.7 Hz), 8.29 (1H, s), 8.31 (1H, s), 11.48 (1H, s).

High resolution mass spectrometry (C₁₃H₁₂N₂O)

Theoretical value: 212.0950 [M⁺]

Measured value: 212.0947 [M⁺]

Melting point: 263.4 to 267.5° C.

### Example 24

### (1) 3-Iodo-2-methyl-5-nitrobenzoic acid

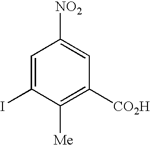

2-Methyl-5-nitrobenzoic acid (10.00 g, 55.2 mmol), iodine (5.60 g, 22.1 mmol, 0.4 equiv) and sodium iodate (4.37 g, 22.1 mmol, 0.4 equiv) were dissolved in 96% sulfuric acid (40 ml), and the solution was stirred at 30 to 40° C. for 2 hours. The reaction solution was cooled to near room temperature. While adjusting the internal temperature of the reaction solution not to exceed 50° C., sodium sulfite (17.40 g, 138.0 mmol, 2.5 equiv)/water (100 ml) and methanol (40 ml) were added dropwise sequentially. The reaction solution was stirred for 2 hours while maintaining the internal temperature of the reaction solution at 20 to 30° C. Precipitated crystals were collected by filtration, and washed twice with 67% hydrous ethanol (20 ml). The crystals were dried in a vacuum at 50° C., to yield the title compound (15.80 g) (yield 93.2%).

¹H-NMR (300 MHz, TMS, DMSO-d₆) δ (ppm): 2.69 (s, 3H), 8.44 (d, J=2.2 Hz, 1H), 8.70 (d, J=2.3 Hz, 1H).

¹³C-NMR (300 MHz, TMS, DMSO-d₆) δ (ppm): 26.5, 104.5, 124.0, 133.5, 135.3, 145.3, 148.4, 167.

Mass analysis (C₈H₆INO₄)

Theoretical value: 306.9342

Measured value: 306.9333

Melting point: 178.3° C.

### (2) Methyl 3-iodo-2-methyl-5-nitrobenzoate

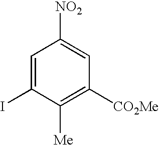

Methanol (75 mL) was added dropwise at 50° C. or lower to 3-iodo-2-methyl-5-nitrobenzoic acid (15.00 g, 48.85 mmol) dissolved in 96% sulfuric acid (10.4 ml, 195.4 mmol, 4.0 equiv). The reaction solution was stirred for 6 hours, while maintaining the internal temperature of the reaction solution at 60±5° C. The solution was stirred for 30 minutes while maintaining the internal temperature at 40 to 50° C. Next, sodium sulfite (1.23 g, 9.77 mmol, 0.2 equiv)/water (30 ml) were added dropwise thereto. The pH of the reaction solution was adjusted to 8 to 9 by adding 5% aqueous ammonia at 40-50° C. Water (30 ml) was added thereto, and the mixture was stirred at 40 to 50° C. for 30 minutes and at 5 to 10° C. for 1 hour. Then, precipitated crystals were collected by filtration and washed twice with 67% hydrous methanol (20 mL). The crystals were dried in a vacuum at 50° C., to yield the title compound (14.77 g) (yield 94.2%).

¹H-NMR (300 MHz, TMS, DMSO-d₆) δ (ppm): 2.65 (s, 3H), 3.90 (s, 3H), 8.46 (d, J=2.4 Hz, 1H), 8.73 (d, J=2.4 Hz, 1H).

¹³C-NMR (300 MHz, TMS, DMSO-d₆) δ (ppm): 26.4, 53.2, 105.5, 124.1, 132.0, 135.8, 145.5, 148.3, 165.7.

Mass analysis (C₉H₈INO₄)

Theoretical value: 320.9498

Measured value: 320.9492

Melting point: 64.9° C.

### (3) Methyl 3-[(3-bromo-5-methylpyridin-2-yl)amino]-2-methyl-5-nitrobenzoate

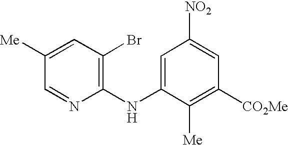

In a 100-ml four-necked flask, palladium acetate (33.7 mg, 0.15 mmol) and 4,5-bis(diphenylphosphino)-9,9-dimethylxanthene (Xantphos) (86.8 mg, 0.15 mmol) were dissolved in toluene (3 ml), and the solution was stirred at room temperature for 30 minutes. 2-Amino-3-bromo-5-methylpyridine (561.1 mg, 3 mmol), methyl 3-iodo-2-methyl-5-nitrobenzoate (963.3 mg, 3 mmol) and cesium carbonate (1.37 g, 4.2 mmol) were dissolved in toluene (3 ml), and this solution was added to the mixture prepared above. The mixture was stirred for 30 minutes at room temperature, and then for 2.5 hours at 100° C. The reaction solution was cooled to near room temperature, tetrahydrofuran (60 ml) and a 1N aqueous hydrochloric acid solution (4.2 ml) were added thereto, and the mixture was stirred at room temperature for 1 hour. The reaction solution was filtered through Celite, and Celite was washed twice with tetrahydrofuran (6 ml). The filtrate was washed three times with a 10% aqueous sodium bicarbonate solution (30 ml), and the organic layer was concentrated under reduced pressure. An ethanol/acetone solution (10/1, 22 ml) was added to the concentration residue, and the mixture was stirred at room temperature for 30 minutes. The crystals were collected by filtration, washed with ethanol/acetone (10/1, 11 ml), and dried in a vacuum at 60° C., to yield 1.07 g of the title compound (yield 93.6%).

¹H-NMR (300 MHz, TMS, DMSO-d₆) δ (ppm): 2.19 (s, 3H), 2.36 (s, 3H), 3.89 (s, 3H), 7.87 (d, J=2.9 Hz, 1H), 7.92 (d, J=2.9 Hz, 1H), 8.12 (s, 1 H), 8.26 (d, J=2.6 Hz, 1H), 8.55 (d, J=2.6 Hz, 1H).

¹³C-NMR (300 MHz, TMS, DMSO-d₆) δ (ppm): 16.0, 16.7, 52.9, 106.5, 119.2, 120.7, 126.7, 132.4, 140.4, 141.9, 142.1, 145.3, 146.2, 150.5, 166.5.

Mass analysis (C₁₅H₁₄BrN₃O₄)

Theoretical value: 379.0168

Measured value: 379.0159

Melting point: 187.3° C.

### (4) Methyl 3-[(3-bromo-5-methylpyridin-2-yl)amino]-5-amino-2-methylbenzoate

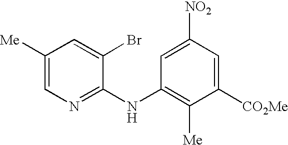

Methyl 3-[(3-bromo-5-methylpyridin-2-yl)amino]-2-methyl-5-nitrobenzoate (15.21 g, 40 mmol), tin (II) chloride (27.98 g, 120 mmol), methanol (200 ml) and 36% hydrochloric acid (20 ml) were mixed, and the mixture was stirred at 50° C. for 3 hours. While maintaining the internal temperature of the reaction solution not to exceed 30° C., a 5N aqueous sodium hydroxide solution (110 ml) was added dropwise thereto. Tetrahydrofuran (1.5 L) was added to the reaction solution, and the mixture was washed twice with saturated brine (150 ml). The organic layer was separated and concentrated under reduced pressure. Ethanol (80 ml) was added to the concentration residue, and the mixture was stirred for 30 minutes. The crystals were collected by filtration, and washed with ethanol (20 ml). The crystals were dried in a vacuum at 60° C., to yield 11.90 g of the title compound (yield 84.9%).

¹H-NMR (300 MHz, TMS, DMSO-d₆) δ (ppm): 2.10 (s, 3H), 2.15 (s, 3H), 3.79 (s, 3H), 5.10 (brs, 2H), 6.82 (d, J=2.4 Hz, 1H), 6.89 (d, J=2.4 Hz, 1H), 7.48 (s, 1H), 7.74 (d, J=1.7 Hz, 1H), 7.83 (d, J=1.7 Hz, 1H).

¹³C-NMR (300 MHz, TMS, DMSO-d₆) δ (ppm): 14.3, 16.6, 51.9, 105.4, 111.8, 115.0, 120.3, 124.4, 131.5, 140.7, 141.3, 146.2, 146.5, 151.6, 168.7.

Mass analysis (C₁₅H₁₆BrN₃O₂)

Theoretical value: 349.0426

Measured value: 349.0424

Melting point: 122.9° C.

### (5) Methyl 5-amino-3,8-dimethyl-9H-pyrido[2,3-b]indole-7-carboxylate

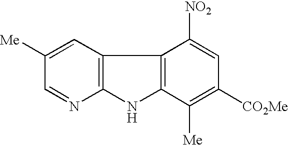

Palladium acetate (202.1 mg, 0.9 mmol) and 2-(dicyclohexylphosphino)biphenyl (630.9 mg, 1.8 mmol) were dissolved in N,N-dimethylacetamide (10 ml), and the solution was degassed in a vacuum for 5 minutes at room temperature. Methyl 3-[(3-bromo-5-methylpyridin-2-yl)amino]-5-amino-2-methylbenzoate (10.51 g), 1,8-diazabicyclo[5.4.0]-7-undecene (0.91 g, 60 mmol) and degassed N,N-dimethylacetamide (10 ml) were added to the solution, and under a nitrogen atmosphere, the mixture was stirred for 30 minutes at room temperature and for 1 hour at 130° C. The reaction solution was cooled to room temperature, water (40 ml) was added dropwise thereto, and the mixture was stirred at room temperature for 1 hour. Precipitated crystals were collected by filtration, and the crystals were washed twice with water (10 ml). The crystals were dried in a vacuum at 60° C., to yield 7.36 g of the title compound (yield 91.1%).

¹H-NMR (300 MHz, TMS, DMSO-d₆) δ (ppm): 2.45 (s, 3H), 2.59 (s, 3H), 3.83 (s, 3H), 5.65 (s, 2H), 6.98 (s, 2H), 8.23 (d, J=1.4 Hz, 1H), 8.48 (d, J=1.4 Hz, 1H), 11.59 (brs, 1H).

¹³C-NMR (300 MHz, TMS, DMSO-d₆) δ (ppm): 14.3, 18.3, 51.8, 106.3, 109.7, 110.1, 115.0, 123.6, 127.6, 130.0, 140.2, 142.0, 145.7, 150.9, 168.6.

Mass analysis (C₁₅H₁₅N₃O₂)

Theoretical value: 269.1164

Measured value: 269.1151

Melting point: 295.1° C.

### (6) Methyl 5-iodo-3,8-dimethyl-9H-pyrido[2,3-b]indole-7-carboxylate

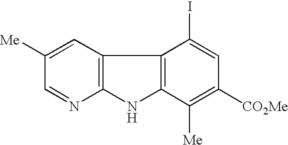

Methyl 5-amino-3,8-dimethyl-9H-pyrido[2,3-b]indole-7-carboxylate (2.69 g, 10 mmol) was suspended in 6N hydrochloric acid (54 ml), and the internal temperature was adjusted to 0 to 10° C. Sodium nitrite (0.72 g, 10.5 mmol) dissolved in water (27 ml) was added dropwise, while maintaining the internal temperature of the reaction solution at 0 to 10° C. After completion of the dropwise addition, the mixture was stirred at room temperature for 2 hours. Ethanol (16 ml) and a 10% aqueous sodium nitrite solution (54 ml) were sequentially added to the reaction solution. While maintaining the internal temperature of the reaction solution not to exceed 20° C., 5N aqueous sodium hydroxide solution (55 ml) was added dropwise. After completion of the dropwise addition, the mixture was stirred at 0 to 10° C. for 1 hour. Precipitated crystals were collected by filtration and washed twice with water (10 ml). The crystals were dried in a vacuum at 60° C., to yield 3.51 g of the title compound (yield 92.5%).

¹H-NMR (300 MHz, TMS, DMSO-d₆) δ (ppm): 2.50 (s, 3H), 2.73 (s, 3H), 3.87 (s, 3H), 8.04 (s, 1H), 8.46 (s, 1H), 8.89 (s, 1H), 12.23 (brs, 1H).

¹³C-NMR (300 MHz, TMS, DMSO-d₆) δ (ppm): 14.9, 18.4, 52.3, 84.2, 115.4, 123.4, 123.7, 123.9, 127.8, 129.2, 130.8, 139.7, 149.0, 151.4, 166.7.

Mass analysis (C₁₅H₁₃IN₂O₂)

Theoretical value: 380.0022

Measured value: 380.0027

Melting point: 270.5° C.

### (7) Methyl 5-[3-(ethylsulfonyl)phenyl]-3,8-dimethyl-9H-pyrido[2,3-b]indole-7-carboxylate

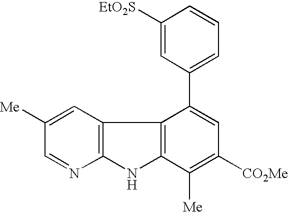

Tetrakis(triphenylphosphine)palladium (34.7 mg, 0.03 mmol, 10 mol %), methyl 5-iodo-3,8-dimethyl-9H-pyrido[2,3-b]indole-7-carboxylate (114.1 g, 0.3 mmol) and [3-(ethylsulfonyl)phenyl]boronic acid (128.5 g, 0.6 mmol, 2 equiv) were dissolved in N,N-dimethylacetamide (1 ml), and the solution was degassed in a vacuum for 5 minutes at room temperature. To the solution, potassium carbonate (82.9 mg, 0.6 mmol, 2.0 eq)/water (0.5 ml), and then degassed N,N-dimethylacetamide (1 ml) were added, and under a nitrogen atmosphere, the mixture was stirred at room temperature for 30 minutes and at 90° C. for 30 minutes. The reaction solution was cooled to room temperature, water (4 ml) was added thereto, and the mixture was stirred at room temperature for 30 minutes. Precipitated crystals were collected by filtration, and the crystals were washed twice with water (2 ml). The crystals were dried in a vacuum at 60° C., to yield 100.2 mg of the title compound (yield 79.5%).

¹H-NMR (300 MHz, TMS, DMSO-d₆) δ (ppm): 1.17 (t, J=7.3 Hz, 3H), 2.27 (s, 3H), 2.84 (s, 3H), 3.42 (q, J=7.3 Hz, 2H), 3.88 (s, 3H), 7.50 (s, 1H), 7.58 (s, 1H), 7.89 (dd, J=7.7 Hz, 1H), 8.00-8.07 (m, 2H), 8.11 (s, 1H), 8.36 (d, J=1.7 Hz, 1H), 12.21 (s, 1H).

¹³C-NMR (300 MHz, TMS, DMSO-d₆) δ (ppm): 7.4, 15.0, 18.1, 49.1, 52.2, 113.8, 119.4, 122.2, 123.2, 124.0, 126.9, 127.5, 127.9, 129.9, 130.4, 132.5, 134.2, 139.2, 139.6, 140.7, 148.4, 151.6, 167.8.

Mass analysis (C₂₃H₂₂N₂O₄S)

Theoretical value: 422.1300

Measured value: 422.1300

Melting point: 252.1° C.

### (8) 5-[3-(ethylsulfonyl)phenyl]-3,8-dimethyl-9H-pyrido[2,3-b]indole-7-carboxylic acid

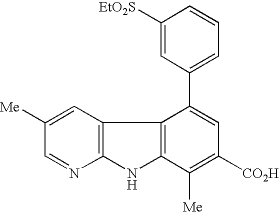

Methyl 5-[3-(ethylsulfonyl)phenyl]-3,8-dimethyl-9H-pyrido[2,3-b]indole-7-carboxylate (422.5 mg, 1 mmol) and a 2 mol/L aqueous sodium hydroxide solution were added to the mixed solvent of tetrahydrofuran (25 ml) and N,N-dimethylacetamide (10 ml). The mixture was refluxed for 3.5 hours. The reaction solution was cooled to room temperature. A 6 mol/L aqueous hydrochloric acid solution (5 ml) was added dropwise while maintaining the temperature at 30° C. or below to adjust the pH of the reaction solution to 6 to 7. Water (40 ml) was added to the reaction solution and the mixture was stirred for 2 hours at 0 to 10° C. Precipitated crystals were collected by filtration, and the crystals were washed twice with water (10 ml). The crystals were dried in a vacuum at 60° C., to yield 451.0 mg of the title compound (apparent yield 110.5%).

¹H-NMR (300 MHz, TMS, DMSO-d₆) δ (ppm): 1.18 (t, J=7.3 Hz, 3H), 2.28 (s, 3H), 2.85 (s, 3H), 3.42 (q, J=7.3 Hz, 2H), 7.55 (s, 1H), 7.61 (s, 1H), 7.89 (dd, J=7.7 Hz, 1H), 8.01-8.07 (m, 2H), 8.11 (s, 1H), 8.36 (s, 1H), 12.23 (s, 1H).

¹³C-NMR (300 MHz, TMS, DMSO-d₆) δ (ppm): 7.4, 15.1, 18.1, 49.2, 115.0, 119.0, 123.0, 123.3, 124.1, 127.5, 127.9, 128.7, 130.5, 131.2, 132.5, 134.2, 139.2, 139.9, 140.7, 146.2, 150.2, 169.0.

Mass analysis (C₂₂H₂₀N₂O₄S)

Theoretical value: 407.1055

Measured value: 407.1066

### Example 25

### (1) 3-Iodo-5-methyl-N-phenylpyridine-2-amine

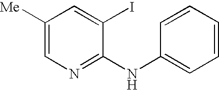

In a 20-ml flask, 2-fluoro-3-iodo-5-methylpyridine (1.0 g, 4.2 mmol), potassium acetate (828 mg, 8.4 mmol), acetic acid (1 ml) and aniline (393 mg, 4.2 mmol) were mixed, and the solution was heated to reflux for 10 hours. After allowing the solution to stand overnight at room temperature, aniline (393 mg, 4.2 mmol) was added, and the mixture was further heated to reflux for 10 hours. After allowing the mixture again to stand overnight at room temperature, aniline (393 mg, 4.2 mmol) was added, and the mixture was further heated to reflux for 8 hours. This reaction solution was cooled to room temperature, ethyl acetate (20 ml) was added thereto. The organic layer was separated and washed with water (5 ml) two times. The organic layer was washed twice with a saturated aqueous sodium bicarbonate solution (10 ml) and further washed with saturated brine (10 ml). The organic layer was dried over anhydrous sodium sulfate and concentrated under reduced pressure, diisopropyl ether (about 50 ml) was added to the residue, and a precipitate was filtered off. The filtrate was concentrated under reduced pressure, and the residue was purified by silica gel chromatography (hexane:ethyl acetate=4:1). The obtained extract was concentrated under reduced pressure, and dried under reduced pressure at 50° C., to yield the title compound (539 mg, 1.7 mmol) as a yellow oily material (yield 41.2%).

¹H-NMR (CDCl₃, TMS, 300 MHz) δ (ppm): 2.17 (s, 3H), 6.77 (brs, 1H), 6.98-7.03 (m, 1H), 7.28-7.34 (m, 2H), 7.53-7.57 (m, 2H), 7.77-7.79 (s, 2H), 7.96-7.97 (m, 1H).

¹³C-NMR (CDCl₃, TMS, 300 MHz) δ (ppm): 17.04, 81.16, 119.57, 122.48, 125.72, 128.99, 140.60, 147.20, 147.93, 151.92.

High resolution mass spectrometry (C₁₂H₁₁IN₂)

Theoretical value: 308.9889 [M−H]⁺

Measured value: 309.9892 [M−H]⁺

### Example 26

### (1) 3-Bromo-N-(2-methoxyphenyl)pyridine-2-amine

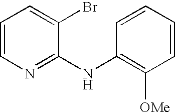

In a 5-ml screw-capped vial, palladium acetate (5.1 mg, 0.021 mmol), 4,5-bis(diphenylphosphino)-9,9-dimethylxanthene (Xantphos) (24.4 mg, 0.042 mmol), cesium carbonate (275 mg, 0.84 mmol), 2,3-dibromopyridine (100 mg, 0.42 mmol) and degassed toluene (1 ml) were mixed, and to this, o-anisidine (52.4 mg, 0.42 mmol) was added. Then, argon gas was enclosed in the vial, and the vial was stoppered tightly and the mixture was stirred with heating at an external temperature of 115° C. for 1.5 hours. After completion of the reaction, the reaction solution was cooled to room temperature, toluene (5 ml) and water (5 ml) were added thereto, and the insoluble was filtered off through Celite. The filtrate was washed with toluene (10 ml), and then the obtained organic layer was washed with saturated brine (5 ml), dried over anhydrous sodium sulfate and concentrated under reduced pressure. To the residue, a mixture (6 ml) of hexane/ethyl acetate (5/1) was added. The mixture was stirred at room temperature for 10 minutes, and then the insoluble was filtered off. The filtrate was concentrated under reduced pressure, and dried under reduced pressure at 50° C., to yield the title compound (107 mg, 0.38 mmol) as a white solid (yield 90.8%).

¹H-NMR (CDCl₃, TMS, 300 MHz) δ (ppm): 3.95 (s, 3H), 6.61 (dd, 1H, J=7.7, 4.8 Hz), 6.90-7.01, 7.73 (dd, 1H, J=7.7, 1.6 Hz), 7.83 (brs, 1H), 8.14 (dd, 1H, J=4.8, 1.6 Hz), 8.57-8.63 (m, 1H).

¹³C-NMR (CDCl₃, TMS, 300 MHz) δ (ppm): 56.03, 107.20, 110.08, 115.35, 118.33, 121.02, 121.66, 129.97, 140.11, 146.43, 148.28, 151.96.

High resolution mass spectrometry (C₁₂H₁₁BrN₂O)

Theoretical value: 278.0055 [M⁺]

Measured value: 278.0048 [M⁺]

Melting point: 93.8° C.

### (2) 8-Methoxy-9H-pyrido[2,3-b]indole

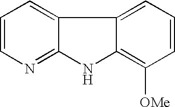

In a 5-ml screw-capped vial, 3-bromo-N-(2-methoxyphenyl)pyridine-2-amine (97 mg, 0.35 mmol), palladium acetate (4.2 mg, 0.017 mmol), 2-(dicyclohexylphosphino)biphenyl (12.2 mg, 0.035 mmol), N,N-dimethylacetamide (350 μl) and 1,8-diazabicyclo[5.4.0]-7-undecene (106 mg, 0.70 mmol) were mixed. Argon gas was enclosed in the vial, and then the vial was stoppered and the mixture was stirred for 2 hours at an external temperature of 130° C. After completion of the reaction, the reaction solution was cooled to room temperature, water (348 μl) was added thereto, and the mixture was stirred at room temperature for 30 minutes. A precipitate was collected by filtration, washed once with ethanol/water (1/10, 300 μl) and then three times with water (300 μl), and dried under reduced pressure at 50° C., to yield the title compound (47.3 mg, 0.24 mmol) (yield 68.6%).

¹H-NMR (CDCl₃, TMS, 300 MHz) δ (ppm): 3.98 (s, 3H), 7.04-7.07 (m, 1H), 7.13-7.21 (m, 2H), 7.73 (dd, 1H, J=7.7, 0.8 Hz), 8.41 (d, 1H, J=4.8, 1.6 Hz), 8.46 (dd, 1H, J=7.7, 1.6).

¹³C-NMR (CDCl₃, TMS, 300 MHz) δ (ppm): 55.86, 107.09, 113.43, 115.11, 116.82, 120.29, 122.01, 128.93, 129.45, 145.65, 146.13, 152.07.

High resolution mass spectrometry (C₁₂H₁₀N₂O)

Theoretical value: 198.0793 [M⁺]

Measured value: 198.0799 [M⁺]

Melting point: 185.4° C.

### Example 27

### (1) 3,5-Dichloro-N-(2-methoxyphenyl)pyridine-2-amine

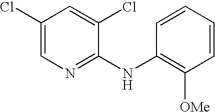

In a 300-ml four-necked flask, under a nitrogen atmosphere, 2,3,5-trichloropyridine (10.0 g, 54.81 mmol), palladium acetate (383 mg, 16.44 mmol), 4,5-bis(diphenylphosphino)-9,9-dimethylxanthene (Xantphos) (906 mg, 16.44 mmol), potassium carbonate (14.4 g, 109.62 mmol), 1,2-dimethoxyethane (100 ml) and o-anisidine (6.43 g, 52.21 mmol) were mixed. The mixture was stirred for 2.5 hours at an external temperature of 85° C. After completion of the reaction, the reaction solution was cooled to room temperature. Ethyl acetate (100 ml) and water (100 ml) were added thereto, and the insoluble was dissolved. This solution was moved to a separating funnel, and the organic layer was separated by extraction. The aqueous layer was re-extracted twice with ethyl acetate (100 ml). The organic layers were combined and washed with saturated brine (100 ml), dried over anhydrous sodium sulfate, and concentrated under reduced pressure. Methanol (100 ml) was added to the concentrate residue, and the mixture was heated to reflux for 1 hour, and then water (100 ml) was added dropwise thereto. The mixture was stirred for 1 hour at room temperature and with ice cooling for 45 minutes. A precipitate was collected by filtration with a glass filter, washed with methanol/water (1/1, 100 ml), and dried under reduced pressure at 50° C., to yield the title compound (13.86 g, 51.49 mmol) as a ocher solid (yield 94.7%).

¹H-NMR (CDCl₃, TMS, 300 MHz) δ (ppm): 3.96 (s, 3H), 6.92-6.96 (m, 1H), 7.00-7.03 (m, 2H), 7.60 (d, 1H, J=2.3 Hz), 7.80 (m, 1H), 8.13 (d, 1H, J=2.3 Hz), 8.51-8.54 (m, 1H).

¹³C-NMR (CDCl₃, TMS, 300 MHz) δ (ppm): 55.97, 110.46, 116.94, 118.36, 120.64, 121.01, 122.04, 129.35, 136.10, 144.07, 148.21, 149.72.

High resolution mass spectrometry (C₁₂H₁₀Cl₂N₂O)

Theoretical value: 268.0171 [M⁺]

Measured value: 268.0173 [M⁺]

Melting point: 123.4° C.

### (2) 3-Chloro-8-methoxy-9H-pyrido[2,3-b]indole

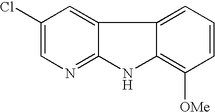

In a 100-ml flask, under a nitrogen atmosphere, 3,5-dichloro-N-(2-methoxyphenyl)pyridine-2-amine (10.00 g, 37.16 mmol), 2-(dicyclohexylphosphino)biphenyl (1.302 g, 3.71 mmol), palladium acetate (417 mg, 1.85 mmol), N,N-dimethylacetamide (20 ml) and 1,8-diazabicyclo[5.4.0]-7-undecene (11.33 g, 74.32 mmol) were mixed. The mixture was stirred for 20 hours at an external temperature of 130° C. After completion of the reaction, the reaction solution was cooled to room temperature, the mixture of water/methanol (1/1, 20 ml) was added dropwise thereto, and the mixture was stirred at room temperature for 1 hour. A precipitate was collected by filtration, washed three times with a mixture of water/methanol (1/1, 20 ml), and then dried under reduced pressure at 50° C., to yield the title compound (6.39 g, 27.58 mmol) as a brown solid (yield 73.9%).

¹H-NMR (DMSO-d₆, TMS, 300 MHz) δ (ppm): 3.98 (s, 3H), 7.08-7.10 (m, 1H), 7.16-7.21 (m, 1H), 7.77 (dd, 1H, J=7.7, 0.8 Hz), 8.41 (d, 1H, J=2.4 Hz), 8.65 (d, 1H, J=2.4 Hz).

¹³C-NMR (DMSO-d₆, TMS, 300 MHz) δ (ppm): 55.98, 110.04, 116.94, 118.36, 120.64, 121.01, 122.04, 129.35, 136.10, 144.07, 148.21, 149.72.

High resolution mass spectrometry (C₁₂H₉ClN₂O)

Theoretical value: 232.0404 [M⁺]

Measured value: 232.0408 [M⁺]

Melting point: 244.6° C.

### (3) 3-Chloro-5-iodo-8-methoxy-9H-pyrido[2,3-b]indole

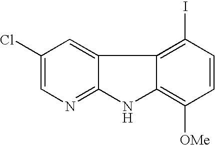

In a 300-ml four-necked flask, 3-chloro-8-methoxy-9H-pyrido[2,3-b]indole (8.3 g, 35.67 mmol) was charged. At room temperature, acetonitrile (83 ml) was added thereto, and the mixture was stirred and suspended. To this, methanesulfonic acid (17.14 g, 178.35 mmol) was added at an internal temperature of 30° C. or below, and then N-iodosuccinimide (8.03 g, 35.67 mmol) was added thereto. The mixture was stirred for 1 hour at room temperature. After confirmation of disappearance of raw materials, methanol (42 ml) was added, and a 5% aqueous sodium sulfite solution (42 ml) was added dropwise over about 30 minutes at an internal temperature of 30° C. or below. The reaction solution was neutralized (pH 7.4) with a 8N aqueous sodium hydroxide solution at an internal temperature of 30° C. or below, and the mixture was stirred for 30 minutes at room temperature. A precipitate was collected by filtration, washed three times with methanol/water (2/1, 42 ml), and then dried under reduced pressure at 50° C., to yield the title compound (11.08 g, 30.99 mmol) as a dark brown solid (yield 86.6%).

¹H-NMR (DMSO-d₆, TMS, 300 MHz) δ (ppm): 4.19 (s, 3H), 7.00 (q, 1H, J=8.4 Hz), 7.65 (d, 1H, J=8.3 Hz), 8.58 (m, 1H), 9.03 (m, 1H), 12.52 (s, 1H).

¹³C-NMR (DMSO-d₆, TMS, 300 MHz) δ (ppm) 25.28, 56.08, 77.06, 110.26, 117.14, 121.02, 121.36, 127.23, 130.48, 131.27, 145.04, 146.37, 149.89.

High resolution mass spectrometry (C₁₂H₇ClIN₂O)

Theoretical value: 357.9292 [M⁺]

Measured value: 357.9370 [M⁺]

Melting point: >300° C.

### (4) 3-Chloro-5-[3-(ethylsulfonyl)phenyl]-8-methoxy-9H-pyrido[2,3-b]indole

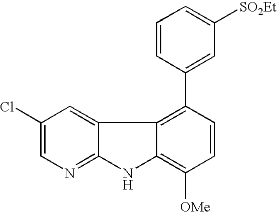

In a 10-ml screw-capped vial, 3-chloro-5-iodo-8-methoxy-9H-pyrido[2,3-b]indole (300 mg, 0.838 mmol), N,N-dimethylacetamide (4.5 ml) and a 2M aqueous sodium carbonate solution (1.5 ml) were mixed, palladium acetate (10.2 mg, 0.042 mmol), triphenylphosphine (44 mg, 0.17 mmol) and [3-(ethylsulfonyl)phenyl]boronic acid (197 mg, 0.92 mmol) were added thereto. Argon gas was enclosed in the vial, and then the vial was stoppered and the mixture was stirred for about 24 hours at an external temperature of 100° C. After cooled to room temperature, the reaction mixture was poured into a 50-ml flask (washed with 1 ml of N,N-dimethylacetamide), water (30 ml) was slowly added thereto, and the mixture was stirred at room temperature for 30 minutes. A precipitate was collected by filtration, washed twice with ethanol/water (1/5, 6 ml), and then dried under reduced pressure at 50° C. (rough weight 329 mg). Diisopropyl ether (5 ml) was added to the obtained solid and the mixture was stirred for 1 hour at room temperature. A precipitate was collected by filtration, washed with diisopropyl ether, and then dried under reduced pressure at 50° C., to yield the title compound (299 mg, 0.746 mmol) as a dark brown solid (yield 89%).

¹H-NMR (DMSO-d₆, TMS, 300 MHz) δ (ppm): 1.18 (t, 3H), 3.41 (q, 2H, J=7.3 Hz), 7.20 (dd, 2H, J=8.1, 20.5 Hz), 7.61 (d, 1H, J=2.2 Hz), 7.87 (t, 1H, J=7.6 Hz), 8.01 (t, 2H, J=7.6 Hz), 8.06 (s, 1H), 8.43 (d, 1H, J=2.2 Hz), 12.4 (s, 1H).

¹³C-NMR (DMSO-d₆, TMS, 300 MHz) δ (ppm) 7.45, 49.20, 56.04, 108.35, 115.63, 117.54, 121.26, 122.10, 127.05, 127.77, 127.88, 128.01, 130.33, 130.45, 134.18, 139.15, 141.04, 144.45, 146.02, 150.18.

High resolution mass spectrometry (C₂₀H₁₇ClN₂O₃S)

Theoretical value: 400.0649 [M⁺]

Measured value: 400.0648 [M⁺]

Melting point: >300° C.

### (5) 3-Chloro-5-[3-(ethylsulfonyl)phenyl]-9H-pyrido[2,3-b]indol-8-ol

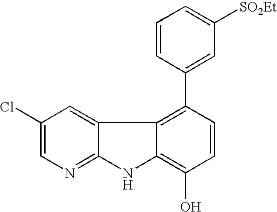

In a 10-ml flask, 3-chloro-5-iodo-8-methoxy-9H-pyrido[2,3-b]indole (50 mg, 0.125 mmol) was charged, and 48% aqueous hydrogen bromide solution (1 ml) was added thereto. The mixture was stirred with heating for 38 hours at an external temperature of 100° C. The reaction solution was cooled to room temperature, and neutralized with sodium hydroxide and hydrochloric acid with ice cooling. Water (4 ml) was added thereto, and the mixture was stirred at the same temperature for 1 hour. A precipitate was collected by filtration, washed three times with ethanol/water (1/9, 10 ml), and then dried under reduced pressure at 50° C. n-Hexane (2 ml) was added to the obtained solid, the mixture was stirred at room temperature for 30 minutes, and the solid was collected by filtration, washed with n-hexane, and dried at room temperature, to yield the ocher title compound (40 mg, 0.103 mmol) (yield 82.9%).

¹H-NMR (DMSO-d₆, TMS, 300 MHz) δ (ppm): 1.17 (t, 3H), 3.41 (q, 2H), 7.04 (s, 2H), 7.61 (d, 1H, J=2.2 Hz), 7.85 (t, 2H, J=7.6 Hz), 7.96-8.00 (m, 2H), 8.04 (s, 1H), 8.40 (d, 1H, J=2.2 Hz), 10.2 (s, 1H), 12.1 (s, 1H).

¹³C-NMR (DMSO-d₆, TMS, 300 MHz) δ (ppm) 7.47, 49.21, 112.02, 115.85, 117.93, 120.99, 122.33, 126.56, 127.75, 127.85, 130.18, 130.28, 134.14, 139.10, 141.39, 143.90, 144.28, 150.16.

High resolution mass spectrometry (C₁₉H₁₅ClN₂O₃S)

Theoretical value: 386.0492 [M⁺]

Measured value: 386.0494 [M⁺]

Melting point: >250° C.

### Example 28

### 3-Chloro-N-(2-methoxyphenyl)pyridine-2-amine

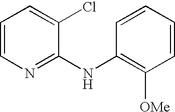

Under a nitrogen atmosphere, in a 20-ml flask, 2,3-dichloropyridine (100 mg, 0.68 mmol), palladium acetate (16 mg, 0.068 mmol), 2,2′-bis(diphenyphosphino)-1,1′-binaphthyl (racemate) (42 mg, 0.068 mmol) and potassium carbonate (1.4 g, 10.2 mmol) were mixed. After toluene (2 ml) was poured thereto, o-anisidine (76 μl, 0.68 mmol) was added, and the mixture was stirred with heating for 2 hours at an external temperature of 120° C. The reaction solution was cooled to room temperature, and then the reaction solution was poured into a separating funnel. The organic layer was separated using toluene (10 ml)/water (10 ml). Further, the aqueous layer was re-extracted twice with ethyl acetate (10 ml). (separation using toluene was difficult so the insoluble was dissolved using ethyl acetate.) The organic layers were combined, washed with saturated brine (10 ml), and dried over sodium sulfate. The solvent was distilled away under reduced pressure, and the residue was purified by silica gel column chromatography (hexane:ethyl acetate=4:1), to yield the title compound (128 mg, 0.55 mmol) as a yellow solid (yield 81%).

¹H-NMR (CDCl₃, TMS, 300 MHz) δ (ppm): 3.93 (s, 3H), 6.68 (dd, 1H, J=7.7, 4.8 Hz), 6.89-7.02 (m, 3H), 7.56 (dd, 1H, J=7.7, 1.6 Hz), 7.78 (brs, 1H), 8.15 (dd, 1H, J=4.8, 1.5 Hz), 8.57-8.62 (m, 1H).

¹³C-NMR (CDCl₃, TMS, 300 MHz) δ (ppm): 55.98, 110.04, 114.88, 116.89, 118.38, 121.04, 121.62, 129.82, 136.52, 145.45, 145.74, 148.23.

High resolution mass spectrometry (C₁₂H₁₁ClN₂O)

Theoretical value: [M⁺] 234.0560

Measured value: [M⁺] 234.0563

Melting point: 91.8° C.

### Example 29

### 3-Bromo-N-(2-methoxyphenyl)-5-pyridine-2-amine

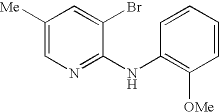

Under a nitrogen atmosphere, in a 30-ml flask, palladium acetate (48.6 mg, 0.057 mmol), 4,5-bis(diphenylphosphino)-9,9-dimethylxanthene (Xantphos) (33 mg, 0.057 mmol) and 1,2-dimethoxyethane (10 ml) were mixed, and the mixture was stirred at room temperature for 15 minutes. To this mixture, o-anisidine (140 mg, 1.14 mmol), 2,3-dibromo-5-methylpyridine (300 mg, 1.20 mmol) and cesium carbonate (750 mg, 2.28 mmol) were added, and the mixture was stirred for 8 hours at an external temperature of 85° C. After completion of the reaction, the reaction solution was cooled to room temperature, and filtered through Celite, and Celite was washed three times with ethyl acetate (6 ml). The filtrate was washed with water (3 ml). The aqueous layer was re-extracted with ethyl acetate (3 ml). The organic layers were combined, washed with saturated brine (3 ml), and dried over anhydrous sodium sulfate, and concentrated under reduced pressure. Hexane (6 ml) was added to the concentrate residue. The mixture was stirred for 30 minutes at room temperature and for another 30 minutes with ice cooling. A precipitate was collected by filtration, and washed twice with hexane (3 ml), and then dried under reduced pressure at 50° C., to yield the title compound (202 mg, 0.69 mmol) as a dark green solid (yield 60%)

¹H-NMR (CDCl₃, TMS, 300 MHz) δ (ppm): 2.22 (s, 3H), 3.94 (s, 3H), 6.88-7.02 (m, 3H), 7.59 (d, 1H, J=1.6 Hz), 7.70 (brs, 1H), 8.01 (s, 1H), 8.54 (dd, 1H, J=1.9, 7.9 Hz).

¹³C-NMR (CDCl₃, TMS, 300 MHz) δ (ppm): 17.19, 55.98, 106.90, 109.99, 117.74, 121.02, 121.13, 124.78, 130.35, 140.89, 146.02, 148.05, 149.95.

High resolution mass spectrometry (C₁₃H₁₃BrN₂O)

Theoretical value: [M⁺] 292.0211

Measured value: [M⁺] 292.0212

Melting point: 112.7° C.

### Example 30

### 3-Chloro-N-(2-methoxyphenyl)-5-(trifluoromethyl)pyridine-2-amine

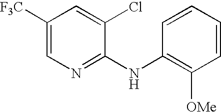

In a 5-ml screw-capped vial, o-anisidine (564 μl, 5.0 mmol), 2,3-dichloro-5-trifluoromethylpyridine (767 μl, 5.5 mmol), palladium acetate (61 mg, 0.25 mmol), 4,5-bis(diphenylphosphino)-9,9-dimethylxanthene (Xantphos) (145 mg, 0.25 mmol), cesium carbonate (3.3 g, 10 mmol), and toluene (5 ml) were mixed. Argon gas was enclosed in the vial, and the vial was stoppered tightly and the mixture was stirred for 7.5 hours at an external temperature of 115° C. After completion of the reaction, the reaction solution was cooled to room temperature. The organic layer was separated by extraction using ethyl acetate (15 ml) and water (20 ml), and the aqueous layer was re-extracted with ethyl acetate (15 ml). The organic layers were combined, washed with saturated brine (15 ml), dried over anhydrous sodium sulfate and concentrated under reduced pressure. Hexane was added to the concentrate residue and the mixture was stirred at room temperature, and a precipitate was filtered off. The solvent of the filtrate was distilled away under reduced pressure, the concentrate residue was dried under reduced pressure at 50° C., to yield the title compound (1.10 g, 3.63 mmol) as a yellow solid (yield 73%).

¹H-NMR (CDCl₃, TMS, 300 MHz) δ (ppm): 3.95 (s, 3H), 6.92-6.96 (m, 1H), 7.01-7.06 (m, 2H), 7.76 (d, 1H, J=1.8 Hz), 8.05 (brs, 1H), 8.41 (d, 1H, 1.0 Hz), 8.56-8.61 (m, 1H).

¹³C-NMR (CDCl₃, TMS, 300 MHz) δ (ppm): 56.01, 110.15, 116.48, 119.26, 121.01, 123.01, 128.62, 133.18, 133.23, 143.43, 143.49, 148.54, 153.17.

High resolution mass spectrometry (C₁₃H₁₀ClF₃N₂O)

Theoretical value: [M⁺] 302.0434

Measured value: [M⁺] 302.0428

Melting point: 82.3° C.

### Example 31

### Ethyl 5-chloro-6-[(2-methoxyphenyl)amino]nicotinate

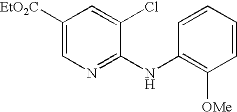

In a 5-ml screw-capped vial, o-anisidine (113 μl, 1.0 mmol), ethyl 5,6-dichloronicotinate (231 mg, 1.05 mmol), palladium acetate (12.2 mg, 0.05 mmol), 4,5-bis(diphenylphosphino)-9,9-dimethylxanthene (Xantphos) (28.9 mg, 0.05 mmol), cesium carbonate (652 mg, 2.0 mmol) and toluene (1 ml) were mixed. Argon gas was enclosed in the vial, and the vial was stoppered tightly and the mixture was stirred for 9 hours at an external temperature of 115° C. After completion of the reaction, the reaction solution was cooled to room temperature. The organic layer was separated by extraction using ethyl acetate (5 ml) and water (3 ml), and the aqueous layer was re-extracted with ethyl acetate (5 ml). The organic layers were combined, washed with saturated brine (15 ml), dried over anhydrous sodium sulfate, and concentrated under reduced pressure. The concentrated residue was purified by silica gel column chromatography (n-hexane:ethyl acetate=4:1→3:1). The obtained fraction was concentrated under reduced pressure and then the residue was dried under reduced pressure at 50° C., to yield the title compound (246.8 mg, 0.80 mmol) as a yellow white solid (yield 80.6%).

¹H-NMR (CDCl₃, TMS, 300 MHz) δ (ppm): 1.39 (t, 3H, J=7.1 Hz), 3.95 (s, 3H), 4.36 (q, 2H, J=7.1 Hz), 6.92-6.95 (m, 1H), 7.01-7.05 (m, 2H), 8.12 (brs, 1H), 8.14 (d, 1H, J=2.0 Hz ), 8.60-8.66 (m, 1H).

¹³C-NMR (CDCl₃, TMS, 300 MHz) δ (ppm): 14.40, 55.99, 61.00, 110.08, 116.12, 117.71, 119.27, 121.01, 122.91, 128.68, 136.79, 148.50, 148.55, 153.42, 164.86.

High resolution mass spectrometry (C₁₅H₁₅ClN₂O₃)

Theoretical value: [M⁺] 306.0772

Measured value: [M⁺] 306.0764

Melting point: 110.9° C.

### Example 32

### (1) 5-Bromo-2-methyl-3-nitrobenzoic acid

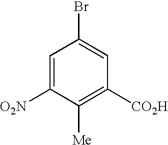

In a 500-ml four-necked flask, 2-methyl-3-nitrobenzoic acid (20 g, 110 mmol) was charged. Thereto, tetrahydrofuran (200 ml) and 64% sulfuric acid (100 ml) were added sequentially. The mixture was heated and stirred at an external temperature of 80° C. in an oil bath. After confirmation of dissolution of 2-methyl-3-nitrobenzoic acid, 1,3-dibromo-5,5-dimethylimidazolidine-2,4-dione (6.3 g, 22 mmol) was added. After this, 1,3-dibromo-5,5-dimethylimidazolidine-2,4-dione was added in 6.3 g (22 mmol) increments every 1 hour (total 18.9 g, 66 mmol). 7.5 hours after the start of reaction, the oil bath was removed, and the solution was cooled to room temperature. Water (200 ml) was added dropwise over about 40 minutes. The mixture was further stirred for 1 hour in an ice bath. Precipitated crystals were collected by filtration, and the crystals were washed with tap water (100 ml). The crystals were dried under reduced pressure at 50° C., to yield the title compound (27.3 g, 105 mmol) (yield 95.4%).

¹H-NMR (MeOH-d₄, TMS, 300 MHz) δ (ppm): 2.53 (s, 3H), 8.08 (d, 1H, J=2.1 Hz), 8.16 (d, 1H, J=2.1 Hz).

¹³C-NMR (MeOH-d₄, TMS, 300 MHz) δ (ppm) 15.87, 120.03, 130.12, 132.47, 137.05, 137.20, 153.94, 167.97.

Melting point: 183.3° C.

### (2) Methyl 5-bromo-2-methyl-3-nitrobenzoate

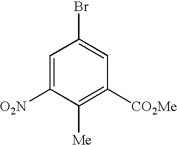

In a 200-ml flask, 5-bromo-2-methyl-3-nitrobenzoic acid (5.0 g, 19.2 mmol) was charged. Tetrahydrofuran (100 ml) was added thereto, and 5-bromo-2-methyl-3-nitrobenzoic acid was dissolved. The reaction solution was ice-cooled, and oxalyl chloride (25 ml) was added. To this, N,N-dimethylformamide (5 ml) was slowly added dropwise (foamed heavily, a white solid was precipitated). After dropwise addition, the mixture was stirred for 1 hour at room temperature, and then the reaction solution was concentrated under reduced pressure. Tetrahydrofuran was added to the residue and suspended. Under ice cooling, methanol (50 ml) was added dropwise thereto. (The reaction solution foamed heavily and the solid was dissolved. Since unreacted raw materials were observed in the reaction solution, the reaction solution was concentrated under reduced pressure again, tetrahydrofuran (50 ml) and oxalyl chloride (25 ml) were added to the concentrate, and the mixture was concentrated under reduced pressure. Tetrahydrofuran (50 ml) was added again, methanol (25 ml) was added dropwise thereto, and the mixture was stirred for 1 hour at room temperature. Raw materials were almost disappeared.)

The reaction solution was neutralized with a 8N aqueous sodium hydroxide solution, tap water (100 ml) was added and the mixture was extracted twice with ethyl acetate (200 ml). The organic layer was washed with saturated aqueous sodium bicarbonate solution (100 ml) and saturated brine (100 ml), and dried over sodium sulfate, and concentrated under reduced pressure. The residue was dried overnight at room temperature under reduced pressure, to yield the title compound (4.87 g, 17.77 mmol) as a yellow white solid (yield 93%).

¹H-NMR (CDCl₃, TMS, 300 MHz) δ (ppm): 2.56 (s, 3H), 3.95 (s, 3H), 7.97 (d, 1H, J=2.1 Hz), 8.11 (d, 1H, J=2.1 Hz).

¹³C-NMR (CDCl₃, TMS, 300 MHz) δ (ppm) 15.96, 52.96, 119.26, 129.55, 132.14, 134.75, 136.54, 152.37, 165.56.

High resolution mass spectrometry (C₉H₈NO₄)

Theoretical value: [M⁺] 272.9637

Measured value: [M⁺] 272.9638

Melting point: 48.6-49.8° C.

### (3) Methyl 3′-(ethylsulfonyl)-4-methyl-5-nitrobiphenyl-3-carboxylate

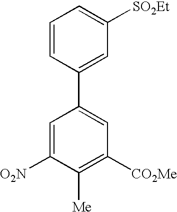

In a 200-ml flask, under a nitrogen atmosphere, methyl 5-bromo-2-methyl-3-nitrobenzoate (1.0 g, 3.65 mmol), palladium acetate (89 mg, 0.37 mmol), triphenylphosphine (382 mg, 1.48 mmol), 1,2-dimethoxyethane (20 ml), [3-(ethylsulfonyl)phenyl]boronic acid (820 mg, 3.38 mmol) and a 2M aqueous sodium carbonate solution (10 ml) were mixed. The mixture was heated and stirred at an external temperature of 90° C. for about 6 hours. The reaction solution was cooled to room temperature. The organic layer was separated by extraction using ethyl acetate (50 ml) and water (50 ml), and the aqueous layer was re-extracted with ethyl acetate (50 ml). (In this stage, separation was difficult, therefore the solution was filtered through Celite.) The organic layers were combined, washed with tap water (50 ml) and saturated brine (50 ml), dried over Na₂SO₄, and concentrated under reduced pressure. The residue was dried under reduced pressure at 50° C., diisopropyl ether/ethyl acetate (4/1) was added to the obtained solid, and the mixture was heated and stirred at room temperature for 1 hour. The solid was collected by filtration with a glass filter, washed with diisopropyl ether and dried under reduced pressure, to yield the title compound (1.03 g, 2.83 mmol) as a white yellow solid (yield 79%).

¹H-NMR (CDCl₃, TMS, 300 MHz) δ (ppm) 1.33 (t, 3H, J=7.4 Hz), 2.68 (s, 3H), 3.18 (q, 2H, J=7.4 Hz), 3.99 (s, 3H), 7.69-7.74 (m, 1H), 7.88-7.91 (m, 1H), 7.96-7.98 (m, 1H), 8.09 (d, 1H, J=2.0 Hz), 8.23-8.24 (m, 1H), 8.24 (d, 1H, J=2.0 Hz).

¹³C-NMR (CDCl₃, TMS, 300 MHz) δ (ppm) 7.49, 16.13, 50.75, 52.91, 124.98, 126.55, 128.32, 130.37, 131.94, 132.10, 132.93, 134.40, 137.76, 139.14, 139.98, 152.62, 166.53.

High resolution mass spectrometry (C₁₇H₁₇NO₆S)

Theoretical value: [M⁺] 363.0777

Measured value: [M⁺] 362.0777

Melting point: 143.1° C.

### (4) Methyl 5-amino-3′-(ethylsulfonyl)-4-methylbiphenyl-3-carboxylate

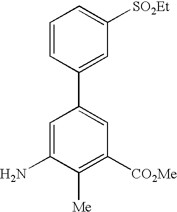

In a 200-ml four-necked flask, methyl 3′-(ethylsulfonyl)-4-methyl-5-nitrobiphenyl-3-carboxylate (7.0 g, 19.26 mmol), zinc powder (37.78 g, 577.8 mmol), calcium chloride (3.2 g, 28.89 mmol) and 78% ethanol water (70 ml) were mixed. The mixture was heated and stirred at an external temperature of 80° C. for 1 hour. After cooling the reaction solution to room temperature, the reaction solution was filtered through Celite, and Celite was washed with ethanol. The solvent was distilled away under reduced pressure. Water (70 ml) was added to the residue, and the mixture was extracted with ethyl acetate (140 ml). The aqueous layer was extracted twice with ethyl acetate (70 ml) (saturated brine 10 ml was added since separation was not good). The organic layers were combined, washed with saturated brine (70 ml) and dried over sodium sulfate. The solvent was distilled away under reduced pressure. The concentrate residue was purified by silica gel column chromatography (n-hexane/ethyl acetate=1/2→ethyl acetate), to yield the title compound (5.56 g, 86.6%) as a pale yellow solid (yield 86.6%).

¹H-NMR (CDCl₃, TMS, 300 MHz) δ (ppm) 1.30 (t, 3H, J=7.4 Hz), 2.39 (s, 2H), 3.16 (q, 2H, J=7.4 Hz), 3.92 (s, 3H), 7.06 (d, 1H, J=1.7 Hz), 7.45 (d, 1H, J=1.7 Hz), 7.61 (t, 1H, J=7.7 Hz), 7.83-7.91 (m, 2H), 8.08-8.09 (m, 1H).

¹³C-NMR (CDCl₃, TMS, 300 MHz) δ (ppm) 7.54, 13.85, 50.74, 52.21, 116.30, 118.96, 123.24, 126.45, 126.94, 129.77, 132.06, 132.49, 137.05, 139.19, 141.91, 146.38, 168.80.

High resolution mass spectrometry (C₁₇H₁₉NO₄S)

Theoretical value: [M⁺] 333.1035

Measured value: [M⁺] 333.1032

Melting point: 110.3 to 112.7° C.

### (5) 2,3-dibromo-5-methylpyridine

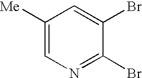

In a 30-ml three-necked flask, 2-amino-3-bromo-5-methylpyridine (187 mg, 1.00 mmol)) was charged, 48% aqueous hydrogen bromide solution (1 ml) was added and 2-amino-3-bromo-5-methylpyridine was dissolved. Then, the mixture was ice-cooled, and bromine (154 μl, 3.00 mmol) was slowly added dropwise thereto at the internal temperature of 2 to 5° C. The mixture was stirred at the same temperature for 10 minutes. To this solution, sodium nitrite (174 mg, 2.5 mmol)/water (500 μl) solution was added dropwise at the same temperature, and the mixture was stirred for 1 hour. Sodium hydroxide (377 mg, 9.4 mmol)/water (2 ml) was slowly added dropwise thereto. The mixture was stirred at room temperature for 1 hour. The solution was poured into a separating funnel. The organic layer was separated by extraction using ethyl acetate (10 ml) and water (10 ml), and the aqueous layer was further extracted with ethyl acetate (10 ml). The organic layers were combined, washed with a 5% aqueous sodium sulfite solution (5 ml) and saturated brine (10 ml), and then dried over sodium sulfate. The solvent was distilled away under reduced pressure, to yield the title compound (244 mg, 0.97 mmol) as a yellow solid (yield 97%)

¹H-NMR (CDCl₃, TMS, 300 MHz) δ (ppm): 2.30 (s, 3H), 7.73 (d, 1H, J=1.5 Hz), 8.14 (d, 1H, J=1.2 Hz).

¹³C-NMR (CDCl₃, TMS, 300 MHz) δ (ppm): 17.41, 123.16, 134.13, 140.39, 142.29, 148.57.

High resolution mass spectrometry (C₆H₅Br₂N)

Theoretical value: [M⁺] 248.8789

Measured value: [M⁺] 248.8786

Melting point: 54.2° C.

### (6) Methyl 5-[(3-bromo-5-methylpyridin-2-yl)amino]-3′-(ethylsulfonyl)-4-methylbiphenyl-3-carboxylate

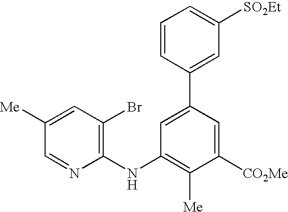

Under a nitrogen atmosphere, in a 50-ml four-necked flask, methyl 5-amino-3′-(ethylsulfonyl)-4-methylbiphenyl-3-carboxylate (2.2 g, 6.6 mmol), 2,3-dibromo-5-picoline (2.0 g, 7.9 mmol), 1,2-dimethoxyethane (25 ml), potassium carbonate (9.2 g, 66 mmol), palladium acetate (149 mg, 0.66 mmol) and 4,5-bis(diphenylphosphino)-9,9-dimethylxanthene (Xantphos) (384 mg, 0.66 mmol) were mixed, and the mixture was stirred at an external temperature of 85° C. for 6.5 hours. To the solution, 2,3-dibromo-5-picoline (83.3 mg, 0.33 mmol) was added, and the mixture was further stirred at an external temperature of 85° C. for 3 hours. After completion of the reaction, the reaction solution was cooled to room temperature. The organic layer was separated by extraction using ethyl acetate (40 ml) and water (20 ml), and the aqueous layer was re-extracted twice with ethyl acetate (40 ml). The organic layers were combined, washed with saturated brine (40 ml), dried over anhydrous sodium sulfate and concentrated under reduced pressure. The obtained residue was purified by silica gel column chromatography (n-hexane:ethyl acetate=2:1→1:3). The obtained fraction was concentrated under reduced pressure, to yield the title compound (2.81 g, 5.58 mmol) as a white yellow solid (yield 84.0%).

¹H-NMR (CDCl₃, TMS, 300 MHz) δ (ppm): 1.30 (t, 3H, J=7.4 Hz), 2.04 (s, 3H), 2.23 (s, 3H), 3.16 (q, 2H, J=7.4 Hz), 6.88 (s, 1H), 7.60-7.66 (m, 2H), 7.82-7.96 (m, 4H), 8.15 (d, 1H, J=1.5 Hz), 8.45 (d, 1H, 1.9 Hz).

¹³C-NMR (CDCl₃, TMS, 300 MHz) δ (ppm): 7.54, 14.78, 17.19, 50.74, 52.30, 106.65, 123.47, 123.65, 125.82, 126.62, 127.02, 129.78, 130.42, 132.25, 132.36, 136.73, 139.23, 140.32, 141.24, 141.79, 146.37, 150.10, 168.36.

High resolution mass spectrometry (C₂₃H₂₃BrN₂O₄S)

Theoretical value: [M⁺] 502.0562

Measured value: [M⁺] 502.0554

Melting point: 143.6-146.2° C.

### (7) Methyl 5-[(3-bromo-5-methylpyridin-2-yl)(t-butoxycarbonyl)amino]-3′(ethylsulfonyl)-4-methylbiphenyl-3-carboxylate

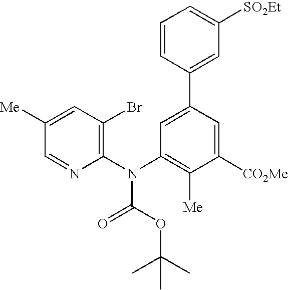

In a 20-ml flask, methyl 5-[(3-bromo-5-methylpyridin-2-yl)amino]-3′-(ethylsulfonyl)-4-methylbiphenyl-3-carboxylate (600 mg, 1.19 mmol), N,N-dimethylpyridine-4-amine (146 mg, 1.19 mmol), potassium carbonate (247 mg, 1.79 mmol), tetrahydrofuran (6 ml) and di-tert-butyl dicarbonate (411 μl, 1.79 mmol) were mixed, and the mixture was heated to reflux for 2.5 hours. Then, di-tert-butyl dicarbonate (137 μl, 0.60 mmol), potassium carbonate (82 mg, 0.60 mmol) and N,N-dimethylpyridine-4-amine (49 mg, 0.60 mmol) were added thereto, and the mixture was heated to reflux for 30 minutes. Water (10 ml) was added to this solution. The organic layer was separated by extraction using ethyl acetate (15 ml), and the aqueous layer was re-extracted with ethyl acetate (15 ml). The organic layers were combined, washed with saturated aqueous ammonia and saturated brine, dried over anhydrous sodium sulfate and concentrated under reduced pressure. The obtained residue was purified by silica gel column chromatography (n-hexane:ethyl acetate=2:1→1:1). The obtained fraction was concentrated under reduced pressure, to yield the title compound (556 mg, 0.92 mmol) as a white solid (yield 77.4%).

¹H-NMR (CDCl₃, TMS, 300 MHz) δ (ppm): 1.29 (t, 3H, J=7.4 Hz), 1.47 (s, 9H), 2.31 (s, 3H), 2.68 (s, 3H), 3.14 (q, 2H, J=7.4 Hz), 3.93 (s, 3H), 7.61 (t, 1H, J=7.7 Hz), 7.70 (s, 1H), 7.80-7.87 (m, 3H), 8.04-8.07 (m, 2H), 8.16 (d, 1H, J=1.3 Hz).

¹³C-NMR (CDCl₃, TMS, 300 MHz) δ (ppm): 7.53, 16.30, 17.57, 28.16, 50.73, 52.28, 82.17, 118.93, 126.48, 127.20, 128.14, 129.84, 130.05, 130.14, 132.16, 132.73, 133.85, 135.80, 136.80, 139.34, 141.00, 142.70, 148.16, 152.61, 167.82, 173.53.

High resolution mass spectrometry (C₂₈H₃₁BrN₂O₆S)

Theoretical value: [M⁺] 602.1084

Measured value: [M⁺] 602.1086

Melting point: 91.2° C.

### (8) Methyl 5-[(3-(ethylsulfonyl)phenyl]-3,8-dimethyl-9H-pyrido[2,3-b]indole-7-carboxylate

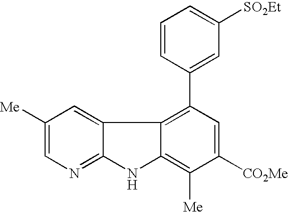

In a 5-ml screw-capped vial, methyl 5-[(3-bromo-5-methylpyridin-2-yl)(t-butoxycarbonyl)amino]-3′-(ethylsulfonyl)-4-methylbiphenyl-3-carboxylate (100 mg, 0.19 mmol), palladium acetate (8.7 mg, 0.038 mmol), 2-(dicyclohexylphosphino)biphenyl (13.6 mg, 0.038 mmol), degassed N,N-dimethylacetamide (400 μl) and potassium carbonate (38 mg, 0.38 mmol) were mixed. Argon gas was enclosed in the vial, and then the vial was stoppered and the mixture was stirred for 1 hour at an external temperature of 130° C. To the reaction solution, methanol:water (1:3, 800 μl) was added, and the mixture was stirred at the same temperature for 30 minutes and cooled to room temperature. The solution was stirred for 30 minutes and for another 30 minutes in an ice bath. A precipitate was collected by filtration, washed with methanol:water (1:3), and dried under reduced pressure at 50° C., to yield the title compound (66 mg, 0.16 mmol) as a dark green solid (yield 80.5%).

¹H-NMR (DMSO-d₆, TMS, 300 MHz) δ (ppm): 1.71 (t, 3H, J=7.3 Hz), 2.27 (s, 3H), 2.84 (s, 3H), 3.42 (q, 2H, J=7.3 Hz), 3.88 (s, 3H), 7.50 (s, 1H), 7.58 (s, 1H), 7.89 (t, 1H, J=7.7 Hz), 8.00-8.07 (m, 2H), 8.11 (s, 1H), 8.36 (d, 1H, J=1.67 Hz), 12.2 (s, 1H).

¹³C-NMR (DMSO-d₆, TMS, 300 MHz) δ (ppm): 7.18, 14.80, 17.95, 48.95, 52.03, 113.66, 119.22, 122.03, 123.05, 123.84, 126.75, 127.30, 127.69, 129.69, 130.24, 132.34, 134.00, 138.99, 139.43, 140.54, 148.25, 151.45, 167.59.

High resolution mass spectrometry (C₂₃H₂₂N₂O₄S)

Theoretical value: [M⁺] 422

Measured value: [M⁺] 422

Melting point: >300° C.

### Example 33

### Methyl 5-[(3,5-dichloropyridin-2-yl)amino]-3′-(ethylsulfonyl)-4-methylbiphenyl-3-carboxylate

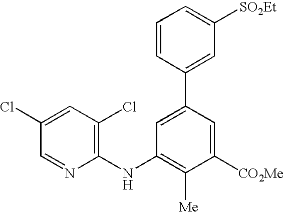

Under a nitrogen atmosphere, in a 50-ml flask, 2,3,5-trichloropyridine (345 mg, 1.09 mmol), palladium acetate (16.8 mg, 0.055 mmol), 4,5-bis(diphenylphosphino)-9,9-dimethylxanthene (Xantphos) (43.3 mg, 0.055 mmol), 1,2-dimethoxyethane (10 ml), methyl 5-amino-3′-(ethylsulfonyl)-4-methylbiphenyl-3-carboxylate monohydrochloride (500 mg, 1.04 mmol) and potassium carbonate (724 mg, 3.64 mmol) were mixed, and the mixture was stirred at an external temperature of 50° C. for 30 minutes and at 85° C. for 6 hours. The reaction solution was cooled to room temperature, and water (5 ml) was added thereto. The organic layer was separated by extraction using ethyl acetate (10 ml), and the aqueous layer was re-extracted twice with ethyl acetate (10 ml). The organic layers were combined, washed with saturated brine (10 ml), dried over anhydrous sodium sulfate and concentrated under reduced pressure. Diisopropyl ether (10 ml)/n-hexane (5 ml) was added to the concentrated residue. The mixture was stirred at room temperature for 1 hour. A precipitate was collected by filtration, washed twice with n-hexane (5 ml), and dried under reduced pressure at 50° C., to yield the title compound (605 mg, 1.26 mmol) as a yellow solid (yield 80.6%).

¹H-NMR (CDCl₃, TMS, 300 MHz) δ (ppm): 1.31 (s, 3H, J=7.4 Hz), 2.54 (s, 3H), 3.17 (q, 2H, J=7.4 Hz), 3.95 (s, 3H), 6.92 (s, 1H), 7.62-7.67 (m, 2H), 7.87-7.92 (m, 3H), 8.05 (d, 1H, J=2.0 Hz), 8.15 (s, 1H), 8.32 (d, 1H, J=1.6 Hz).

¹³C-NMR (CDCl₃, TMS, 300 MHz) δ (ppm): 7.54, 14.85, 50.76, 52.39, 116.57, 121.59, 124.76, 124.80, 126.61, 127.20, 129.90, 131.65, 132.22, 132.54, 136.59, 136.91, 139.28, 139.37, 141.46, 144.48, 150.13, 168.14.

High resolution mass spectrometry (C₂₂H₂₀Cl₂N₂O₄S)

Theoretical value: [M⁺] 478.0521

Measured value: [M⁺] 478.0515

Melting point: 160.4° C.

### Example 34

### Methyl 5-[(3-bromopyridin-2-yl)amino]-3′-(ethylsulfonyl)-4-methylbiphenyl-3-carboxylate

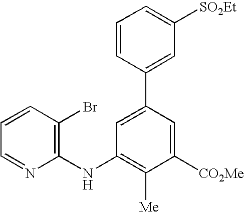

Under a nitrogen atmosphere, in a 20-ml flask, 2,3-dibromopyridine (267 mg, 2.25 mmol), potassium carbonate (311 mg, 4.5 mmol), palladium acetate. (16.8 mg, 0.075 mmol), 4,5-bis(diphenylphosphino)-9,9-dimethylxanthene (Xantphos) (43.4 mg, 0.075 mmol), 1,2-dimethoxyethane (10 ml) and methyl 5-amino-3′-(ethylsulfonyl)-4-methylbiphenyl-3-carboxylate monohydrochloride (250 mg, 1.50 mmol) were mixed, the mixture was stirred at 85° C. for 7 hours. Then, 2,3-dibromopyridine (67 mg, 0.75 mmol) and potassium carbonate (104 mg, 1.50 mmol) were added thereto, and the mixture was stirred for 15 hours. The reaction solution was cooled to room temperature, and water (10 ml) was added thereto. The organic layer was separated by extraction using ethyl acetate (10 ml), and the aqueous layer was re-extracted twice with ethyl acetate (5 ml). The organic layers were combined, washed with saturated brine (5 ml), dried over anhydrous sodium sulfate and concentrated under reduced pressure. The residue was purified by silica gel column chromatography (n-hexane:ethyl acetate=1:1). The obtained fraction was concentrated under reduced pressure and the residue was dried under reduced pressure at 50° C., to yield the title compound (170 mg, 0.35 mmol) as a brown solid (yield 80.6%).

¹H-NMR (CDCl₃, TMS, 300 MHz) δ (ppm): 1.31 (t, 3H, J=7.4 Hz), 2.56 (s, 3H), 3.16 (q, 2H, J=7.4 Hz), 6.67 (dd, 1H, J=7.7, 4.8 Hz), 6.98 (brs, 1H), 7.64 (t, 1H, J=7.8 Hz), 7.78 (dd, 1H, J=7.7, 1.5 Hz), 7.86-7.93 (m, 3H), 8.12 (dd, 1H, J=4.8, 1.5 Hz), 8.15-8.16 (m, 1H), 8.42 (d, 1H, J=1.8 Hz).

¹³C-NMR (CDCl₃, TMS, 300 MHz) δ (ppm): 7.53, 14.90, 50.74, 52.33, 106.77, 116.13, 124.34, 124.49, 126.63, 127.08, 129.82, 131.33, 132.24, 132.42, 136.78, 139.28, 139.91, 140.49, 141.65, 146.73, 152.22, 168.25.

High resolution mass spectrometry (C₂₂H₂₁BrN₂O₄S)

Theoretical value: [M⁺] 488.0405

Measured value: [M⁺] 488.0392

Melting point: 161.9° C.

### Example 35

### Ethyl 5-[(3-chloropyridin-2-yl)amino]-3′-(ethylsulfonyl)-4-methylbiphenyl-3-carboxylate

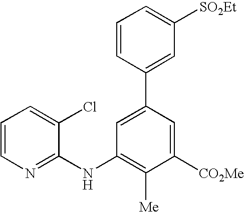

Under a nitrogen atmosphere, in a 20-ml flask, 2,3-dichloropyridine (59 mg, 0.40 mmol), potassium carbonate (830 mg, 6.01 mmol), palladium acetate (6.8 mg, 0.030 mmol), 2,2′-bis(diphenylphosphino)-1,1′-binaphthyl (racemate) (19 mg, 0.030 mmol), toluene (2 ml) and methyl 5-amino-3′-(ethylsulfonyl)-4-methylbiphenyl-3-carboxylate (133.8 mg, 0.40 mmol) were mixed, and the mixture was stirred at an external temperature of 110° C. for 1 hour The reaction solution was cooled to room temperature, water (10 ml) was added thereto, and the organic layer was separated by extraction twice using toluene (10 ml) (due to separation difficulty, the organic layers were combined, ethyl acetate 15 ml was added, the organic layer was separated again, and then the insoluble was dissolved). The organic layers were combined, washed with saturated brine (10 ml), dried over anhydrous sodium sulfate and concentrated under reduced pressure. Diisopropyl ether (20 ml) was added to the concentrated residue. The mixture was stirred at room temperature. A solid was collected by filtration, washed with diisopropyl ether, and dried under reduced pressure at 50° C., to yield the title compound (161 mg, 0.36 mmol) as a yellow solid (yield 90.4%).

¹H-NMR (CDCl₃, TMS, 300 MHz) δ (ppm): 1.31 (t, 3H, J=7.4 Hz), 2.56 (s, 3H), 3.16 (q, 2H, J=7.4 Hz), 3.95 (s, 3H), 6.74 (dd, 1H, J=7.7, 4.8 Hz), 6.95 (s, 1H), 7.60-7.66 (m, 2H), 7.86-7.90 (m, 3H), 8.09 (dd, 1H, J=4.8, 1.5 Hz), 8.15 (t, 1H, J=1.6 Hz), 8.43 (d, 1H, J=1.9 Hz).

¹³C-NMR (CDCl₃, TMS, 300 MHz) δ (ppm): 7.54, 14.81, 50.75, 52.33, 115.70, 116.56, 124.33, 124.52, 126.64, 127.09, 129.83, 131.28, 132.25, 132.43, 136.80, 136.99, 139.29, 139.72, 141.66, 146.05, 151.59, 168.26.

High resolution mass spectrometry (C₂₂H₂₁ClN₂O₄S)

Theoretical value: [M⁺] 444.0911

Measured value: [M⁺] 444.0914

Melting point: 158.6° C.

### Example 36

### Ethyl 5-{[(3-chloro-5-(trifluoromethyl)pyridin-2-yl]amino}-3′-(ethylsulfonyl)-4-methylbiphenyl-3-carboxylate

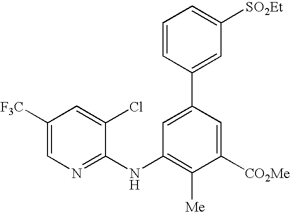

In a 5-ml screw-capped vial, methyl 5-amino-3′-(ethylsulfonyl)-4-methylbiphenyl-3-carboxylate monohydrochloride (100 mg, 0.30 mmol), 1,2-dimethoxyethane (1 ml), triethylamine (84 μl, 0.06 mmol), potassium carbonate (104 mg, 0.75 mmol), palladium acetate (6.7 mg, 0.030 mmol) and 4,5-bis(diphenylphosphino)-9,9-dimethylxanthene (Xantphos) (17.4 mg, 0.030 mmol) were mixed. Argon gas was enclosed in the vial, and the vial was stoppered tightly and the mixture was stirred for 15 hours at an external temperature of 85° C. After completion of the reaction, the reaction solution was cooled to room temperature. The organic layer was separated by extraction using ethyl acetate (5 ml) and water (5 ml), and the aqueous layer was re-extracted with ethyl acetate (5 ml). The organic layers were combined, washed with saturated brine (5 ml), dried over anhydrous sodium sulfate and concentrated under reduced pressure. The residue was purified by silica gel column chromatography (n-hexane:ethyl acetate=2:1→1:1). The obtained fraction was concentrated under reduced pressure and then the residue was dried under reduced pressure at 50° C., to yield the title compound (108 mg, 0.21 mmol) as a brown solid (yield 70.2%).

¹H-NMR (CDCl₃, TMS, 300 MHz) δ (ppm): 1.31 (t, 3H, J=7.4 Hz), 2.55 (s, 3H), 3.16 (q, 2H, J=7.4 Hz), 3.96 (s, 3H), 7.17 (s, 1H), 7.65 (t, 1H, J=7.7 Hz), 7.82 (d, 1H, J=1.9 Hz), 7.88-7.92 (m, 2H), 7.96 (d, 1H, 1.8 Hz), 8.14 (s, 1H), 8.25 (d, 1H, J=2.1 Hz), 8.33 (s, 1H).

¹³C-NMR (CDCl₃, TMS, 300 MHz) δ (ppm): 7.52, 15.00, 50.75, 52.43, 115.99, 125.89, 126.14, 126.59, 127.31, 129.96, 132.20, 132.64, 132.95, 133.82, 133.87, 137.02, 138.55, 139.44, 141.20, 143.77, 153.86, 167.94.

High resolution mass spectrometry (C₂₃H₂₃ClF₃N₂O₄S)

Theoretical value: [M⁺] 512.0785

Measured value: [M⁺] 512.0785

Melting point: 67.5° C.

### Example 37

### (1) 3-[(3-Bromo-5-methylpyridin-2-yl)amino]cyclohex-2-en-1-one

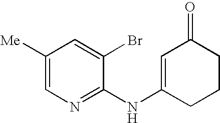

In a 500-ml flask connected to a Dean-Stark trap, 2-amino-3-bromo-5-methylpyridine (134 mmol, 25 g), 1,3-cyclohexanedione (168 mmol, 1.25 eq, 18.8 g), p-toluenesulfonic acid monohydrate (13.4 mmol, 0.1 eq, 2.55 g) and toluene (250 ml) were mixed, and the mixture was heated to reflux for 3.5 hours. After completion of the reaction, the reaction solution was cooled, a 3% aqueous sodium bicarbonate solution (200 ml) was added thereto, and the mixture was extracted three times with ethyl acetate (100 ml). The organic layers were combined and concentrated to approximately a half the original volume. Ethyl acetate (250 ml) was added to the concentrate, and the mixture was concentrated again. This operation was performed three times in total, and the concentrated residual amount was adjusted to 235 g. The concentrated slurry was stirred with heating for 1 hour, and then was stood to cool and ice-cooled. Precipitated crystals were collected by filtration, washed with cold ethyl acetate (50 ml), and dried under reduced pressure at 50° C., to yield the title compound (26.5 g, yield 70%).

¹H-NMR (DMSO-d₆, TMS, 300 MHz): δ (ppm): 1.8-2.0 (2H, m), 2.1-2.3 (2H, m), 2.26 (3H, s), 2.5-2.6 (2H, m), 5.96 (1H, s), 7.97 (1H, d, J=2.0 Hz), 8.21 (1H, d, J=2.0 Hz), 8.44 (1H, br).

### (2) 3-Methyl-6,7,8,9-tetrahydro-5H-pyrido[2,3-b]indol-5-one

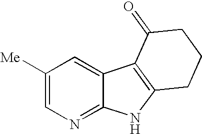

In a 200-ml flask, 3-[(3-bromo-5-methylpyridin-2-yl)amino]cyclohex-2-en-1-one (46.2 mmol, 13.0 g), bis(triphenylphosphine)palladium dichloride (2.31 mmol, 5 mol %, 1.62 g), cesium carbonate (139 mmol, 3.0 eq, 45.3 g) and toluene (130 ml) were mixed. Under an argon atmosphere, the mixture was stirred at room temperature for 1 hour. The mixture was allowed to react with heating to reflux for 7 hours, then the reaction solution was cooled, and 1N-HCl (159 ml, 3.4 eq), water (50 ml) and ethanol (25 ml) were added thereto. After heating to reflux for 1 hour, the reaction solution was stood to cool and ice-cooled. Precipitated crystals were collected by filtration, washed with water/ethanol (1/1, 50 ml), and dried under reduced pressure at 50° C., to yield the title compound (8.52 g, yield 92%).

¹H-NMR (DMSO-d₆, TMS, 300 MHz): δ (ppm): 2.13 (3H, d, J=6.3 Hz), 2.3-2.5 (2H, m), 2.37 (3H, s), 2.96 (2H, t, J=6.2 Hz), 8.05 (1H, d, J=1.9 Hz), 8.08 (1H, d, J=1.9 Hz), 12.2 (1H, br).

Mass analysis: (EI, m/z) (rel intensity): 200 (M+, 90), 172 (100), 144 (55), 86 (10), 28 (6).

### (3) 3-Methyl-6-bromo-6,7,8,9-tetrahydro-5H-pyrido[2,3-b]indol-5-one

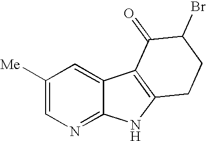

In a 50-ml flask, 3-methyl-6,7,8,9-tetrahydro-5H-pyrido[2,3-b]indol-5-one (8.04 mmol, 1.61 g), tetra-n-butylammonium tribromide (12.9 mmol, 6.22 g) and N,N-dimethylformamide (16 ml) were mixed, and the mixture was allowed to react at an internal temperature of near 80° C. for 3 hours. After cooling, the reaction solution was diluted with ethyl acetate (32 ml), and extracted with 6M HCl (32 ml+16 ml+16 ml). The aqueous layers were combined and neutralized with a 8M aqueous NaOH solution (40 ml), and the mixture was extracted twice with ethyl acetate. The organic layers were combined, washed with a 5% aqueous sodium sulfite solution, passed through a silica gel pad, and then concentrated under reduced pressure. The residual solid was dried under reduced pressure, to yield the title compound.

¹H-NMR (DMSO-d₆, TMS, 300 MHz): δ (ppm): 2.39 (3H, s), 2.3-2.6 (2H, m), 3.0-3.1 (2H, m), 4.7-4.8 (1H, m), 8.06 (1H, s), 8.13 (1H, s), 12.5 (1H, br).

### (4) 3-Methyl-9H-pyrido[2,3-b]indol-5-ol

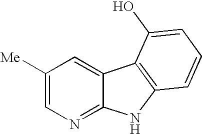

In a 50-ml flask, the entire amount of 3-methyl-6-bromo-6,7,8,9-tetrahydro-5H-pyrido[2,3-b]indol-5-one obtained in (3) above, lithium bromide (17.4 mmol, 1.51 g), lithium carbonate (17.4 mmol, 1.29 g) and N,N-dimethylformamide (16.2 ml) were mixed, and under a nitrogen atmosphere, the mixture was allowed to react at an oil bath temperature of 120° C. for 3 hours. After cooling the reaction solution, water (30 ml) was added thereto, and the mixture was extracted three times with ethyl acetate/tetrahydrofuran (1/1). The organic layers were combined, washed three times with water, and concentrated under reduced pressure. The residue was washed with ethyl acetate/hexane (2/1, 15 ml), to yield the title compound (588 mg). From the mother solution, the title compound (586 mg) was obtained (total yield 74%).

¹H-NMR (DMSO-d₆, TMS, 300 MHz): δ (ppm): 2.45 (3H, s), 6.65 (1H, d, J=7.8 Hz), 6.94 (1H, d, J=7.9 Hz), 7.24 (1H, t, J=7.9 Hz), 8.20 (1H, d, J=1.7 Hz), 8.26 (1H, d, J=1.6 Hz), 10.2 (1H, br), 11.5 (1H, br).

¹³C-NMR (DMSO-d₆, TMS, 300 MHz): δ (ppm): 19.02, 103.08, 105.67, 109.82, 115.78, 124.33, 128.37, 130.71, 141.95, 145.81, 150.90, 155.14.

Mass analysis (EI, m/z) (rel intensity): 198 (M+, 100), 197 (35), 169 (12), 99 (10)

High resolution mass spectrometry (C₁₂H₁₀N₂O)

Theoretical value: 198.0793 (M⁺)

Measured value: 198.0793 (M⁺)

### Example 38

### (1) 3-[(3-Bromo-5-methylpyridin-2-yl)amino]-5-methylcyclohex-2-en-1-one

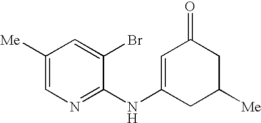

In a 100-ml flask connected to a Dean-Stark trap, 2-amino-3-bromo-5-methylpyridine (39.6 mmol, 5.93 g), 5-methyl-1,3-cyclohexanedione (39.6 mmol, 1.25 eq, 5.00 g), p-toluenesulfonic acid monohydrate (3.17 mmol, 0.1 eq, 603 mg) and toluene (59.3 ml) were mixed, and the mixture was heated to reflux for 7.5 hours. After completion of the reaction, the reaction solution was cooled, a 3% aqueous sodium bicarbonate solution (50 ml) was added thereto, and the mixture was extracted three times with ethyl acetate (50 ml). The organic layers were combined and concentrated to approximately a half the original amount. Ethyl acetate (250 ml) was added to the concentrate, and the mixture was further concentrated. This operation was performed three times in total, to adjust the concentrated residual amount to about 19 g. The concentrated slurry was stirred with heating for 1 hour, and then stood to cool and ice-cooled. Precipitated crystals were collected by filtration, washed with cold ethyl acetate (20 ml), and dried under reduced pressure at 50° C., to yield the title compound (7.29 g, yield 79%).

¹H-NMR (DMSO-d₆, TMS, 300 MHz): δ (ppm): 1.01 (3H, d, J=6.3 Hz), 1.9-2.0 (1H, m), 2.0-2.4 (3H, m), 2.26 (3H, s), 2.5-2.6 (1H, m), 5.96 (1H, s), 7.97 (1H, d, J=1.9 Hz), 8.21 (1H, d, J=2.0 Hz), 8.42 (1H, br).

### (2) 3,7-Dimethyl-6,7,8,9-tetrahydro-5H-pyrido[2,3-b]indol-5-one

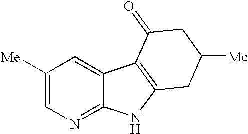

In a 200-ml flask, 3-[(3-bromo-5-methylpyridin-2-yl)amino]-5-methylcyclohex-2-en-1-one (23.7 mmol, 7.00 g), bis(triphenylphosphine)palladium dichloride (1.19 mmol, 5 mol %, 835 mg), cesium carbonate (71.1 mmol, 3.0 eq, 23.2 g) and toluene (70 ml) were mixed, and under an argon atmosphere, the mixture was stirred at room temperature for 1 hour. The mixture was allowed to react for 7 hours while heating to reflux, then the reaction solution was cooled, and 1N HCl (80.6 ml, 3.4 eq) and ethanol (49 ml) were added thereto. After heating to reflux for 1 hour, the reaction solution was stood to cool and ice-cooled. Precipitated crystals were collected by filtration, washed with water/ethanol (1/1, 30 ml), and dried under reduced pressure at 50° C., to yield the title compound (3.71 g). Further, the mother solution was extracted three times with ethyl acetate, the organic layers were combined and concentrated under reduced pressure, and the concentrated residue was washed with methanol (15 ml), to yield a second crystal (total yield 83% (4.22 g)).

¹H-NMR (DMSO-d₆, TMS, 300 MHz): δ (ppm): 1.12 (3H, d, J=6.2 Hz), 2.0-2.5 (3H, m), 2.38 (3H, s), 2.5-2.7 (1H, m), 2.9-3.3 (1H, m), 8.04 (1H, d, J=1.9 Hz), 8.08 (1H, d, J=1.9 Hz), 12.2 (1H, br).

Elemental analysis (C₁₃H₁₄N₂O)

Theoretical value: C: 72.87, H: 6.59, N: 13.07

Measured value: C: 72.69, H: 6.50, N: 13.09

### (3) 3,7-Dimethyl-9H-pyrido[2,3-b]indol-5-ol

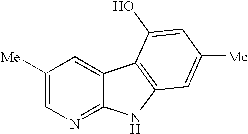

In a 30-ml flask, 3,7-dimethyl-6,7,8,9-tetrahydro-5H-pyrido[2,3-b]indol-5-one (1 mmol, 214 mg), pyridium hydrobromide perbromide (1 mmol, 320 mg) and acetonitrile (4.3 ml) were mixed, and the mixture was allowed to react for 72 hours. After cooling the reaction solution, an aqueous sodium bicarbonate solution was added thereto, and the mixture was extracted three times with a mixture of ethyl acetate/tetrahydrofuran. The organic layers were combined, washed with water, and then concentrated under reduced pressure. The residue was purified by silica gel column chromatography (eluent:hexane/ethyl acetate), to yield the title compound (57.9 mg, yield 27%).

¹H-NMR (DMSO-d₆, TMS, 300 MHz): δ (ppm): 2.37 (3H, s), 2.41 (3H, s), 6.42 (1H, s), 6.70 (1H, s), 8.12 (2H, s), 10.0 (1H, br), 11.3 (1H, br).

¹³C-NMR (DMSO-d₆, TMS, 300 MHz): δ (ppm): 17.96, 21.82, 102.24, 105.97, 106.54, 114.77, 123.07, 128.99, 137.13, 144.13, 144.14, 149.97, 153.65.

High resolution mass spectrometry (C₁₃H₁₂N₂O)

Theoretical value: 212.0941 (M⁺)

Measured value: 212.0950 (M⁺)

### Example 39

### (1) 3-[(3-Bromo-5-methylpyridin-2-yl)amino]-5-phenylcyclohex-2-en-1-one

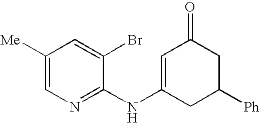

In a 300-ml flask connected to a Dean-Stark trap, 2-amino-3-bromo-5-methylpyridine (65.4 mmol, 12.2 g), 5-phenyl-1,3-cyclohexanedione (81.8 mmol, 1.25 eq, 15.4 g), p-toluenesulfonic acid monohydrate (6.54 mmol, 0.1 eq, 1.24 g) and toluene (122 ml) were mixed, and the mixture was heated to reflux for 9 hours. After completion of the reaction, the reaction solution was cooled, a 3% aqueous sodium bicarbonate solution (120 ml) was added thereto, and the mixture was extracted three times with ethyl acetate (120 ml). The organic layers were combined, washed with a 3% aqueous sodium bicarbonate solution (120 ml), and then concentrated to approximately a half the original amount. Ethyl acetate (100 ml) was added to the concentrate, and the mixture was further concentrated. This operation was performed three times in total, and a slurry with a concentrated weight of 52.5 g was obtained. The concentrated slurry was stirred with heating for 1 hour, and then stood to cool and ice-cooled. Precipitated crystals were collected by filtration, washed with cold ethyl acetate, and dried under reduced pressure at 50° C., to yield the title compound (15.6 g, yield 67%).

¹H-NMR (DMSO-d₆, TMS, 300 MHz): δ (ppm): 2.26 (3H, s), 2.2-2.4 (2H, m), 2.4-2.6 (1H, m), 2.8-2.9 (2H, m), 2.9-3.0 (1H, m), 6.09 (1H, s), 7.98 (1H, d, J=2.0 Hz), 8.22 (1H, d, J=2.0 Hz), 8.54 (1H, br).

### (2) 3-Methyl-7-phenyl-6,7,8,9-tetrahydro-5H-pyrido[2,3-b]indol-5-one

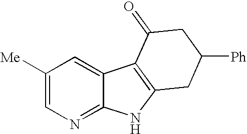

### (Method 1)

In a 50-ml flask, 3-[(3-bromo-5-methylpyridin-2-yl)amino]-5-phenylcyclohex-2-en-1-one (5 mmol, 1.79 g), bis(triphenylphosphine)palladium dichloride (0.5 mmol, 5 mol %, 175 mg), tripotassium phosphate (15 mmol, 3.0 eq, 3.18 g) and toluene (18 ml) were mixed, and under an argon atmosphere, the mixture was stirred at room temperature for 1 hour. The mixture was allowed to react for 10.5 hours under heating to reflux, then the reaction solution was cooled, and 1N HCl (17 ml, 3.4 eq) and ethanol (19.8 ml) were added thereto. The mixture was heated to reflux for 1 hour, and then stood to cool. The aqueous layer was separated, and extracted twice with ethyl acetate. The organic layers were combined, washed with water, and then concentrated under reduced pressure. Ethanol (5 ml) was added to the residue, and the mixture was stirred while heating, and stood to cool and ice-cooled. Precipitated crystals were collected by filtration, washed with cold ethanol, and dried under reduced pressure at 50° C., to yield the title compound (1.01 g, yield 73%).

¹H-NMR (DMSO-d₆, TMS, 300 MHz): δ (ppm): 2.39 (3H, s), 2.4-2.5 (1H, m), 2.8-2.9 (1H, m), 2.9-3.6 (2H, m), 3.6-3.8 (1H, m), 7.2-7.4 (5H, m), 8.08 (1H, s), 8.10 (1H, s), 12.3 (1H, br)

Mass analysis (EI, m/z) (rel intensity): 276 (M⁺, 50), 172 (100), 144 (35).

### (Method 2)

In a 50-ml flask, 3-[(3-bromo-5-methylpyridin-2-yl)amino]-5-phenylcyclohex-2-en-1-one (5 mmol, 1.79 g), bis(triphenylphosphine)palladium dichloride (0.5 mmol, 10 mol %, 175 mg), 1,5-diazabicyclo[2.2.2]octane (DABCO) (15 mmol, 3.0 eq, 1.68 g) and toluene (18 ml) were mixed, and under an argon atmosphere, the mixture was stirred at room temperature for 1 hour. The mixture was allowed to react for 10.5 hours under heating to reflux, then the reaction solution was cooled, and 1N HCl (17 ml, 3.4 eq) and ethanol (19.8 ml) were added thereto. The mixture was heated to reflux for 1 hour, and then stood to cool. The aqueous layer was separated, and extracted twice with ethyl acetate. The organic layers were combined, washed with tap water, and then concentrated under reduced pressure. Ethanol (5 ml) was added to the residue, and the mixture was stirred while heating, and stood to cool and ice-cooled. Precipitated crystals were collected by filtration, washed with cold ethanol, and dried under reduced pressure at 50° C., to yield the title compound (1.30 g, yield 94%).

### (3) 3-Methyl-7-phenyl-9H-pyrido[2,3-b]indol-5-ol

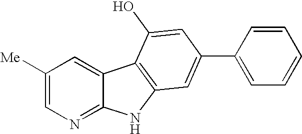

In a 30-ml flask, 3-methyl-7-phenyl-6,7,8,9-tetrahydro-5H-pyrido[2,3-b]indol-5-one (1 mmol, 276 mg), tetra-n-butylammonium tribromide (1 mmol, 482 mg) and N,N-dimethylformamide (2.8 ml) were mixed, and the mixture was allowed to react in an oil bath at 120° C. for 68 hours. The reaction solution was cooled, then a 5% aqueous sodium sulfite solution was added thereto, and the mixture was extracted twice with ethyl acetate. The organic layers were combined, washed with water, and then concentrated under reduced pressure. The residue was purified by silica gel column chromatography (eluent:hexane/ethyl acetate), and washed with hexane/ethyl acetate (1/1), to yield the title compound (77.8 mg, yield 36%).

¹H-NMR (DMSO-d₆, TMS, 300 MHz): δ (ppm): 2.43 (3H, s), 6.87 (1H, d, J=1.3 Hz), 7.13 (1H, d, J=1.3 Hz), 7.3-7.4 (1H, m), 7.4-7.6 (2H, m), 7.6-7.7 (2H, m), 8.18 (1H, d, J=1.6 Hz), 8.19 (1H, d, J=1.6 Hz), 10.4 (1H, br), 11.6 (1H, br).

¹³C-NMR (DMSO-d₆, TMS, 300 MHz): δ (ppm): 18.22, 100.68, 104.02, 108.40, 114.72, 123.77, 127.05, 127.40, 129.10, 129.78, 140.17, 141.49, 141.59, 145.23, 150.54, 154.44.

Mass analysis (EI, m/z) (rel intensity): 274 (M+, 100), 273 (15), 245 (10), 137 (7).

High resolution mass spectrometry (C₁₈H₁₄N₂O)

Theoretical value: 274.1101 (M⁺)

Measured value: 274.1106 (M⁺)

### Example 40

### (1) 3-[(3-Bromo-5-methylpyridin-2-yl)amino]-5-(2-furyl)cyclohex-2-en-1-one

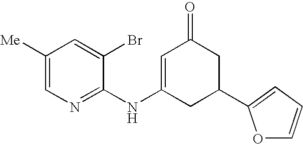

In a 100-ml flask connected to a Dean-Stark trap, 2-amino-3-bromo-5-methylpyridine (22.9 mmol, 4.28 g), 5-(2-furyl)-1,3-cyclohexanedione (28.6 mmol, 1.25 eq, 5.10 g), p-toluenesulfonic acid monohydrate (2.29 mmol, 0.1 eq, 436 mg) and toluene (42.8 ml) were mixed, and the mixture was heated to reflux for 9 hours. After completion of the reaction, the reaction solution was cooled, a 3% aqueous sodium bicarbonate solution (50 ml) was added thereto, and the mixture was extracted three times with ethyl acetate (50 ml). The organic layers were combined and washed with a 3% aqueous sodium bicarbonate solution (50 ml), and concentrated to approximately a half the original amount. Ethyl acetate (100 ml) was added to the concentrate, and the mixture was further concentrated. This operation was performed three times in total, to obtain the title compound as an oily product.

### (2) 3-Methyl-7-(2-furyl)-6,7,8,9-tetrahydro-5H-pyrido[2,3-b]indol-5-one

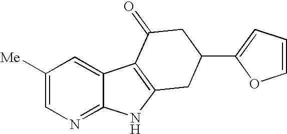

In a 200-ml flask, the entire amount of 3-[(3-bromo-5-methylpyridin-2-yl)amino]-5-(2-furyl)cyclohex-2-en-1-one obtained in (1) above, bis(triphenylphosphine)palladium dichloride (1.15 mmol, 5 mol %, 807 mg), cesium carbonate (68.7 mmol, 3.0 eq, 22.4 g) and toluene (79.5 ml) were mixed, and under an argon atmosphere, the mixture was stirred at room temperature for 1 hour. The mixture was allowed to react under heating to reflux for 18 hours, then the reaction solution was cooled, and 1N HCl (77.9 ml, 3.4 eq) and ethanol (40 ml) were added thereto. The mixture was heated to reflux for 1 hour, and then stood to cool. The aqueous layer was separated and extracted twice with ethyl acetate. The organic layers were combined, washed with water, and then concentrated under reduced pressure. Methanol (20 ml) was added to the residue, and the mixture was stirred while heating and then stood to cool and ice-cooled. Precipitated crystals were collected by filtration, washed with methanol, and dried under reduced pressure at 50° C., to yield the title compound (1.79 g, yield starting from 2-amino-3-bromo-5-methylpyridine 29%).

¹H-NMR (DMSO-d₆, TMS, 300 MHz): δ (ppm): 2.38 (3H, s), 2.7-2.8 (2H, m), 3.1-3.4 (2H, m), 3.7-3.8 (1H, m), 6.19 (1H, d, J=3.2 Hz), 6.38 (1H, dd, J=3.2 and 1.9 Hz), 7.57 (1H, d, J=1.1 Hz), 8.05 (1H, d, J=1.7 Hz), 8.10 (1H, d, J=1.3 Hz), 12.4 (1H, br).

Mass analysis (EI, m/z) (rel intensity): 266 (M+, 60), 172 (100), 144 (35), 117 (5).

High resolution mass spectrometry (C₁₆H₁₄N₂O₂)

Theoretical value: 266.1063 (M⁺)

Measured value: 266.1055 (M⁺)

### Example 41

### (1) 3-[(3-Bromo-5-chloropyridin-2-yl)amino]cyclohex-2-en-1-one

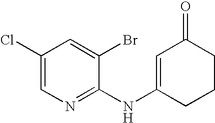

In a 200-ml flask connected to a Dean-Stark trap, 2-amino-3-bromo-5-chloropyridine (48.7 mmol, 10.1 g), 1,3-cyclohexanedione (73.1 mmol, 1.5 eq, 8.20 g), p-toluenesulfonic acid monohydrate (4.87 mmol, 0.1 eq, 926 mg) and toluene (101 ml) were mixed, and the mixture was heated to reflux for 7 hours. After completion of the reaction, the reaction solution was cooled, a 3% aqueous sodium bicarbonate solution (100 ml) was added, and the mixture was extracted three times with ethyl acetate (100 ml). The organic layers were combined, washed three times with a 3% aqueous sodium bicarbonate solution (100 ml) and once with water (100 ml), and then concentrated under reduced pressure. Ethyl acetate (25 ml) was added to the residue, and the mixture was stirred with heating for 1 hour, and then stood to cool and ice-cooled. Precipitated crystals were collected by filtration, washed with cold ethyl acetate, and dried under reduced pressure at 50° C., to yield the title compound (9.11 g, yield 62%).

¹H-NMR (DMSO-d₆, TMS, 300 MHz): δ (ppm): 1.8-2.0 (2H, m), 2.2-2.3 (2H, m), 2.6-2.7 (2H, m), 6.20 (1H, s), 8.33 (1H, d, J=4.5 Hz), 8.41 (1H, d, J=4.5 Hz), 8.49 (1H, br).

### (2) 3-Chloro-6,7,8,9-tetrahydro-5H-pyrido[2,3-b]indol-5-one

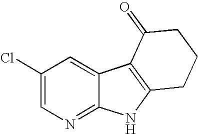

In a 200-ml flask, 3-[(3-bromo-5-chloropyridin-2-yl)amino]cyclohex-2-en-1-one (23.2 mmol, 7.00 g), bis(triphenylphosphine)palladium dichloride (1.16 mmol, 5 mol %, 814 mg), cesium carbonate (69.6 mmol, 3.0 eq, 22.7 g) and toluene (70 ml) were mixed, and under an argon atmosphere, the mixture was stirred at room temperature for 1 hour. The reaction solution was allowed to react under heating to reflux for 9.5 hours and then cooled, and 1N HCl (78.9 ml, 3.4 eq), water (25 ml) and ethanol (12.5 ml) were added thereto. The mixture was heated to reflux for 1 hour, and stood to cool and ice-cooled. Precipitated crystals were collected by filtration, washed with water/ethanol (1/1, 17 ml), and dried under reduced pressure at 50° C., to yield the title compound (4.30 g, yield 84%).

¹H-NMR (DMSO-d₆, TMS, 300 MHz): δ (ppm): 2.1-2.2 (2H, m), 2.4-2.5 (2H, m), 2.9-3.0 (2H, m), 8.18 (1H, d, J=2.4 Hz), 8.26 (1H, d, J=2.4 Hz), 12.6 (1H, br).

Mass analysis (EI, m/z) (rel intensity): 222 (M+2, 20), 220 (M+, 75), 194 (25), 192 (100), 166 (12), 164 (62).

High resolution mass spectrometry (C₁₁H₉ClN₂O)

Theoretical value: 220.0401 (M⁺)

Measured value: 220.0404 (M⁺)

### (3) 3-Chloro-6-bromo-6,7,8,9-tetrahydro-5H-pyrido[2,3-b]indol-5-one

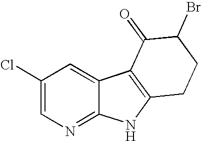

In a 50-ml flask, 3-chloro-6,7,8,9-tetrahydro-5H-pyrido[2,3-b]indol-5-one (10 mmol, 2.21 g), tetra-n-butylammonium tribromide (16.0 mmol, 7.71 g) and N,N-dimethylformamide (22 ml) were mixed, and the mixture was allowed to react at an internal temperature of near 80° C. for 2 hours. The reaction solution was cooled, then 1M HCl (44 ml) was added thereto, and the mixture was stirred with heating at 80° C. for 20 minutes. The mixture was cooled to room temperature, and the crystals were collected by filtration. The crystals were washed by sprinkling with 50% ethanol in water (20 ml), and dried under reduced pressure at 50° C., to yield the title compound (2.77 g, yield 92%).

### (4) 3-Chloro-9H-pyrido[2,3-b]indol-5-ol

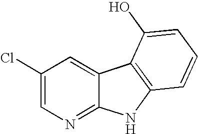

In a 50-ml flask, 3-chloro-6-bromo-6,7,8,9-tetrahydro-5H-pyrido[2,3-b]indol-5-one (9.01 mmol, 2.70 g), lithium bromide (27.0 mmol, 3 eq, 2.34 g), lithium carbonate (27.0 mmol, 4 eq, 2.00 g) and N,N-dimethylformamide (27 ml) were mixed, and under a nitrogen atmosphere, the mixture was allowed to react at an oil bath temperature of 120° C. for 2 hours. The reaction solution was cooled, then water (54 ml) was added thereto, and precipitated solids were collected by filtration. These solids were suspended in water/ethanol (1/1, 20 ml), and the suspension was stirred with heating. The suspension was cooled to room temperature, and the crystals were collected by filtration, washed with water/ethanol (1/1), and dried under reduced pressure at 50° C., to yield the title compound (1.61 g, yield 82%).

¹H-NMR (DMSO-d₆, TMS, 300 MHz): δ (ppm): 6.65 (1H, d, J=7.9 Hz), 6.94 (1H, d, J=8.0 Hz), 7.28 (1H, t, J=8.0 Hz), 8.1-8.3 (2H, m), 10.5 (1H, br), 11.9 (1H, br).

¹³C-NMR (DMSO-d₆, TMS, 300 MHz): δ (ppm): 102.56, 105.41, 108.47, 116.20, 121.59, 128.37, 128.80, 141.66, 142.66, 149.89, 154.57.

Mass analysis (EI, m/z) (rel intensity): 220 (M+2, 30), 218 (100), 189 (10), 155 (21).

High resolution mass spectrometry (C₁₁H₁₀N₂O)

Theoretical value: 218.0251 (M⁺)

Measured value: 218.0247 (M⁺)

### (5) 3-Chloro-9H-pyrido[2,3-b]indol-5-yl trifluoromethanesulfonate

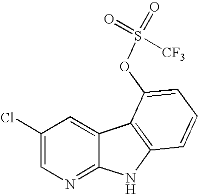

3-Chloro-9H-pyrido[2,3-b]indol-5-ol (22.9 mmol, 5.00 g) was suspended in pyridine (50 ml), and trifluoromethanesulfonic anhydride (1.47 eq, 5.65 ml) was added dropwise to the suspension under ice cooling with stirring. The mixture was stirred at the same temperature for 2 hours, and a saturated aqueous ammonium chloride solution (50 ml) was added thereto. Precipitated crystals were collected by filtration, washed with water (100 ml), and the crystals were dried under reduced pressure at 50° C., to yield the title compound (7.20 g, yield 90%).

Mass analysis (EI, m/z) (rel intensity): 350 (65), 217 (100), 189 (80), 153 (10).

High resolution mass spectrometry (C₁₂H₆ClF₃N₂O₃S)

Theoretical value: 349.9740 (M⁺)

Measured value: 340.9749 (M⁺)

### (6) 3-Chloro-5-(4-fluorophenyl)-9H-pyrido[2,3-b]indol

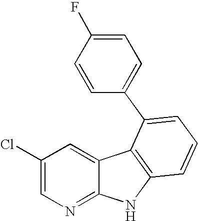

In a 50-ml flask, 3-chloro-9H-pyrido[2,3-b]indol-5-yl trifluoromethanesulfonate (1 mmol, 351 mg), 4-fluorophenylboronic acid (1.1 mmol, 1.1 eq, 154 mg), 1,1′-bis(diphenylphosphino)ferrocene palladium dichloride-dichloromethane complex (0.5 mmol, 5 mol %, 40.8 mg), sodium carbonate (3 mmol, 3.0 eq, 318 mg), toluene (3.5 ml), ethanol (3.5 ml) and water (1.2 ml) were mixed, and under an argon atmosphere, the mixture was stirred at room temperature for 1 hour. The mixture was allowed to react while heating to reflux for 2 hours, then the reaction solution was cooled, and 2M HCl (1.7 ml), water (5.2 ml) and ethanol (7 ml) were added thereto. The mixture was stirred with heating for 1 hour, and then stood to cool and ice-cooled. Precipitated crystals were collected by filtration, washed with cold hydrous ethanol (ethanol:water=1:1, 15 ml), and dried under reduced pressure at 50° C., to yield the title compound (176 mg, yield 59%).

¹H-NMR (DMSO-d₆, TMS, 300 MHz): δ (ppm): 7.1-7.2 (1H, m), 7.4-7.5 (2H, m), 7.5-7.6 (3H, m), 7.6-7.7 (2H, m), 8.4-8.5 (1H, m), 12.3 (1H, br).

¹³C-NMR (CDCl₃, TMS, 300 MHz): δ (ppm): 111.05, 115.77, 116.06, 116.98, 121.13, 121.28, 127.71, 128.01, 130.78, 130.88, 136.29, 136.84, 140.37, 144.21, 150.35.

Mass analysis (EI, m/z) (rel intensity): 296 (M+, 100), 260 (15), 232 (7), 130 (5).

High resolution mass spectrometry (C₁₇H₁₀ClFN₂)

Theoretical value: 296.0517 (M⁺)

Measured value: 296.0522 (M⁺)

### Example 42

### 1-[3-(3-Chloro-9H-pyrido[2,3-b]indol-5-yl)phenyl]ethanone

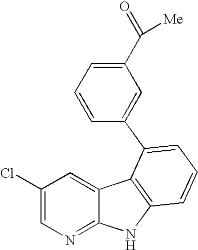

In a 50-ml flask, 3-chloro-9H-pyrido[2,3-b]indol-5-yl trifluoromethanesulfonate (1 mmol, 351 mg), 3-acetylphenylboronic acid (1.1 mmol, 1.1 eq, 180 mg), 1,1′-bis(diphenylphosphino)ferrocene palladium dichloride-dichloromethane complex (0.5 mmol, 5 mol %, 40.8 mg), sodium carbonate (3 mmol, 3.0 eq, 318 mg), toluene (3.5 ml), ethanol (3.5 ml) and water (1.2 ml) were mixed, and under an argon atmosphere, the mixture was stirred at room temperature for 1 hour. The mixture was allowed to react while heating to reflux for 2 hours, then the reaction solution was cooled, and 2M HCl (1.7 ml), water (5.2 ml) and ethanol (3.5 ml) were added thereto. The mixture was stirred with heating for 1 hour, and then stood to cool and ice-cooled. Precipitated crystals were collected by filtration, washed with cold hydrous ethanol (ethanol:water=1:1, 15 ml), and dried under reduced pressure at 50° C., to yield the title compound (253 mg, yield 79%).

¹H-NMR (DMSO-d₆, TMS, 300 MHz): δ (ppm): 2.59 (3H, s), 7.1-7.2 (1H, m), 7.5-7.6 (3H, m), 7.7-7.8 (1H, m), 7.9-8.0 (1H, m), 8.1-8.2 (2H, m), 8.4-8.5 (1H, m).

¹³C-NMR (CDCl₃, TMS, 300 MHz): δ (ppm): 111.38, 115.65, 116.82, 121.16, 121.26, 127.84, 128.06, 128.37, 129.50, 133.46, 136.89, 137.56, 140.27, 140.47, 144.30, 150.40, 198.03.

Mass analysis (EI, m/z) (rel intensity): 322 (30), 320 (M+, 100), 307 (10), 305 (30), 277 (20), 242 (30), 214 (10).

High resolution mass spectrometry (C₁₇H₁₃ClN₂O)

Theoretical value: 320.0717 (M⁺)

Measured value: 320.0721 (M⁺)

### Example 43

### 3-Chloro-5-(3,4-dimethoxyphenyl)-9H-pyrido[2,3-b]indol

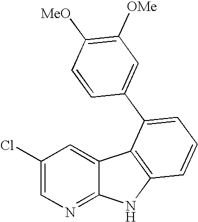

In a 50-ml flask, 3-chloro-9H-pyrido[2,3-b]indol-5-yl trifluoromethanesulfonate (1 mmol, 351 mg), 3,4-dimethoxyphenylboronic acid (1.1 mmol, 1.1 eq, 200 mg), 1,1′-bis(diphenylphosphino)ferrocene palladium dichloride-dichloromethane complex (0.5 mmol, 5 mol %, 40.8 mg), sodium carbonate (3 mmol, 3.0 eq, 318 mg), toluene (3.5 ml), ethanol (3.5 ml) and water (1.2 ml) were mixed, and under an argon atmosphere, the mixture was stirred at room temperature for 1 hour. The mixture was allowed to react while heating to reflux for 2 hours, then the reaction solution was cooled, and 2M HCl (1.7 ml), water (5.2 ml) and ethanol (3.5 ml) were added thereto. The mixture was stirred with heating for 1 hour, and then stood to cool and ice-cooled. Precipitated crystals were collected by filtration, washed with cold hydrous ethanol (ethanol:water=1:1, 15 ml), and dried under reduced pressure at 50° C., to yield the title compound (248 mg, yield 73%).

¹H-NMR (DMSO-d₆, TMS, 300 MHz): δ (ppm): 7.1-7.3 (4H, m), 7.5-7.8 (2H, m), 7.80 (1H, d, J=1.9 Hz), 8.41 (1H, d, J=1.9 Hz), 12.2 (1H, br).

¹³C-NMR (CDCl₃, TMS, 300 MHz): δ (ppm): 111.05, 115.77, 116.06, 116.98, 121.13, 121.28, 127.71, 128.01, 130.78, 130.88, 136.29, 136.84, 140.37, 144.21, 150.35.

Mass analysis (EI, m/z) (rel intensity): 340 (30), 338 (M+, 100), 323 (10), 295 (10), 260 (10), 245 (10).

High resolution mass spectrometry (C₁₇H₁₅ClN₂O₂)

Theoretical value: 338.0822 (M⁺)

Measured value: 338.0822 (M⁺)

### Example 44

### 3-Chloro-5-pyridin-3-yl-9H-pyrido[2,3-b]indol

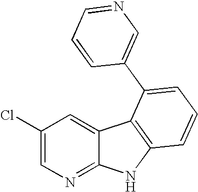

In a 50-ml flask, 3-chloro-9H-pyrido[2,3-b]indol-5-yl trifluoromethanesulfonate (1 mmol, 351 mg), 3-pyridylboronic acid (1.1 mmol, 1.1 eq, 135 mg), 1,1′-bis(diphenylphosphino)ferrocene palladium dichloride-dichloromethane complex (0.5 mmol, 5 mol %, 40.8 mg), sodium carbonate (3 mmol, 3.0 eq, 318 mg), toluene (3.5 ml), ethanol (3.5 ml) and water (1.2 ml) were mixed, and under an argon atmosphere, the mixture was stirred at room temperature for 1 hour. The mixture was allowed to react while heating to reflux for 2 hours, then the reaction solution was cooled, and 2M HCl (1.7 ml), water (5.2 ml) and ethanol (3.5 ml) were added thereto. The mixture was stirred with heating for 1 hour, and then stood to cool and ice-cooled. Precipitated crystals were collected by filtration, washed with cold hydrous ethanol (ethanol:water=1:1, 15 ml), and dried under reduced pressure at 50° C., to yield the title compound (220 mg, yield 79%).

¹H-NMR (DMSO-d₆, TMS, 300 MHz): δ (ppm): 7.1-7.2 (1H, m), 7.5-7.7 (4H, m), 8.0-8.1 (1H, m), 8.4-8.5 (1H, m), 8.7-8.9 (2H, m), 12.3 (1H, br).

¹³C-NMR (CDCl₃, TMS, 300 MHz): δ (ppm): 111.38, 115.65, 116.82, 121.16, 121.26, 127.84, 128.06, 128.37, 129.50, 133.46, 136.89, 137.56, 140.27, 140.47, 144.30, 150.40, 198.03.

Mass analysis (EI, m/z) (rel intensity): 281 (30), 279 (M+, 100), 243 (10), 216 (7).

High resolution mass spectrometry (C₁₆H₁₀ClN₃)

Theoretical value: 279.0556 (M⁺)

Measured value: 279.0564 (M⁺)

### Example 45

### 3-Chloro-5-[3-(ethylsulfonyl)phenyl]-9H-pyrido[2,3-b]indole

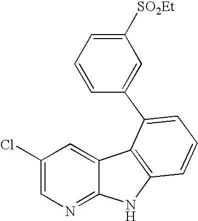

In a 50-ml flask, 3-chloro-9H-pyrido[2,3-b]indol-5-yl trifluoromethanesulfonate (1 mmol, 351 mg), 3-(ethylsulfonyl)phenylboronic acid (1.1 mmol, 1.1 eq, 235 mg), 1,1′-bis(diphenylphosphino)ferrocene palladium dichloride-dichloromethane complex (0.5 mmol, 5 mol %, 40.8 mg), sodium carbonate (3 mmol, 3.0 eq, 318 mg), toluene (3.5 ml), ethanol (3.5 ml) and water (1.2 ml) were mixed, and under an argon atmosphere, the mixture was stirred at room temperature for 1 hour. The mixture was allowed to react while heating to reflux for 2 hours, then the reaction solution was cooled, and 2M HCl (1.7 ml), tap water (5.2 ml) and ethanol (3.5 ml) were added thereto. The mixture was stirred with heating for 1 hour, and then stood to cool and ice-cooled. Precipitated crystals were collected by filtration, washed with cold hydrous ethanol (ethanol:water=1:1, 15 ml), and dried under reduced pressure at 50° C., to yield the title compound (313 mg, yield 84%).

¹H-NMR (DMSO-d₆, TMS, 300 MHz): δ (ppm): 1.19 (3H, t, J=7.3 Hz), 3.44 (2H, q, J=7.3 Hz), 7.2-7.3 (1H, m), 7.5-7.7 (3H, m), 7.8-8.0 (1H, m), 8.0-8.2 (3H, m), 8.44 (1H, d, J=2.2 Hz), 12.3 (1H, br).

¹³C-NMR (CDCl₃, TMS, 75.4 MHz): δ (ppm): 7.46, 49.19, 111.77, 115.42, 116.72, 121.28, 121.50, 127.57, 127.73, 127.87, 127.97, 130.48, 134.10, 135.85, 139.27, 140.48, 141.04, 144.48, 150.38.

Mass analysis (EI, m/z) (rel intensity): 370 (M+, 100), 277 (20), 276 (10), 242 (20), 241 (10), 214 (10).

High resolution mass spectrometry (C₁₉H₁₅ClN₂O₂S)

Theoretical value: 370.0543 (M⁺)

Measured value: 370.0527 (M⁺)

### Example 46

### 3-Chloro-5-[3-(ethylsulfonyl)phenyl]-6-iodo-9H-pyrido[2,3-b]indole

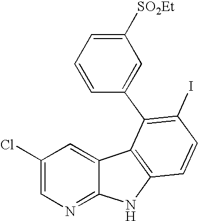

In a 25-ml flask, 3-chloro-5-[3-(ethylsulfonyl)phenyl]-9H-pyrido[2,3-b]indole (2 mmol, 742 mg) and acetonitrile (14.8 ml) were mixed. While stirring under ice cooling, N-iodosuccinimide (2.4 mmol, 1.2 eq, 540 mg), methanesulfonic acid (10 mmol, 5 eq, 0.65 g) and N-iodosuccinimide (180 mg) were sequentially added to the mixture, which was allowed to react. After completion of the reaction, a 5% aqueous sodium sulfite solution (30 ml) was added thereto, and the mixture was extracted three times with a mixture of ethyl acetate/THF (1/1). The combined organic layers were washed twice with a 5% aqueous sodium sulfite solution (30 ml) and once with 10% brine (30 ml), and concentrated under reduced pressure. EtOH (50 ml) was added to the residue, and the insoluble was filtered off. The filtrate was concentrated and dried under reduced pressure, the concentrate residue was washed with a mixture of ethyl acetate/hexane (4/3, 7 ml), and dried under reduced pressure at 50° C., to yield the title compound (545 mg, yield 55%).

¹H-NMR (DMSO-d₆, TMS, 300 MHz): δ (ppm): 1.12 (3H, t, J=7.4 Hz), 3.40 (2H, q, J=7.4 Hz), 6.60 (1H, d, J=2.4 Hz), 7.44 (1H, d, J=8.6 Hz), 7.77 (1H, d, J=7.7 Hz), 7.82 (1H, s), 7.95 (1H, t, J=7.7 Hz), 8.04 (1H, d, J=8.6 Hz), 8.14 (1H, d, J=8.0 Hz), 8.40 (1H, d, J=2.3 Hz), 12.1 (1H, br).

¹³C-NMR (CDCl₃, TMS, 75.4 MHz): δ (ppm): 7.73, 49.21, 88.94, 113.94, 115.25, 119.23, 121.66, 127.71, 128.09, 128.24, 130.81, 134.58, 136.99, 139.28, 139.44, 139.65, 143.22, 145.01, 150.07.

Mass analysis (EI, m/z) (rel intensity): 498 (40), 496 (M+, 100), 403 (10), 276 (22), 241 (10), 214 (10).

High resolution mass spectrometry (C₁₉H₁₅ClIN₂O₂S)

Theoretical value: 495.9514 (M⁺)

Measured value: 495.9509 (M⁺)

### Example 47

### (1) 3-[(3,5-Dichloropyridin-2-yl)amino]cyclohex-2-en-1-one

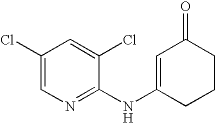

In a 300-ml flask connected to a Dean-Stark trap, 2-amino-3,5-dichloropyridine (155 mmol, 25.2 g), 1,3-cyclohexanedione (194 mmol, 1.25 eq, 21.8 g), p-toluenesulfonic acid monohydrate (15.5 mmol, 0.1 eq, 2.95 g) and toluene (252 ml) were mixed, and the mixture was heated to reflux for 8 hours. After completion of the reaction, the reaction solution was cooled, a 3% aqueous sodium bicarbonate solution (250 ml) was added thereto, and the mixture was extracted three times with ethyl acetate (125 ml). The organic layers were combined, washed with a 3% aqueous sodium bicarbonate solution (250 ml), and then concentrated under reduced pressure. Ethyl acetate (250 ml) was added to the concentrate, and the mixture was further concentrated. This operation was performed three times in total, and the amount of the concentration residue was adjusted to about 80 g. The concentrated slurry was stirred with heating for 1 hour, and then stood to cool and ice-cooled. Precipitated crystals were collected by filtration, washed with cold ethyl acetate (20 ml), and dried under reduced pressure at 50° C., to yield the title compound (19.8 g, yield 50%).

¹H-NMR (DMSO-d₆, TMS, 300 MHz): δ (ppm): 1.8-2.0 (2H, m), 2.2-2.3 (2H, m), 2.6-2.7 (2H, m), 6.34 (1H, s), 8.21 (1H, d, J=4.2 Hz), 8.37 (1H, d, J=4.1 Hz), 8.63 (1H, br).

### (2) 3-Chloro-6,7,8,9-tetrahydro-5H-pyrido[2,3-b]indol-5-one

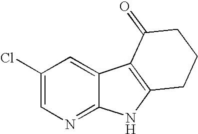

### (Synthetic Method 1)

In a 50-ml flask, 3-[(3,5-dichloropyridin-2-yl)amino]cyclohex-2-en-1-one (5.64 mmol, 1.45 g), tris(dibenzylideneacetone)dipalladium (0.282 mmol, 5 mol %, 258 mg), tricyclohexylphosphonium tetrafluoroborate (0.564 mmol, 10 mol %, 208 mg), tripotassium phosphate (16.9 mmol, 3.0 eq, 3.59 g) and toluene (14.5 ml) were mixed, and under an argon atmosphere, the mixture was stirred at room temperature for 1 hour. The mixture was allowed to react under heating to reflux for 14 hours, then the reaction solution was cooled, and 1N HCl (19.2 ml, 3.4 eq), water (5.5 ml) and ethanol (2.8 ml) were added thereto. The mixture was heated to reflux for 1 hour, and then stood to cool and ice-cooled. The aqueous layer was separated, and was extracted twice with a mixture of ethyl acetate and tetrahydrofuran. The organic layers were combined, washed with water, and then concentrated under reduced pressure. Ethyl acetate (5 ml) was added to the residue, and the mixture was stirred with heating, and then stood to cool and ice-cooled. Precipitated crystals were collected by filtration, washed with cold ethanol, and dried under reduced pressure at 50° C., to yield the title compound (585 mg, yield 47%).

### (Synthetic Method 2)

In a 50-ml flask, 3-[(3,5-dichloropyridin-2-yl)amino]cyclohex-2-en-1-one (4.8 mmol, 1.23 g), 1,1′-bis(diphenylphosphino)ferrocene palladium dichloride-dichloromethane complex (0.24 mmol, 5 mol %, 198 mg), tripotassium phosphate (14.4 mmol, 3.0 eq, 3.06 g) and toluene (12.3 ml) were mixed, and under an argon atmosphere, the mixture was stirred at room temperature for 1 hour. The mixture was allowed to react while heating to reflux for 29 hours, then the reaction solution was cooled, and 1N HCl (16.3 ml, 3.4 eq), water (4.7 ml) and ethanol (2.3 ml) were added. The mixture was heated to reflux for 1 hour, and then stood to cool and ice-cooled. Precipitated crystals were collected by filtration, washed with water/ethanol (1/1, 5 ml), and dried under reduced pressure at 50° C., to yield the title compound (600 mg, yield 57%).

¹H-NMR (DMSO-d₆, TMS, 300 MHz) δ (ppm): 2.1-2.2 (2H, m), 2.4-2.5 (2H, m), 2.9-3.0 (2H, m), 8.18 (1H, d, J=2.4 Hz), 8.26 (1H, d, J=2.4 Hz), 12.6 (1H, br).

### Example 48

### (1) 3-[(3-Chloro-5-trifluoromethylpyridin-2-yl)amino]cyclohex-2-en-1-one

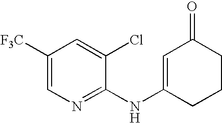

In a 100-ml flask connected to a Dean-Stark trap, 2-amino-3-chloro-5-trifluoromethylpyridine (26.8 mmol, 5.26 g), 1,3-cyclohexanedione (33.5 mmol, 1.25 eq, 3.76 g), p-toluenesulfonic acid monohydrate (2.68 mmol, 0.1 eq, 511 mg) and toluene (52.6 ml) were mixed, and the mixture was heated to reflux for 6 hours. After completion of the reaction, the reaction solution was cooled, a 3% aqueous sodium bicarbonate solution (50 ml) was added thereto, and the mixture was extracted with ethyl acetate (twice with 50 ml and once with 25 ml). The organic layers were combined, washed three times with a 3% aqueous sodium bicarbonate solution (50 ml) and twice with water (50 ml), and then concentrated under reduced pressure. Cold ethanol (12.5 ml) was added to the residue, and the mixture was stirred with heating for 1 hour, and then stood to cool and ice-cooled. Precipitated crystals were collected by filtration, washed with cold ethanol (12.5 ml), and dried under reduced pressure at 50° C., to yield the title compound (3.65 g, yield 47%).

¹H-NMR (DMSO-d₆, TMS, 300 MHz): δ (ppm): 1.8-2.0 (2H, m), 2.2-2.3 (2H, m), 2.7-2.8 (2H, m), 6.64 (1H, s), 8.37 (1H, d, J=1.9 Hz), 8.67 (1H, d, J=1.0 Hz), 8.74 (1H, br).

### (2) 3-Trifluoromethyl-6,7,8,9-tetrahydro-5H-pyrido[2,3-b]indol-5-one

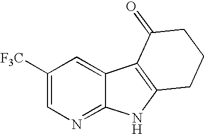

In a 50-ml flask, 3-[(3-chloro-5-trifluoromethylpyridin-2-yl)amino]cyclohex-2-en-1-one (5 mmol, 1.45 g), 1,1′-bis(diphenylphosphino)ferrocene palladium dichloride-dichloromethane complex (0.25 mmol, 5 mol %, 204 mg), tripotassium phosphate (15 mmol, 3.0 eq, 3.18 g) and toluene (14.5 ml) were mixed, and under an argon atmosphere, the mixture was stirred at room temperature for 1 hour. The mixture was allowed to react while heating to reflux for 13 hours, then the reaction solution was cooled, and 1N HCl (17 ml, 3.4 eq), water (5.5 ml) and ethanol (2.8 ml) were added thereto. The mixture was heated to reflux for 1 hour, and then stood to cool and ice-cooled. Precipitated crystals were collected by filtration, washed with water/ethanol (1/1, 5 ml), and dried under reduced pressure at 50° C., to yield the title compound (860 mg, yield 68%).

¹H-NMR (DMSO-d₆, TMS, 300 MHz): δ (ppm): 2.1-2.2 (2H, m), 2.4-2.5 (2H, m), 3.0-3.1 (2H, m), 8.44 (1H, d, J=1.9 Hz), 8.63 (1H, d, J=0.6 Hz), 12.8 (1H, br).

¹³C-NMR (DMSO-d₆, TMS, 300 MHz): δ (ppm): 22.58, 22.89, 37.37, 110.57, 115.93, 119.46, 124.54, 126.55, 140.12, 150.37, 155.93, 193.06.

Mass analysis (EI, m/z) (rel intensity): 254 (M⁺, 75), 226 (100), 198 (56), 171 (10).

High resolution mass spectrometry (C₁₂H₉F₃N₂O)

Theoretical value: 254.0668 (M⁺)

Measured value: 254.0667 (M⁺)

### Example 49

### (1) 3-[(3,5-Dibromopyridin-2-yl)amino]cyclohex-2-en-1-one

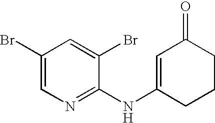

In a 500-ml flask connected to a Dean-Stark trap, 2-amino-3,5-dibromopyridine (100 mmol, 25.2 g), 1,3-cyclohexanedione (125 mmol, 1,25 eq, 14.0 g), p-toluenesulfonic acid monohydrate (10 mmol, 0.1 eq, 1.90 g) and toluene (252 ml) were mixed, and the mixture was heated to reflux for 3 hours. After completion of the reaction, the reaction solution was cooled, a 3% aqueous sodium bicarbonate solution (250 ml) was added, and the mixture was extracted once with ethyl acetate (200 ml) and twice with ethyl acetate (100 ml). The organic layers were combined, washed with a 3% aqueous sodium bicarbonate solution (250 ml), and then concentrated under reduced pressure. Ethyl acetate (50 ml) was added to the concentrated residue, and the mixture was stirred with heating, and then stood to cool and ice-cooled. Precipitated crystals were collected by filtration, washed with cold ethyl acetate (40 ml), and dried under reduced pressure at 50° C., to yield the title compound (25.6 g, yield 74%).

### (2) 3-Bromo-6,7,8,9-tetrahydro-5H-pyrido[2,3-b]indol-5-one

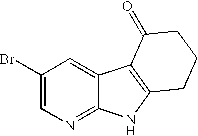

In a 200-ml flask, 3-[(3,5-dibromopyridin-2-yl)amino]cyclohex-2-en-1-one (25 mmol, 8.65 g), bis(triphenylphosphine)palladium dichloride (1.25 mmol, 5 mol %, 877 mg), 1,5-diazabicyclo[2.2.2]octane (DABCO) (75 mmol, 3.0 eq, 8.41 g) and toluene (from a can, 86.5 ml) were mixed, and under an argon atmosphere, the mixture was stirred at room temperature for 1 hour. The reaction solution was allowed to react under heating to reflux for 33 hours and then cooled, and dilute hydrochloric acid was added thereto. The mixture was extracted three times with a mixture of ethyl acetate and tetrahydrofuran. The organic layers were combined, washed with water, and then concentrated under reduced pressure. Ethanol (30 ml) was added to the concentrated residue. The mixture was heated to reflux for 1 hour, and stood to cool and ice-cooled. Precipitated crystals were collected by filtration, washed with cold ethanol, and dried under reduced pressure at 50° C., to yield the title compound (3.08 g, yield 47%).

¹H-NMR (DMSO-d₆, TMS, 300 MHz): δ (ppm): 2.1-2.2 (2H, m), 2.4-2.5 (2H, m), 2.9-3.0 (2H, m), 8.31 (1H, d, J=2.1 Hz), 8.34 (1H, d, J=2.2 Hz), 12.6 (1H, br).

### (3) 3-(4-Fluorophenyl)-6,7,8,9-tetrahydro-5H-pyrido[2,3-b]indol-5-one

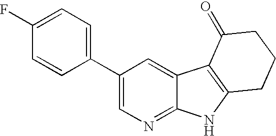

In a 50-ml flask, 3-bromo-6,7,8,9-tetrahydro-5H-pyrido[2,3-b]indol-5-one (4 mmol, 1.06 g), 4-fluorophenylboronic acid (4.4 mmol, 1.1 eq, 616 mg), 1,1′-bis(diphenylphosphino)ferrocene palladium dichloride-dichloromethane complex (2 mmol, 5 mol %, 163 mg), sodium carbonate (12 mmol, 3.0 eq, 1.27 g), toluene (10.6 ml), ethanol (10.6 ml) and water (3.5 ml) were mixed, and under an argon atmosphere, the mixture was stirred at room temperature for 1 hour. The mixture was allowed to react while heating to reflux for 12 hours, then the reaction solution was cooled, and 2M HCl (12 ml) was added thereto. The mixture was stirred with heating for 1 hour, and then stood to cool and ice-cooled. Precipitated crystals were collected by filtration, washed with cold hydrous ethanol (ethanol:water=1:1, 20 ml), and dried under reduced pressure at 50° C., to yield the title compound (786 mg, yield 70%).

¹H-NMR (DMSO-d₆, TMS, 300 MHz): δ (ppm): 2.1-2.2 (2H, m), 2.4-2.5 (2H, m), 2.9-3.0 (2H, m), 7.2-7.3 (2H, m), 7.7-7.8 (2H, m), 8.3-8.4 (1H, m), 8.5-8.6 (1H, m), 8.90 (1H, m).

Mass analysis (EI, m/z) (rel intensity): 280 (M⁺, 100), 252 (50), 224 (30), 126 (5).

High resolution mass spectrometry (C₁₇H₁₃FN₂O)

Theoretical value: 280.1012 (M⁺)

Measured value: 280.0996 (M⁺)

### (4) 3-(4-Fluorophenyl)-9H-pyrido[2,3-b]indol-5-ol

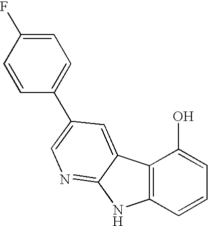

In a 50-ml flask, 3-(4-fluorophenyl)-6,7,8,9-tetrahydro-5H-pyrido[2,3-b]indol-5-one (2 mmol, 561 mg), tetra-n-butylammonium tribromide (3.2 mmol, 1.54 g) and N,N-dimethylformamide (5.6 ml) were mixed, and the mixture was allowed to react at an internal temperature of near 80° C. for 7 hours. The reaction solution was cooled, then 1M HCl (44 ml) was added thereto, and the mixture was stirred with heating at 80° C. for 20 minutes. The mixture was cooled to room temperature, and the precipitated crystals were collected by filtration. The crystals were washed by sprinkling with 50% ethanol in water (20 ml).

Lithium bromide (6 mmol, 3 eq, 521 mg), lithium carbonate (7.05 mmol, 521 mg) and N,N-dimethylformamide (6.1 ml) were added to the solid obtained above, and under a nitrogen atmosphere, the mixture was allowed to react at an oil bath temperature of 120° C. for 2 hours. The reaction solution was cooled, then water was added thereto, and the mixture was extracted twice with ethyl acetate. The organic layers were combined, washed with water, and then concentrated under reduced pressure. The residue was purified by silica gel column chromatography (eluent:hexane/ethyl acetate), to yield the title compound (81.6 mg, yield 15%).

¹H-NMR (DMSO-d₆, TMS, 300 MHz): δ (ppm): 6.68 (1H, d, J=7.8 Hz), 6.98 (1H, d, J=7.8 Hz), 7.2-7.4 (3H, m), 7.7-7.8 (2H, m), 8.57 (1H, d, J=2.1 Hz), 8.63 (1H, d, J=2.1 Hz), 10.3 (1H, br), 11.7 (1H, br).

Mass analysis (EI, m/z) (rel intensity): 278 (M⁺, 100), 249 (15), 139 (10).

High resolution mass spectrometry (C₁₇H₁₁FN₂O)

Theoretical value: 278.0855 (M⁺)

Measured value: 278.0863 (M⁺)

### Example 50

### 3-Phenyl-6,7,8,9-tetrahydro-5H-pyrido[2,3-b]indol-5-one

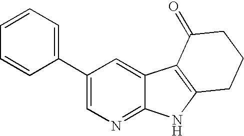

In a 50-ml flask, 3-bromo-6,7,8,9-tetrahydro-5H-pyrido[2,3-b]indol-5-one (4 mmol, 1.06 g), phenylboronic acid (4.4 mmol, 1.1 eq, 536 mg), 1,1′-bis(diphenylphosphino)ferrocene palladium dichloride-dichloromethane complex (2 mmol, 5 mol %, 163 mg), sodium carbonate (12 mmol, 3.0 eq, 1.27 g), toluene (10.6 ml), ethanol (10.6 ml) and water (3.5 ml) were mixed, and under an argon atmosphere, the mixture was stirred at room temperature for 1 hour. The mixture was allowed to react while heating to reflux for 8.5 hours, then the reaction solution was cooled, and 1M HCl (12 ml) and tetrahydrofuran (12 ml) were added thereto. The aqueous layer was separated, and was extracted twice with a mixture of ethyl acetate (6 ml)/tetrahydrofuran (6 ml). The organic layers were combined, washed with water (24 ml), and then concentrated under reduced pressure. Ethanol (12 ml) was added to the concentrated residue, and the mixture was heated to reflux for 1 hour, and then stood to cool and ice-cooled. Precipitated crystals were collected by filtration, washed with cold ethanol (12 ml), and dried under reduced pressure at 50° C., to yield the title compound (650 mg, yield 75%).

¹H-NMR (DMSO-d₆, TMS, 300 MHz): δ (ppm): 2.1-2.2 (2H, m), 2.4-2.5 (2H, m), 2.9-3.0 (2H, m), 7.3-7.5 (3H, m), 7.6-7.7 (2H, m), 8.42 (1H, d, J=2.0 Hz), 8.55 (1H, d, J=2.0 Hz), 12.5 (1H, br).

¹³C-NMR (CDCl₃, TMS, 300 MHz): δ (ppm): 23.18, 23.65, 38.11, 111.04, 117.47, 126.28, 127.49, 127.82, 129.62, 131.04, 138.97, 142.72, 148.83, 154.59, 193.53.

Mass analysis (EI, m/z) (rel intensity): 262 (M+, 100), 234 (75), 206 (45), 117 (15).

High resolution mass spectrometry (C₁₇H₁₄N₂O)

Theoretical value: 262.1105 (M⁺)

Measured value: 262.1106 (M⁺)

### Example 51

### 3-Pyridin-3-yl-6,7,8,9-tetrahydro-5H-pyrido[2,3-b]indol-5-one

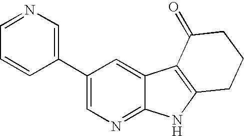

In a 50-ml flask, 3-bromo-6,7,8,9-tetrahydro-5H-pyrido[2,3-b]indol-5-one (4 mmol, 1.06 g), 3-pyridineboronic acid (4.4 mmol, 1.1 eq, 541 mg), 1,1′-bis(diphenylphosphino)ferrocene palladium dichloride-dichloromethane complex (2 mmol, 5 mol %, 163 mg), sodium carbonate (12 mmol, 3.0 eq, 1.27 g), toluene (10.6 ml), ethanol (10.6 ml) and water (3.5 ml) were mixed, and under an argon atmosphere, the mixture was stirred at room temperature for 1 hour. The mixture was allowed to react while heating to reflux for 8.5 hours, then the reaction solution was cooled, and 2M HCl (12 ml) was added thereto. The mixture was heated to reflux for 1 hour, and then stood to cool and ice-cooled. Precipitated crystals were collected by filtration, washed with cold hydrous ethanol (ethanol:water=1:1, 20 ml), and dried under reduced pressure at 50° C., to yield the title compound (650 mg, yield 62%).

¹H-NMR (DMSO-d₆, TMS, 300 MHz): δ (ppm): 2.1-2.2 (2H, m), 2.4-2.5 (2H, m), 2.8-2.9 (2H, m), 7.4-7.5 (1H, m), 7.9-8.0 (1H, m), 8.1-8.2 (1H, m), 8.4-8.5 (2H, m), 8.90 (1H, m).

Mass analysis (EI, m/z) (rel intensity): 263 (M⁺, 100), 235 (90), 207 (55), 179 (10).

High resolution mass spectrometry (C₁₆H₁₃N₃O)

Theoretical value: 263.1059 (M⁺)

Measured value: 263.1062 (M⁺)

### Example 52

### 3-[3-(Ethylsulfonyl)phenyl]-6,7,8,9-tetrahydro-5H-pyrido[2,3-b]indol-5-one

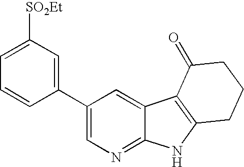

In a 50-ml flask, 3-bromo-6,7,8,9-tetrahydro-5H-pyrido[2,3-b]indol-5-one (4 mmol, 1.06 g), 3-(ethylsulfonyl)phenylboronic acid (4.4 mmol, 1.1 eq, 940 mg), 1,1′-bis(diphenylphosphino)ferrocene palladium dichloride-dichloromethane complex (2 mmol, 5 mol %, 163 mg), sodium carbonate (12 mmol, 3.0 eq, 1.27 g), toluene (10.6 ml), ethanol (10.6 ml) and water (3.5 ml) were mixed, and under an argon atmosphere, the mixture was stirred at room temperature for 1 hour. The mixture was allowed to react while heating to reflux for 8.5 hours, then the reaction solution was cooled, and 2M HCl (12 ml) was added thereto. The mixture was heated to reflux for 1 hour, and then stood to cool and ice-cooled. Precipitated crystals were collected by filtration, washed with cold hydrous ethanol (ethanol:water=1:1, 20 ml), and dried under reduced pressure at 50° C., to yield the title compound (902 mg, yield 69%).

¹H-NMR (DMSO-d₆, TMS, 300 MHz): δ (ppm): 1.16 (3H, t, J=7.3 Hz), 2.1-2.2 (2H, m), 3.0-3.1 (2H, m), 3.3-3.5 (4H, m), 7.7-7.8 (1H, m), 7.8-7.9 (1H, m), 8.1-8.2 (1H, m), 8.50 (1H, d, J=1.9 Hz), 8.65 (1H, d, J=1.9 Hz).

Mass analysis (EI, m/z) (rel intensity): 354 (M⁺, 100), 326 (50), 298 (5), 261 (5), 205 (10).

High resolution mass spectrometry (C₁₉H₁₈N₂O₃S)

Theoretical value: 354.1038 (M⁺)

Measured value: 354.1034 (M⁺)

### Example 53

### 3-(3-Acetylphenyl)-6,7,8,9-tetrahydro-5H-pyrido[2,3-b]indol-5-one

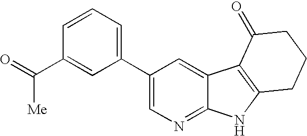

In a 50-ml flask, 3-bromo-6,7,8,9-tetrahydro-5H-pyrido[2,3-b]indol-5-one (4 mmol, 1.06 g), 3-acetylphenylboronic acid (4.4 mmol, 1.1 eq, 721 mg), 1,1′-bis(diphenylphosphino)ferrocene palladium dichloride-dichloromethane complex (2 mmol, 5 mol %, 163 mg), sodium carbonate (12 mmol, 3.0 eq, 1.27 g), toluene (10.6 ml), ethanol (10.6 ml) and water (3.5 ml) were mixed, and under an argon atmosphere, the mixture was stirred at room temperature for 1 hour. The mixture was allowed to react while heating to reflux for 8.5 hours, then the reaction solution was cooled, and 2M HCl (12 ml) was added thereto. The mixture was heated to reflux for 1 hour, and then stood to cool and ice-cooled. Precipitated crystals were collected by filtration, washed with cold hydrous ethanol (ethanol:water=1:1, 15 ml), and dried under reduced pressure at 50° C., to yield the title compound (1.00 g, yield 82%).

¹H-NMR (DMSO-d₆, TMS, 300 MHz): δ (ppm): 2.1-2.2 (2H, m), 2.4-2.5 (2H, m), 2.68 (3H, s), 2.9-3.0 (2H, m), 7.6-7.7 (1H, m), 7.9-8.0 (2H, m), 8.20 (1H, br), 8.44 (1H, d, J=1.8 Hz), 8.58 (1H, d, J=1.8 Hz), 12.4 (1H, br).

Mass analysis (EI, m/z) (rel intensity): 304 (M⁺, 100), 289 (25), 276 (60), 248 (15), 205 (10).

High resolution mass spectrometry (C₁₉H₁₆N₂O₂)

Theoretical value: 304.1212 (M⁺)

Measured value: 304.1213 (M⁺)

### Example 54

### 3-(3,4-Dimethoxyphenyl)-6,7,8,9-tetrahydro-5H-pyrido[2,3-b]indol-5-one

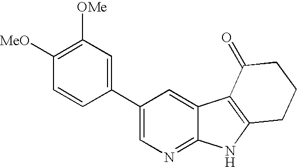

In a 50-ml flask, 3-bromo-6,7,8,9-tetrahydro-5H-pyrido[2,3-b]indol-5-one (4 mmol, 1.06 g), 3,4-dimethoxyphenylboronic acid (4.4 mmol, 1.1 eq, 801 mg), 1,1′-bis(diphenylphosphino)ferrocene palladium dichloride-dichloromethane complex (2 mmol, 5 mol %, 163 mg), sodium carbonate (12 mmol, 3.0 eq, 1.27 g), toluene (10.6 ml), ethanol (10.6 ml) and water (3.5 ml) were mixed, and under an argon atmosphere, the mixture was stirred at room temperature for 1 hour. The mixture was allowed to react while heating to reflux for 8.5 hours, then the reaction solution was cooled, and 1M HCl (12 ml) and tetrahydrofuran (12 ml) were added thereto. The aqueous layer was separated, and was extracted twice with a mixture of ethyl acetate (6 ml)/tetrahydrofuran (6 ml). The organic layers were combined, washed with water (24 ml), and then concentrated under reduced pressure. Ethanol (12 ml) was added to the concentrated residue, and the mixture was heated to reflux for 1 hour, and then stood to cool and ice-cooled. Precipitated crystals were collected by filtration, washed with cold ethanol (12 ml), and dried under reduced pressure at 50° C., to yield the title compound (729 mg, yield 56%)

¹H-NMR (DMSO-d₆, TMS, 300 MHz): δ (ppm): 2.1-2.2 (2H, m), 2.4-2.5 (2H, m), 2.9-3.0 (2H, m), 3.81 (3H, s), 3.88 (3H, s), 7.0-7.1 (1H, m), 7.1-7.3 (2H, m), 8.38 (1H, br), 8.53 (1H, br), 12.4 (1H, br).

Mass analysis (EI, m/z) (rel intensity): 322 (M⁺, 100), 307 (10), 294 (5), 279 (10).

High resolution mass spectrometry (C₁₉H₁₈N₂O₃)

Theoretical value: 322.0668 (M⁺)

Measured value: 322.1317 (M⁺)

### INDUSTRIAL APPLICABILITY

Since the method for preparation of the present invention does not necessitate expensive starting compounds or special reaction apparatuses such as those used in conventional methods, α-carboline derivatives (II), (IX), (XV), (XVII), and (XIX) having various substituents can be prepared in few steps, as well as conveniently and industrially advantageously. Also, based on the development of the novel compounds using this method, novel intermediates (XI), (XII), (XIII), and (XX) for establishing efficient methods for preparing known pharmaceutical products, can be provided.

This application is based on patent application Nos. 2006-211472 and 2007-106067 filed in Japan, the contents of which are incorporated in full herein by this reference.

1. A method for preparation of a compound represented by the following formula: 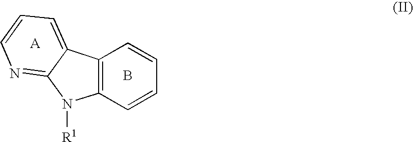
wherein ring A represents a pyridine ring which may be substituted; R¹ represents a hydrogen atom, a C₁₋₁₀ alkyl group which may be substituted, or an acyl group; ring B represents a benzene ring which may be substituted, or a pyridine ring which may be substituted; and at least one of ring A and ring B is substituted;
or a salt thereof, by subjecting a compound represented by the following formula: 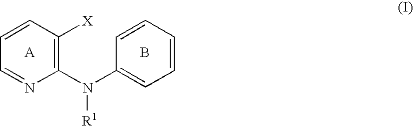
wherein ring A, R¹, and ring B, respectively represent the same meaning as defined above; X represents a leaving group; and at least one of ring A and ring B is substituted;
or a salt thereof to a ring closure reaction in the presence of a palladium catalyst, a ligand, and a base.

2. The method according to claim 1, wherein X is a halogen atom, a C₁₋₄ alkanesulfonyloxy group which may be halogenated, or a benzenesulfonyloxy group which may be substituted.

3. A method for preparation of a compound represented by the following formula: 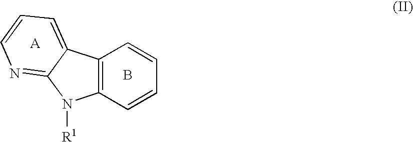
wherein ring A represents a pyridine ring which may be substituted; R¹ represents a hydrogen atom, a C₁₋₁₀ alkyl group which may be substituted, or an acyl group; ring B represents a benzene ring which may be substituted, or a pyridine ring which may be substituted; and at least one of ring A and ring B is substituted;
or a salt thereof, by reacting a compound represented by the following formula: 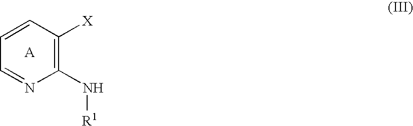
wherein ring A and R¹ respectively represent the same meaning as defined above; and X represents a leaving group;
with a compound represented by the following formula: 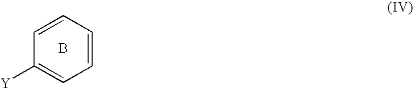
wherein ring B represents the same meaning as defined above; and Y represents a halogen atom, a C₁₋₄ alkanesulfonyloxy group which may be halogenated, or a benzenesulfonyloxy group which may be substituted;
in the presence of a metal catalyst to obtain a compound represented by the following formula: 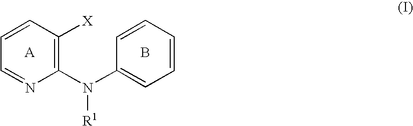
wherein the symbols respectively represent the same meaning as defined above; and at least one of ring A and ring B is substituted;
and subsequently subjecting the compound represented by Formula (I) or a salt thereof to a ring closure reaction in the presence of a palladium catalyst, a ligand, and a base.

4. A method for preparation of a compound represented by the following formula: 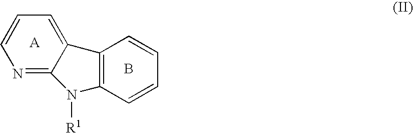
wherein ring A represents a pyridine ring which may be substituted; R¹ represents a hydrogen atom, a C₁₋₁₀ alkyl group which may be substituted, or an acyl group; ring B represents a benzene ring which may be substituted, or a pyridine ring which may be substituted; and at least one of ring A and ring B is substituted;
or a salt thereof, by reacting a compound represented by the following formula: 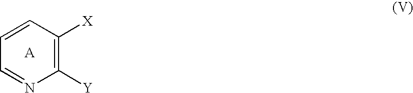
wherein ring A represents the same meaning as defined above; X represents a leaving group; and Y represents a halogen atom, a C₁₋₄ alkanesulfonyloxy group which may be halogenated, or a benzenesulfonyloxy group which may be substituted;
with a compound represented by the following formula: 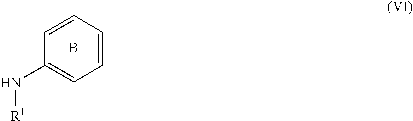
wherein the symbols respectively represent the same meaning as defined above;
to obtain a compound represented by the following formula: 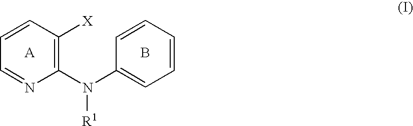
wherein the symbols respectively represent the same meaning as defined above; and at least one of ring A and ring B is substituted;
and subsequently subjecting the compound represented by Formula (I) or a salt thereof to a ring closure reaction in the presence of a palladium catalyst, a ligand, and a base.

5. The method according to claim 1, wherein the substituent of ring A or ring B is a halogen atom, a hydroxyl group, an amino group which may be substituted, a C₁₋₁₀ alkoxycarbonyl group which may be substituted, an aminocarbonyl group which may have one or two substituents on the nitrogen atom, a C₆₋₁₀ aryl group which may be substituted, or a C₅₋₁₀ heteroaryl group which may be substituted.

6. The method according to claim 1, wherein the ligand is 2-(dicyclohexylphosphino)biphenyl or 1,1′-bis(diphenylphosphino)ferrocene.

7. The method according to claim 1, wherein the base is 1,5-diazabicyclo[4.3.0]non-5-ene (DBN) or 1,8-diazabicyclo[5.4.0]undec-7-ene (DBU).

8. A method for preparation of a compound represented by the following formula: 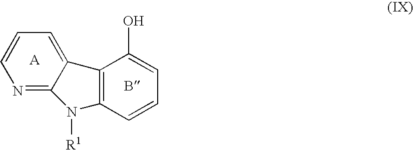
wherein ring A represents a pyridine ring which may be substituted; R¹ represents a hydrogen atom, a C₁₋₁₀ alkyl group which may be substituted, or an acyl group; ring B″ represents a benzene ring which may be substituted; and at least one of ring A and ring B″ is substituted;
or a salt thereof, by subjecting a compound represented by the following formula: 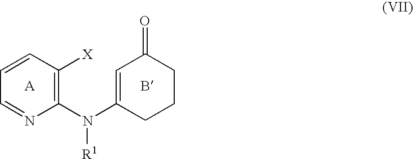
wherein ring A and R¹ respectively represent the same meaning as defined above; X represents a leaving group; ring B′ represents a cyclohexenone ring which may be substituted; and at least one of ring A and ring B′ is substituted;
or a salt thereof to a ring closure reaction in the presence of a palladium catalyst, a ligand, and a base, to obtain a compound represented by the following formula: 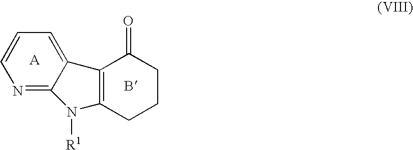
wherein the symbols respectively represent the same meaning as defined above; and at least one of ring A and ring B′ is substituted;
or a salt thereof, and subsequently aromatizing ring B′ of the compound represented by Formula (VIII) or a salt thereof.

9. A method for preparation of a compound represented by the following formula: 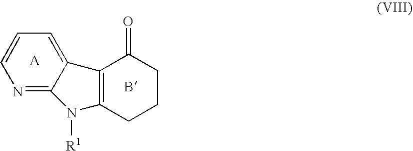
wherein ring A represents a pyridine ring which may be substituted; R¹ represents a hydrogen atom, a C₁₋₁₀ alkyl group which may be substituted, or an acyl group; ring B′ represents a cyclohexenone ring which may be substituted; and at least one of ring A and ring B′ is substituted;
or a salt thereof, by reacting a compound represented by the following formula: 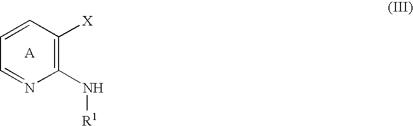
wherein ring A and R¹ respectively represent the same meaning as defined above; and X represents a leaving group;
with a compound represented by the following formula: 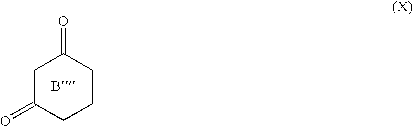
wherein ring B″″ represents a 1,3-cyclohexanedione ring which may be substituted;
to obtain a compound represented by the following formula: 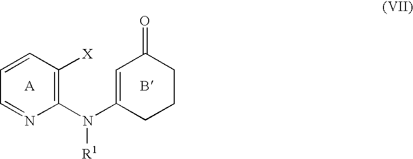
wherein the symbols respectively represent the same meaning as defined above; and at least one of ring A and ring B′ is substituted;
and subsequently subjecting the compound represented by Formula (VII) to a ring closure reaction in the presence of a palladium catalyst, a ligand, and a base.

10. A method for preparation of a compound represented by the following formula: 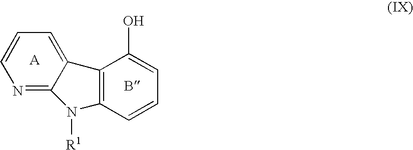
wherein ring A represents a pyridine ring which may be substituted; R¹ represents a hydrogen atom, a C₁₋₁₀ alkyl group which may be substituted, or an acyl group; ring B″ represents a benzene ring which may be substituted; and at least one of ring A and ring B″ is substituted;
or a salt thereof, by reacting a compound represented by the following formula: 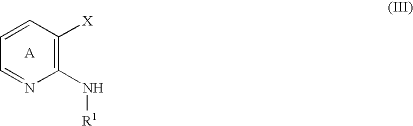
wherein ring A and R¹ respectively represent the same meaning as defined above; and X represents a leaving group;
with a compound represented by the following formula: 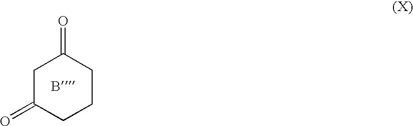
wherein ring B″″ represents a 1,3-cyclohexanedione ring which may be substituted;
to obtain a compound represented by the following formula: 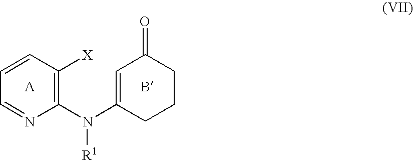
wherein ring A, R¹, and X respectively represent the same meaning as defined above; ring B′ represents a cyclohexenone ring which may be substituted; and at least one of ring A and ring B′ is substituted;
subsequently subjecting the compound represented by Formula (VII) to a ring closure reaction in the presence of a palladium catalyst, a ligand, and a base to obtain a compound represented by the following formula: 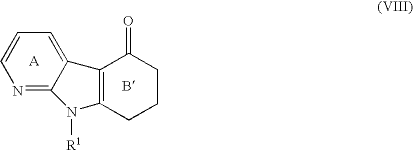
wherein the symbols respectively represent the same meaning as defined above; and at least one of ring A and ring B′ is substituted;
or a salt thereof, and subsequently aromatizing ring B′ of the compound represented by Formula (VIII) or a salt thereof.

11. The method according to claim 8, wherein the base is cesium carbonate, tripotassium phosphate, or 1,5-diazabicyclo[2.2.2]octane (DABCO).

12. A compound represented by the following formula: 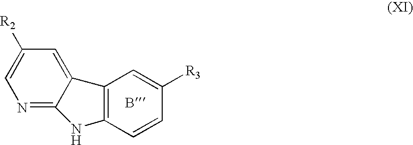
wherein ring B′″ represents a benzene ring which may be further substituted in addition to R³; R² represents a halogen atom, a nitro group, a C₁₋₁₀ alkyl group which may be substituted, an amino group which may be substituted, or a C₁₋₁₀ alkylthio group which may be substituted; R³ represents a halogen atom, a C₁₋₁₀ alkoxy group which may be substituted, an amino group which may be substituted, or a C₁₋₁₀ alkylthio group which may be substituted;
or a salt thereof.

13. A compound represented by the following formula: 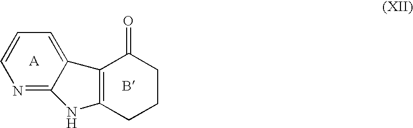
wherein ring A represents a pyridine ring which may be substituted; ring B′ represents a cyclohexenone ring which may be substituted; and at least one of ring A and ring B′ is substituted;
or a salt thereof, provided that the following compounds are excluded:

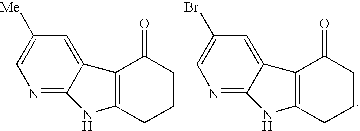

14. A compound represented by the following formula: 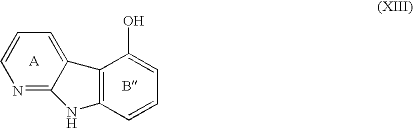
wherein ring A represents a pyridine ring which may be substituted; ring B″ represents a benzene ring which may be substituted; and at least one of ring A and ring B″ is substituted;
or a salt thereof.

15. A method for preparation of a compound represented by the following formula: 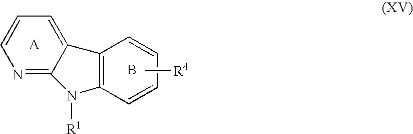
wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B represents a benzene ring which may be substituted or a pyridine ring which may be substituted; and at least one of ring A and ring B is substituted;
or a salt thereof, by reacting a compound represented by the following formula: 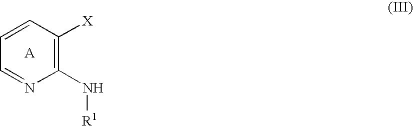
wherein ring A and R¹ respectively represent the same meaning as defined above; and X represents a leaving group;
with a compound represented by the following formula: 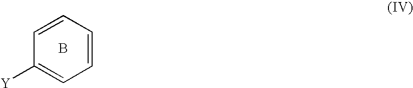
wherein ring B represents the same meaning as defined above; and Y represents a halogen atom, a C₁₋₄ alkanesulfonyloxy group which may be halogenated, or a benzenesulfonyloxy group which may be substituted;
in the presence of a metal catalyst to obtain a compound represented by the following formula: 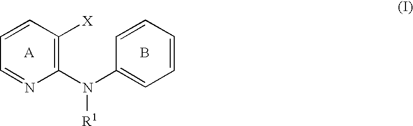
wherein the symbols respectively represent the same meaning as defined above; and at least one of ring A and ring B is substituted;
subsequently subjecting the compound represented by Formula (I) or a salt thereof to a ring closure reaction in the presence of a palladium catalyst, a ligand, and a base, to obtain a compound represented by the following formula: 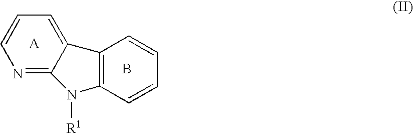
wherein the symbols respectively represent the same meaning as defined above; and at least one of ring A and ring B is substituted;
or a salt thereof, then subsequently subjecting the compound represented by Formula (II) or a salt thereof to a reaction for introducing a leaving group when necessary, to obtain a compound represented by the following formula: 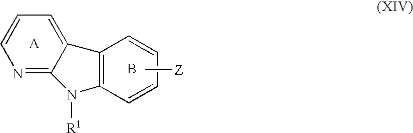
wherein Z represents a leaving group; other symbols respectively represent the same meaning as defined above; and at least one of ring A and ring B is substituted;
or a salt thereof, and subsequently subjecting the compound represented by Formula (II) or a salt thereof or the compound represented by Formula (XIV) or a salt thereof, to a coupling reaction.

16. A method for preparation of a compound represented by the following formula: 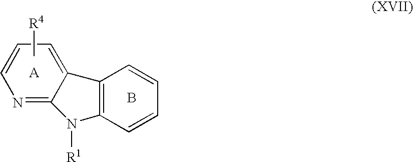
wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B represents a benzene ring which may be substituted or a pyridine ring which may be substituted; and at least one of ring A and ring B is substituted;
or a salt thereof, by reacting a compound represented by the following formula: 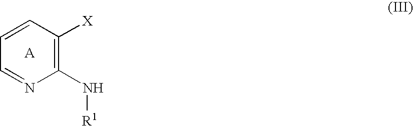
wherein ring A and R¹ respectively represent the same meaning as defined above; and X represents a leaving group;
with a compound represented by the following formula: 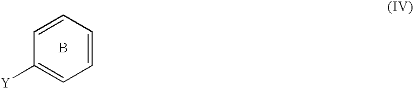
wherein ring B represents the same meaning as defined above; and Y represents a halogen atom, a C₁₋₄ alkanesulfonyloxy group which may be halogenated, or a benzenesulfonyloxy group which may be substituted;
in the presence of a metal catalyst to obtain a compound represented by the following formula: 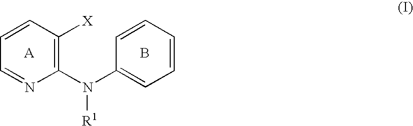
wherein the symbols respectively represent the same meaning as defined above; and at least one of ring A and ring B is substituted;
subsequently subjecting the compound represented by Formula (I) or a salt thereof to a ring closure reaction in the presence of a palladium catalyst, a ligand, and a base, to obtain a compound represented by the following formula: 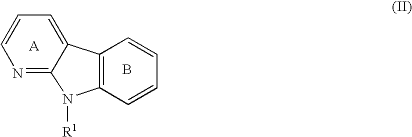
wherein the symbols respectively represent the same meaning as defined above; and at least one of ring A and ring B is substituted;
or a salt thereof, then subsequently subjecting the compound represented by Formula (II) or a salt thereof to a reaction for introducing a leaving group when necessary, to obtain a compound represented by the following formula: 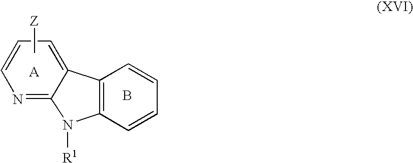
wherein Z represents a leaving group; other symbols respectively represent the same meaning as defined above; and at least one of ring A and ring B is substituted;
or a salt thereof, and subsequently subjecting the compound represented by Formula (II) or a salt thereof or the compound represented by Formula (XVI) or a salt thereof, to a coupling reaction.

17. A method for preparation of a compound represented by the following formula: 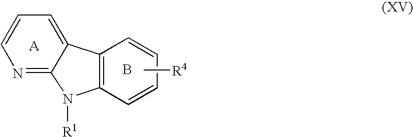
wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B represents a benzene ring which may be substituted or a pyridine ring which may be substituted; and at least one of ring A and ring B is substituted;
or a salt thereof, by reacting a compound represented by the following formula: 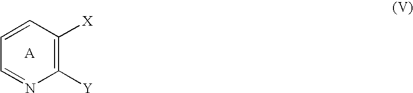
wherein ring A represents the same meaning as defined above; X represents a leaving group; and Y represents a halogen atom, a C₁₋₄ alkanesulfonyloxy group which may be halogenated, or a benzenesulfonyloxy group which may be substituted;
with a compound represented by the following formula: 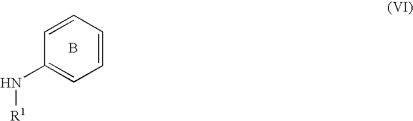
wherein the symbols respectively represent the same meaning as defined above;
to obtain a compound represented by the following formula: 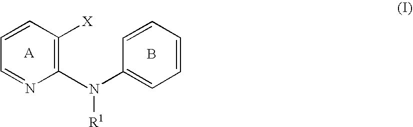
wherein the symbols respectively represent the same meaning as defined above; and at least one of ring A and ring B is substituted;
subsequently subjecting the compound represented by Formula (I) or a salt thereof to a ring closure reaction in the presence of a palladium catalyst, a ligand, and a base, to obtain a compound represented by the following formula: 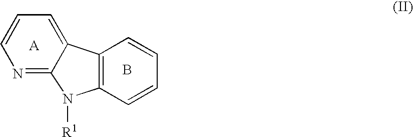
wherein the symbols respectively represent the same meaning as defined above; and at least one of ring A and ring B is substituted;
or a salt thereof, then subsequently subjecting the compound represented by Formula (II) or a salt thereof to a reaction for introducing a leaving group when necessary, to obtain a compound represented by the following formula: 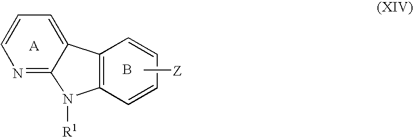
wherein Z represents a leaving group; other symbols respectively represent the same meaning as defined above; and at least-one of ring A and ring B is substituted;
or a salt thereof, and subsequently subjecting the compound represented by Formula (II) or a salt thereof or the compound represented by Formula (XIV) or a salt thereof, to a coupling reaction.

18. A method for preparation of a compound represented by the following formula: 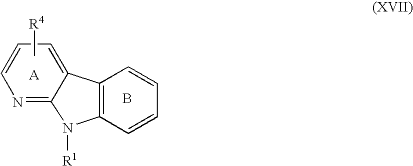
wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B represents a benzene ring which may be substituted or a pyridine ring which may be substituted; and at least one of ring A and ring B is substituted;
or a salt thereof, by reacting a compound represented by the following formula: 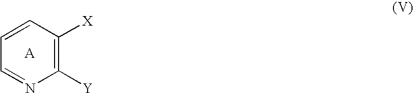
wherein ring A represents the same meaning as defined above; X represents a leaving group; and Y represents a halogen atom, a C₁₋₄ alkanesulfonyloxy group which may be halogenated, or a benzenesulfonyloxy group which may be substituted;
with a compound represented by the following formula: 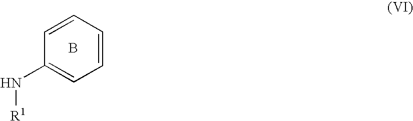
wherein the symbols respectively represent the same meaning as defined above;
to obtain a compound represented by the following formula: 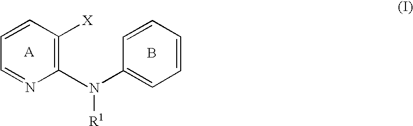
wherein the symbols respectively represent the same meaning as defined above; and at least one of ring A and ring B is substituted;
subsequently subjecting the compound represented by Formula (I) or a salt thereof to a ring closure reaction in the presence of a palladium catalyst, a ligand, and a base, to obtain a compound represented by the following formula: 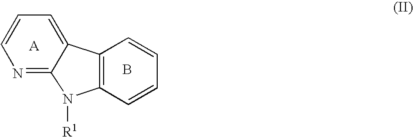
wherein the symbols respectively represent the same meaning as defined above; and at least one of ring A and ring B is substituted;
or a salt thereof, then subsequently subjecting the compound represented by Formula (II) or a salt thereof to a reaction for introducing a leaving group when necessary, to obtain a compound represented by the following formula: 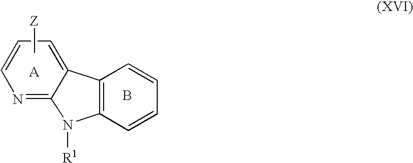
wherein Z represents a leaving group; other symbols respectively represent the same meaning as defined above; and at least one of ring A and ring B is substituted;
or a salt thereof, and subsequently subjecting the compound represented by Formula (II) or a salt thereof or the compound represented by Formula (XVI) or a salt thereof, to a coupling reaction.

19. A method for preparation of a compound represented by the following formula: 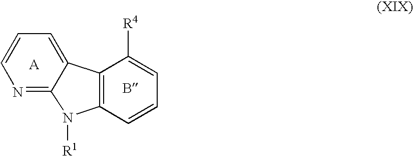
wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B″ represents a benzene ring which may be substituted; and at least one of ring A and ring B″ is substituted;
or a salt thereof, by reacting a compound represented by the following formula: 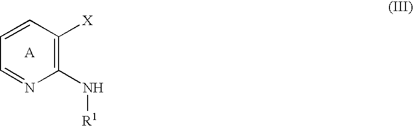
wherein ring A and R¹ respectively represent the same meaning as defined above; and X represents a leaving group;
with a compound represented by the following formula: 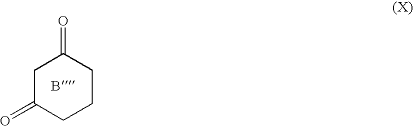
wherein ring B″″ represents a 1,3-cyclohexanedione ring which may be substituted;
to obtain a compound represented by the following formula: 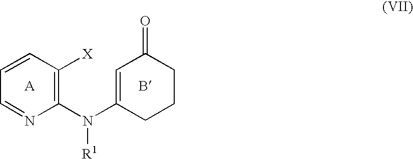
wherein ring A, X, and R¹ respectively represent the same meaning as defined above; and ring B′ represents a cyclohexenone ring which may be substituted; and at least one of ring A and ring B′ is substituted;
subsequently subjecting the compound represented by Formula (VII) to a ring closure reaction in the presence of a palladium catalyst, a ligand, and a base, to obtain a compound represented by the following formula: 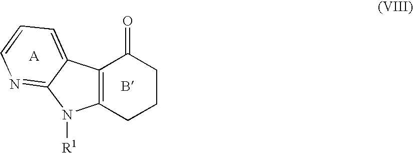
wherein the symbols respectively represent the same meaning as defined above; and at least one of ring A and ring B′ is substituted;
or a salt thereof, then subsequently subjecting ring B′ of the compound represented by Formula (VIII) or a salt thereof to an aromatization reaction to obtain a compound represented by the following formula: 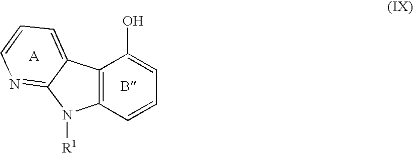
wherein the symbols respectively represent the same meaning as defined above; and at least one of ring A and ring B″ is substituted;
or a salt thereof, subsequently converting a hydroxyl group of the compound represented by Formula (IX) or a salt thereof to a leaving group to obtain a compound represented by the following formula: 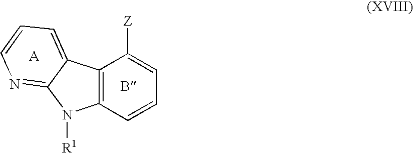
wherein Z represents a leaving group; other symbols respectively represent the same meaning as defined above; and at least one of ring A and ring B″ is substituted;
or a salt thereof, and subsequently subjecting the compound represented by Formula (XVIII) or a salt thereof to a coupling reaction.

20. A method for preparation of a compound represented by the following formula: 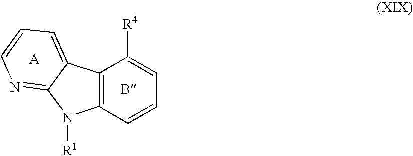
wherein R⁴ represents a C₁₋₁₀ alkyl group which may be substituted, a C₂₋₁₀ alkenyl group which may be substituted, a C₂₋₁₀ alkynyl group which may be substituted, a C₆₋₁₀ aryl group which may be substituted, a C₅₋₁₀ heteroaryl group which may be substituted, an acyl group, a C₁₋₁₀ alkylthio group which may be substituted, a C₇₋₁₃ aralkylthio group which may be substituted, a C₆₋₁₄ arylthio group which may be substituted, an amino group which may be substituted, a C₁₋₁₀ alkoxy group which may be substituted, a C₃₋₁₀ cycloalkoxy group which may be substituted, a C₇₋₁₃ aralkyloxy group which may be substituted, a C₆₋₁₄ aryloxy group which may be substituted, a C₁₋₁₃ alkylcarbonyloxy group which may be substituted, a hydroxyl group, a thiol group, or a cyano group; ring A represents a pyridine ring which may be substituted; R¹ represents a hydrogen atom, a C₁₋₁₀ alkyl group which may be substituted, or an acyl group; ring B″ represents a benzene ring which may be substituted; and at least one of ring A and ring B″ is substituted;
or a salt thereof, by reacting a compound represented by the following formula: 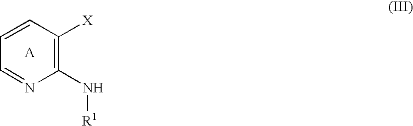
wherein ring A and R¹ respectively represent the same meaning as defined above; and X represents a leaving group;
with a compound represented by the following formula: 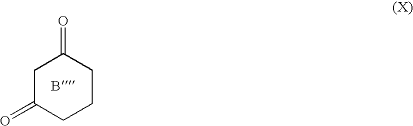
wherein ring B″″ represents a 1,3-cyclohexanedione ring which may be substituted;
to obtain a compound represented by the following formula: 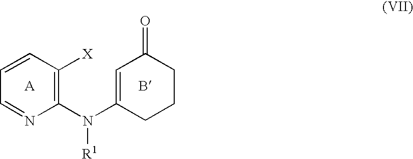
wherein ring A, X, and R¹ respectively represent the same meaning as defined above; and ring B′ represents a cyclohexenone ring which may be substituted; and at least one of ring A and ring B′ is substituted;
subsequently subjecting the compound represented by Formula (VII) to a ring closure reaction in the presence of a palladium catalyst, a ligand, and a base, to obtain a compound represented by the following formula: 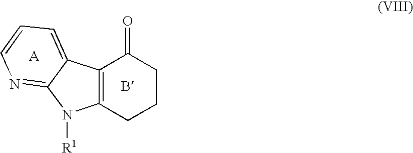
wherein the symbols respectively represent the same meaning as defined above; and at least one of ring A and ring B′ is substituted;
or a salt thereof, then subsequently subjecting ring B′ of the compound represented by Formula (VIII) or a salt thereof to an aromatization reaction to obtain a compound represented by the following formula: 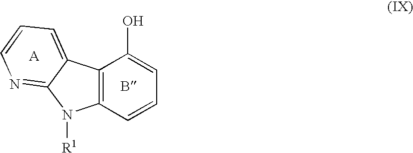
wherein the symbols respectively represent the same meaning as defined above; and at least one of ring A and ring B″ is substituted;
or a salt thereof, subsequently subjecting a hydroxyl group of the compound represented by Formula (IX) or a salt thereof to a coupling reaction.

21. The method according to claim 8, wherein the substituent of ring A or ring B″ is a C₆₋₁₀ aryl group which may be substituted or a C₅₋₁₀ heteroaryl group which may be substituted.

22. The compound according to claim 13, wherein the substituent of ring A or ring B′ is a C₆₋₁₀ aryl group which may be substituted or a C₅₋₁₀ heteroaryl group which may be substituted.

23. The compound according to claim 14, wherein the substituent of ring A or ring B″ is a C₆₋₁₀ aryl group which may be substituted or a C₅₋₁₀ heteroaryl group which may be substituted.

24. A compound represented by the following formula: 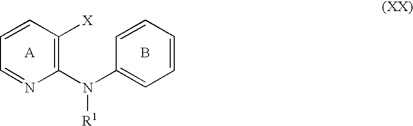
wherein ring A represents a pyridine ring which may be substituted; X represents a leaving group; R¹ represents a hydrogen atom, a C₁₋₁₀ alkyl group which may be substituted, or an acyl group; ring B represents a benzene ring which may be substituted or a pyridine ring which may be substituted; at least one of ring A and ring B is substituted; and the substituent(s) of ring A and/or ring B is(are) a substituent (substituents) selected from a halogen atom, an amino group which may be substituted, a C₁₋₁₀ alkoxy group which may be substituted, a C₁₋₁₀ alkoxy-carbonyl group which may be substituted, an aminocarbonyl group optionally having one or two substituent(s) on a nitrogen atom; a C₆₋₁₀ aryl group which may be substituted; and a C₅₋₁₀ heteroaryl group which may be substituted;
or a salt thereof.

25. The compound according to claim 24, wherein R¹ is a hydrogen atom, at least one of ring A and ring B is substituted, and the substitutents of ring A and/or ring B are at least two kinds of substituents selected from a halogen atom, an amino group which may be substituted, a C₁₋₁₀ alkoxy group which may be substituted, a C₁₋₁₀ alkoxy-carbonyl group which may be substituted, an aminocarbonyl group optionally having one or two substituent(s) on a nitrogen atom, a C₆₋₁₀ aryl group which may be substituted, and a C₅₋₁₀ heteroaryl group which may be substituted.

26. The compound according to claim 24, wherein R¹ is a hydrogen atom, at least one of ring A and ring B is substituted, and the substitutents of ring A and/or ring B are (i) at least one kind of a substituent selected from an amino group which may be substituted, a C₁₋₁₀ alkoxy group which may be substituted, a C₁₋₁₀ alkoxy-carbonyl group which may be substituted, and an aminocarbonyl group optionally having one or two substituent(s) on a nitrogen atom, and (ii) at least one kind of a substituent selected from an amino group which may be substituted, a C₆₋₁₀ aryl group which may be substituted, and a C₅₋₁₀ heteroaryl group which may be substituted.

27. The compound according to claim 12, wherein R² is a halogen atom, provided that the following compounds are excluded;

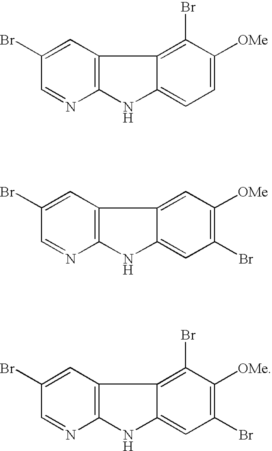

28. The compound according to claim 12, wherein R³ is a halogen atom, provided that the following compounds are excluded; [chemical structure]
